(19) United States
(12) Patent Application Publication
(10) Pub. No.: US 2011/0052562 A1
(43) Pub. Date: Mar. 3, 2011

(54) BENZIMIDAZOLES AND ANALOGS AS RHO KINASE INHIBITORS

(75) Inventors: Yangbo Feng, Palm Beach Gardens, FL (US); Philip LoGrasso, Jupiter, FL (US); Thomas Bannister, Palm Beach Gardens, FL (US); Thomas Schroeter, Riviera Beach, FL (US); Hampton Sessions, Orlando, FL (US); Lei Yao, Tianjin (CN); Bo Wang, Collegeville, PA (US); Michael P. Smolinski, Port St. Lucie, FL (US); Yen Ting Chen, Palm Beach Gardens, FL (US); Yan Yin, Jupiter, FL (US); Bozena Frackowiak-Wojtasek, Dobrzen Wielki (PL); Sarwat Chowdhury, Palm Beach Gardens, FL (US)

(73) Assignee: The Scripps Research Institute, La Jolla, CA (US)

(21) Appl. No.: 12/745,357

(22) PCT Filed: Dec. 18, 2008
(86) PCT No.: PCT/US08/13865

§ 371 Date: Oct. 26, 2010

(30) Foreign Application Priority Data
Dec. 19, 2007 (US) 61/008493

(51) Int. Cl. A61K 38/49; C07D 405/04; A61K 31/4184; A61P 9/10; A61P 9/12; A61P 25/28; A61P 25/16; A61P 11/06; A61P 19/02; A61P 19/10; A61P 17/06; A61P 27/06; A61P 3/10; A61P 13/00; A61P 31/12; C07D 405/14; A61K 31/4439; C07D 471/04; A61K 31/437; C07D 487/04; A61K 31/519; A61K 31/506; A61K 31/5377; C07D 413/14

(52) U.S. Cl. 424/94.64; 548/305.1; 514/394; 546/273.4; 514/338; 546/113; 514/300; 544/280; 514/265.1; 546/122; 544/279; 514/264.1; 544/331; 514/275; 514/234.5; 544/124; 544/122

(57) ABSTRACT

Compounds useful as Rho kinase inhibitors according to formula IA or IB: wherein A, B, D, E, R¹, R² and Ar¹ are as defined herein, and any tautomer, salt, stereoisomer, hydrate, solvent, or prodrug thereof, pharmaceutical compositions, methods of treatment, and synthetic methods are provided.

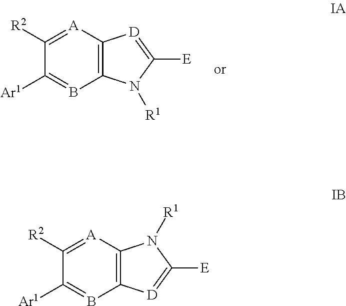

### CROSS-REFERENCE TO RELATED APPLICATIONS

This application claims the priority of U.S. Ser. No. 61/008,493, filed Dec. 19, 2007, which is incorporated herein by reference in its entirety.

### BACKGROUND

Rho kinases, also known as Rho-associated kinases, are serine/threonine kinases that function downstream of Rho which is a low molecular GTP-binding protein. Two Rho kinase isoforms, termed ROCK I and ROCK II, have been identified. The enzymes are believed to be involved in a variety of biological events such as smooth muscle contraction, apoptosis, cell growth, cell migration, cell proliferation, cytokinesis, cytoskeletal control, and inflammation, and to be involved in pathology of various diseases including cardiovascular disease, tumor infiltration, osteogenesis, chondrocyte differentiation and neurogenic pain. See, e.g., H. Satoh, et al., Jpn. J. Pharmacol., 1999, 79, Suppl I, 211, K. Kuwahara, et al., FEBS Lett., 1999, 452, 314-18; N. Sawada, et al., Circulation, 2000, 101, 2030-33; C. Kataoka, et al., Hypertension, 2002, 39(2), 245-50; F. Imamura, et al., Jpn. J. Cancer Res., 2000, 91, 811-16, K. Itoh et al, Nature Medicine, 1999, 5, 221-5, M. Nakajima, et al., Clin. Exp. Pharmacol. Physiol., 2003; 30(7): 457-63; W. Guoyan, et al., J. Biol. Chem., 2004, 279(13), 13205-14; S. Tatsumi, Neuroscience, 2005, 131(2) 491-98.

It is therefore believed that Rho kinase inhibitors have utility in the treatment of diseases and conditions such as hypertension, atherosclerosis, stroke, angina, arterial obstruction, peripheral arterial disease, peripheral circulation disorder, erectile dysfunction, acute and chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, multiple sclerosis, diabetes, urinary organ diseases such as overactive bladder (OAB) and benign prostatic hypertrophy (BPH), metastasis, cancer, glaucoma, ocular hypertension, retinopathy, autoimmune disease and viral infection, and myocardial protection.

Various compounds have been described in the literature as Rho kinase inhibitors. See, e.g. WO98/06433; WO00/09162; WO00/78351; WO01/17562; WO02/076976; EP1256574; WO02/100833; WO03/082808; WO2004/009555; WO2004/024717; WO2004/041813; WO2004/108724; WO2005/003101; WO2005/035501; WO2005/035503; WO2005/035506; WO2005/037198; WO2005/058891; WO2005/074642; WO2005/074643; WO2005/080934; WO2005/082367; WO2005/082890; WO2005/097790; WO2005/100342; WO2005/103050; WO2005/105780; WO2005/108397; WO2006/044753; WO2006/051311; WO2006/057270; WO2006/058120; WO2006/065946; WO2006/099268; WO2006/072792; WO2007/026920; WO2008011560; A. Takami, et al., Bioorg. Med. Chem., 2004, 12, 2115-37; M. Iwakubo, et al., Bioorg. Med. Chem., 2007, 15, 350-64; M. Iwakubo, et al., Bioorg. Med. Chem., 2007, 15, 1022-33.

### SUMMARY

The present invention is directed to certain compounds and compositions that are effective Rho kinase inhibitors, to methods of their use in the treatment of diseases for which inhibition of Rho kinase is therapeutically indicated, and to methods for their preparation.

In various embodiments, the invention provides a compound of formula IA or IB:

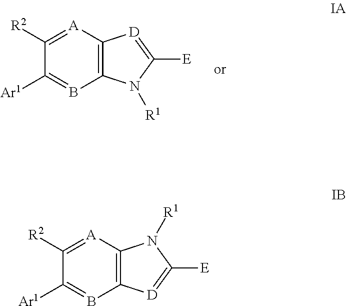

wherein:

A is CR² or N;

B is CR² or N;

D is CR² or N;

Ar¹ is an optionally substituted 5- or 6-membered monocyclic or 8-, 9-, or 10-membered fused bicyclic heterocycle, the ring atoms of which are carbon atoms and one, two, three, or four nitrogen atoms, wherein the optional substitutents are independently at each occurrence selected from the group consisting of (C₁-C₆)alkyl, (C₂-C₆)alkenyl; (C₂-C₆)alkynyl; halogen; —C≡N; —NO₂; —C(═O)R³; —C(═O)OR³; —C(═O)NR³₂; —OR³; —OC(═O)(C₁-C₆)alkyl; —NR³₂, —NR³C(═O)R³; and (C₁-C₃)perfluoroalkyl;

E is selected from the group consisting of, wherein a wavy line signifies a point of attachment,

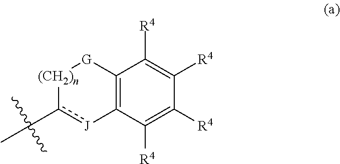

wherein
n is 0 to 2;
G is CH₂, O, S, NR⁵, or CHNHR⁵;
J is CH, CH₂, O, S, NR⁵, CNHR⁵, or CHNHR⁵; and
a dashed line indicates a double bond is present or absent, provided that when J is O, S, NR⁵, or CHNR⁵, the double bond is absent, and when J is CH or CNHR⁵, the double bond is present;

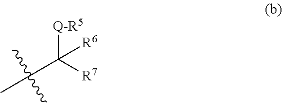

wherein Q is NH or O;

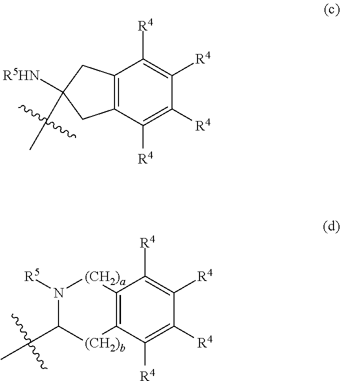

wherein
a is 2 and b is 0; or
a is 1 and b is 1;

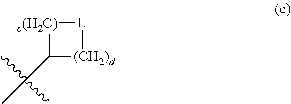

wherein
L is NR⁵, or CHNHR⁵;
c is 0, 1, or 2;
d is 1, 2, 3, 4, or 5;
provided that the sum of c and d is 3, 4, or 5;

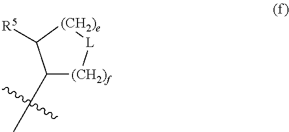

wherein
L is NR⁵, or CHNHR⁵;
e is 0 or 1;
f is 1 or 2;
provided that the sum of e and f is 2 or 3; and

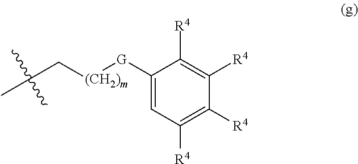

wherein
m is 0 to 2;
G is CH₂, O, S, NR⁵, or CHNHR⁵;

R¹ is hydrogen, (C₁-C₆)alkyl, (C₂-C₆)alkenyl, cycloalkyl, (C₁-C₆)alkylene-cycloalkyl, Ar², —(C₁-C₆)alkylene-Ar², —(C₁-C₆)alkylene-NR³₂, —(C₁-C₆)alkylene-OR³, heterocyclyl, or (C₁-C₆)alkylene-heterocyclyl;

Ar² is unsubstituted aryl, unsubstituted heteroaryl, aryl substituted with one or more substituents selected from R^{a}, or heteroaryl substituted with one or more substituents selected from R^{a};

R^{a} is (C₁-C₆)alkyl, (C₂-C₆)alkenyl; (C₂-C₆)alkynyl; halogen; —C≡N; —NO₂; —C(═O)R³; —C(═O)OR³; —C(═O)NR³₂; —C(═NR³)NR³₂; —OR³; —OC(═O)(C₁-C₆)alkyl; —OC(═O)O(C₁-C₆)alkyl; —OC(═O)NR³₂; —NR³₂; —NR³C(═O)R³; —NR³C(═O)O(C₁-C₆)alkyl; —NR³C(═O)NR³₂; —NR³SO₂R³; —SR³; —S(O)R³; —SO₂R³; —OSO₂(C₁-C₆)alkyl; —SO₂NR³₂; phenyl; pyridyl; 1H-pyrazolyl; 3,5-dimethyl-1H-pyrazolyl; or (C₁-C₃)perfluoroalkyl;

each R² is independently hydrogen, (C₁-C₆)alkyl, (C₂-C₆)alkenyl; (C₂-C₆)alkynyl; halogen; —C≡N; —NO₂; —C(═O)R³; —C(═O)OR³; —C(═O)NR³₂; —C(═NR³)NR³₂; —OR³; —OC(═O)(C₁-C₆)alkyl; —OC(═O)O(C₁-C₆)alkyl; —OC(═O)NR³₂; —NR³₂; —NR³C(═O)R³; —NR³C(═O)O(C₁-C₆)alkyl; —NR³C(═O)NR³₂; —NR³SO₂R³; —SR³; —S(O)R³; —SO₂R³; —OSO₂(C₁-C₆)alkyl; —SO₂NR³₂; or (C₁-C₃)perfluoroalkyl;

each R³ is independently hydrogen, (C₁-C₆)alkyl, OR³, (C₁-C₆)alkylene-OR³, N(R³)₂, (C₁-C₆)alkylene-N(R³)₂, (C₁-C₆)alkylene-C(═O)OR³, (C₁-C₆)alkylene-C(═O)N(R³)₂, (C₃-C₇)cycloalkyl, (C₁-C₆)alkylene-(C₃-C₇)cycloalkyl, (C₃-C₇)-heterocyclyl, (C₁-C₆)alkylene-(C₃-C₇)-heterocyclyl, aryl, (C₁-C₆)alkylene-aryl, heteroaryl, or (C₁-C₆)alkylene-heteroaryl, wherein any alkyl, cycloalkyl, heterocyclyl, aryl, or heteroaryl is substituted with 0-3 J; wherein two R³ groups connected a nitrogen atom in an —NR³₂ moiety may in combination be —(CH₂)ₑ— or —(CH₂)_{f}M(CH₂)₂—; wherein e is 4, 5, or 6; each f is 2 or 3; and M is O, S, NH, N(C₁-C₆)alkyl or NC(═O)(C₁-C₆)alkyl;

each R⁴ is independently selected from the group consisting of hydrogen, (C₁-C₆)alkyl, hydroxy(C₁-C₆)alkyl, (C₂-C₆)alkenyl; (C₂-C₆)alkynyl; halogen; —C≡N; —NO₂; —C(═O)R³; —C(═O)OR³; (C₁-C₆)alkylene-C(═O)OR³; —C(═O)NR³₂; (C₁-C₆)alkylene-C(═O)NR³₂; —C(═NR³)NR³₂; —OR³; (C₁-C₆)alkylene-OR³; —OC(═O)(C₁-C₆)alkyl; —OC(═O)O(C₁-C₆)alkyl; —OC(═O)NR³₂; —NR³₂; —NR³C(═O)R³; —NR³C(═O)O(C₁-C₆)alkyl; —NR³C(═O)NR³₂; —NR³(C₁-C₆)alkylene-NR³₂; —NR³(C₁-C₆)alkylene-OR³; —NR³(C₁-C₆)alkylene-Ar²; —NR³SO₂R³; —SR³; —S(O)R³; —SO₂R³; —OSO₂(C₁-C₆)alkyl; —SO₂NR³₂; (C₁-C₃)perfluoroalkyl; —O (C₁-C₃)perfluoroalkyl; pyrazolyl; triazolyl; and tetrazolyl; or two R⁴ groups taken together form a fused cycloalkyl, heterocyclyl, aryl or heteroaryl ring;

R⁵ is hydrogen, (C₁-C₆)alkyl, (C₁-C₆)alkenyl, C(═O)(C₁-C₆)alkyl, C(═O)O(C₁-C₆)alkyl, Ar², —(C₁-C₆)alkylene-Ar², —(C₁-C₆)C(═O)OR³, or —(C₁-C₆)C(═O)N(R³)₂;

R⁶ is Ar² or —(C₁-C₆)alkylene-Ar²;

R⁷ is hydrogen or (C₁-C₆)alkyl;

or any tautomer, salt, stereoisomer, hydrate, solvent, or prodrug thereof.

In various embodiments, the invention provides methods of synthesis of compounds of the invention.

In various embodiments, the invention provides a pharmaceutical composition comprising a compound of the invention and a suitable excipient.

In various embodiments, the invention provides a pharmaceutical combination comprising a compound of the invention and a second medicament.

In various embodiments, the invention provides a method of treatment of a malcondition in a patient comprising administering a therapeutically effective amount of a compound, pharmaceutical composition, or pharmaceutical combination of the invention to the patient at a frequency of administration and for a duration of time sufficient to provide a beneficial effect to the patient.

In various embodiments, the inventive method can further comprises administration of an effective a second medicament to the patient at a frequency and for a duration sufficient to provide a beneficial effect to the patient. The second medicament can be an anti-proliferative agent, an anti-glaucoma agent, an anti-hypertensive agent, an anti-atherosclerotic agent, an anti-multiple sclerosis agent, an anti-angina agent, an anti-erectile dysfunction agent, an anti-stroke agent, or an anti-asthma agent.

In various embodiments, the invention provides a method of treatment of a malcondition in a patient, comprising administering to the patient the pharmaceutical combination of the invention or a pharmaceutical composition comprising the inventive combination in a therapeutically effective amount at a frequency of administration and for a duration of time sufficient to provide a beneficial effect to the patient.

The malcondition can comprise cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof. The malcondition can be one for the treatment of which binding of a ligand to a Rho kinase or inhibition of a bioactivity of a Rho kinase, or both, is medically indicated.

In various embodiments, the invention provides a use of a compound, composition, or combination of the invention in the preparation of a medicament for treatment of a malcondition. The malcondition can be one wherein binding of a ligand to a Rho kinase or inhibition of a bioactivity of a Rho kinase, or both, is medically indicated. The malcondition can include cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof.

In various embodiments, the invention provides a compound of the invention for use in treatment of cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof.

In various embodiments, the invention provides a compound of any the invention for use in combination with an effective amount of a second bioactive agent in treatment of cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof. The second medicament can be an anti-proliferative agent, an anti-glaucoma agent, an anti-hypertensive agent, an anti-atherosclerotic agent, an anti-multiple sclerosis agent, an anti-angina agent, an anti-erectile dysfunction agent, an anti-stroke agent, or an anti-asthma agent.

### DETAILED DESCRIPTION

### Definitions

As used in the specification and the appended claims, the singular forms “a,” “an” and “the” include plural referents unless the context clearly dictates otherwise.

As used herein, “individual” (as in the subject of the treatment) means both mammals and non-mammals. Mammals include, for example, humans; non-human primates, e.g. apes and monkeys; cattle; horses; sheep; and goats. Non-mammals include, for example, fish and birds.

The term “Rho-kinase-mediated disease” or “Rho-kinase-mediated disorder” are used interchangeably, and are used to refer to diseases or conditions wherein a Rho-kinase (ROCK) plays a role in the biochemical mechanisms involved in the diseases such that a therapeutically beneficial effect can be achieved by inhibiting a Rho-kinase.

The expression “effective amount”, when used to describe therapy to an individual suffering from Rho-kinase-mediated disorder, refers to the amount of a compound of the invention that is effective to inhibit or otherwise act on a Rho kinase in the individual's tissues wherein the Rho-kinase involved in the disorder is active, wherein such inhibition or other action occurs to an extent sufficient to produce a beneficial therapeutic effect.

“Treating” or “treatment” within the meaning herein refers to an alleviation of symptoms associated with a disorder or disease, or inhibition of further progression or worsening of those symptoms, or prevention or prophylaxis of the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects. By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim.

When a substituent is specified to be an atom or atoms of specified identity, “or a bond”, a configuration is referred to when the substituent is “a bond” that the groups that are immediately adjacent to the specified substituent are directly connected to each other by a chemically feasible bonding configuration.

All chiral, diastereomeric, racemic forms of a structure are intended, unless a particular stereochemistry or isomeric form is specifically indicated. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

The term “amino protecting group” or “N-protected” as used herein refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

The term “hydroxyl protecting group” or “O-protected” as used herein refers to those groups intended to protect an OH group against undesirable reactions during synthetic procedures and which can later be removed to reveal the amine. Commonly used hydroxyl protecting groups are disclosed in Protective Groups in Organic Synthesis, Greene, T. W.; Wuts, P. G. M., John Wiley & Sons, New York, N.Y., (3rd Edition, 1999). Hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, a-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. It is well within the skill of the ordinary artisan to select and use the appropriate hydroxyl protecting group for the synthetic task at hand.

In general, “substituted” refers to an organic group as defined herein in which one or more bonds to a hydrogen atom contained therein are replaced by one or more bonds to a non-hydrogen atom such as, but not limited to, a halogen (i.e., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboyxlate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R)₂, OC(O)N(R′)₂, C(S)N(R′)₂, (CH₂)₀₋₂NHC(O)R′, N(R′)N(R′)C(O)R′, N(R)N(R′)C(O)OR′, N(R′)N(R′)CON(R′)₂, N(R′)SO₂R′, N(R′)SO₂N(R′)₂, N(R)C(O)OR, N(R′)C(O)R′, N(R′)C(S)R′, N(R′)C(O)N(R′)₂, N(R)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted. When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, which can also be written as “CO”, “C(O)”, or “C(═O)”, wherein the C and the O are double bonded. When a carbon atom is substituted with a double-bonded oxygen (═O) group, the oxygen substituent is termed an “oxo” group. Alternatively, a divalent substituent such as O, S, C(O), S(O), or S(O)₂ can be connected by two single bonds to two different carbon atoms. For example, O, a divalent substituent, can be bonded to each of two adjacent carbon atoms to provide an epoxide group, or the O can form a bridging ether group, termed an “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR′₂)ₙ wherein n is 1, 2, 3, or more, and each R′ is independently selected.

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups can also be substituted with alkyl, alkenyl, and alkynyl groups as defined herein.

By a “ring system” as the term is used herein is meant a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. By “spirocyclic” is meant the class of structures wherein two rings are fused at a single tetrahedral carbon atom, as is well known in the art.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. Representative substituted alkyl groups can be substituted one or more times with any of the groups listed above, for example, amino, hydroxy, cyano, carboxy, nitro, thio, alkoxy, and halogen groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic” and “carbocycle” denote a ring structure wherein the atoms of the ring are carbon. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N−1 substituents wherein N is the size of the carbocyclic ring with, for example, alkyl, alkenyl, alkynyl, amino, aryl, hydroxy, cyano, carboxy, heteroaryl, heterocyclyl, nitro, thio, alkoxy, and halogen groups, or other groups as are listed above.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like, provided they include at least one double bond within a ring. Cycloalkenyl groups also include rings that are substituted with straight or branched chain alkyl groups as defined above.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂CH₂—OH, —CH₂—CH₂—NH—CH₃, —CH₂—S—CH₂—CH₃, —CH₂CH₂—S(═O)—CH₃, and —CH₂CH₂—O—CH₂CH₂-β-CH₃. Up to two heteroatoms may be consecutive, such as, for example, —CH₂—NH—OCH₃, or —CH₂—CH₂—S—S—CH₃.

The term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, —CH₂—CH═CH—CH₂—SH, and —CH═CH—O—CH₂CH₂—O—CH₃.

Aryl groups are cyclic aromatic hydrocarbons that do not contain heteroatoms. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups include aromatic and non-aromatic ring compounds containing 3 or more ring members, of which, one or more is a heteroatom such as, but not limited to, N, O, and S. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. A heterocyclyl ring can also include one or more double bonds. A heteroaryl ring is an embodiment of a heterocyclyl group. The phrase “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S; for instance, heteroaryl rings can have 5 to about 8-12 ring members. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed above. Representative substituted heteroaryl groups can be substituted one or more times with groups such as those listed above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Heterocyclylalkyl groups are alkyl groups as defined above in which a hydrogen or carbon bond of an alkyl group as defined above is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroarylalkyl groups are alkyl groups as defined above in which a hydrogen or carbon bond of an alkyl group is replaced with a bond to a heteroaryl group as defined above.

The term “alkoxy” refers to an oxygen atom connected to an alkyl group, including a cycloalkyl group, as are defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein.

“Halo” as the term is used herein includes fluoro, chloro, bromo, and iodo. A “haloalkyl” group includes mono-halo alkyl groups, and poly-halo alkyl groups wherein all halo atoms can be the same or different. Examples of haloalkyl include trifluoromethyl, 1,1-dichloroethyl, 1,2-dichloroethyl, 1,3-dibromo-3,3-difluoropropyl and the like.

The terms “halo” or “halogen” or “halide” by themselves or as part of another substituent mean, unless otherwise stated, a fluorine, chlorine, bromine, or iodine atom, preferably, fluorine, chlorine, or bromine.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is —CF₃.

The term “(Cₓ-C_{y})perfluoroalkylene,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkylene, more preferred is —(C₁-C₃)perfluoroalkylene, most preferred is —CF₂—.

The terms “aryloxy” and “arylalkoxy” refer to, respectively, an aryl group bonded to an oxygen atom and an aralkyl group bonded to the oxygen atom at the alkyl moeity. Examples include but are not limited to phenoxy, naphthyloxy, and benzyloxy.

An “acyl” group as the term is used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-20 additional carbon atoms bonded to the carbonyl group. An acyl group can include double or triple bonds within the meaning herein. An acryloyl group is an example of an acyl group. An acyl group can also include heteroatoms within the meaning here. A nicotinoyl group (pyridyl-3-carbonyl) group is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “amine” includes primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R3N wherein each R is independently selected, such as trialkylamines, dialkylarylamines, alkyldiarylamines, triarylamines, and the like. The term “amine” also includes ammonium ions as used herein.

An “amino” group is a substituent of the form —NH₂, —NHR, —NR₂, —NR₃⁺, wherein each R is independently selected, and protonated forms of each. Accordingly, any compound substituted with an amino group can be viewed as an amine.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions, and amines, within the meaning herein.

The term “amide” (or “amido”) includes C- and N-amide groups, i.e., —C(O)NR₂, and —NRC(O)R groups, respectively. Amide groups therefore include but are not limited to carbamoyl groups (—C(O)NH₂) and formamide groups (—NHC(O)H). A “carboxamido” group is a group of the formula C(O)NR₂, wherein R can be H, alkyl, aryl, etc.

The term “urethane” (or “carbamyl”) includes N- and O-urethane groups, i.e., —NRC(O)OR and —OC(O)NR₂ groups, respectively.

The term “sulfonamide” (or “sulfonamido”) includes S- and N-sulfonamide groups, i.e., —SO₂NR₂ and —NRSO₂R groups, respectively. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

The term “amidine” or “amidino” includes groups of the formula —C(NR)NR₂. Typically, an amidino group is —C(NH)NH₂.

The term “guanidine” or “guanidino” includes groups of the formula —NRC(NR)NR₂. Typically, a guanidino group is —NHC(NH)NH₂.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein.

A “hydrate” is a compound that exists in a composition with water molecules. The composition can include water in stoichiometic quantities, such as a monohydrate or a dihydrate, or can include water in random amounts.

A “solvate” is a similar composition except that a solvent other that water replaces the water. For example, methanol or ethanol can form an “alcoholate”, which can again be stoichiometic or non-stoichiometric.

“Tautomers” are two forms of a substance differing only by the position of a hydrogen atom in the molecular structures.

A “prodrug” as is well known in the art is a substance that can be administered to a patient where the substance is converted in vivo by the action of biochemicals within the patients body, such as enzymes, to the active pharmaceutical ingredient. Examples of prodrugs include esters of carboxylic acid groups, which can be hydrolyzed by endogenous esterases as are found in the bloodstream of humans and other mammals.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

In various embodiments, the compound or set of compounds, such as are used in the inventive methods, can be any one of any of the combinations and/or sub-combinations of the above-listed embodiments.

### DESCRIPTION

### Compounds of the Invention

In various embodiments, the invention provides a compound of formula IA or IB:

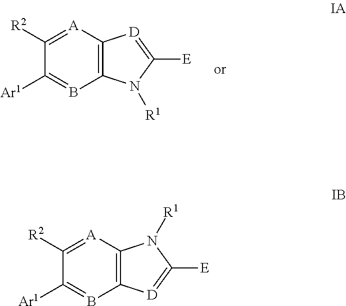

wherein:

A is CR² or N;

B is CR² or N;

D is CR² or N;

Ar¹ is an optionally substituted 5- or 6-membered monocyclic or 8-, 9-, or 10-membered fused bicyclic heterocycle, the ring atoms of which are carbon atoms and one, two, three, or four nitrogen atoms, wherein the optional substitutents are independently at each occurrence selected from the group consisting of (C₁-C₆)alkyl, (C₂-C₆)alkenyl; (C₂-C₆)alkynyl; halogen; —C≡N; —NO₂; —C(═O)R³; —C(═O)OR³; —C(═O)NR³₂; —OR³; —OC(═O)(C₁-C₆)alkyl; —NR³₂, —NR³C(═O)R³; and (C₁-C₃)perfluoroalkyl;

E is selected from the group consisting of, wherein a wavy line signifies a point of attachment,

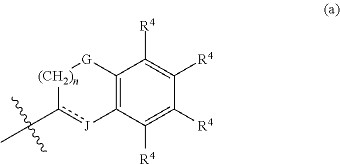

wherein
n is 0 to 2;
G is CH₂, O, S, NR⁵, or CHNHR⁵;
J is CH, CH₂, O, S, NR⁵, CNHR⁵, or CHNHR⁵; and
a dashed line indicates a double bond is present or absent, provided that when J is O, S, NR⁵, or CHNR⁵, the double bond is absent, and when J is CH or CNHR⁵, the double bond is present;

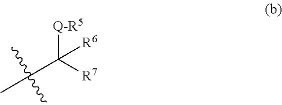

wherein Q is NH or O;

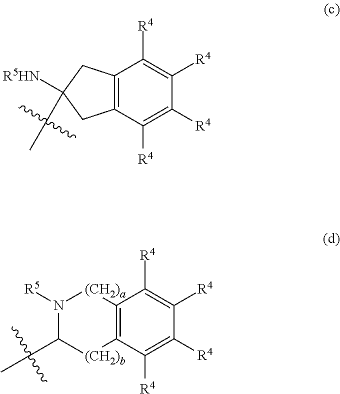

wherein
a is 2 and b is 0; or
a is 1 and b is 1;

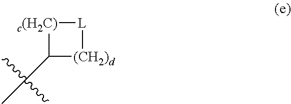

wherein
L is NR⁵, or CHNHR⁵;
c is 0, 1, or 2;
d is 1, 2, 3, 4, or 5;
provided that the sum of c and d is 3, 4, or 5;

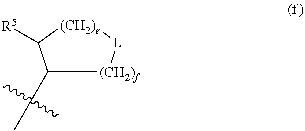

wherein
L is NR⁵, or CHNHR⁵;
e is 0 or 1;
f is 1 or 2;
provided that the sum of e and f is 2 or 3; and

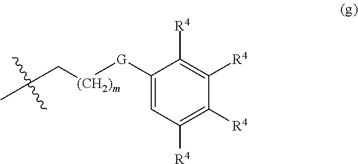

wherein
m is 0 to 2;
G is CH₂, O, S, NR⁵, or CHNHR⁵;

R¹ is hydrogen, (C₁-C₆)alkyl, (C₂-C₆)alkenyl, cycloalkyl, (C₁-C₆)alkylene-cycloalkyl, Ar², —(C₁-C₆)alkylene-Ar², —(C₁-C₆)alkylene-NR³₂, —(C₁-C₆)alkylene-OR³, heterocyclyl, or (C₁-C₆)alkylene-heterocyclyl;

Ar² is unsubstituted aryl, unsubstituted heteroaryl, aryl substituted with one or more substituents selected from R^{a}, or heteroaryl substituted with one or more substituents selected from R^{a};

R^{a} is (C₁-C₆)alkyl, (C₂-C₆)alkenyl; (C₂-C₆)alkynyl; halogen; —C≡N; —NO₂; —C(═O)R³; —C(═O)OR³; —C(═O)NR³₂; —C(═NR³)NR³₂; —OR³; —OC(═O)(C₁-C₆)alkyl; —OC(═O)O(C₁-C₆)alkyl; —OC(═O)NR³₂; —NR³₂; —NR³C(═O)R³; —NR³C(═O)O(C₁-C₆)alkyl; —NR³C(═O)NR³₂; —NR³SO₂R³; —SR³; —S(O)R³; —SO₂R³; —OSO₂(C₁-C₆)alkyl; —SO₂NR³₂; phenyl; pyridyl; 1H-pyrazolyl; 3,5-dimethyl-1H-pyrazolyl; or (C₁-C₃)perfluoroalkyl;

each R² is independently hydrogen, (C₁-C₆)alkyl, (C₂-C₆)alkenyl; (C₂-C₆)alkynyl; halogen; —C≡N; —NO₂; —C(═O)R³; —C(═O)OR³; —C(═O)NR³₂; —C(═NR³)NR³₂; —OR³; —OC(═O)(C₁-C₆)alkyl; —OC(═O)O(C₁-C₆)alkyl; —OC(═O)NR³₂; —NR³₂; —NR³C(═O)R³; —NR³C(═O)O(C₁-C₆)alkyl; —NR³C(═O)NR³₂; —NR³SO₂R³; —SR³; —S(O)R³; —SO₂R³; —OSO₂(C₁-C₆)alkyl; —SO₂NR³₂; or (C₁-C₃)perfluoroalkyl;

each R³ is independently hydrogen, (C₁-C₆)alkyl, OR³, (C₁-C₆)alkylene-OR³, N(R³)₂, (C₁-C₆)alkylene-N(R³)₂, (C₁-C₆)alkylene-C(═O)OR³, (C₁-C₆)alkylene-C(═O)N(R³)₂, (C₃-C₇)cycloalkyl, (C₁-C₆)alkylene-(C₃-C₇)cycloalkyl, (C₃-C₇)-heterocyclyl, (C₁-C₆)alkylene-(C₃-C₇)-heterocyclyl, aryl, (C₁-C₆)alkylene-aryl, heteroaryl, or (C₁-C₆)alkylene-heteroaryl, wherein any alkyl, cycloalkyl, heterocyclyl, aryl, or heteroaryl is substituted with 0-3 J; wherein two R³ groups connected a nitrogen atom in an —NR³₂ moiety may in combination be —(CH₂)ₑ— or —(CH₂)_{f}M(CH₂)₂—; wherein e is 4, 5, or 6; each f is 2 or 3; and M is O, S, NH, N(C₁-C₆)alkyl or NC(═O)(C₁-C₆)alkyl;

each R⁴ is independently selected from the group consisting of hydrogen, (C₁-C₆)alkyl, hydroxy(C₁-C₆)alkyl, (C₂-C₆)alkenyl; (C₂-C₆)alkynyl; halogen; —C≡N; —NO₂; —C(═O)R³; —C(═O)OR³; (C₁-C₆)alkylene-C(═O)OR³; —C(═O)NR³₂; (C₁-C₆)alkylene-C(═O)NR³₂; —C(═NR³)NR³₂; —OR³; (C₁-C₆)alkylene-OR³; —OC(═O)(C₁-C₆)alkyl; —OC(═O)O(C₁-C₆)alkyl; —OC(═O)NR³₂; —NR³₂; —NR³C(═O)R³; —NR³C(═O)O(C₁-C₆)alkyl; —NR³C(═O)NR³₂; —NR³(C₁-C₆)alkylene-NR³₂; —NR³(C₁-C₆)alkylene-OR³; —NR³(C₁-C₆)alkylene-Ar²; —NR³SO₂R³; —SR³; —S(O)R³; —SO₂R³; —OSO₂(C₁-C₆)alkyl; —SO₂NR³₂; (C₁-C₃)perfluoroalkyl; —O(C₁-C₃)perfluoroalkyl; pyrazolyl; triazolyl; and tetrazolyl; or two R⁴ groups taken together form a fused cycloalkyl, heterocyclyl, aryl or heteroaryl ring;

R⁵ is hydrogen, (C₁-C₆)alkyl, (C₁-C₆)alkenyl, C(═O)(C₁-C₆)alkyl, C(═O)O(C₁-C₆)alkyl, Ar², —(C₁-C₆)alkylene-Ar², —(C₁-C₆)C(═O)OR³, or —(C₁-C₆)C(═O)N(R³)₂;

R⁶ is Ar² or —(C₁-C₆)alkylene-Ar²; R⁷ is hydrogen or (C₁-C₆)alkyl; or any tautomer, salt, stereoisomer, hydrate, solvent, or prodrug thereof.

The compounds of formula IA and the compounds of formula IB are isomeric. When R¹ is H, the compounds of formula IA and the compounds of formula IB are tautomeric. All are included among the compounds of the invention.

For example, A and B can both be CR² and D be N. Or, one of A and B can be N, one of A and B can be CR², and D be N. Or, A can be N, or B can be N. Various embodiments also provide other combinations of A, B, and D.

For example, Ar¹ can be an optionally substituted heterocycle selected from the group consisting of optionally substituted pyridyl, pyrimidinyl, 1H-pyrazolyl, 1H-pyrrolo[2,3-b]pyridinyl, 7H-pyrrolo[2,3-d]pyrimidinyl, 1H-pyrazolo[3,4-b]pyridinyl and 1H-pyrazolo[3,4-d]pyrimidinyl.

More specifically, Ar¹ can be an optionally substituted heterocycle selected from the group consisting of optionally substituted 4-pyridyl, pyrimidin-4-yl, 1H-pyrazol-4-yl, 1H-pyrrolo[2,3-b]pyridin-4-yl, 7H-pyrrolo[2,3-d]pyrimidin-4-yl, 1H-pyrazolo[3,4-b]pyridin-4-yl and 1H-pyrazolo[3,4-d]pyrimidin-4-yl.

More specifically, Ar¹ can be substituted with (C₁-C₆)alkyl, (C₂-C₆)alkenyl; (C₂-C₆)alkynyl; halogen; —C≡N; —NO₂; —C(═O)R³; —C(═O)OR³; —C(═O)NR³₂; —OR³; —OC(═O)(C₁-C₆)alkyl; —NR³₂, —NR³C(═O)R³; or (C₁-C₃)perfluoroalkyl.

More specifically, R′ can be hydrogen or (C₁-C₆)alkyl.

More specifically, each R² can independently be hydrogen.

For example, E can be:

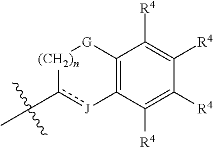

wherein:

n is 0 to 2; G is CH₂, O, S, NR⁵, or CHNHR⁵; J is CH, CH₂, O, S, NR⁵, CNHR⁵, or CHNHR⁵; and a dashed line indicates a double bond is present or absent, provided that when J is O, S, NR⁵, or CHNR⁵, the double bond is absent, and when J is CH or CNHR⁵, the double bond is present;

More specifically, G can be O. If G is O, J can be CH₂, O, or CHNHR⁵ and the double bond be absent. For example, J can be O and the double bond be absent.

More specifically, G can be CH₂, or CHNHR⁵. When G is CHNHR⁵, R⁵ can be hydrogen.

Or, J can be CH or CNHR⁵ and the double bond be present. When J is CH or CNHR⁵ and the double bond is present, G can be O or CH₂. Or, G can be NR⁵, in which case R⁵ can, for example, be hydrogen, (C₁-C₆)alkyl, (C₁-C₆)alkenyl, or —(C₁-C₆)alkylene-Ar².

For example, E can be:

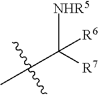

wherein Q is NH or O; R⁵ is hydrogen, (C₁-C₆)alkyl, (C₁-C₆)alkenyl, C(═O)(C₁-C₆)alkyl, C(═O)O(C₁-C₆)alkyl, Ar², —(C₁-C₆)alkylene-Ar², —(C₁-C₆)C(═O)OR³, or —(C₁-C₆)C(═O)N(R³)₂; R⁶ is Ar² or —(C₁-C₆)alkylene-Ar²; and R⁷ is hydrogen or (C₁-C₆)alkyl.

More specifically, R⁷ can be hydrogen. More specifically, Ar² can be unsubstituted or substituted phenyl. More specifically, R⁶ can CH₂Ar², and Ar² can be unsubstituted or substituted phenyl.

For example, E can be:

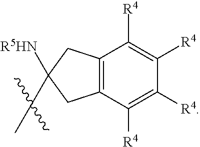

More specifically, each R⁴ can independently be hydrogen.

Or, for example, E can be:

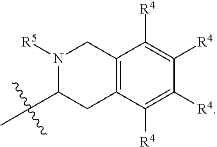

More specifically, each R⁴ can independently be hydrogen, (C₁-C₆)alkyl, hydroxy(C₁-C₆)alkyl, (C₂-C₆)alkenyl; (C₂-C₆)alkynyl; halogen; —C≡N; —NO₂; —C(═O)R³; —C(═O)OR³; (C₁-C₆)alkylene-C(═O)OR³; —C(═O)NR³₂; (C₁-C₆)alkylene-C(═O)NR³₂; —C(═NR³)NR³₂; —OR³; (C₁-C₆)alkylene-OR³; —OC(═O)(C₁-C₆)_{alkyl}; —OC(═O)O(C₁-C₆)alkyl; —OC(═O)NR³₂; —NR³₂; —NR³C(═O)R³; —NR³C(═O)O(C₁-C₆)alkyl; —NR³C(═O)NR³₂; —NR³(C₁-C₆)alkylene-NR³₂; —NR³(C₁-C₆)alkylene-OR³; —NR³(C₁-C₆)alkylene-Ar²; —NR³SO₂R³; —SR³; —S(O)R³; —SO₂R³; —OSO₂(C₁-C₆)alkyl; —SO₂NR³₂; (C₁-C₃)perfluoroalkyl; or —O(C₁-C₃)perfluoroalkyl.

Or, for example, E can be:

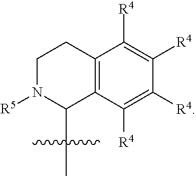

More specifically, each R⁴ can independently be hydrogen, (C₁-C₆)alkyl, hydroxy(C₁-C₆)alkyl, (C₂-C₆)alkenyl; (C₂-C₆)alkynyl; halogen; —C≡N; —NO₂; —C(═O)R³; —C(═O)OR³; (C₁-C₆)alkylene-C(═O)OR³; —C(═O)NR³₂; (C₁-C₆)alkylene-C(═O)NR³₂; —C(═NR³)NR³₂; —OR³; (C₁-C₆)alkylene-OR³; —OC(═O)(C₁-C₆)alkyl; —OC(═O)O(C₁-C₆)alkyl; —OC(═O)NR³₂; —NR³₂; —NR³C(═O)R³; —NR³C(═O)O(C₁-C₆)alkyl; —NR³C(═O)NR³₂; —NR³(C₁-C₆)alkylene-NR³₂; —NR³(C₁-C₆)alkylene-OR³; —NR³(C₁-C₆)alkylene-Ar²; —NR³SO₂R³; —SR³; —S(O)R³; —SO₂R³; —OSO₂(C₁-C₆)alkyl; —SO₂NR³₂; (C₁-C₃)perfluoroalkyl; or —O(C₁-C₃)perfluoroalkyl.

Or, for example, E can be:

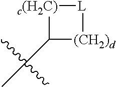

wherein L is NR⁵, or CHNHR⁵; and c is 0, 1, or 2; d is 1, 2, 3, 4, or 5; provided that the sum of c and d is 3, 4, or 5. More specifically, L can be NR⁵ or CHNHR⁵. For example, R⁵ can be hydrogen.

Or, for example, E can be

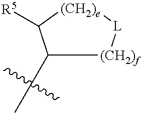

wherein L is NR⁵, or CHNHR⁵; e is 0 or 1; and f is 1 or 2; provided that the sum of e and f is 2 or 3.

Or, for example, E can be

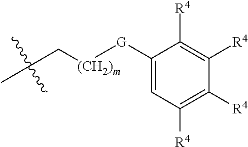

wherein m is 0 to 2; and G is CH₂, O, S, NR⁵, or CHNHR⁵;

For example, the inventive compound can comprise a compound of formula I-1, or a salt thereof:

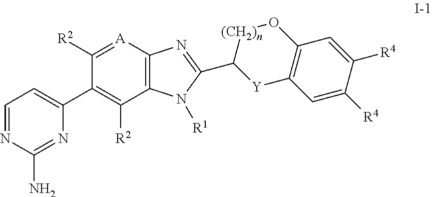

wherein A is selected from the group consisting of CR² and N; Y is CH₂, O, S, or NR⁵; and n is 0 to 2.

For example, the inventive compound can comprise a compound of formula I-2, or a salt thereof:

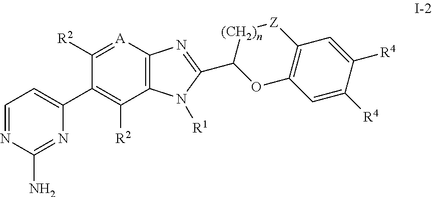

wherein A is selected from the group consisting of CR² and N; Z is CH₂, O, S, or NR⁵; and n is 0 to 2.

In various embodiments, the invention provides a compound of any of the examples 1-359, or any tautomer, salt, stereoisomer, hydrate, solvent, or prodrug thereof.

It is to be understood that other particular and preferred embodiments of the compounds of the invention will combine the features of the particular and preferred embodiments of the invention explicitly described above. Embodiments defined by such combinations are contemplated as particular embodiments of the invention.

In other preferred embodiments the compound of formula IA or IB, or any of the embodiments thereof, is an isolated compound. In other preferred embodiments, the compound of formula IA or IB, and compositions containing the compound, including pharmaceutical compositions, are substantially free of pharmaceutically unacceptable contaminants. A pharmaceutically unacceptable contaminant is a compound which, if present in more than an insubstantial amount, would render the compound unsuitable for use as a pharmaceutical for therapeutic administration.

### Methods for Preparing Compounds of the Invention

There are provided processes for preparing compounds according to the isomeric formulas IA or IB, intermediates that are useful in the preparation of such compounds, and processes for preparing such intermediates. The compounds can be prepared by a variety of synthetic routes. Representative procedures are shown in Schemes 1-12. It will be readily apparent that the compounds can be synthesized by substitution of the appropriate starting materials, reactants, and reagents in the syntheses shown below. It will also be apparent that the selective protection and deprotection steps, as well as the order of the steps themselves, can be carried out in varying order, depending on the nature of the reactions. Precursor compounds, intermediates, and reagents are commercially available or can be prepared from commercially available starting materials. The following schemes are representative, and are in no way intended to limit the scope of the compounds in the embodiments of the present invention.

In the text, formulae and schemes that follow, unless otherwise indicated, the variables are as defined above for formulas IA and IB.

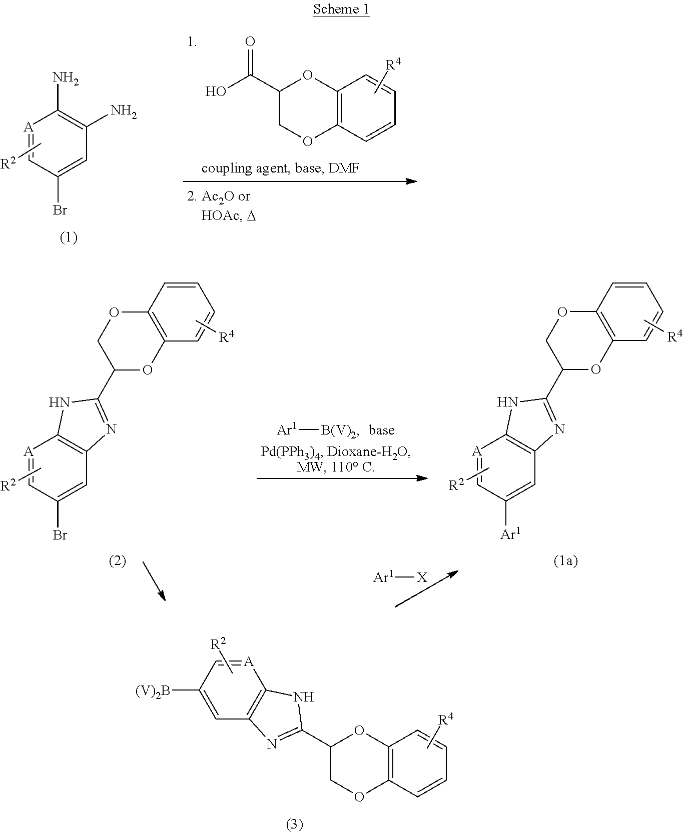

A synthesis of the compounds of the series (Ia) is shown in Scheme 1. A, R², R⁴ and Ar¹ are as defined above; X is halogen. Diamine compounds of formula (1) can be condensed with 1,4-benzodioxan-2-carboxylic acid (or a substituted derivative thereof) by activation of the acid portion with coupling agents in a polar or aprotic solvent. Useful coupling agents include EDC, HOBt, O-(7-azabenzotriazol-1-yl)-N,N,N′,N′-tetramethyluronium hexafluorophosphate (HATU), and the like, in the presence of an organic base, for example, diisopropylethylamine (DIEA) or triethylamine. Cyclization to the desired bromobenzimidazole compounds of formula (2) can be carried out in acetic anhydride or acetic acid, for example, under heat, or alternatively, in the same reaction medium as in the coupling step, under heat, with p-toluenesulfonic acid (p-TsOH). Useful heating ranges for the cyclization step are from about 60° C. to about 160° C. Next, a Suzuki-type coupling can be employed using a palladium(0) or palladium(II) catalyst under standard conditions well known in the art to yield compounds of formula (Ia). Useful bases for the coupling include sodium carbonate, potassium carbonate, sodium bicarbonate, and the like. Useful solvents for the coupling include p-dioxane, 1,2-dimethoxyethane (DME), and the like, and include mixtures of these solvents with water. Microwave energy (“MW” as used in the synthetic schemes) can be usefully employed as a stimulant or initiator of the reaction. The Suzuki-type coupling can optionally be carried out in a sealed tube. The organoboron intermediates Ar¹-B(V)₂, can include compounds wherein V is (C₁-C₆)alkyl, (C₁-C₆)alkenyl, or OW where R^{a} is hydrogen, (C₁-C₆)alkyl, or optionally two R^{a} groups connecting the two oxygen atoms may in combination be (C₁-C₆)alkylene, thus forming a ring. Alternatively, the organoboron compounds of formula (3) can be prepared from bromo-intermediate compounds of formula (2) by known methods. Finally, the compounds of formula (3) can be reacted with aryl or heteroaryl halides using Suzuki-type coupling to provide compounds of formula (Ia). In addition, Ar¹ can be protected prior to coupling, then deprotected as appropriate using standard known procedures. For example, Ar¹ can be 1H-pyrazol-4-yl protected by t-BOC.

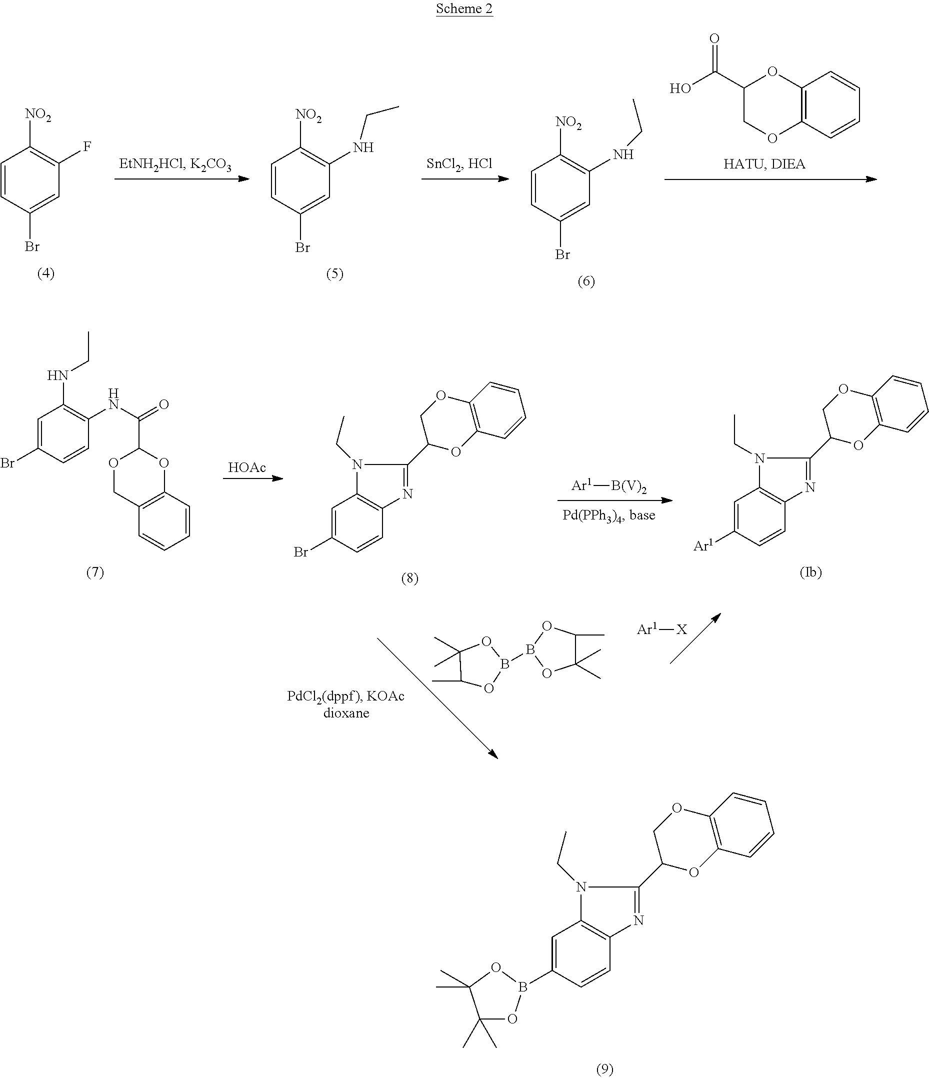

A synthesis of the compounds of the series (Ib) is shown in Scheme 2. 4-bromo-2-fluoro-1-nitrobenzene (4) is condensed with ethylamine to give 5-bromo-N-ethyl-2-nitroaniline (5), which is followed by reduction with tin(II) dichloride to give 5-bromo-N1-ethylbenzene-1,2-diamine (6). Diamine (6) can be condensed with 4-benzodioxan-2-carboxylic acid by activation of the acid portion with HATU as a preferred coupling agent in the presence of an amine base, such as DIEA, for example to give a compound of formula (7). Cyclization of (7) to the desired bromobenzimidazole compound of formula (8) can be carried out in acetic acid. Next, a Suzuki-type coupling can be employed as in Scheme 1 to yield compounds of formula (Ib). Alternatively, the compound of formula (8) can be treated with bis-pinacolatoboronic ester in a polar solvent, such as dioxane, for example and a palladium catalyst to provide an organoboron compound of formula (9). Finally, the compounds of formula (9) can be reacted with aryl or heteroaryl halides using Suzuki-type coupling to provide compounds of formula (Ib).

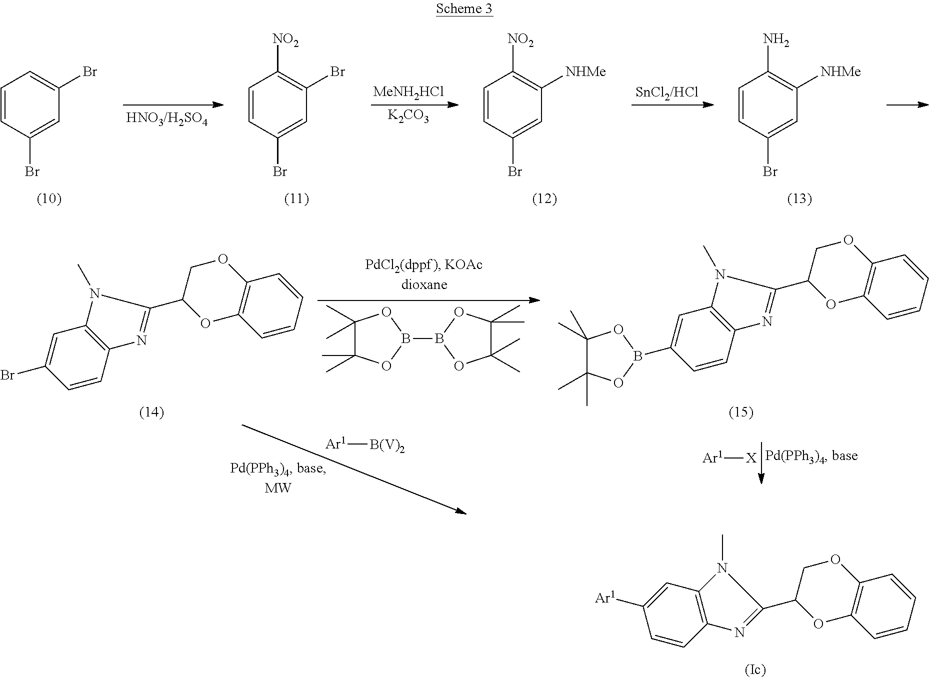

A synthesis of the compounds of the series (Ic) is shown in Scheme 3. 1,3-dibromobenzene (10) is nitrated to give 2,4-dibromo-1-nitrobenzene (11), then is condensed with methylamine to give 5-bromo-N-methyl-2-nitroaniline (12), which is followed by reduction with tin(II) dichloride to give 5-bromo-N1-methylbenzene-1,2-diamine (13). Diamine (13) can be condensed with 4-benzodioxan-2-carboxylic acid and cyclized as in Scheme 2 to give a compound of formula (14). Finally, Suzuki-type couplings can be carried out as in Schemes 1 and 2 to give a compound of formula (Ic).

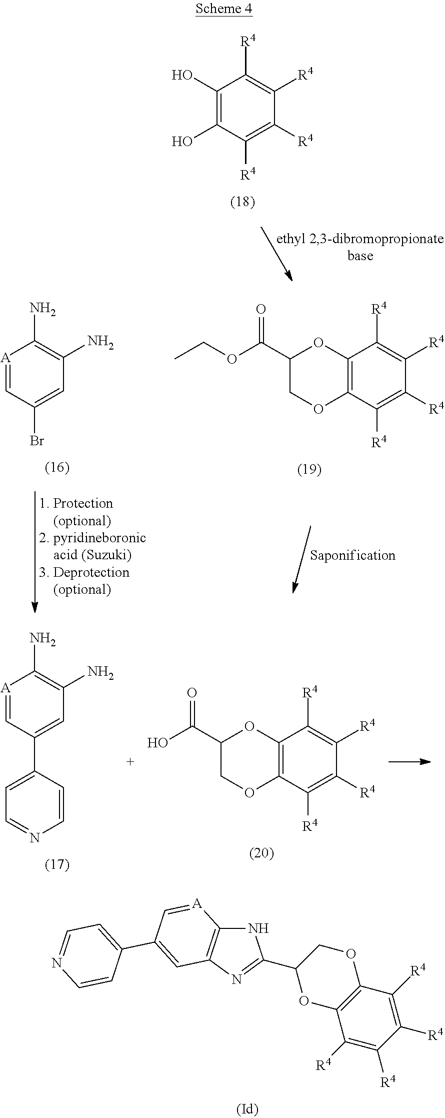

A synthesis of the compounds of the series (Id) is shown in Scheme 4. Diamine compounds of formula (16) can optionally be treated with a protecting group reagent, for example, (Boc)₂O, in order to introduce protecting groups on both nitrogens, followed by Suzuki reaction under standard conditions as discussed above, and finally can optionally be deprotected by known methods to give the diamine (17). In the case of bis-BOC protection, deprotection can be carried out using trifluoroacetic acid (TFA) in dichloromethane. Alternatively, compounds of formula (16) can be coupled directly under Suzuki conditions, without amino protective groups. In a parallel fashion, substituted catechols of formula (18), R⁴ as defined above, is alkylated with, for example, ethyl 2,3-dibromopropionate in the presence of an appropriate base to give compounds of formula (19). Depending on the substitution pattern of the substituted catechol of formula (18) used, the product compounds of formula (19) may consist of a mixture of regioisomers. Saponification of the ethyl benzodioxanecarboxylate (19) under standard conditions gives compounds of formula (20), substituted benzodioxanecarboxylic acid. Useful bases for saponification include, but are not limited to, lithium hydroxide and sodium hydroxide. Useful solvents include THF, p-dioxane, and mixtures of such polar solvents with water. Finally, in a convergent step, diamine (17) and substituted benzodioxanecarboxylic acid (20) can be condensed, and further cyclized, as in the previous Schemes, to afford a compound of formula (Id).

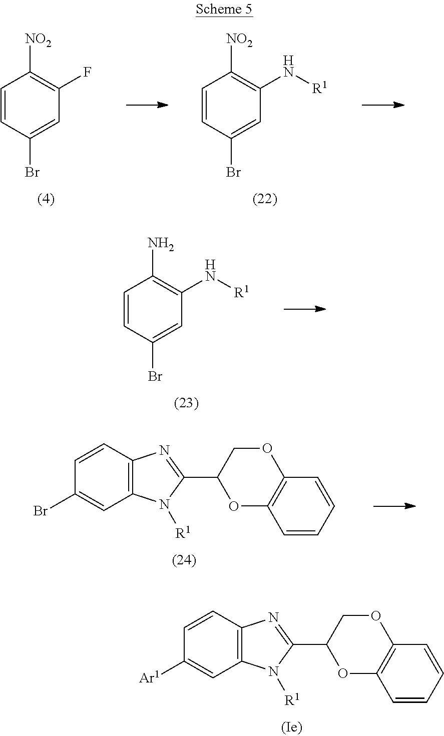

A synthesis of the compounds of the series (Ie) is shown in Scheme 5. As in Scheme 2 above, 4-bromo-2-fluoro-1-nitrobenzene (4) is condensed with a substituted amine to give compounds of formula (22), which is followed by reduction with tin(II) dichloride to give diamine compounds of formula (23). Diamines (23) can be condensed with 4-benzodioxan-2-carboxylic acid by activation of the acid portion with HATU as a preferred coupling agent in the presence of an amine base, such as DIEA, followed by cyclization of to the desired bromobenzimidazole compounds of formula (24), as discussed in Schemes 1-3. Next, a Suzuki-type coupling can be employed as in Scheme 1-3 to yield compounds of formula (Ie).

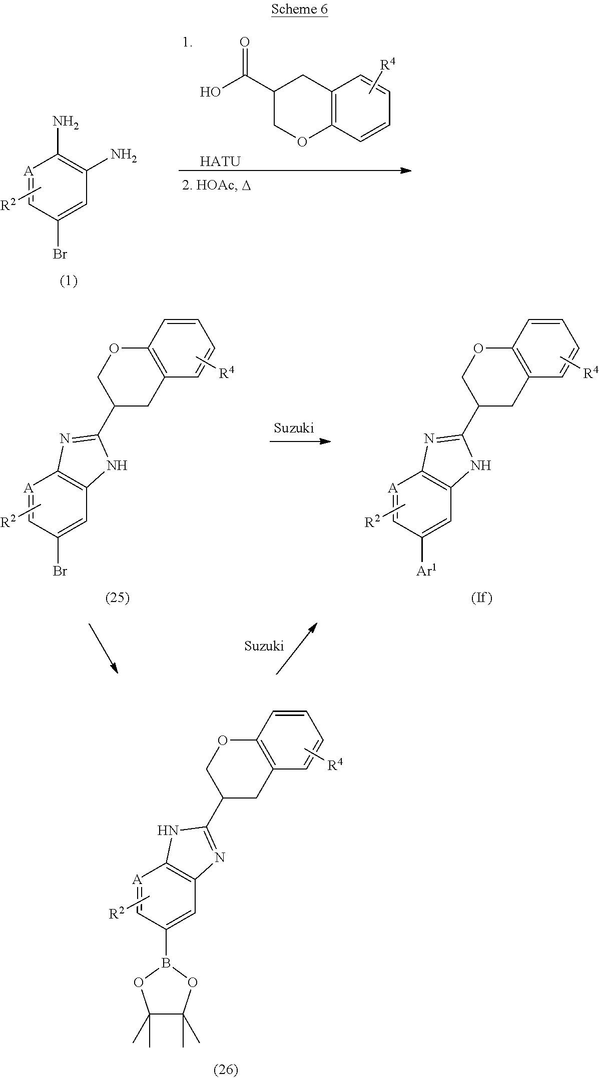

A synthesis of the compounds of the series (It) is shown in Scheme 6. A, R², R⁴ and Ar¹ are as defined above. Compounds of formula (1) are condensed with chroman-3-carboxylic acid (or a substituted derivative thereof) and cyclized according to the previous Schemes to afford compounds of formula (25). Bromo intermediate (25) can be coupled as in the previous Schemes to give the compounds of formula (If). Alternatively, bromo intermediate (25) can be treated with a bis-boronic ester as in Scheme 2 to give compounds of formula (26), which can be can be reacted with aryl or heteroaryl halides using Suzuki-type coupling as in the previous Schemes to provide compounds of formula (If). Chroman-2-carboxylic acid (or a substituted derivative thereof) can also be employed to provide regioisomeric compounds of formula (If).

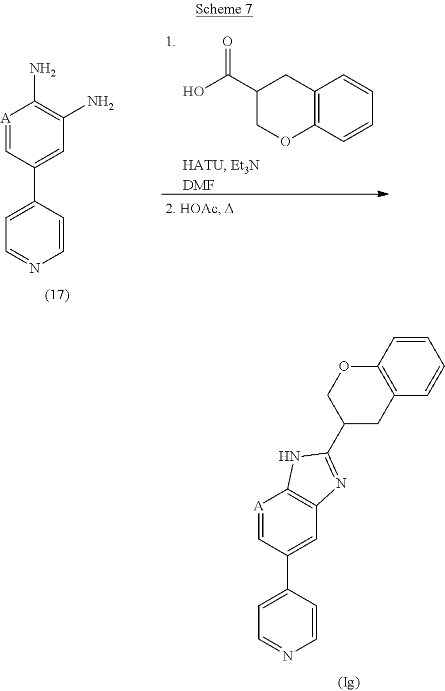

A synthesis of the compounds of the series (Ig) is shown in Scheme 7. Diamine (17) from Scheme 4, or a substituted derivative thereof, is condensed with chroman-3-carboxylic acid (or a substituted derivative thereof) by activation of the acid with HATU in a polar or aprotic solvent, such as, for example DMF, in the presence of a tertiary amine base. Useful amine bases include triethylamine and DIEA. Cyclization can be carried out in acetic acid with heating to afford the compounds of formula (Ig).

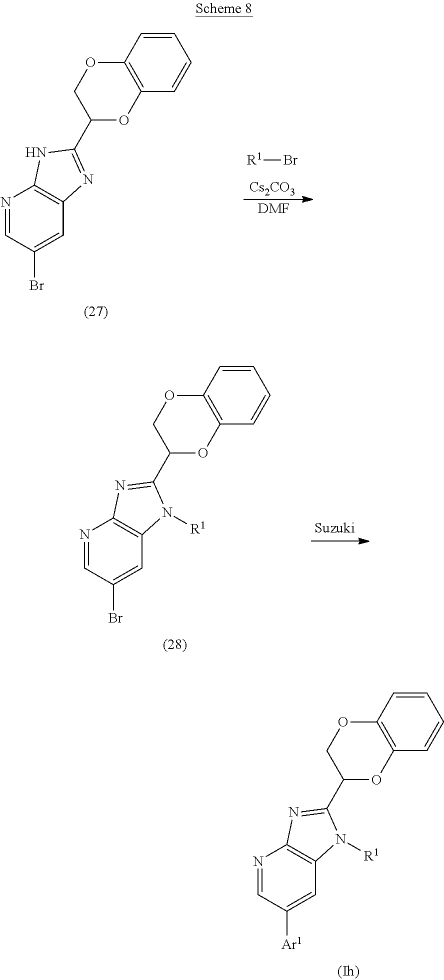

A synthesis of the compounds of the series (Ih) is shown in Scheme 8. A bromo compound of formula (27), or an appropriately substituted derivative thereof, prepared according to Scheme 1, can be treated with an alkyl halide in the presence of an inorganic base in a polar or aprotic solvent. A useful base is cesium carbonate. A useful solvent is DMF. Alkylation can be performed at temperatures ranging from about ambient temperature (25° C.) to about 125° C. Both possible N-alkylated regioisomers can be formed, but they can easily be separated using standard chromatographic techniques, such as flash silica gel chromatography, to give the desired compounds of formula (28). Suzuki couplings, carried out as in the foregoing Schemes, yield the compounds of formula (Ih).

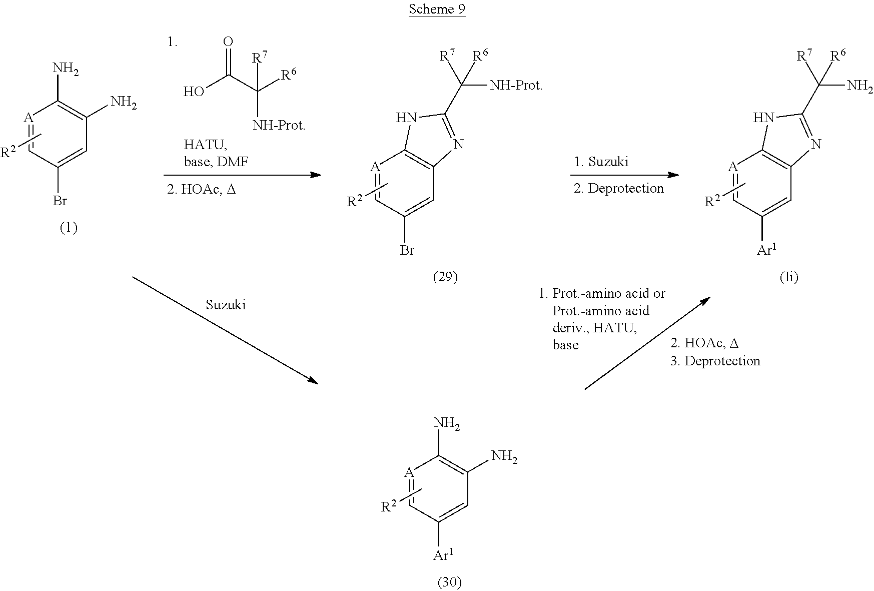

A synthesis of the compounds of the series (Ii) is shown in Scheme 9. Diamine compounds of formula (1) can be condensed with a protected amino acid or amino acid derivative by activation of the carboxylic acid portion with coupling agents, preferably HATU, as discussed in the previous Schemes. Cyclization to the desired bromobenzimidazole compounds of formula (29) can be carried out in acetic acid, for example, under heat, as discussed above. Next, Suzuki coupling is carried out as discussed above, followed by deprotection using appropriate chemoselective agents to give compounds of formula (Ii). For example, if the protecting group is t-BOC, deprotection can be carried out using TFA in dichloromethane, or a mineral acid, such as HCl in an appropriate solvent. As another example, if the protecting group is acetyl, deprotection can be carried out using HCl in methanol. It will be appreciated by those skilled in the art that many types of protecting groups, and many types of protection/deprotection protocols are available. The choice of an appropriate protecting groups depends on a number of factors including, but not limited to, ease of synthesis and isolation, reactivity of other key functional groups, additional protected or protectable functional groups, and cost.

Alternatively, diamine compounds of formula (1) can be coupled using Suzuki-type conditions as in the previous Schemes to give the compounds of formula (30). As discussed above, condensation of diamine (30) with condensed with a protected amino acid or amino acid derivative, followed by cyclization, and finally deprotection as appropriate, to give compounds of formula (Ii).

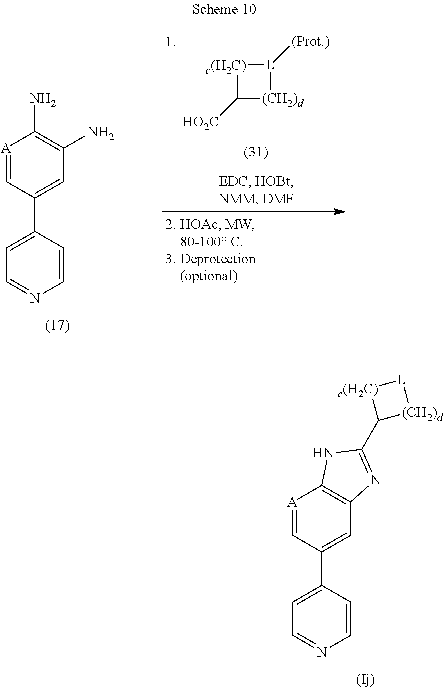

A synthesis of the compounds of the series (Ij) is shown in Scheme 10. A, L, c and d are as defined above. Diamine (17) from Scheme 4, or a substituted derivative thereof, is condensed with a compound of formula (31), or a substituted derivative thereof, by activation of the acid with one or more coupling agents in a polar or aprotic solvent, such as, for example DMF, in the presence of a tertiary amine base. Useful coupling agents include EDC, HOBt, HATU, and the like. Useful amine bases include N-methylmorpholine (NMM), triethylamine and DIEA. Cyclization can be carried out in acetic acid with heating to afford the compounds of formula (Ij). Optionally, microwave energy can be employed to provide heating. In the case of BOC protection of the L group, deprotection can be carried out using trifluoroacetic acid (TFA) in dichloromethane. As a further option for the compounds of formula (Ij), where L=NH, alkylation of this nitrogen can be carried out using R⁵—X (X is halogen) under heating in the presence of a tertiary amine base, for example triethylamine. A useful solvent for the alkylation is acetonitrile. A useful temperature for the alkylation is 70° C. R⁵ is as defined above.

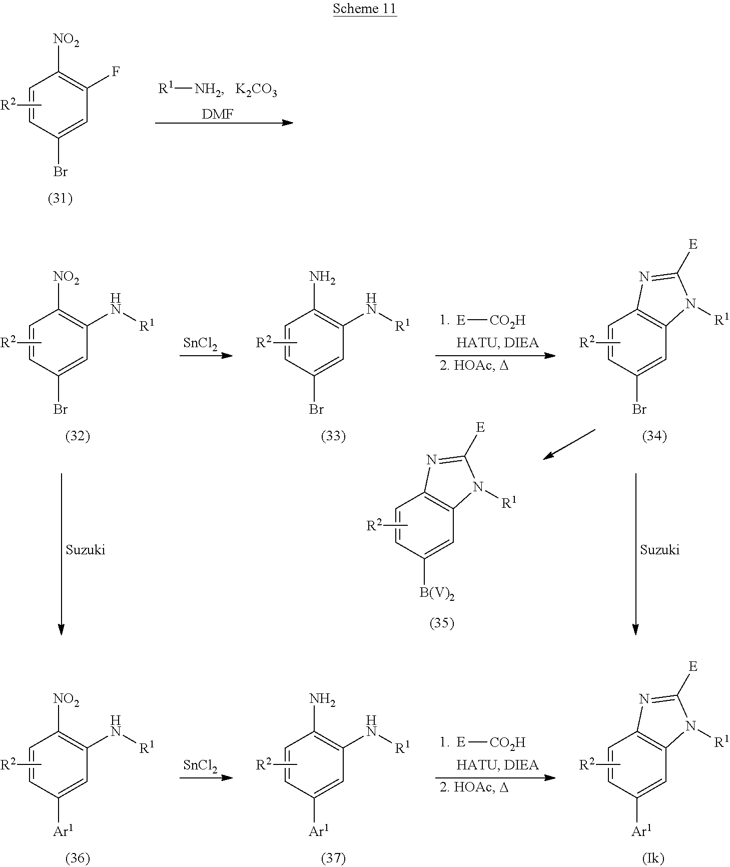

A regiospecific synthesis of the compounds of the series (Ik) is shown in Scheme 11. E, R¹, R² and Ar¹ are as defined above. Amination of the compounds of formula (31) can be carried out in a polar, aprotic solvent such as DMF or DMSO, in the presence of an inorganic base, as shown in Schemes 2 and 3 above, to give the compounds of formula (32), which optionally can be Suzuki-coupled under standard conditions to give the compounds of formula (36). Reduction of the nitro compounds (32 or 36) can be carried out with tin(II) dichloride, with or without heating, as appropriate, in an appropriate solvent or solvent mixture, for example, ethanol, isopropanol, ethyl acetate, p-dioxane, and mixtures thereof, gives the compounds of formula (33) or (37), respectively. Diamine compounds (33) or (37) can be condensed with an appropriate carboxylic acid and cyclized as discussed in the previous Schemes to give compounds of formula (34) or (Ik), respectively. Alternatively, compounds of formula (34) can be Suzuki-coupled as discussed in the previous Schemes to give compounds of formula (Ik). In a final variant, organoboron intermediate compounds of formula (35) can be prepared from the compounds of formula (34), which can subsequently be Suzuki-coupled as discussed in the previous Schemes to give compounds of formula (Ik). The Suzuki couplings can also be carried out in a pressure tube or a high pressure reactor.

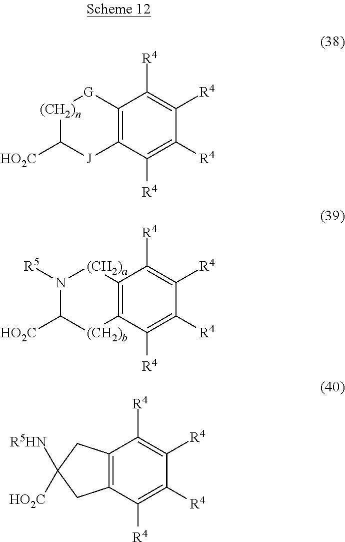

Various carboxylic acids can be employed in the condensation with an appropriate aromatic-1,2-diamine (e.g. compound I) as shown in the previous Schemes, and subsequent cyclization, followed by Suzuki-type coupling provides fused aromatic imidazole compounds of the invention. Useful carboxylic acids include, but are not limited to compounds of formula (38), (39) and (40). G, J, R⁴, R⁵, a, b and n are as defined above. Additional carboxylic acids can be used in the embodiments of the present invention.

In the compounds described above, some functional groups on the aromatic rings, in particular aromatic amine nitrogens, are further derivatizable. Derivatives of aromatic amino groups which are useful in the present invention include, for example: acylation to form carboxamide, carbamate, and urea derivatives; sulfonylation to form sulfonamides, sulfonyl ureas, and sulfamoyl esters; imine formation for formation of imines and for alkylation or arylation (or heteroarylation) via reductive amination; alkylation to form mono- or di-alkylamino derivatives, palladium catalyzed cross coupling to form N-aryl (or N-heteroaryl) derivatives by coupling with aromatic halides or aromatic pseudo halides such as aromatic triflates. Derivatives may also include conjugates to biological molecules such as antibodies to yield macro molecules capable of being directed to a desired site of action thereby reducing or precluding side effects associated with interaction of a drug prepared from a compound of the present invention with tissues and cells which are not proliferating abnormally.

The above-described reactions, unless otherwise noted, are usually conducted at a pressure of about one to about three atmospheres, preferably at ambient pressure (about one atmosphere).

The present invention further embraces isolated compounds of the invention according to formula IA or IB. The expression “isolated compound” refers to a preparation of a compound of the invention, or a mixture of compounds of the invention, wherein the isolated compound has been separated from the reagents used, and/or byproducts formed, in the synthesis of the compound or compounds. “Isolated” does not mean that the preparation is technically pure (homogeneous), but it is sufficiently pure to compound in a form in which it can be used therapeutically. Preferably an “isolated compound” refers to a preparation of a compound of the invention or a mixture of compounds of the invention, which contains the named compound or mixture of compounds of the invention in an amount of at least 10 percent by weight of the total weight. Preferably the preparation contains the named compound or mixture of compounds in an amount of at least 50 percent by weight of the total weight; more preferably at least 80 percent by weight of the total weight; and most preferably at least 90 percent, at least 95 percent or at least 98 percent by weight of the total weight of the preparation.

The compounds of the invention and intermediates may be isolated from their reaction mixtures and purified by standard techniques such as filtration, liquid-liquid extraction, solid phase extraction, distillation, recrystallization or chromatography, including flash column chromatography, or HPLC.

The synthetic methods described above reflect a convergent synthesis strategy. Thus, for example, the carboxylic acid component (e.g. a compound 20) and the aromatic diamine component (e.g compounds 1 or 17) may be synthesized and elaborated separately prior to condensing the two components to form the target compounds (see Scheme 4). The same approach can be employed for the aromatic coupling partners of the Suzuki reaction as described in the above Schemes These convergent synthetic schemes allow for arrangement of the assembly steps of the backbone of the target compounds and derivatization of derivatizable functionalities to accommodate functional group sensitivity and/or to allow for functional groups or elements to be introduced either before or after the assembly of the backbone of the target compounds via the condensation and coupling reactions described.

It will be appreciated by one skilled in the art that certain aromatic substituents in the compounds of the invention, intermediates used in the processes described above, or precursors thereto, may be introduced by employing aromatic substitution reactions to introduce or replace a substituent, or by using functional group transformations to modify an existing substituent, or a combination thereof. Such reactions may be effected either prior to or immediately following the processes mentioned above, and are included as part of the process aspect of the invention. The reagents and reaction conditions for such procedures are known in the art. Specific examples of procedures which may be employed include, but are not limited to, electrophilic functionalization of an aromatic ring, for example via nitration, halogenation, or acylation; transformation of a nitro group to an amino group, for example via reduction, such as by metal/acid or catalytic hydrogenation; acylation, alkylation, or sulfonylation of an amino or hydroxyl group; replacement of an amino group by another functional group via conversion to an intermediate diazonium salt followed by nucleophilic or free radical substitution of the diazonium salt; or replacement of a halogen by another group, for example via nucleophilic or organometallically-catalyzed substitution reactions.

Additionally, in the aforesaid processes, certain functional groups which would be sensitive to the reaction conditions may be protected by protecting groups. A protecting group is a derivative of a chemical functional group which would otherwise be incompatible with the conditions required to perform a particular reaction which, after the reaction has been carried out, can be removed to re-generate the original functional group, which is thereby considered to have been “protected”. Any chemical functionality that is a structural component of any of the reagents used to synthesize compounds of this invention may be optionally protected with a chemical protecting group if such a protecting group is useful in the synthesis of compounds of this invention. The person skilled in the art knows when protecting groups are indicated, how to select such groups, and processes that can be used for selectively introducing and selectively removing them, because methods of selecting and using protecting groups have been extensively documented in the chemical literature. Techniques for selecting, incorporating and removing chemical protecting groups may be found, for example, in Protective Groups in Organic Synthesis, Third Ed. by Theodora W. Greene, Peter G. M. Wuts (John Wiley & Sons, Inc., 1999), the entire disclosure of which is incorporated herein by reference.

In addition to use of a protecting group, sensitive functional groups may be introduced as synthetic precursors to the functional group desired in the intermediate or final product. An example of this is an aromatic nitro (—NO₂) group. The aromatic nitro group goes not undergo any of the nucleophilic reactions of an aromatic amino group. However, the nitro group can serve as the equivalent of a protected amino group because it is readily reduced to the amino group under mild conditions that are selective for the nitro group over most other functional groups.

It will be appreciated by one skilled in the art that the processes described are not the exclusive means by which compounds of the invention may be synthesized and that an extremely broad repertoire of synthetic organic reactions is available to be potentially employed in synthesizing compounds of the invention. The person skilled in the art knows how to select and implement appropriate synthetic routes. Suitable synthetic methods may be identified by reference to the literature, including reference sources such as Comprehensive Organic Synthesis, Ed. B. M. Trost and I. Fleming (Pergamon Press, 1991), Comprehensive Organic Functional Group Transformations, Ed. A. R. Katritzky, O. Meth-Cohn, and C. W. Rees (Pergamon Press, 1996), Comprehensive Organic Functional Group Transformations II, Ed. A. R. Katritzky and R. J. K. Taylor (Editor) (Elsevier, 2^{nd} Edition, 2004), Comprehensive Heterocyclic Chemistry, Ed. A. R. Katritzky and C. W. Rees (Pergamon Press, 1984), and Comprehensive Heterocyclic Chemistry II, Ed. A. R. Katritzky, C. W. Rees, and E. F. V. Scriven (Pergamon Press, 1996).

### Treatment of Rho-Kinase Medicated Disorders Using Compounds of the Invention

According to another embodiment of the invention, a method of treating a patient suffering from Rho-kinase-mediated disorder is provided, comprising administering to the patient an effective amount of at least one compound of the invention, or any tautomer, salt, stereoisomer, hydrate, solvent, or prodrug thereof, either alone, or in combination with a pharmaceutically acceptable carrier.

The invention is also directed to the use of a compound of the invention, or a tautomer, salt, stereoisomer, hydrate, solvent, or prodrug thereof, in the preparation of a medicament for treatment of a Rho-Kinase mediated disorder

The compounds of the present invention or a tautomer, salt, stereoisomer, hydrate, solvent, or prodrug thereof can inhibit or otherwise influence an activity of any Rho kinase such as ROCK I and/or ROCK II. Therefore, the compounds of the present invention are useful for the treatment and/or prevention of a variety of Rho-kinase-mediated diseases.

Rho-kinase-mediated diseases which can be treated and/or prevented by using the compound of the present invention include, but are not limited to, hypertension, pulmonary hypertension, atherosclerosis, stroke, angina, heart failure, arterial obstruction, peripheral arterial disease, peripheral circulation disorder, vasospasm, erectile dysfunction, acute and chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, multiple sclerosis, diabetes, urinary organ diseases such as overactive bladder (OAB) and benign prostatic hypertrophy (BPH), metastasis, cancer, glaucoma, ocular hypertension, retinopathy, autoimmune disease, viral infection, and myocardial protection.

Rho-kinase inhibitors of the present will also be effective for pain alleviation and cartilage protection and will therefore also be effective to treat osteoarthritis, rheumatoid arthritis. osteoporosis, and osteoarthritis.

Particular and preferred embodiments of this aspect of the invention are those wherein the compound of the invention used in the method of treatment, either alone or as part of a composition is a particular or preferred embodiment of the compound of the invention in the description of the compounds and compositions of the invention as provided herein.

The compounds according to the invention may be administered to individuals (mammals, including animals and humans) afflicted with Rho-kinase-mediated disorders as identified herein.

### Salts of Compounds According to the Invention

The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula I compounds (i.e., IA or IB), for example in their purification by recrystallization. All of these salts may be prepared by conventional means from the corresponding compound according to Formula I by reacting, for example, the appropriate acid or base with the compound according to Formula I.

### Isomerism and Tautomerism in Compounds of the Invention

### A. Tautomerism

Within the present invention it is to be understood that a compound of the formula I or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the invention encompasses any tautomeric form which inhibits Rho-kinase activity, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein.

By way of example, it is to be particularly understood that the compounds of the invention wherein R¹ is hydrogen may exist in tautomeric equilibrium with the form of the compounds wherein the R¹ hydrogen exchanges with the nitrogen at the position represented by D in the generic formula IA or IB represented above. Thus, the compounds of formula IA and the compounds of formula IB are tautomeric when R¹ is hydrogen. The equilibrium is illustrated graphically below. It is to be particularly understood that both isomeric (tautomeric when R¹ is H) are within the compounds of the invention. When R¹ is other than hydrogen, the compounds of formula IA are isomeric with the respective compounds of formula IB, and all are within the compounds of the invention.

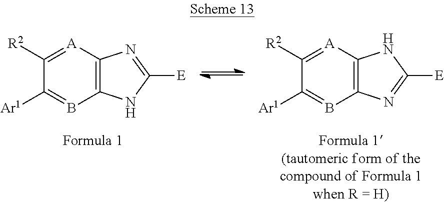

### B. Optical Isomerism

It will be understood that when compounds of the present invention contain one or more chiral centers, the compounds may exist in, and may be isolated as pure enantiomeric or diastereomeric forms or as racemic mixtures. The present invention therefore includes any possible enantiomers, diastereomers, racemates or mixtures thereof of the compounds of the invention which are biologically active in the treatment of Rho-kinase mediated diseases. The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 14, the Cahn-Ingold-Prelog ranking is A>B>C>D. The lowest ranking atom, D is oriented away from the viewer.

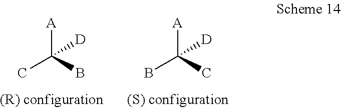

The present invention is meant to encompass diastereomers as well as their racemic and resolved, diastereomerically and enantiomerically pure forms and salts thereof.

Diastereomeric pairs may be resolved by known separation techniques including normal and reverse phase chromatography, and crystallization.

“Isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound of the invention, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

### C. Rotational Isomerism

It is understood that due to chemical properties (i.e., resonance lending some double bond character to the C—N bond) of restricted rotation about the amide bond linkage (as illustrated below) it is possible to observe separate rotamer species and even, under some circumstances, to isolate such species (Scheme 15). It is further understood that certain structural elements, including steric bulk or substituents on the amide nitrogen, may enhance the stability of a rotamer to the extent that a compound may be isolated as, and exist indefinitely, as a single stable rotamer. The present invention therefore includes any possible stable rotamers of formula I which are biologically active in the treatment of cancer or other proliferative disease states.

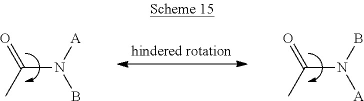

### D. Regioisomerism

The preferred compounds of the present invention have a particular spatial arrangement of substituents on the aromatic rings, which is related to the structure activity relationship demonstrated by the compound class. Often such substitution arrangement is denoted by a numbering system; however, numbering systems are often not consistent between different ring systems. In six-membered aromatic systems, the spatial arrangements are specified by the common nomenclature “para” for 1,4-substitution, “meta” for 1,3-substitution and “ortho” for 1,2-substitution as shown below (Scheme 16).

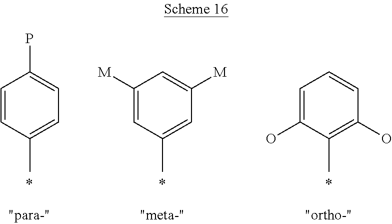

Another example of regioisomerism is pertinent to the compounds of formula I. As discussed in Scheme 13, imidazoles can exist in two isomeric forms. Further derivatization of imidazoles can produce regioisomers. As discussed in Scheme 8, alkylation can provide two N-alkylated regioisomers, which can be separated to provide the compounds of formula I.

### Pharmaceutical Compositions

Another aspect of an embodiment of the invention provides compositions of the compounds of the invention, alone or in combination with another medicament. As set forth herein, compounds of the invention include stereoisomers, tautomers, solvates, prodrugs, pharmaceutically acceptable salts and mixtures thereof. Compositions containing a compound of the invention can be prepared by conventional techniques, e.g. as described in Remington: The Science and Practice of Pharmacy, 19th Ed., 1995, incorporated by reference herein. The compositions can appear in conventional forms, for example capsules, tablets, aerosols, solutions, suspensions or topical applications.

Typical compositions include a compound of the invention and a pharmaceutically acceptable excipient which can be a carrier or a diluent. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration can be any route which effectively transports the active compound of the invention to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

If a solid carrier is used for oral administration, the preparation can be tabletted, placed in a hard gelatin capsule in powder or pellet form or it can be in the form of a troche or lozenge. If a liquid carrier is used, the preparation can be in the form of a syrup, emulsion, soft gelatin capsule or sterile injectable liquid such as an aqueous or non-aqueous liquid suspension or solution.

Injectable dosage forms generally include aqueous suspensions or oil suspensions which can be prepared using a suitable dispersant or wetting agent and a suspending agent Injectable forms can be in solution phase or in the form of a suspension, which is prepared with a solvent or diluent. Acceptable solvents or vehicles include sterilized water, Ringer's solution, or an isotonic aqueous saline solution. Alternatively, sterile oils can be employed as solvents or suspending agents. Preferably, the oil or fatty acid is non-volatile, including natural or synthetic oils, fatty acids, mono-, di- or tri-glycerides.

For injection, the formulation can also be a powder suitable for reconstitution with an appropriate solution as described above. Examples of these include, but are not limited to, freeze dried, rotary dried or spray dried powders, amorphous powders, granules, precipitates, or particulates. For injection, the formulations can optionally contain stabilizers, pH modifiers, surfactants, bioavailability modifiers and combinations of these. The compounds can be formulated for parenteral administration by injection such as by bolus injection or continuous infusion. A unit dosage form for injection can be in ampoules or in multi-dose containers.

The formulations of the invention can be designed to provide quick, sustained, or delayed release of the active ingredient after administration to the patient by employing procedures well known in the art. Thus, the formulations can also be formulated for controlled release or for slow release.

Compositions contemplated by the present invention can include, for example, micelles or liposomes, or some other encapsulated form, or can be administered in an extended release form to provide a prolonged storage and/or delivery effect. Therefore, the formulations can be compressed into pellets or cylinders and implanted intramuscularly or subcutaneously as depot injections. Such implants can employ known inert materials such as silicones and biodegradable polymers, e.g., polylactide-polyglycolide. Examples of other biodegradable polymers include poly(orthoesters) and poly(anhydrides).

For nasal administration, the preparation can contain a compound of the invention, dissolved or suspended in a liquid carrier, preferably an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet that can be prepared by conventional tabletting techniques can contain:

| Core: | Active compound (as free compound or salt | 250 | mg |
|---|---|---|---|
| | thereof) | | |
| | Colloidal silicon dioxide (Aerosil) ® | 1.5 | mg |
| | Cellulose, microcryst. (Avicel) ® | 70 | mg |
| | Modified cellulose gum (Ac-Di-Sol) ® | 7.5 | mg |
| | Magnesium stearate | Ad. | |
| Coating: | HPMC approx. | 9 | mg |
| | *Mywacett 9-40 T approx. | 0.9 | mg |

*Acylated monoglyceride used as plasticizer for film coating.

A typical capsule for oral administration contains compounds of the invention (250 mg), lactose (75 mg) and magnesium stearate (15 mg). The mixture is passed through a 60 mesh sieve and packed into a No. 1 gelatin capsule. A typical injectable preparation is produced by aseptically placing 250 mg of compounds of the invention into a vial, aseptically freeze-drying and sealing. For use, the contents of the vial are mixed with 2 mL of sterile physiological saline, to produce an injectable preparation.

The compounds of the invention can be administered to a mammal, especially a human in need of such treatment, prevention, elimination, alleviation or amelioration of a malcondition. Such mammals include also animals, both domestic animals, e.g. household pets, farm animals, and non-domestic animals such as wildlife.

The compounds of the invention are effective over a wide dosage range. For example, in the treatment of adult humans, dosages from about 0.05 to about 5000 mg, preferably from about 1 to about 2000 mg, and more preferably between about 2 and about 2000 mg per day can be used. A typical dosage is about 10 mg to about 1000 mg per day. In choosing a regimen for patients it can frequently be necessary to begin with a higher dosage and when the condition is under control to reduce the dosage. The exact dosage will depend upon the activity of the compound, mode of administration, on the therapy desired, form in which administered, the subject to be treated and the body weight of the subject to be treated, and the preference and experience of the physician or veterinarian in charge.

Generally, the compounds of the invention are dispensed in unit dosage form including from about 0.05 mg to about 1000 mg of active ingredient together with a pharmaceutically acceptable carrier per unit dosage.

Usually, dosage forms suitable for oral, nasal, pulmonal or transdermal administration include from about 125 μg to about 1250 mg, preferably from about 250 μg to about 500 mg, and more preferably from about 2.5 mg to about 250 mg, of the compounds admixed with a pharmaceutically acceptable carrier or diluent.

Dosage forms can be administered daily, or more than once a day, such as twice or thrice daily. Alternatively dosage forms can be administered less frequently than daily, such as every other day, or weekly, if found to be advisable by a prescribing physician. The compounds of the invention may be administered in the form of a pharmaceutical composition, in combination with a pharmaceutically acceptable carrier. The active ingredient in such formulations may comprise from 0.1 to 99.99 weight percent. “Pharmaceutically acceptable carrier” means any carrier, diluent or excipient which is compatible with the other ingredients of the formulation and not deleterious to the recipient. The active agent is preferably administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, ed., Remington's Pharmaceutical Sciences, 18th Edition (1990), Mack Publishing Co., Easton, Pa. Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water soluble salt of the active agent. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or non-aqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. According to one tablet embodiment, the active agent may be combined with carboxymethylcellulose calcium, magnesium stearate, mannitol and starch, and then formed into tablets by conventional tableting methods.

The specific dose of a compound according to the invention to obtain therapeutic benefit for treatment of Rho-kinase mediated disorder will, of course, be determined by the particular circumstances of the individual patient including the size, weight, age and sex of the patient, the nature and stage of the cellular proliferative disorder, the aggressiveness of the cellular proliferative disorder, and the route of administration of the compound.

For example, a daily dosage from about 0.05 to about 50 mg/kg/day may be utilized, more preferably from about 0.1 to about 10 mg/kg/day, particularly for the treatment of humans. Higher or lower doses are also contemplated as it may be necessary to use dosages outside these ranges in some cases. The daily dosage may be divided, such as being divided equally into two to four times per day daily dosing. The compositions are preferably formulated in a unit dosage form, each dosage containing from about 1 to about 500 mg, more typically, about 10 to about 100 mg of active agent per unit dosage. The term “unit dosage form” refers to physically discrete units suitable as a unitary dosage for human subjects and other mammals, each unit containing a predetermined quantity of active material calculated to produce the desired therapeutic effect, in association with a suitable pharmaceutical excipient.

The pharmaceutical compositions of the present invention may also be formulated so as to provide slow or controlled release of the active ingredient therein using, for example, hydropropylmethyl cellulose in varying proportions to provide the desired release profile, other polymer matrices, gels, permeable membranes, osmotic systems, multilayer coatings, microparticles, liposomes and/or microspheres.

In general, a controlled-release preparation is a pharmaceutical composition capable of releasing the active ingredient at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations. U.S. Pat. No. 5,674,533 discloses controlled-release pharmaceutical compositions in liquid dosage forms for the administration of moguisteine, a potent peripheral antitussive. U.S. Pat. No. 5,059,595 describes the controlled-release of active agents by the use of a gastro-resistant tablet for the therapy of organic mental disturbances. U.S. Pat. No. 5,591,767 describes a liquid reservoir transdermal patch for the controlled administration of ketorolac, a non-steroidal anti-inflammatory agent with potent analgesic properties. U.S. Pat. No. 5,120,548 discloses a controlled-release drug delivery device comprised of swellable polymers. U.S. Pat. No. 5,073,543 describes controlled-release formulations containing a trophic factor entrapped by a ganglioside-liposome vehicle. U.S. Pat. No. 5,639,476 discloses a stable solid controlled-release formulation having a coating derived from an aqueous dispersion of a hydrophobic acrylic polymer. Biodegradable microparticles are known for use in controlled-release formulations. U.S. Pat. No. 5,354,566 discloses a controlled-release powder that contains the active ingredient. U.S. Pat. No. 5,733,566, describes the use of polymeric microparticles that release antiparasitic compositions. The controlled-release of the active ingredient may be stimulated by various inducers, for example pH, temperature, enzymes, water, or other physiological conditions or compounds.

Various mechanisms of drug release exist. For example, in one embodiment, the controlled-release component may swell and form porous openings large enough to release the active ingredient after administration to a patient. The term “controlled-release component” in the context of the present invention is defined herein as a compound or compounds, such as polymers, polymer matrices, gels, permeable membranes, liposomes and/or microspheres, that facilitate the controlled-release of the active ingredient in the pharmaceutical composition. In another embodiment, the controlled-release component is biodegradable, induced by exposure to the aqueous environment, pH, temperature, or enzymes in the body. In another embodiment, sol-gels may be used, wherein the active ingredient is incorporated into a sol-gel matrix that is a solid at room temperature. This matrix is implanted into a patient, preferably a mammal, having a body temperature high enough to induce gel formation of the sol-gel matrix, thereby releasing the active ingredient into the patient.

One or more compounds useful in the practice of the present inventions may be administered simultaneously, by the same or different routes, or at different times during treatment. The compounds may be administered before, along with, or after other medications. The treatment may be carried out for as long a period as necessary, either in a single, uninterrupted session, or in discrete sessions. The treating physician will know how to increase, decrease, or interrupt treatment based on patient response. The treatment schedule may be repeated as required.

### Pharmaceutical Combinations

In various embodiments, a pharmaceutical combination comprising a compound of the invention in a therapeutically effective dose and a second medicament in a therapeutically effective dose is provided. More specifically, the second medicament can comprise an anti-proliferative agent, an anti-glaucoma agent, an anti-hypertensive agent, an anti-atherosclerotic agent, an anti-multiple sclerosis agent, an anti-angina agent, an anti-erectile dysfunction agent, an anti-stroke agent, or an anti-asthma agent. For example, the anti-proliferative agent can comprise an alkylating agent, an anti-metabolite, a vinca alkaloid, a terpenoid, a topoisomerase inhibitor, a monoclonal antibody, a kinase inhibitor, carboplatin, cisplatin, taxol, leucovorin, 5-fluorouracil, eloxatin, cyclophosphamide, chlorambucil, avastin, or imatinib mesylate. For example, the anti-glaucoma agent can comprise a beta receptor-blocker, a prostaglandin, an alpha-adrenergic agonist, a parasympathomimetic (cholinergic agonist), or a carbonic anhydrase inhibitor. For example, the anti-hypertensive agent can comprise a beta receptor-blocker, a calcium channel blocker, a diueretic, an angiotensin converting enzyme (ACE) inhibitor, a renin inhibitor, or an angiotensin receptor antagonist. For example, the anti-atherosclerotic agent can comprise a 3-HMG-coA-reductase inhibitor, a statin, atorvastatin, simvastatin, niacin, or a combination drug such as vytorin. For example, the anti-multiple sclerosis agent can comprise beta-inteferon, tysabri, or glatirimar acetate. For example, the anti-angina agent can comprise a beta receptor-blocker, a calcium channel blocker, nitroglycerin, isosorbide mononitrate, nicorandil, or ranolanzine. For example, the anti-erectile dysfunction agent can comprise a phosphodiesterase-5 inhibitor. For example, the anti-stroke agent can comprise tissue plasminogen activator. For example, the anti-asthma agent can comprise a bronchodilator, an inhaled corticosteroid, a leukotrine blockers, cromolyn, nedocromil, or theophylline.

In various embodiments, a pharmaceutical combination of the invention can further comprise a suitable excipient as outlined above to provide a pharmaceutical composition comprising both medicaments.

In various embodiments, a method of treatment of a malcondition is provided comprising administering an effective amount of a compound of the invention and co-administering an effective amount of an additional medicament. The malcondition can comprise cardiovascular disease, neurogenic pain, hypertension, atherosclerosis, angina, stroke, arterial obstruction, peripheral arterial disease, peripheral circulation disorder, erectile dysfunction, acute and chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof.

In various embodiments, the additional medicament that can be co-administered can comprise an anti-proliferative agent, an anti-glaucoma agent, an anti-hypertensive agent, an anti-atherosclerotic agent, an anti-multiple sclerosis agent, an anti-angina agent, an anti-erectile dysfunction agent, an anti-stroke agent, or an anti-asthma agent. By “co-administered” is meant that the patient is provided with an effective dose of an inventive compound and with an effective dose of the second medicament during the course of treatment, such as concurrently, consecutively, intermittently, or in other regimens. The compound of the invention and the second medicament can be administered in separate dosage forms. For example, the anti-proliferative agent can comprise an alkylating agent, an anti-metabolite, a vinca alkaloid, a terpenoid, a topoisomerase inhibitor, a monoclonal antibody, a kinase inhibitor, carboplatin, cisplatin, taxol, leucovorin, 5-fluorouracil, eloxatin, cyclophosphamide, chlorambucil, avastin, or imatinib mesylate. For example, the anti-glaucoma agent can comprise a beta receptor-blocker, a prostaglandin, an alpha-adrenergic agonist, a parasympathomimetic (cholinergic agonist), or a carbonic anhydrase inhibitor. For example, the anti-hypertensive agent can comprise a beta receptor-blocker, a calcium channel blocker, a diueretic, an angiotensin converting enzyme (ACE) inhibitor, a renin inhibitor, or an angiotensin receptor antagonist. For example, the anti-atherosclerotic agent can comprise a 3-HMG-coA-reductase inhibitor, a statin, atorvastatin, simvastatin, niacin, or a combination drug such as vytorin. For example, the anti-multiple sclerosis agent can comprise beta-inteferon, tysaberai, or glatirimar acetate. For example, the anti-angina agent can comprise a beta receptor-blocker, a calcium channel blocker, nitroglycerin, isosorbide mononitrate, nicorandil, or ranolanzine. For example, the anti-erectile dysfunction agent can comprise a phosphodiesterase-5 inhibitor. For example, the anti-stroke agent can comprise tissue plasminogen activator. For example, the anti-asthma agent can comprise a bronchodilator, an inhaled corticosteroid, a leukotrine blockers, cromolyn, nedocromil, or theophylline.

### EXAMPLES

The compounds and processes of the present invention will be better understood in connection with the following examples, which are intended as an illustration of and not a limitation upon the scope of the invention. In the synthetic pathways and methods that follow, reference to the term “aryl” is intended to include substituted and unsubstituted aryl, and also substituted and unsubstituted heteroaryl. The illustrated synthetic pathways are applicable to other embodiments of the invention. The synthetic procedures described as “general methods” describe what it is believed will be typically effective to perform the synthesis indicated. However, the person skilled in the art will appreciate that it may be necessary to vary the procedures for any given embodiment of the invention. For example, reaction monitoring, such as by using thin layer chromatography (TLC), or HPLC may be used to determine the optimum reaction time. Products may be purified by conventional techniques that will vary, for example, according to the amount of side products produced and the physical properties of the compounds. On a laboratory scale, recrystallisation from a suitable solvent, column chromatography, normal or reverse phase HPLC, or distillation are all techniques which may be useful. The person skilled in the art will appreciate how to vary the reaction conditions to synthesize any given compound within the scope of the invention without undue experimentation. See, e.g., Vogel's Textbook of Practical Organic Chemistry, by A. I. Vogel, et al, Experimental Organic Chemistry: Standard and Microscale, by L. M. Harwood et al. (2^{nd} Ed., Blackwell Scientific Publications, 1998), and Advanced Practical Organic Chemistry, by J. Leonard, et al. (2^{nd} Edition, CRC Press 1994).

Commercially available palladium catalysts include [1,1′-bis(diphenylphosphino)ferrocene]dichloropalladium(II) (“PdCl₂(dppf)”), dichlorobis(triphenylphosphine)palladium(II) (“PdCl₂(PPh₃)₂”), and tetrakis(triphenylphosphine)palladium(0) (“Pd(PPh₃)₄”), which are available from Aldrich Chemical Co., Milwaukee, Wis., and Strem Chemical Co., Newburyport, Mass. 1,2-diamino-4-bromobenzene and 2,3-diamino-5-bromopyridine are available from Aldrich Chemical Co., Milwaukee, Wis. 1,4-benzodioxan-2-carboxylic acid, (R)-1,4-benzodioxan-2-carboxylic acid, (S)-1,4-benzodioxan-2-carboxylic acid, and (S)—N-Boc-1,2,3,4-tetrahydroisoquinoline-3-carboxylic acid are available from Aldrich Chemical Co., Milwaukee, Wis. Chroman-3-carboxylic acid is available from Maybridge Chemical Co. Trevillet, Tintagel, Cornwall, UK. Pyridine-4-boronic acid, isoquinoline-4-boronic acid, isoquinoline-5-boronic acid, quinoline-4-boronic acid, 4-(4,4,5,5-tetramethyl-1,3,2-dioxaborolan-2-yl)pyridine, 4-(4,4,5,5-tetramethyl-1,3,2-dioxaborolan-2-yl)-1H-pyrazole, 3,5-dimethyl-4-(4,4,5,5-tetramethyl-1,3,2-dioxaborolan-2-yl)-1H-pyrazole, and N1-BOC-4-(4,4,5,5-tetramethyl-1,3,2-dioxaborolan-2-yl)-1H-pyrazole are available from Aldrich Chemical Co., Milwaukee, Wis. 4-chloropyrimidin-2-amine is available from Reddy Chemtech, Inc., Woodstock, Ga.

### Example 1

### 5-bromo-2-(2,3-dihydrobenzo[b][1,4]dioxin-2-yl)-1H-benzo[d]imidazole

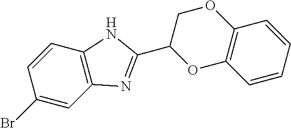

A mixture of 1,2-diamino-4-bromobenzene (0.47 g, 2.50 mmol) and 1,4-benzodioxan-2-carboxylic acid (0.54 g, 3.00 mmol) in DMF (15 mL) was treated with EDC (0.72 g, 3.75 mmol) and HOBt (0.57 g, 4.25 mmol). The mixture was stirred overnight. Next, it was diluted with ethyl acetate, washed with sodium bicarbonate, and dried over sodium sulfate. The solvent was removed by evaporation under reduced pressure. LC-MS: single peak at 254 nm, MH⁺ calcd. for C₁₅H₁₃BrN₂O₃: 349, obtained: 349 and 351. The crude amino amide was dissolved in acetic anhydride and stirred at 60° C. until complete conversion of starting material was observed (monitored by LC-MS). Acetic anhydride was removed by evaporation under reduced pressure. Next, the crude product was diluted with ethyl acetate, washed with sodium bicarbonate, and dried over sodium sulfate. The solvent was removed by evaporation under reduced pressure to obtain the desired product (0.51 g, 62%). LC-MS: single peak at 254 nm, MH⁺ calcd. for C₁₅H₁₁BrN₂O₂: 331, obtained: 331 and 333. ¹H-NMR (DMSO-d₆, 400 MHz), δ: 12.93 (s, 1H), 7.78 (bs, 1H), 7.75 (d, J=8.0 Hz, 1H), 7.36 (dd, J=2.0 Hz, J=8.4 Hz, 1H), 7.02 (m, 1H), 6.91 (m, 3H), 5.63 (dd, J=2.8 Hz, J=7.2 Hz, 1H), 4.66 (dd, J=2.8 Hz, J=11.6 Hz, 1H), 4.47 (dd, J=3.2 Hz, J=11.6 Hz, 1H).

### Example 2

### 2-(2,3-dihydrobenzo[b][1,4]dioxin-2-yl)-5-(pyridin-4-yl)-1H-benzo[d]imidazole

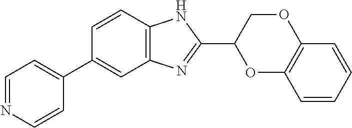

A vial was charged with potassium carbonate (0.083 g, 0.6 mmol), 4-pyridineboronic acid, pinacol ester (0.05 g, 0.24 mmol) and Example 1 (0.066 g, 0.2 mmol), dioxane (3.0 mL) and water (0.5 mL) followed by purging with argon and degassing in an ultrasound bath. Next, Pd(PPh₃)₄ (0.012 g, 0.01 mmol) was added and the mixture was stirred in Biotage “Initator” microwave, available from Biotage USA (Charlottesville, Va.) at 110° C. for 2 hours. The mixture was diluted with ethyl acetate, washed with sodium bicarbonate, and the solvent was removed by evaporation under reduced pressure. The crude mixture was dissolved in DMF and 10% aqueous solution of TFA (1 mL) was added. This solution was subjected to preparative HPLC to obtain the desired product (0.039 g, 59%). LC-MS: single peak at 254 nm, MH⁺ calcd. for C₂₀H₁₅N₃O₂: 329, obtained: 330. ¹H-NMR (DMSO-d₆, 400 MHz), δ: 8.86 (bs, 2H), 8.30 (m, 3H), 7.87 (dd, J=2.0, J=8.4 Hz, 1H), 7.77 (d, J=8.4 Hz, 1H), 7.04 (m, 1H), 6.95 (m, 3H), 5.71 (dd, J=3.0 Hz, J=6.8 Hz, 1H), 4.69 (dd, J=2.8 Hz, J=11.6 Hz, 1H), 4.54 (dd, J=6.8 Hz, J=11.6 Hz, 1H).

### Example 3

### (R)-2-(2,3-dihydrobenzo[b][1,4]dioxin-2-yl)-5-(pyridin-4-yl)-1H-benzo[d]imidazole

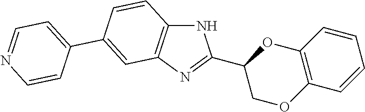

A mixture of (R)-5-Bromo-2-(2,3-dihydrobenzo[b][1,4]dioxin-2-yl)-1H-benzo[d]imidazole (1.0 equiv.), (obtained as in Example 1 using (S)-1,4-benzodioxan-2-carboxylic acid, available from SpeedChemical, Shanghai, China), 4-pyridineboronic acid (1.1 equiv.), Pd(Ph₃P)₄ (0.03 equiv.) and Na₂CO₃ (2.0 M, 3.0 equiv.) in THF (20 mL/mmol) was heated to 120° C. for 30 min in a microwave vial. The reaction mixture was purified by HPLC to afford the title compound. ¹H NMR (CDCl₃, 400 MHz) δ 4.45-4.49 (dd, J=6.8, 11.6 Hz, 1H), 4.60-4.64 (dd, J=2.8, 11.6 Hz, 1H), 5.63-5.66 (dd, J=2.8, 6.8 Hz, 1H), 6.84-6.88 (complex, 3H), 6.97-6.99 (m, 1H), 7.70-7.72 (m, 1H), 7.80-7.83 (dd, J=2.0, 8.8 Hz, 1H), 8.23 (br s, 1H), 8.28-8.30 (m, 2H), 8.80-8.82 (m, 2H); LC/MS: C₂₀H₁₆N₃O₂ (M+1) 330.19.

### Example 4

### (R)-2-(2,3-dihydrobenzo[b][1,4]dioxin-2-yl)-5-(1H-pyrazol-4-yl)-1H-benzo[d]imidazole

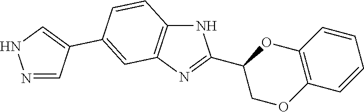

The desired product was prepared by substituting N1-BOC-4-(4,4,5,5-tetramethyl-1,3,2-dioxaborolan-2-yl)-1H-pyrazole for 4-pyridineboronic acid in Example 3. ¹H NMR (DMSO, 400 MHz) δ 4.45-4.50 (dd, J=6.8, 11.6 Hz, 1H), 4.60-4.64 (dd, J=2.8, 11.6 Hz, 1H), 5.68-5.70 (dd, J=2.8, 6.8 Hz, 1H), 6.83-6.89 (complex, 3H), 6.97-7.00 (m, 1H), 7.52-7.57 (m, 2H), 7.74 (s, 1H), 8.04 (m, 2H); LC/MS: C₁₈H₁₅N₄O₂ (M+1) 319.09.

### Example 5

### 2-(2,3-dihydrobenzo[b][1,4]dioxin-2-yl)-5-(1H-pyrrolo[2,3-b]pyridin-4-yl)-1H-benzo[d]imidazole

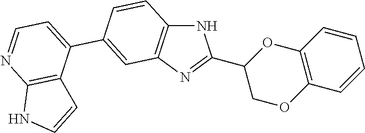

The boronic ester prepared as in Example 21A (1.5 equiv.) and 4-chloro-1H-pyrrolo[2,3-b]pyridine (1.0 equiv.) were dissolved in THF (20 mL/mmol) in a sealed tube. Pd(PPh₃)₄ (0.03 equiv) and 2M solution of Na₂CO₃ (3 equiv) were added sequentially. The resulting mixture was heated to 100° C. for one hour in a microwave reactor. After cooling to room temperature, the mixture was diluted with water and extracted with ethyl acetate. The organic layers were combined, dried over sodium sulfate and concentrated in vacuo. The residue thus produced was purified by preparative HPLC to give the desired product. ¹H NMR (DMSO, 400 MHz) δ 4.47-4.52 (dd, J=6.8, 11.6 Hz, 1H), 4.61-4.66 (dd, J=2.8, 11.6 Hz, 1H), 5.69-5.71 (dd, J=2.8, 6.8 Hz, 1H), 6.69-6.70 (m, 1H), 6.82-6.90 (complex, 3H), 6.97-7.04 (m, 1H), 7.30-7.34 (m, 1H), 7.59-7.68 (complex, 3H), 7.97-7.98 (m, 1H), 8.32-8.33 (m, 1H), 12.15 (s, 1H); LC/MS: C₂₂H₁₇N₄O₂ (M+1) 369.16.

### Example 6

### 4-(2-(2,3-dihydrobenzo[b][1,4]dioxin-2-yl)-1H-benzo[d]imidazol-5-yl)-7H-pyrrolo[2,3-d]pyrimidine

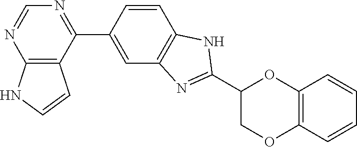

The desired product was prepared by substituting 4-chloro-7H-pyrrolo[2,3-d]pyrimidine for 4-chloro-1H-pyrrolo[2,3-b]pyridine in Example 5. ¹H NMR (DMSO, 400 MHz) δ 4.45-4.50 (dd, J=6.8, 11.6 Hz, 1H), 4.60-4.64 (dd, J=2.8, 11.6 Hz, 1H), 5.66-5.68 (dd, J=2.8, 6.8 Hz, 1H), 6.83-6.90 (complex, 3H), 6.94-6.95 (m, 1H), 6.97-7.01 (m, 1H), 7.74-7.77 (m, 2H), 7.97-8.00 (m, 1H), 8.30 (m, 1H), 8.88 (s, 1H), 12.6 (s, 1H); LC/MS: C₂₁H₁₆N₅O₂ (M+1) 370.18.

### Example 7

### 3-(2-(2,3-dihydrobenzo[b][1,4]dioxin-2-yl)-1H-benzo[d]imidazol-5-yl)-1,8-naphthyridine

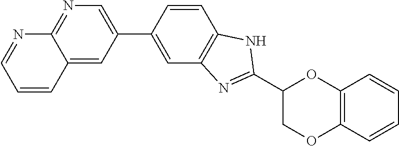

The desired product was prepared by substituting 3-chloro-1,8-naphthyridine for 4-chloro-1H-pyrrolo[2,3-b]pyridine in Example 5. ¹H NMR (DMSO, 400 MHz) δ 4.47-7.52 (dd, J=6.8, 11.6 Hz, 1H), 4.63-4.66 (dd, J=2.8, 11.6 Hz, 1H), 5.67-5.69 (dd, J=2.8, 6.8 Hz, 1H), 6.82-6.90 (complex, 3H), 6.99-7.01 (m, 1H), 7.72-7.78 (complex, 3H), 8.09 (m, 1H), 8.62-8.64 (m, 1H), 8.85-8.86 (m, 1H), 9.09-9.11 (m, 1H), 9.50-9.51 (m, 1H); LC/MS: C₂₃H₁₇N₄O₂ (M+1) 381.14.

### Example 8

### 4-(2-(2,3-dihydrobenzo[b][1,4]dioxin-2-yl)-1H-benzo[d]imidazol-5-yl)-pyrido[2,3-d]pyrimidine

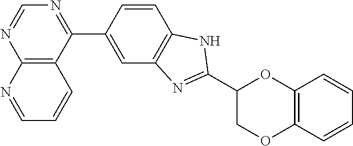

The desired product was prepared by substituting 4-chloropyrido[2,3-d]pyrimidine for 4-chloro-1H-pyrrolo[2,3-b]pyridine in Example 5. ¹H NMR (DMSO, 400 MHz) δ 4.47-7.52 (dd, J=6.8, 11.6 Hz, 1H), 4.63-4.66 (dd, J=2.8, 11.6 Hz, 1H), 5.67-5.69 (dd, J=2.8, 6.8 Hz, 1H), 6.86-6.90 (complex, 3H), 6.97-7.01 (m, 1H), 7.64-7.77 (complex, 3H), 7.99-8.00 (m, 1H), 8.54-8.56 (m, 1H), 9.23-9.25 (m, 1H), 9.48 (s, 1H); LC/MS: C₂₂H₁₆N₅O₂ (M+1) 382.13.

### Example 9

### 2-(2,3-dihydrobenzo[b][1,4]dioxin-2-yl)-1-ethyl-6-(pyridin-4-yl)-1H-benzo[d]imidazole

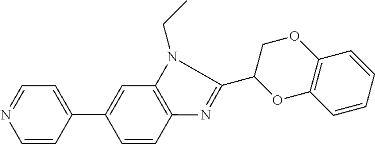

To a stirred solution of 4-bromo-2-fluoro-1-nitrobenzene (1 equiv.) in DMF (10 mL/mmol) was added MeCH₂NH₂.HCl (2 equiv) and K₂CO₃ (5 equiv). The resulting mixture was stirred at room temperature for 3 hours. Water was added and the mixture was extracted with EtOAc. The organic layers were combined, dried over sodium sulfate and concentrated in vacuo to give the ethylaniline (90%). ¹H-NMR (CDCl₃, 400 MHz), δ 7.95 (d, J=9.0 Hz, 1H), 6.94 (d, J=2.0 Hz, 1H), 6.67 (dd, J=9.0, 2.0 Hz, 1H), 3.26 (m, 2H), 1.31 (t, J=7.2 Hz, 3H). The amine was dissolved in concentrated HCl (15 mL/mmol) and SnCl₂.2H₂O (6 equiv.) was added portionwise. The resulting mixture was heated to 50° C. for 1 hour and cooled to room temperature. 10% NaOH solution was added and the mixture was extracted with EtOAc. The organic layers were combined, dried over anhydrous sodium sulfate and concentrated in vacuo (Yield 92%). ¹H-NMR (CDCl₃, 400 MHz), δ 6.76 (m, 2H), 6.58 (d, J=8.0 Hz, 1H), 3.14 (m, 2H), 1.28 (t, J=7.2 Hz, 3H). To a solution of the diamine (1 equiv.) and 1,4-benzodioxane-2-carboxylic acid (1.2 equiv.) in DMF (10 mL/mmol) were added HATU (1 equiv) and DIEA (3 equiv). The resulting mixture was stirred at room temperature for one hour. The solution was diluted with EtOAc and washed with saturated aqueous sodium bicarbonate solution. The organic layer was dried over anhydrous sodium sulfate and concentrated in vacuo. The residue was dissolved in glacial acetic acid and heated to 60° C. for 2 hours. Saturated aqueous sodium bicarbonate solution was added and the resulting solution was extracted three times with EtOAc. The organic layers were combined, dried over sodium sulfate and concentrated in vacuo to give the desired bromide (77%). This bromide was used for the following Suzuki coupling reaction without further purification. Thus the bromide (1 equiv) and pyridine-4-boronic acid (1 equiv) were dissolved in THF (15 mL/mmol) in a sealed tube. Pd(PPh₃)₄ (0.03 equiv) and 2M solution of Na₂CO₃ (3 equiv) were added sequentially. The resulting mixture was heated to 100° C. for one hour in a microwave reactor. After cooling to room temperature, the mixture was diluted with water and extracted with ethyl acetate. The organic layers were combined, dried over sodium sulfate and concentrated in vacuo. The residue thus produced was purified by preparative HPLC to give the desired product as a solid (15%). LC-MS: single peak at 254 nm, MH⁺ calcd. for C₂₂H₁₉N₃O₂: 358, obtained: 358. ¹H-NMR (DMSO-d₆, 400 MHz), δ 8.81 (d, J=6.6 Hz, 2H), 8.35 (s, 1H), 8.29 (d, J=6.5 Hz, 2H), 7.82 (s, 2H), 6.85 (m, 4H), 5.74 (dd, J=7.6, 2.5 Hz, 1H), 4.74 (dd, J=11.6, 2.5 Hz, 1H), 4.59 (dd, J=11.6, 7.6 Hz, 1H), 4.51 (m, 2H), 1.43 (t, J=7.1 Hz, 3H).

### Example 10

### 2-(2,3-dihydrobenzo[b][1,4]dioxin-2-yl)-1-ethyl-6-(1H-pyrazol-4-yl)-1H-benzo[d]imidazole

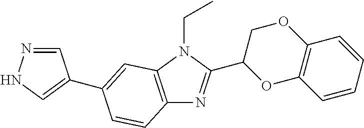

The bromide (1 equiv) prepared as in Example 9 and N1-BOC-4-(4,4,5,5-tetramethyl-1,3,2-dioxaborolan-2-yl)-1H-pyrazole (1 equiv) were dissolved in THF (15 mL/mmol) in a sealed tube. Pd(PPh₃)₄ (0.03 equiv) and 2M solution of Na₂CO₃ (3 equiv) were added sequentially. The resulting mixture was heated to 100° C. for one hour in a microwave reactor. After cooling to room temperature, the mixture was diluted with water and extracted with ethyl acetate. The organic layers were combined, dried over sodium sulfate and concentrated in vacuo. The residue thus produced was purified by preparative HPLC to give the desired product as a solid (21%). LC-MS: single peak at 254 nm, MH⁺ calcd. for C₂₀H₁₈N₄O₂: 347, obtained: 347. ¹H-NMR (DMSO-d₆, 400 MHz), δ 8.08 (s, 2H), 7.87 (s, 1H), 7.59 (d, J=8.4 Hz, 1H), 7.49 (dd, J=8.4, 1.4 Hz, 1H), 6.89 (m, 2H), 6.83 (m, 3H), 5.71 (dd, J=7.7, 2.5 Hz 1H), 4.71 (dd, J=11.7, 2.5 Hz, 1H), 4.55 (dd, J=11.7, 7.7 Hz, 1H), 4.42 (m, 2H), 1.39 (t, J=7.1 Hz, 3H).

### Example 11

### 4-(2-(2,3-dihydrobenzo[b][1,4]dioxin-2-yl)-1-ethyl-1H-benzo[d]imidazol-6-yl)-7H-pyrrolo[2,3-d]pyrimidine

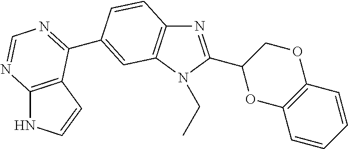

The bromide prepared as in Example 9 (1 equiv.) and bis(pinacolato)diboron (bispinacolatoboronic ester, 1.2 equiv.) in dioxane (10 mL/mmol) was treated with PdCl₂(dppf) (0.05 equiv.) and KOAc(5 equiv.). The resulting mixture was stirred at 80° C. overnight. Water was added and the mixture was extracted with EtOAc. The organic layers were combined, dried over sodium sulfate and concentrated in vacuo to give the desired boronic ester. Thus 4-chloro-7H-pyrrolo[2,3-d]pyrimidine (1 equiv) and the boronic ester (1.5 equiv) were dissolved in THF (15 mL/mmol) in a sealed tube. Pd(PPh₃)₄ (0.03 equiv) and 2M solution of Na₂CO₃ (3 equiv) were added sequentially. The resulting mixture was heated to 100° C. for one hour in a microwave reactor. After cooling to room temperature, the mixture was diluted with water and extracted with ethyl acetate. The organic layers were combined, dried over sodium sulfate and concentrated in vacuo. The residue thus produced was purified by preparative HPLC to give the desired product as a solid (15%). LC-MS: single peak at 254 nm, MH⁺ calcd. for C₂₃H₁₉N₅O₂: 398, obtained: 398. ¹H-NMR (DMSO-d₆, 400 MHz), δ 8.86 (s, 1H), 8.33 (d, J=1.2 Hz, 1H), 8.01 (dd, J=8.5, 1.6 Hz, 1H), 7.83 (d, J=8.5 Hz, 1H), 7.69 (m, 1H), 6.97-6.80 (m, 5H), 5.75 (dd, J=7.7, 2.4 Hz, 1H), 4.75 (dd, J=11.6, 2.4 Hz, 1H), 4.61 (dd, J=11.6, 7.7 Hz, 1H), 4.54 (m, 2H), 1.43 (t, J=7.2 Hz, 3H).

### Example 12

### 2-(2,3-dihydrobenzo[b][1,4]dioxin-2-yl)-1-ethyl-6-(1H-pyrrolo[2,3-b]pyridin-4-yl)-1H-benzo[d]imidazole

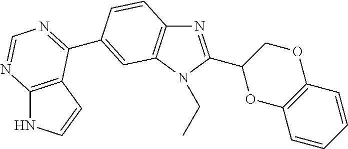

The desired product was prepared by substituting 4-chloro-1H-pyrrolo[2,3-b]pyridine (25 mg) for 4-chloro-7H-pyrrolo[2,3-d]pyrimidine in Example 11, and scaling appropriately. Preparative HPLC gave 17 mg of the title compound (26%). LC-MS: single peak at 254 nm, MH⁺ calcd. for C₂₄H₂₀N₄O₂: 397, obtained: 397. ¹H-NMR (DMSO-d₆, 400 MHz), δ 11.9 (s, 1H), 8.29 (d, J=5.1 Hz, 1H), 7.99 (d, J=1.0 Hz, 1H), 7.79 (d, J=8.4 Hz, 1H), 7.63 (dd, J=8.4, 1.6 Hz, 1H), 7.55 (m, 1H), 7.30 (d, J=5.1 Hz, 1H), 6.93-6.67 (m, 4H), 6.68 (dd, J=3.5, 1.9 Hz, 1H), 5.75 (dd, J=7.7, 2.5 Hz, 1H), 4.74 (dd, J=11.6, 2.5 Hz, 1H), 4.61 (dd, J=11.6, 7.7 Hz, 1H), 4.49 (qd, J=7.1, 3.0 Hz, 2H), 1.42 (t, J=7.1 Hz, 3H).

### Example 13

### 4-(2-(2,3-dihydrobenzo[b][1,4]dioxin-2-yl)-1-methyl-1H-benzo[d]imidazol-6-yl)-7H-pyrrolo[2,3-d]pyrimidine

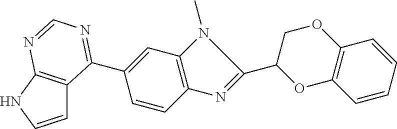

A stirred solution of 1,3-dibromobenzene (1 g) in sulfuric acid (10 mL) cooled to 0° C. was treated with 70% nitric acid (5 mL) dropwise. The resulting mixture was stirred at 0° C. for 10 min. and poured to ice in a beaker. The solution was extracted with dichloromethane (DCM) and the organic layer was washed with saturated sodium bicarbonate and saturated NaCl solution. Solvent was evaporated and the solid was used for the next reaction without further purification (76%). ¹H-NMR (CDCl₃, 400 MHz), δ 7.94 (d, J=2.0 Hz, 1H), 7.77 (d, J=8.6 Hz, 1H), 7.61 (dd, J=8.6, 2.0 Hz, 1H). To a stirred solution of 2,4-dibromo-1-nitrobenzene in 20 mL ethanol was added MeNH₂.HCl (2 equiv) and K₂CO₃ (5 equiv). The resulting mixture was stirred at reflux for 4 hours. Water was added and filtration provided product as an orange solid (98%). ¹H-NMR (CDCl₃, 400 MHz), δ 7.96 (d, J=9.1 Hz, 2H), 6.94 (d, J=2.0 Hz, 1H), 6.70 (dd, J=9.1, 2.0 Hz, 1H), 2.95 (d, J=5.1 Hz, 3H). The amine was dissolved in concentrated HCl (20 mL) and SnCl₂.2H₂O (6 equiv.) was added portionwise. The resulting mixture was heated to 50° C. for 1 hour and cooled to room temperature. 10% NaOH solution was added and extracted with EtOAc. The organic layers were combined, dried over anhydrous sodium sulfate and concentrated in vacuo to give the desired diamine (54%). ¹H-NMR (CDCl₃, 400 MHz), δ 6.77 (dd, J=8.0, 2.1 Hz, 1H), 6.73 (d, J=2.1 Hz, 1H), 6.57 (d, J=8.0 Hz, 1H), 2.84 (s, 3H). To a solution of the diamine (1 equiv) and 1,4-benzodioxane-2-carboxylic acid (1 equiv) in DMF (10 mL) were added HATU (1 equiv) and DIEA (3 equiv). The resulting mixture was stirred at room temperature for one hour. The solution was diluted with EtOAc and washed with saturated aqueous sodium bicarbonate solution. The organic layer was dried over anhydrous sodium sulfate and concentrated in vacuo. The residue was dissolved in glacial acetic acid and heated to 60° C. for 2 hours. Saturated aqueous sodium bicarbonate solution was added and the resulting solution was extracted three times with EtOAc. The organic layers were combined, dried over sodium sulfate and concentrated in vacuo to give the desired bromide. To a solution of the bromide and bis(pinacolato)diboron (bispinacolatoboronic ester, 1.2 equiv.) in dioxane (10 mL) was added PdCl₂(dppf) (15% by weight) and KOAc (5 equiv.). The resulting mixture was stirred at 80° C. overnight. Water was added and the mixture was extracted with EtOAc. The organic layers were combined, dried over sodium sulfate and concentrated in vacuo to give the desired boronic ester. Thus 4-chloro-7H-pyrrolo[2,3-d]pyrimidine (1 equiv) and the boronic ester (1.5 equiv) were dissolved in THF (10 mL) in a sealed tube. Pd(PPh₃)₄ (0.03 equiv) and 2M solution of Na₂CO₃ (3 equiv) were added sequentially. The resulting mixture was heated to 100° C. for one hour in a microwave reactor. After cooling to room temperature, the mixture was diluted with water and extracted with ethyl acetate. The organic layers were combined, dried over sodium sulfate and concentrated in vacuo. The residue thus produced was purified by preparative HPLC to give the desired product as solid (32%). LC-MS: single peak at 254 nm, MH⁺ calcd. for C₂₂H₁₇N₅O₂: 384, obtained: 384. ¹H-NMR (DMSO-d₆, 400 MHz), δ 12.64, (br, 1H), 8.96 (s, 1H), 8.40 (d, J=1.1 Hz, 1H), 8.07 (dd, J=8.5, 1.6 Hz, 1H), 7.91 (d, J=8.5 Hz, 1H), 7.81 (m, 1H), 7.12 (m, 1H), 6.99 (m, 2H), 6.90 (m, 2H), 5.86 (dd, J=7.7, 2.5 Hz, 1H), 4.81 (dd, J=11.7, 2.5 Hz, 1H), 4.66 (dd, J=11.7, 7.7 Hz, 1H), 4.09 (s, 3H).

### Example 14

### 2-(2,3-dihydrobenzo[b][1,4]dioxin-2-yl)-1-methyl-6-(1H-pyrazol-4-yl)-1H-benzo[d]imidazole

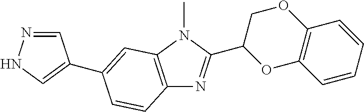

Bromide as prepared in Example 13 (1 equiv.) and N1-BOC-4-(4,4,5,5-tetramethyl-1,3,2-dioxaborolan-2-yl)-1H-pyrazole (1 equiv) were dissolved in THF (10 mL) in a sealed tube. Pd(PPh₃)₄ (0.03 equiv) and 2M solution of Na₂CO₃ (3 equiv) were added sequentially. The resulting mixture was heated to 100° C. for one hour in a microwave reactor. After cooling to room temperature, the mixture was diluted with water and extracted with ethyl acetate. The organic layers were combined, dried over sodium sulfate and concentrated in vacuo. The residue thus produced was purified by preparative HPLC to give the desired product as a solid (36%). LC-MS: single peak at 254 nm, MH⁺ calcd. for C₁₉H₁₆N₄O₂: 333, obtained: 333. ¹H-NMR (DMSO-d₆, 400 MHz), δ 8.07 (s, 2H), 7.86 (d, J=0.9 Hz, 1H), 7.58 (dd, J=8.4, 0.3 Hz, 1H), 7.50 (dd, J=8.4, 1.5 Hz, 1H), 6.91 (m, 2H), 6.83 (m, 2H), 5.72 (dd, J=7.8, 2.5 Hz 1H), 4.71 (dd, J=11.7, 2.5 Hz, 1H), 4.55 (dd, J=11.7, 7.8 Hz, 1H), 3.92 (s, 3H).

### Example 15

### 2-(2,3-dihydrobenzo[b][1,4]dioxin-2-yl)-1-methyl-6-(pyridin-4-yl)-1H-benzo[d]imidazole

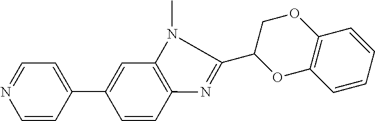

Bromide as prepared in Example 13 (166 mg) and pyridine-4-boronic acid (1 equiv) were dissolved in THF (15 mL) in a sealed tube. Pd(PPh₃)₄ (0.03 equiv) and 2M solution of Na₂CO₃ (3 equiv) were added sequentially. The resulting mixture was heated to 100° C. for one hour in a microwave reactor. Work-up was performed as in Example 14. Preparative HPLC gave the desired compound (40 mg, 24%). LC-MS: single peak at 254 nm, MH⁺ calcd. for C₂₁H₁₇N₃O₂: 344, obtained: 344. ¹H-NMR (DMSO-d₆, 400 MHz), δ 8.97 (d, J=6.7 Hz, 2H), 8.48 (s, 1H), 8.43 (d, J=6.2 Hz, 2H), 7.96 (dd, J=8.5, 1.6 Hz, 1H), 7.93 (dd, J=8.5, 0.4 Hz, 1H), 7.05 (m, 2H), 6.96 (m, 2H), 5.90 (dd, J=7.6, 2.5 Hz, 1H), 4.86 (dd, J=11.7, 2.5 Hz, 1H), 4.71 (dd, J=11.7, 7.6 Hz, 1H), 4.14 (s, 3H).

### Example 16

### 2-(2,3-dihydrobenzo[b][1,4]dioxin-2-yl)-1-methyl)-6-1H-pyrrolo[2,3-b]pyridin-4-yl)-1H-benzo[d]imidazole

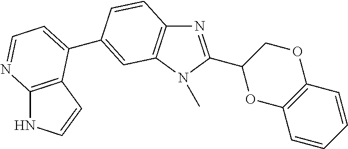

The desired product was prepared by substituting 4-chloro-1H-pyrrolo[2,3-b]pyridine (32 mg) for 4-chloro-7H-pyrrolo[2,3-d]pyrimidine in Example 13, and scaling appropriately. Preparative HPLC gave the desired compound (21 mg, 26%). LC-MS: single peak at 254 nm, MH⁺ calcd. for C₂₃H₁₈N₄O₂: 383, obtained: 383. ¹H-NMR (DMSO-d₆, 400 MHz), δ 11.96 (br, 1H), 8.30 (d, J=5.2 Hz, 1H), 8.10 (d, J=5.2 Hz, 1H), 8.00 (d, J=1.1 Hz, 1H), 7.79 (d, J=8.4 Hz, 1H), 7.64 (dd, J=8.4, 1.6 Hz, 1H), 7.54 (m, 1H), 7.31 (d, J=5.1 Hz, 1H), 7.13 (d, J=5.2 Hz, 1H), 6.95-6.72 (m, 2H), 6.44 (dd, J=3.5, 2.0 Hz, 1H), 5.78 (dd, J=7.7, 2.5 Hz, 1H), 4.74 (dd, J=11.7, 2.5 Hz, 1H), 4.58 (dd, J=11.7, 7.7 Hz, 1H), 3.99 (s, 3H).

### Example 17

### 4-(2-(2,3-dihydrobenzo[b][1,4]dioxin-2-yl)-1-methyl-1H-benzo[d]imidazol-6-yl)pyrimidin-2-amine

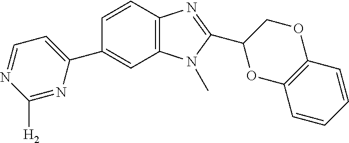

The desired product was prepared by substituting 4-chloropyrimidin-2-amine (39 mg) for 4-chloro-7H-pyrrolo[2,3-d]pyrimidine in Example 13, and scaling appropriately. Preparative HPLC gave the desired compound (21 mg, 34%). LC-MS: single peak at 254 nm, MH⁺ calcd. for C₂₀H₁₇N₅O₂: 360, obtained: 360. ¹H-NMR (DMSO-d₆, 400 MHz), δ 8.45 (d, J=1.2 Hz, 1H), 8.35 (d, J=6.1 Hz, 1H), 8.05 (dd, J=8.6, 1.6 Hz, 1H), 7.75 (d, J=8.7 Hz, 1H), 7.49 (d, J=6.1 Hz, 1H), 6.93-6.79 (m, 5H), 5.75 (dd, J=7.6, 2.5 Hz, 1H), 4.72 (dd, J=11.6, 2.5 Hz, 1H), 4.56 (dd, J=11.6, 7.6 Hz, 1H), 3.98 (s, 3H).

### Example 18

### 2-(2,3-dihydrobenzo[b][1,4]dioxin-2-yl)-7-methyl-5-(pyridin-4-yl)-1H-benzo[d]imidazole

### 18A. 5-bromo-2-(2,3-dihydrobenzo[b][1,4]dioxin-2-yl)-7-methyl-1H-benzo[d]imidazole

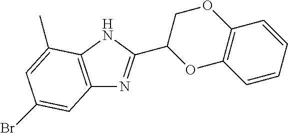

The desired product was prepared by substituting 5-bromo-3-methylbenzene-1,2-diamine (500 mg, available from Maybridge Chemical Co. Trevillet, Tintagel, Cornwall, UK) for 1,2-diamino-4-bromobenzene in Example 1, and scaling appropriately. 701 mg of the desired bromo-benzimidazole was obtained (82%). LC-MS: single peak at 254 nm, MH⁺ calcd. for C₁₆H₁₃BrN₂O₂: 345, obtained 345.

### 18B. 2-(2,3-dihydrobenzo[b][1,4]dioxin-2-yl)-7-methyl-5-(pyridin-4-yl)-1H-benzo[d]imidazole

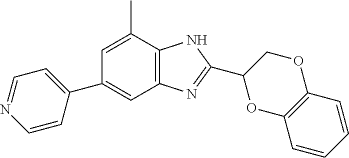

Example 18A bromide was treated as in Example 2 to give the desired product. LC-MS: single peak at 254 nm, MH⁺ calcd. for C₂₁H₁₇N₃O₂: 344, obtained 344. HPLC: single peak by analytical HPLC. ¹H-NMR (MeOD- d₄, 400 MHz): 8.58 (d, 2H, J=4.8 Hz), 7.82 (s, 1H), 7.77 (d, 2H, 4.8 Hz), 7.52 (s, 1H), 7.52 (m, 1H), 6.94 (m, 3H), 5.57 (dd, 1H, J=7.9 Hz, 2.5 Hz), 4.70 (dd, 1H, J=11.5 Hz, 2.5 Hz), 4.44 (dd, 1H, J=11.5 Hz, 8.0 Hz), 2.70 (s, 3H).

### Example 19

### 2-(2,3-dihydrobenzo[b][1,4]dioxin-2-yl)-5-(pyridin-4-yl)-7-(trifluoromethyl)-1H-benzo[d]imidazole

### 19A. 5-bromo-2-(2,3-dihydrobenzo[b][1,4]dioxin-2-yl)-7-(trifluoromethyl)-1H-benzo[d]imidazole

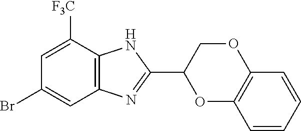

The desired product was prepared by substituting 5-bromo-3-(trifluoromethyl)benzene-1,2-diamine (255 mg, available from Sunshine ChemLab, Inc., Thorndale, Pa.) for 1,2-diamino-4-bromobenzene in Example 1, and scaling appropriately. 199 mg of the desired bromo-benzimidazole was obtained (50%). LC-MS: single peak at 254 nm, MH⁺ calcd. for C₁₆H₁₀BrF₃N₂O₂: 399, obtained 399. ¹H-NMR (DMSO-d₆, 400 MHz): 8.06 (s, 1H), 7.72 (s, 1H), 6.99 (m, 4H), 5.71 (m, 1H), 4.70 (m, 1H), 4.47 (m, 1H).

### 19B. 2-(2,3-dihydrobenzo[b][1,4]dioxin-2-yl)-5-(pyridin-4-yl)-7-(trifluoromethyl)-1H-benzo[d]imidazole

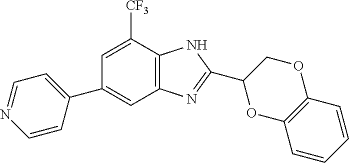

Example 19A bromide (60 mg) was treated as in Example 2 to give the desired product (30 mg, 50%). LC-MS: single peak at 254 nm, MH⁺ calcd. for C₂₁H₁₄F₃N₃O₂: 398, obtained 398. HPLC: single peak by analytical HPLC. ¹H-NMR (CDCl₃, 400 MHz): 8.72 (m, 2H), 8.23 (s, 1H), 7.83 (s, 1H), 7.66 (m, 1H), 7.54 (m, 2H), 7.44 (m, 1H), 7.08 (m, 1H), 6.97 (m, 3H), 5.60 (dd, 1H, J=2.2 Hz, 7.5 Hz), 4.84 (dd, 1H, J=2.3 Hz, 11.5 Hz), 4.40 (dd, 1H, J=7.4 Hz, 11.7 Hz).

### Example 20

### 2-(2,3-dihydrobenzo[b][1,4]dioxin-2-yl)-5-(pyridin-4-yl)-6-(trifluoromethyl)-1H-benzo[d]imidazole

### 20A. 5-bromo-2-(2,3-dihydrobenzo[b][1,4]dioxin-2-yl)-6-(trifluoromethyl)-1H-benzo[d]imidazole

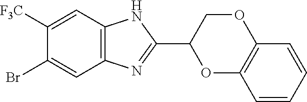

The desired product was prepared by substituting 4-bromo-5-(trifluoromethyl)benzene-1,2-diamine (255 mg, available from Sunshine ChemLab, Inc., Thorndale, Pa.) for 1,2-diamino-4-bromobenzene in Example 1, and scaling appropriately. 299 mg of the desired bromo-benzimidazole was obtained (75%). LC-MS: single peak at 254 nm, MH⁺ calcd. for C₁₆H₁₀BrF₃N₂O₂: 399, obtained 399. ¹H-NMR (DMSO-d₆, 400 MHz): 8.00 (s, 2H), 6.97 (m, 1H), 6.85 (m, 3H), 5.65 (dd, 1H, J=2.7 Hz, 6.8 Hz), 4.60 (dd, 1H, J=2.6 Hz, 11.6 Hz), 4.44 (dd, 1H, J=6.8 Hz, 11.7 Hz).

20B. 2-(2,3-dihydrobenzo[b][1,4]dioxin-2-yl)-5-(pyridin-4-yl)-6-(trifluoromethyl)-1H-benzo[d]imidazole

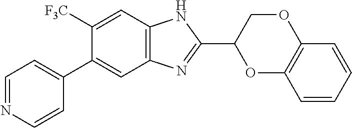

Example 20A bromide (60 mg) was treated as in Example 2 to give the desired product (20 mg, 34%). LC-MS: single peak at 254 nm, MH⁺ calcd. for C₂₁H₁₄F₃N₃O₂: 398, obtained 398. HPLC: single peak by analytical HPLC.

### Example 21

### 4-(2-(2,3-dihydrobenzo[b][1,4]dioxin-2-yl)-1H-benzo[d]imidazol-5-yl)-N-ethylpyrimidin-2-amine

### 21A. 2-(2,3-dihydrobenzo[b][1,4]dioxin-2-yl)-5-(4,4,5,5-tetramethyl-1,3,2-dioxaborolan-2-yl)-1H-benzo[c]imidazole

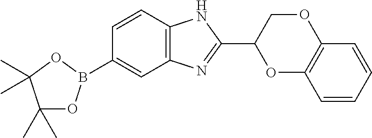

A 250 mL round-bottom flask containing Example 1 (2.48 g, 7.49 mmol), bis(pinacolato)diboron (4.75 g, 2.5 eq.) and potassium acetate (3.67 g, 5 eq.) was put under an argon atmosphere. To this was added 70 mL of argon-purged anhydrous 1,4-dioxane. The solution was stirred until dissolution occurred. To this solution was added Pd(dppf)Cl₂ (4.89 mg, 0.08 eq.). The reaction was heated at 80° C. for 48 hours. LC-MS indicated complete disappearance of aryl bromide. The reaction was cooled and filtered through filter paper. The dioxane solution was concentrated and the resulting residue was taken up in DCM. This was purified on a 120 g silica gel column (DCM:EtOAc gradient) to give 1.52 g of the desired product as a light brown oil (54% yield). LC-MS (found 379.2, MH+ calculated for C₂₁H₂₄BN₂O₄: 379.2). ¹H-NMR (MeOH-d₄, 400 MHz) δ 1.36 (s, 12H), 4.40 (dd, 7.2 Hz, 11.6 Hz, 1H), 4.66 (dd, 2.4 Hz, 11.6 Hz, 1H), 5.54 (dd, 2.4 Hz, 7.6 Hz, 1H), 6.86-6.94 (m, 3H), 7.03-7.07 (m, 1H), 7.58 (d, 8.0 Hz, 1H), 7.66 (dd, 1.2 Hz, 8.0 Hz, 1H), 8.03 (s, 1H). Single peak by HPLC.

### 21B. 4-chloro-N-ethylpyrimidin-2-amine

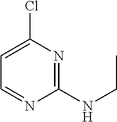

2,4-dichloropyrimidine (700 mg, 4.70 mmol) and ethylamine hydrochloride (525 mg, 1.4 equiv.) were suspended in 15 mL n-butanol. Triethylamine (1.96 mL, 3.0 equiv.) was added and the reaction was heated at 90° C. overnight. The reaction was poured into 200 mL water and extracted 2× with EtOAc. The organic layers were washed once each with water and brine. The organic layer was dried with sodium sulfate and concentrated. The residue was put on a high vacuum pump to remove remaining n-butanol. The residue was then purified on a 40 g silica gel column (hexanes:EtOAc gradient) to give 340 mg of 2-chloro-4-ethylaminopyrimidine (49% yield) and 145 mg of the desired product (21% yield) as a colorless solid. LC-MS (found 158.1, MH+ calculated for C₆H₁₀ClN3: 158.1). ¹H-NMR (CDCl₃, 400 MHz) δ 1.21 (t, 7.2 Hz, 3H), 3.42 (pentet, 7.2 Hz, 2H), 5.32 (bs, 1H), 6.51 (s, 1H), 8.11 (s, 1H). Single peak by HPLC.

### 21C. 4-(2-(2,3-dihydrobenzo[b][1,4]dioxin-2-yl)-1H-benzo[d]imidazol-5-yl)-N-ethylpyrimidin-2-amine

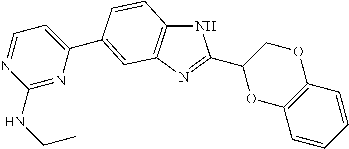

In a microwave pressure vial Example 21A (55 mg, 0.150 mmol), Example 21B (23 mg, 1.0 eq.), and sodium carbonate (31 mg, 2.0 equiv.) were put under an argon atmosphere. Tetrakis(triphenylphosphino)palladium(0) (17 mg, 0.05 equiv.) was added and the vial was sealed. The contents were dissolved in 1 mL degassed 2:1 1,4-dioxane:water and heated in the microwave for 30 minutes at 130° C. The solvent was removed in vacuo and residue was taken up in equal portions of DCM and water. The layers were separated and the aqueous phase was washed 2× with DCM. Organic layers were combined, dried with sodium sulfate, concentrated, and purified on a 4 g silica gel column (DCM:EtOAc) to give 41 mg of the desired product (73% yield). LC-MS (found 374.2, MH+ calculated for C₂₁H₂₀N₅O₂: 374.2). Single peak by HPLC.

### Example 22

### 2-(2,3-dihydrobenzo[b][1,4]dioxin-2-yl)-5-(pyrimidin-4-yl)-1H-benzo[d]imidazole

### 22A. 4-Chloropyrimidine

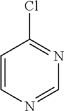

4(3H)-Pyrimidone (400 mg, 4.16 mmol) was dissolved in 4 mL POCl₃ in a 10 mL sealed vial and heated at 100° C. for one hour. A yellow precipitate formed which was filtered off after the reaction had cooled. The precipitate was washed with hexanes and dried on the high vacuum pump to give 107 mg of the desired product (23% yield). ¹H-NMR (DMSO-d₆, 400 MHz) δ 7.75 (dd, 1.2 Hz, 5.6 Hz, 1H), 8.80 (dd, 0.4 Hz, 5.6 Hz, 1H), 9.05 (s, 1H).

### 22B. 2-(2,3-dihydrobenzo[b][1,4]dioxin-2-yl)-5-(pyrimidin-4-yl)-1H-benzo[d]imidazole

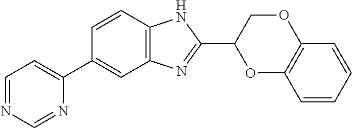

Example 21A boronic ester (132 mg, 0.350 mmol) and Example 22A (40 mg, 1.0 eq.) were treated as in Example 21C to give 110 mg of the desired product (85% yield). LCMS (found 331.1, MH+ calculated for C₁₉H₁₅N₄O₂: 331.1). ¹H-NMR (DMSO-d₆, 400 MHz) δ 4.51 (dd, 7.2 Hz, 11.6 Hz, 1H), 4.69 (dd, 2.8 Hz, 11.6 Hz, 1H), 5.63-5.69 (m, 1H), 6.86-6.95 (m, 3H), 7.00-7.06 (m, 1H), 7.62 (d, 8.4 Hz, 0.5H), 7.77 (d, 8.4 Hz, 0.5H), 8.02-8.20 (m, 2H), 8.38 (s, 0.5H), 8.53 (s, 0.5H), 8.78-8.84 (m, 1H), 9.21 (d, 4.4 Hz, 1H). Single peak by HPLC.

### Example 23

### 2-(6,7-dichloro-2,3-dihydrobenzo[b][1,4]dioxin-2-yl)-5-(pyridin-4-yl)-1H-benzo[d]imidazole, trifluoroacetic acid salt

### 23A. bis(tert-butyl) 4-bromo-1,2-phenylenedicarbamate

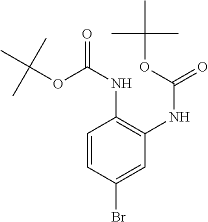

Di-tert-butyl dicarbonate (6.21 g, 2.2 eq.) was dissolved in 30 mL ethanol. To this solution was added 1,2-diamino-4-bromobenzene (2.42 g, 12.9 mmol). The reaction mixture was stirred overnight, then the solvent was removed. The residue was taken up in EtOAc and washed 2× with 1N HCl. The organic layer was dried with sodium sulfate and concentrated to give 4.2 g of the desired product as a brown solid. ¹H-NMR (DMSO-d₆, 400 MHz) δ 1.43 (s, 18H), 7.21 (dd, 2.4 Hz, 8.8 Hz, 1H), 4.43 (d, 8.8 Hz, 1H), 7.72 (s, 1H), 8.53-8.73 (m, 2H). Single peak by HPLC.

### 23B. bis(tert-butyl) 4-(pyridin-4-yl)-1,2-phenylenedicarbamate

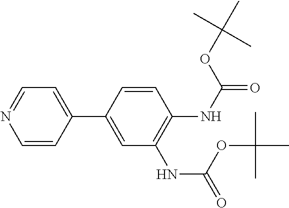

In a microwave pressure vial the Bis-Boc-protected Example 23A (700 mg, 1.81 mmol), 4-pyridine boronic acid (250 mg, 1.2 equiv.), and sodium carbonate (750 mg, 4.0 equiv.) were put under an argon atmosphere. Tetrakis(triphenylphosphino)palladium(0) (150 mg, 0.05 equiv.) was added and the vial was sealed. The contents were dissolved in 15 mL degassed 2:1 1,2-dimethoxyethane:water and heated in an oil bath at 85° C. overnight. The solvent was removed in vacuo and residue was taken up in equal portions DCM and water. The layers were separated and the aqueous phase was washed 2× with DCM. Organic layers were combined, dried with sodium sulfate, concentrated, and purified on a 40 g silica gel column (DCM:EtOAc gradient) to give 438 mg of the desired product as a colorless oil (63% yield). LC-MS (found 386.2, MH+ calculated for C₂₁H₂₈N₃O₄: 386.2). ¹H-NMR (MeOH-d₄, 400 MHz) δ 1.53 (s, 18H), 7.51 (dd, 2.4 Hz, 8.8 Hz, 1H), 7.61-7.69 (m, 3H), 7.88 (s, 1H), 8.51-8.57 (m, 2H).

### 23C. 4-(pyridin-4-yl)benzene-1,2-diamine

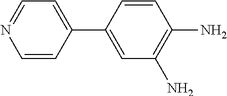

The starting material (270 mg, 0.698 mmol) was dissolved in a solution of 50% TFA in methylene chloride (5 mL) and the mixture was stirred at room temperature for 1 hour. The solvent was removed under reduced pressure and excess acid was removed by repeated evaporation from toluene in vacuo. The crude amine was used without further purification.

### 23D. ethyl 6,7-dichloro-2,3-dihydrobenzo[b][1,4]dioxine-2-carboxylate

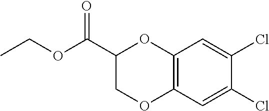

A solution of 4,5-dichlorocatechol (400 mg) in acetone (10 mL) was treated with potassium carbonate (2.5 equiv.) followed by ethyl 2,3-dibromopropionate (1.0 equiv.). The reaction was heated to reflux for 24-48 hours. The solvent was removed in vacuo and the residue taken up in equal portions EtOAc and water. The layers were separated and the aqueous phase was washed 2× with EtOAc. Organic layers were combined, dried with sodium sulfate, and concentrated. The residue was purified by silica gel chromatography (hexanes:EtOAc) to give the desired benzodioxane derivative in 62% yield (405 mg) as a colorless oil. ¹H-NMR (MeOH-d₄, 400 MHz) δ 1.26 (t, 7.2 Hz, 3H), 4.23 (q, 7.2 Hz, 2H), 4.31 (dd, 2.8 Hz, 11.6 Hz, 1H), 4.49 (dd, 3.6 Hz, 11.6 Hz, 1H), 5.03 (t, 3.4 Hz, 1H), 7.01 (s, 1H), 7.11 (s, 1H). Single peak by HPLC.

### 23E. 6,7-dichloro-2,3-dihydrobenzo[b][1,4]dioxine-2-carboxylic acid

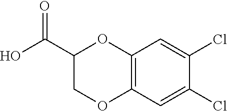

A solution of Example 23D (386 mg) in THF (10 mL) was treated with lithium hydroxide monohydrate (3.0 equiv.) and the reaction mixture was refluxed until LC-MS indicated that the ester had been consumed. The solvent was removed in vacuo and the residue taken up in water. With vigorous stirring and cooling by an ice bath the solution was acidified with 1N HCl. The aqueous phase was extracted 3× with EtOAc. The organic layers were combined, dried with sodium sulfate, and concentrated to give the desired carboxylic acid in 89% yield (310 mg) as a bright yellow solid. ¹H-NMR (MeOH-d₄, 400 MHz) δ 4.33 (dd, 2.8 Hz, 11.4 Hz, 1H), 4.48 (dd, 4.0 Hz, 11.4 Hz, 1H), 4.67 (dd, 3.0 Hz, 3.4 Hz, 1H), 7.02 (s, 1H), 7.11 (s, 1H). Single peak by HPLC.

### 23F. 2-(6,7-dichloro-2,3-dihydrobenzo[b][1,4]dioxin-2-yl)-5-(pyridin-4-yl)-1H-benzo[d]imidazole, trifluoroacetic acid salt

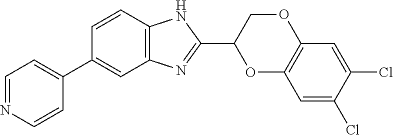

A solution of Example 23E (48 mg) and Example 23C (1.0 equiv.) in DMF (1 mL) was treated with HATU (1.1 equiv.) followed by DIEA (1.1 equiv.). The reaction mixture was stirred for 1-6 hours and then poured into distilled water. The water was extracted 2× with DCM. The organic layers were combined, washed 3× with saturated sodium bicarbonate solution, dried with sodium sulfate, and concentrated to give the amide intermediate. Presence of the amide intermediate was confirmed by LC-MS. This crude product was dissolved in glacial acetic acid and heated at 60-65° C. for 1-24 hours. Upon complete consumption of the amide intermediate, indicated by LC-MS, the reaction was concentrated in vacuo. The residue was taken up in water and neutralized with saturated sodium bicarbonate. The aqueous solution was then extracted 3× with DCM. The organic layers were combined, dried with sodium sulfate, and concentrated to give the crude benzimidazole product. This product was purified by preparative HPLC (gradient; mobile phase: solvent A: 0.1% TFA in water, solvent B: CH₃CN) to obtain the desired product as the trifluoroacetate salt in 61% yield (60 mg). ¹H-NMR (MeOH-d₄, 400 MHz) δ 4.56 (dd, 7.0 Hz, 7.8 Hz, 1H), 4.73 (dd, 2.6 Hz, 7.8 Hz, 1H), 5.73 (dd, 2.8 Hz, 6.8 Hz, 1H), 7.13 (s, 1H), 7.27 (s, 1H), 7.83 (d, 8.4 Hz, 1H), 7.94 (dd, 2.0 Hz, 8.6 Hz, 1H), 8.27 (s, 1H), 8.43 (d, 6.0 Hz, 2H), 8.83 (d, 6.0 Hz, 2H). LCMS (found 398.0, 400.0, MH+ calculated for C₂₀H₁₄Cl₂N₃O₂: 398.0, 400.0). Single peak by HPLC.

### Example 24

### 2-(7-chloro-2,3-dihydrobenzo[b][1,4]dioxin-2-yl)-5-(pyridin-4-yl)-1H-benzo[d]imidazole, trifluoroacetic acid salt and 2-(6-chloro-2,3-dihydrobenzo[b][1,4]dioxin-2-yl)-5-(pyridin-4-yl)-1H-benzo[d]imidazole, trifluoroacetic acid salt 24A. ethyl 7-chloro-2,3-dihydrobenzo[b][1,4]dioxine-2-carboxylate and ethyl 6-chloro-2,3-dihydrobenzo[b][1,4]dioxine-2-carboxylate

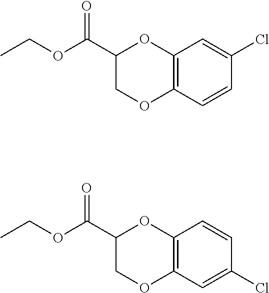

The desired product was prepared by substituting 4-chlorocatechol (500 mg) for 4,5-dichlorocatechol in Example 23D to give a regioisomeric mixture of its benzodioxane derivative in 51% yield (424 mg) as a colorless oil. Single peak by HPLC.

### 24B. 7-chloro-2,3-dihydrobenzo[b][1,4]dioxine-2-carboxylic acid and 6-chloro-2,3-dihydrobenzo[b][1,4]dioxine-2-carboxylic acid

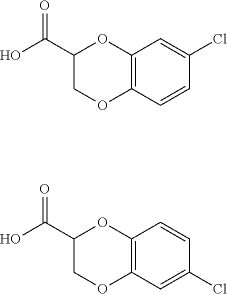

The desired product was prepared by substituting Example 24A (424 mg) for Example 23D in Example 23E to give the desired carboxylic acid mixture in 84% yield (316 mg) as a colorless solid. ¹H-NMR (DMSO-d₆, 400 MHz) δ 4.23-4.31 (m, 1H), 4.41-4.48 (m, 1H), 5.05-5.12 (m, 1H), 6.85-6.97 (m, 2H), 7.02-7.04 (m, 1H), 13.48 (bs, 1H).

### 24C. 2-(7-chloro-2,3-dihydrobenzo[b][1,4]dioxin-2-yl)-5-(pyridin-4-yl)-1H-benzo[d]imidazole, trifluoroacetic acid salt and 2-(6-chloro-2,3-dihydrobenzo[b][1,4]dioxin-2-yl)-5-(pyridin-4-yl)-1H-benzo[d]imidazole, trifluoroacetic acid salt

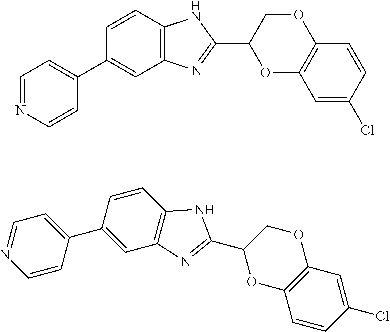

The desired product was prepared by substituting Example 24B (42 mg) for Example 23E in Example 23F to give the desired benzimidazoles in 69% yield (66 mg). ¹H-NMR (MeOH-d₄, 400 MHz) δ 4.49-4.57 (m, 1H), 4.68-4.75 (m, 1H), 5.66-5.73 (m, 1H), 6.90-7.15 (m, 3H), 7.84 (d, 8.6 Hz, 1H), 7.94 (dd, 1.6 Hz, 8.8 Hz, 1H), 8.27 (d, 1.6 Hz, 1H), 8.43 (d, 5.6 Hz, 2H), 8.83 (d, 5.6 Hz, 2H).). LCMS (found 364.1, 366.1, MH+ calculated for C₂₀H₁₅ClN₃O₂: 364.1, 366.1). Single peak by HPLC.

### Example 25

### 2-(5-methoxy-2,3-dihydrobenzo[b][1,4]dioxin-2-yl)-5-(pyridin-4-yl)-1H-benzo[d]imidazole, trifluoroacetic acid salt and 2-(8-methoxy-2,3-dihydrobenzo[b][1,4]dioxin-2-yl)-5-(pyridin-4-yl)-1H-benzo[d]imidazole, trifluoroacetic acid salt

### 25A. ethyl 8-methoxy-2,3-dihydrobenzo[b][1,4]dioxine-2-carboxylate and ethyl 5-methoxy-2,3-dihydrobenzo[b][1,4]dioxine-2-carboxylate

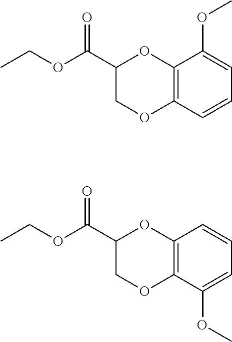

The desired product was prepared by substituting 3-methoxycatechol (1.00 g) for 4,5-dichlorocatechol in Example 23D to give a regioisomeric mixture of its benzodioxane derivative in 68% yield (1.15 g) as a colorless solid. ¹H-NMR (MeOH-d₄, 400 MHz) δ 1.25 (t, 7.2 Hz, 3H), 4.15-4.30 (m, 3H), 4.37 (dd, 3.8 Hz, 11.2 Hz), 4.87-4.92 (m, 1H), 6.60-6.81 (m, 3H). Two peaks of equal intensity by HPLC.

### 25B. 8-methoxy-2,3-dihydrobenzo[b][1,4]dioxine-2-carboxylic acid and 5-methoxy-2,3-dihydrobenzo[b][1,4]dioxine-2-carboxylic acid

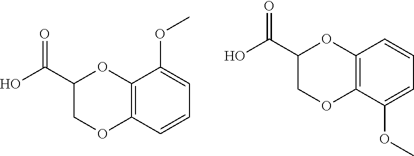

The desired product was prepared by substituting Example 25A (1.14 g) for Example 23D in Example 23E to give the desired carboxylic acid mixture in 79% yield (800 mg) as a colorless solid. Two equal peaks were observed by HPLC.

### 25C. 2-(5-methoxy-2,3-dihydrobenzo[b][1,4]dioxin-2-yl)-5-(pyridin-4-yl)-1H-benzo[d]imidazole, trifluoroacetic acid salt and 2-(8-methoxy-2,3-dihydrobenzo[b][1,4]dioxin-2-yl)-5-(pyridin-4-yl)-1H-benzo[d]imidazole, trifluoroacetic acid salt

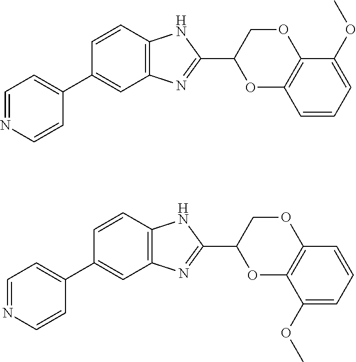

The desired product was prepared by substituting Example 25B (41 mg) for Example 23E in Example 23F to give the desired benzimidazoles in 71% overall yield (66 mg total). One fraction contains 11 mg of a single regioisomer. ¹H-NMR (MeOH-d₄, 400 MHz) δ 3.90 (s, 3H), 4.44 (dd, 3.6 Hz, 7.6 Hz, 1H), 4.71 (dd, 2.6 Hz, 11.2 Hz, 1H), 5.61 (dd, 2.6 Hz, 7.9 Hz, 1H), 6.59 (dd, 1.2 Hz, 8.4 Hz, 1H), 6.67 (dd, 1.2 Hz, 8.4 Hz, 1H), 6.86 (t, 8.0 Hz, 1H), 7.84 (d, 8.8 Hz, 1H), 7.92 (dd, 2.0 Hz, 10.4 Hz, 1H), 8.26 s (1H), 8.41 (d, 8.6 Hz, 2H), 8.82 (d, 8.6 Hz, 2H). LC-MS (found 360.1, MH+ calculated for C₂₁H₁₈N₃O₃: 360.1). Single peak by HPLC. Another fraction contains 55 mg of a 3:1 mixture of regioisomers. ¹H-NMR (MeOH-d₄, 400 MHz) δ 3.83 (s, 2.25H), 3.89 (s, 0.75H), 4.40-4.52 (m, 1H), 4.68-4.75 (m, 1H), 5.58-5.63 (m, 0.25H), 5.63-5.68 (m, 0.75H), 6.55-6.77 (m, 2H), 6.82-6.90 (m, 1H), 7.81-7.87 (m, 1H), 7.91-7.93 (m, 1H), 8.24-8.29 (m, 1H), 8.38-8.45 (m, 2H), 8.78-8.85 (m, 2H). LC-MS (found 360.1, MH+ calculated for C₂₁H₁₈N₃O₃: 360.1). Two peaks by HPLC (3:1 ratio).

### Example 26

### 2-(7-methyl-2,3-dihydrobenzo[b][1,4]dioxin-2-yl)-5-(pyridin-4-yl)-1H-benzo[d]imidazole, trifluoroacetic acid salt and 2-(6-methyl-2,3-dihydrobenzo[b][1,4]dioxin-2-yl)-5-(pyridin-4-yl)-1H-benzo[d]imidazole, trifluoroacetic acid salt

### 26A. ethyl 7-methyl-2,3-dihydrobenzo[b][1,4]dioxine-2-carboxylate and ethyl 6-methyl-2,3-dihydrobenzo[b][1,4]dioxine-2-carboxylate

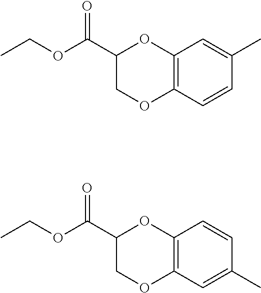

The desired product was prepared by substituting 4-methylcatechol (1.00 g) for 4,5-dichlorocatechol in Example 23D to give a regioisomeric mixture of its benzodioxane derivative in 61% yield (1.09 g) as a colorless solid. ¹H-NMR (MeOH-d₄, 400 MHz) δ 1.20-1.33 (m, 3H), 3.79 (s, 1.6H), 3.84 (s, 1.4H), 4.18-4.32 (m, 3H), 4.42-4.49 (m, 1H), 4.93-4.98 (m, 1H), 6.44-6.48 (m, 0.5H), 6.54-6.61 (m, 1.5H), 6.73-6.83 (m, 1H). Single peak by HPLC.

### 26B. 7-methyl-2,3-dihydrobenzo[b][1,4]dioxine-2-carboxylic acid and 6-methyl-2,3-dihydrobenzo[b][1,4]dioxine-2-carboxylic acid

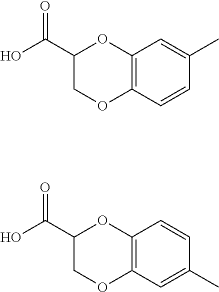

The desired product was prepared by substituting Example 26A (1.18 g) for Example 23D in Example 23E to give the desired carboxylic acid mixture in 97% yield (996 mg) as a colorless solid. Single peak by HPLC.

### 26C. 2-(7-methyl-2,3-dihydrobenzo[b]benzo[d]imidazole, trifluoroacetic acid salt and 2-(6-methyl-2,3-dihydrobenzo[b][1,4]dioxin-2-yl)-5-(pyridin-4-yl)-1H-benzo[d]imidazole, trifluoroacetic acid salt

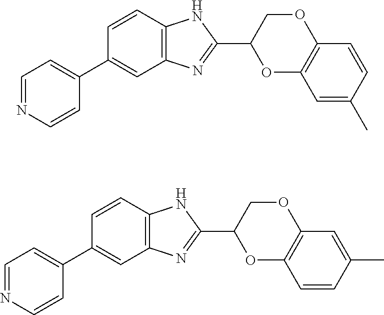

The desired product was prepared by substituting Example 26B (38 mg) for Example 23E in Example 23F to give the desired benzimidazoles in 66% yield (60 mg). ¹H-NMR (MeOH-d₄, 400 MHz) δ 2.24 (s, 1.5H), 2.72 (s, 1.5H), 4.43-4.51 (m, 1H), 4.62-4.69 (m, 1H), 5.60-5.67 (m, 1H), 6.70-6.82 (m, 2H), 6.90-6.99 (m, 1H), 7.84 (d, 8.8 Hz, 1H), 7.91-7.97 (m, 1H), 8.25-8.28 (m, 1H), 8.41 (d, 5.6 Hz, 2H), 8.83 (d, 5.6 Hz, 2H). LCMS (found 344.1, MH+ calculated for C₂₁H₁₈N₃O₂: 344.1). Single peak by HPLC.

### Example 27

### 2-(5-fluoro-2,3-dihydrobenzo[b][1,4]dioxin-2-yl)-5-(pyridin-4-yl)-1H-benzo[d]imidazole, trifluoroacetic acid salt and 2-(8-fluoro-2,3-dihydrobenzo[b][1,4]dioxin-2-yl)-5-(pyridin-4-yl)-1H-benzo[di]imidazole, trifluoroacetic acid salt

### 27A. ethyl 8-fluoro-2,3-dihydrobenzo[b][1,4]dioxine-2-carboxylate and ethyl 5-fluoro-2,3-dihydrobenzo[b][1,4]dioxine-2-carboxylate

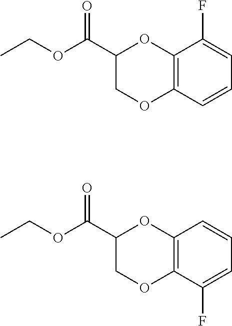

The desired product was prepared by substituting 3-fluorocatechol (500 mg) for 4,5-dichlorocatechol in Example 23D to give a regioisomeric mixture of its benzodioxane derivative in 41% yield (366 mg) as a colorless solid. ¹H-NMR (MeOH-d₄, 400 MHz) δ 1.22-1.30 (m, 3H), 4.20-4.28 (m, 2H), 4.30-4.37 (m, 1H), 4.48-4.54 (m, 1H), 5.01-5.06 (m, 1H), 6.61-6.87 (m, 3H). Two equal peaks were observed by HPLC.

### 27B. 8-fluoro-2,3-dihydrobenzo[b][1,4]dioxine-2-carboxylic acid and 5-fluoro-2,3-dihydrobenzo[b][1,4]dioxine-2-carboxylic acid

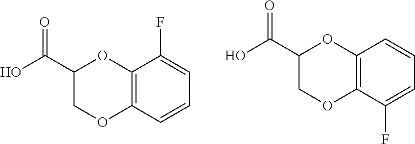

The desired product was prepared by substituting Example 27A (366 mg) for Example 23D in Example 23E to give the desired carboxylic acid mixture in 88% yield (283 mg) as a colorless solid. LC-MS (found 197.0, M− calculated for C₉H₆FO₄: 197.0). Single peak by HPLC.

### 27C. 2-(5-fluoro-2,3-dihydrobenzo[b][1,4]dioxin-2-yl)-5-(pyridin-4-yl)-1H-benzo[d]imidazole, trifluoroacetic acid salt and 2-(8-fluoro-2,3-dihydrobenzo[b][1,4]dioxin-2-yl)-5-(pyridin-4-yl)-1H-benzo[d]imidazole, trifluoroacetic acid salt

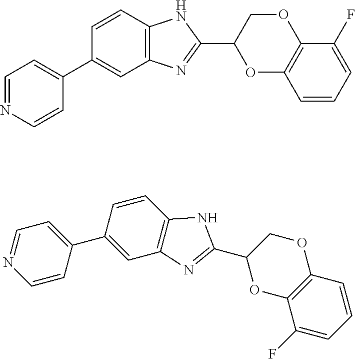

The desired product was prepared by substituting Example 27B (36 mg) for Example 23E in Example 23F to give the desired benzimidazoles in 65% yield (55 mg). ¹H-NMR (MeOH-d₄, 400 MHz) δ 4.52-4.59 (m, 1H), 4.72-4.80 (m, 1H), 5.68-5.75 (m, 1H), 6.74-6.94 (m, 3H), 7.81-7.88 (m, 1H), 7.91-7.97 (m, 1H), 8.26-8.29 (m, 1H), 8.43 (d, 5.6 Hz, 2H), 8.83 (d, 5.6 Hz, 2H). LC-MS (found 348.1, MH+ calculated for C₂₀H₁₅FN₃O₂: 348.1). Single peak by HPLC.

### Example 28

### 2-(7-fluoro-2,3-dihydrobenzo[b][1,4]dioxin-2-yl)-5-(pyridin-4-yl)-1H-benzo[d]imidazole trifluoroacetic acid salt and 2-(6-fluoro-2,3-dihydrobenzo[b][1,4]dioxin-2-yl)-5-(pyridin-4-yl)-1H-benzo[d]imidazole, trifluoroacetic acid salt

### 28A. ethyl 7-fluoro-2,3-dihydrobenzo[b][1,4]dioxine-2-carboxylate and ethyl 6-fluoro-2,3-dihydrobenzo[b][1,4]dioxine-2-carboxylate

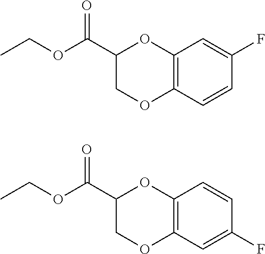

The desired product was prepared by substituting 4-fluorocatechol (500 mg) for 4,5-dichlorocatechol in Example 23D to give a regioisomeric mixture of its benzodioxane derivative in 40% yield (350 mg) as a colorless solid. ¹H-NMR (MeOH-d₄, 400 MHz) δ 1.26 (t, 7.2 Hz, 3H), 4.19-4.34 (m, 3H), 4.41-4.47 (m, 1H), 4.93-5.00 (m, 1H), 6.55-6.65 (m, 1.3H), 6.67-6.73 (m, 0.7H), 6.77-6.83 (m, 0.7H), 6.88-6.94 (m, 0.3H). Single peak by HPLC.

### 28B. 7-fluoro-2,3-dihydrobenzo[b][1,4]dioxine-2-carboxylic acid and 6-fluoro-2,3-dihydrobenzo[b][1,4]dioxine-2-carboxylic acid

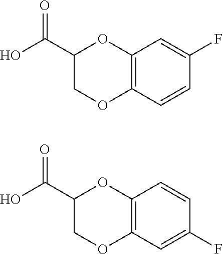

The desired product was prepared by substituting Example 28A (350 mg) for Example 23D in Example 23E to give the desired carboxylic acid mixture in 99% yield (305 mg) as a colorless solid. LCMS (found 197.0, M− calculated for C₉H₆FO₄: 197.0). Single peak by HPLC.

### 28C. 2-(7-fluoro-2,3-dihydrobenzo[b][1,4]dioxin-2-yl)-5-(pyridin-4-yl)-1H-benzo[d]imidazole, trifluoroacetic acid salt and 2-(6-fluoro-2,3-dihydrobenzo[b][1,4]dioxin-2-yl)-5-(pyridin-4-yl)-1H-benzo[d]imidazole, trifluoroacetic acid salt

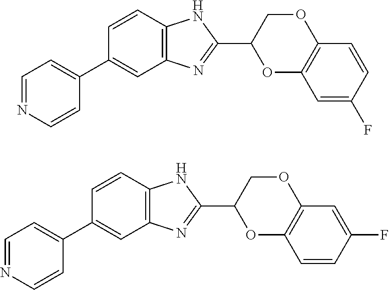

The desired product was prepared by substituting Example 28B (34 mg) for Example 23E in Example 23F to give the desired benzimidazoles in 71% yield (57 mg). ¹H-NMR (MeOH-d₄, 400 MHz) δ 4.46-4.56 (m, 1H), 4.65-4.74 (m, 1H), 5.62-5.72 (m, 1H), 7.81-7.85 (m, 1H), 7.92-7.98 (m, 1H), 8.25-8.28 (m, 1H), 8.42 (d, 5.6 Hz, 2H), 8.83 (d, 5.6 Hz, 2H). LC-MS (found 348.1, MH+ calculated for C₂₀H₁₅FN₃O₂: 348.1). Single peak by HPLC.

### Example 29

### 2-(2,3-dihydrobenzo[b][1,4]dioxin-2-yl)-1-phenyl-6-(pyridin-4-yl)-1H-benzo[d]imidazole

### 29A. 5-bromo-N1-phenylbenzene-1,2-diamine

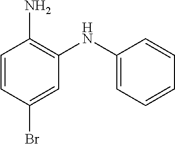

2-fluoro-4-bromo-1-nitrobenzene (100 mg, 0.455 mmol), potassium carbonate (1.1 eq.), and aniline (1.0 equiv.) were combined in DMSO. The reaction mixture was stirred at room temperature until HPLC indicated complete consumption of the 2-fluoro-4-bromonitrobenzene (30 min-48 hr), in this case 48 hours. The reaction mixture was poured into water and the resulting precipitate was collected by filtration. The precipitate was washed with water to give the desired product as an orange solid (107 mg, 80% yield). Single peak by HPLC. The nitro intermediate was treated with SnCl₂.2H₂O as in Example 9 to give the desired diamine product, which was used without further purification.

### 29B. 6-bromo-2-(2,3-dihydrobenzo[b][1,4]dioxin-2-yl)-1-phenyl-1H-benzo[d]imidazole

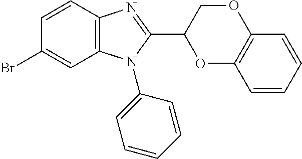

A solution of Example 29A (0.373 mmol, 1.0 equiv.) and 1,4-benzodioxan-2-carboxylic acid (0.373 mmol, 1.0 equiv.) in DMF (2 mL) was treated with HATU (1.1 equiv.) followed by DIEA (1.1 equiv.). The reaction was stirred for 1-6 hours and then poured into distilled water. The aqueous phase was extracted 2× with DCM. The organic layers were combined, washed 3× with saturated sodium bicarbonate solution, dried with sodium sulfate, and concentrated to give the amide intermediate. Presence of the amide intermediate was confirmed by LC-MS. This crude product was dissolved in glacial acetic acid and heated at 60-65° C. for 1-24 hours. Upon complete consumption of the amide intermediate, indicated by LC-MS, the reaction was concentrated in vacuo. The residue was taken up in water which was then neutralized with saturated sodium bicarbonate. The aqueous solution was then extracted 3× with DCM. The organic layers were combined, dried with sodium sulfate, and concentrated to give the crude benzimidazole product, which was purified by silica gel chromatography (hexanes:EtOAc gradient) to give the desired product as a colorless solid (116 mg, 76% yield). LC-MS (found 407.0, 409.0, MH+ calculated for C₂₁H₁₆BrNO₂: 407.0, 409.0). Single peak by HPLC.

### 29C. 2-(2,3-dihydrobenzo[b][1,4]dioxin-2-yl)-1-phenyl-6-(pyridin-4-yl)-1H-benzo[d]imidazole

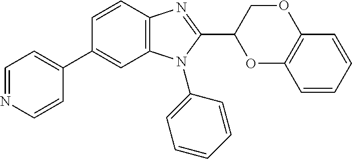

Example 29B (0.111 mmol, 1.0 equiv.), 4-pyridine boronic acid (1.25 equiv.), and sodium carbonate (750 mg, 3.0 equiv.) in a microwave pressure vial were put under an argon atmosphere. Tetrakis(triphenylphosphino)palladium(0) (0.05 equiv.) was added and the vial was sealed. The contents were dissolved in 15 mL degassed 2:1 1,2-dimethoxyethane:water and heated on a microwave reactor for 30 minutes at 100° C. The solvent was removed in vacuo and the residue was taken up in equal portions DCM and water. The layers were separated and the aqueous phase was washed 2× with DCM. Organic layers were combined, dried with sodium sulfate, concentrated, and purified by silica gel chromatography (DCM:MeOH gradient) to give the desired product as a colorless solid (33 mg, 73% yield). LC-MS (found 406.2, MH+ calculated for C₂₆H₂₀N₃O₂: 406.2). ¹H-NMR (MeOH-d₄, 400 MHz) δ 4.58-4.67 (m, 2H), 5.30 (dd, 3.6 Hz, 7.8 Hz, 1H), 6.73-6.89 (m, 4H), 7.50-7.73 (m, 8H), 7.77 (dd, 1.6 Hz, 8.8 Hz, 1H), 7.91 (d, 8.8 Hz, 1H), 8.54 (d, 8.8 Hz, 2H). Single peak by HPLC.

### Example 30

### 1-benzyl-2-(2,3-dihydrobenzo[b][1,4]dioxin-2-yl)-6-(pyridin-4-yl)-1H-benzo[d]imidazole

### 30A. N1-benzyl-5-bromobenzene-1,2-diamine

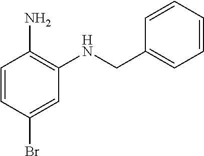

The desired product was prepared by substituting benzylamine for aniline in Example 29A, the substitution reaction run for 1.5 hours to give 125 mg of the desired product as a yellow solid (89% yield). ¹H-NMR (DMSO-d₆, 400 MHz) δ 4.63 (d, 6.0 Hz, 2H), 6.81 (dd, 2.2 Hz, 9.0 Hz, 1H), 7.09 (d, 2.2 Hz, 1H), 7.22-7.30 (m, 1H), 7.32-7.38 (m, 4H), 7.99 (d, 9.2 Hz, 1H), 8.70 (t, 6.0 Hz, 1H). Single peak by HPLC. The nitro intermediate was treated with SnCl₂.2H₂O as in Example 9 to give the desired diamine product, which was used without further purification.

### 30B. 1-benzyl-6-bromo-2-(2,3-dihydrobenzo[b][1,4]dioxin-2-yl)-1H-benzo[d]imidazole

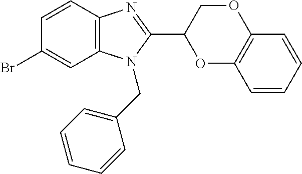

The desired product was prepared by substituting Example 30A (0.859 mmol) for Example 29A in Example 29B, and scaling appropriately. Purification by silica gel chromatography (hexanes:EtOAc gradient) gave 303 mg of the desired product as a colorless solid (84% yield). LCMS (found 421.1, 423.1, MH+ calculated for C₂₂H₁₈BrN₂O₂: 421.0, 423.0). Single peak by HPLC.

### 30C. 1-benzyl-2-(2,3-dihydrobenzo[b][1,4]dioxin-2-yl)-6-(pyridin-4-yl)-1H-benzo[d]imidazole

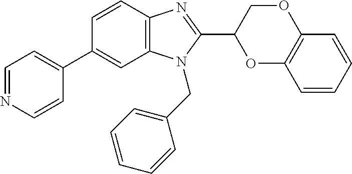

The desired product was prepared by substituting Example 30B (0.119 mmol) for Example 29B in Example 29C, and scaling appropriately. Purification by silica gel chromatography (DCM:MeOH gradient) gave 41 mg of the desired product as a colorless solid (82% yield). LC-MS (found 420.2, MH+ calculated for C₂₇H₂₂N₃O₂: 420.2). ¹H-NMR (MeOH-d₄, 400 MHz) δ 4.55-4.70 (m, 2H), 5.56-5.63 (m, 1H), 5.70-5.90 (m, 2H), 6.70-6.95 (m, 4H), 7.20-7.91 (m, 10H), 8.52-8.61 (m, 2H).

### Example 31

### 2-(2,3-dihydrobenzo[b][1,4]dioxin-2-yl)-1-phenethyl-6-(pyridin-4-yl)-1H-benzo[d]imidazole

### 31A. 5-bromo-N1-phenethylbenzene-1,2-diamine

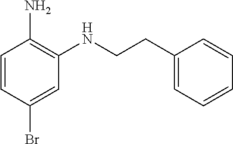

The desired product was prepared by substituting phenethylamine (1.14 mmol) for aniline in Example 29A, and scaling appropriately, the substitution reaction run for 2 hours to give 203 mg of the desired product as a yellow-orange solid (55% yield). ¹H-NMR (DMSO-d₆, 400 MHz) δ 2.93 (t, 7.2 Hz, 2H), 3.56-3.65 (m, 2H), 6.81 (dd, 2.0 Hz, 9.2 Hz, 1H), 7.15-7.34 (m, 6H), 7.95 (d, 9.2 Hz, 1H), 8.13 (t, 6.0 Hz, 1H). Single peak by HPLC. The nitro intermediate was treated with SnCl₂.2H₂O as in Example 9 to give the desired diamine product, which was used without further purification.

### 31B. 6-bromo-2-(2,3-dihydrobenzo[b][1,4]dioxin-2-yl)-1-phenethyl-1H-benzo[d]imidazole

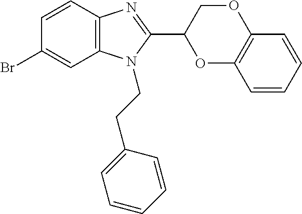

The desired product was prepared by substituting Example 31A (0.326 mmol) for Example 29A in Example 29B, and scaling appropriately. Purification by silica gel chromatography (hexanes:EtOAc gradient) gave 75 mg of the desired product as a colorless solid (53% yield). LC-MS (found 435.1, 437.1, MH+ calculated for C₂₃H₂₀BrN₂O₂: 435.1, 437.1). Single peak by HPLC.

### 31C. 2-(2,3-dihydrobenzo[b][1,4]dioxin-2-yl)-1-phenethyl-6-(pyridin-4-yl)-1H-benzo[d]imidazole

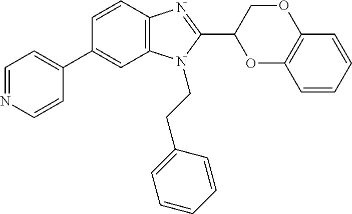

The desired product was prepared by substituting Example 31B (0.129 mmol) for Example 29B in Example 29C, and scaling appropriately. Purification by silica gel chromatography (DCM:MeOH gradient) gave 44 mg of the desired product as a colorless solid (79% yield). LC-MS (found 434.2, MH+ calculated for C₂₈H₂₄N₃O₂: 434.2). ¹H-NMR (MeOH-d₄, 400 MHz) δ 3.20-3.27 (m, 2H), 4.26 (dd, 2.4 Hz, 7.6 Hz, 1H), 4.45 (dd, 4.0 Hz, 11.6 Hz, 1H), 4.65-4.80 (m, 2H), 5.05 (dd, 2.4 Hz, 8.0 Hz, 1H), 6.82-7.01 (m, 7H), 7.13-7.20 (m, 3H), 7.60-7.78 (m, 6H), 8.55-8.60 (m, 2H). Single peak by HPLC.

### Example 32

### 1-cyclohexyl-2-(2,3-dihydrobenzo[b][1,4]dioxin-2-yl)-6-(pyridin-4-yl)-1H-benzo[d]imidazole

### 32A. 5-bromo-N1-cyclohexylbenzene-1,2-diamine

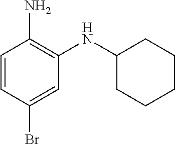

The desired product was prepared by substituting cyclohexylamine (1.14 mmol) for aniline in Example 29A, and scaling appropriately, the substitution reaction run for 30 min. to give 206 mg of the desired product as a yellow solid (61% yield). ¹H-NMR (DMSO-d₆, 400 MHz) δ 1.16-1.62 (m, 6H), 1.63-1.73 (m, 2H), 1.87-1.97 (m, 2H), 3.64-3.75 (m, 1H), 6.80 (dd, 2.0 Hz, 8.8 Hz, 1H), 7.31 (d, 2.0 Hz, 1H), 7.96 (d, 8.8 Hz, 1H), 8.00 (d, 8.0 Hz, 1H). Single peak by HPLC. The nitro intermediate was treated with SnCl₂.2H₂O as in Example 9 to give the desired diamine product, which was used without further purification.

### 32B. 6-bromo-1-cyclohexyl-2-(2,3-dihydrobenzo[b][1,4]dioxin-2-yl)-1H-benzo[c]imidazole

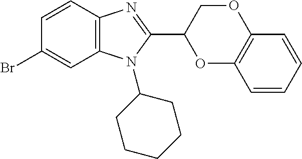

The desired product was prepared by substituting Example 32A (0.344 mmol) for Example 29A in Example 29B, and scaling appropriately. Purification by silica gel chromatography (hexanes:EtOAc gradient) gave 50 mg of the desired product as a colorless solid (35% yield). LCMS (found 413.1, 415.1, MH+ calculated for C₂₁H₂₁BrN₂O₂: 413.1, 415.1). Single peak by HPLC.

### 32C. 1-cyclohexyl-2-(2,3-dihydrobenzo[b][1,4]dioxin-2-yl)-6-(pyridin-4-yl)-1H-benzo[d]imidazole

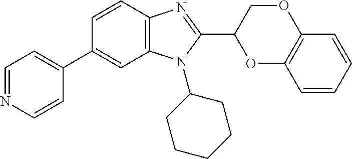

The desired product was prepared by substituting Example 32B (0.121 mmol) for Example 29B in Example 29C, and scaling appropriately. Purification by silica gel chromatography (DCM:MeOH gradient) gave 41 mg of the desired product as a colorless solid (82% yield). LC-MS (found 412.2, MH+ calculated for C₂₆H₂₆N₃O₂: 412.2). ¹H-NMR (MeOH-d₄, 400 MHz) δ 1.43-1.68 (m, 3H), 1.77-1.87 (m, 1H), 1.95-2.20 (m, 4H), 2.30-2.50 (m, 2H), 4.65-4.84 (m, 3H), 5.70 (dd, 2.8 Hz, 7.6 Hz, 1H), 6.83-6.98 (m, 4H), 7.45-7.84 (m, 9H), 8.07 (s, 1H), 8.55-8.64 (m, 2H). Single peak by HPLC.

### Example 33

### 2-(2,3-dihydrobenzo[b][1,4]dioxin-2-yl)-1-(2-morpholinoethyl)-6-(pyridin-4-yl)-1H-benzo[d]imidazole

### 33A. 5-bromo-N1-(2-morpholinoethyl)benzene-1,2-diamine

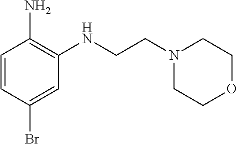

The desired product was prepared by substituting 2-(morpholino)ethylamine (1.14 mmol) for aniline in Example 29A, and scaling appropriately, the substitution reaction run for 2 hours to give 203 mg of the desired product as an orange solid (54% yield). ¹H-NMR (DMSO-d₆, 400 MHz) δ 2.43 (bs, 4H), 2.60 (t, 6.0 Hz, 2H), 3.36-3.44 (m, 2H), 3.55-3.62 (m, 4H), 6.82 (dd, 2.0 Hz, 9.2 Hz, 1H), 7.25 (d, 2.0 Hz, 1H), 7.97 (d, 9.2 Hz, 1H), 8.46 (t, 4.4 Hz, 1H). Single peak by HPLC. The nitro intermediate was treated with SnCl₂.2H₂O as in Example 9 to give the desired diamine product, which was used without further purification.

### 33B. 6-bromo-2-(2,3-dihydrobenzo[b][1,4]dioxin-2-yl)-1-(2-morpholinoethyl)-1H-benzo[d]imidazole

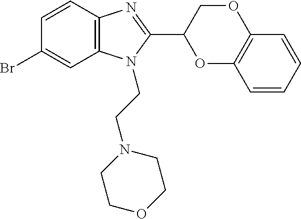

The desired product was prepared by substituting Example 33A (0.360 mmol) for Example 29A in Example 29B, and scaling appropriately. Purification by silica gel chromatography (hexanes:EtOAc gradient) gave 90 mg of the desired product as a colorless solid (56% yield). LC-MS (found 435.1, 437.1, MH+ calculated for C₂₃H₁₉BrN₂O₂: 435.1, 437.1). Single peak by HPLC.

### 33C. 2-(2,3-dihydrobenzo[b][1,4]dioxin-2-yl)-1-(2-morpholinoethyl)-6-(pyridin-4-yl)-1H-benzo[d]imidazole

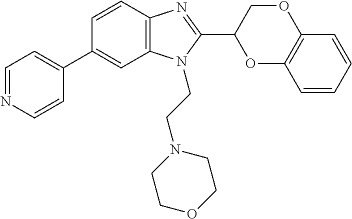

The desired product was prepared by substituting Example 33B (0.133 mmol) for Example 29B in Example 29C, and scaling appropriately. Purification by silica gel chromatography (DCM:MeOH gradient) gave 33 mg of the desired product as a colorless solid (56% yield). LC-MS (found 443.2, MH+ calculated for C₂₆H₂₇N₄O₃: 443.2). ¹H-NMR (MeOH-d₄, 400 MHz) δ 2.42-2.50 (m, 2H), 2.56-2.66 (m, 2H), 2.80-2.95 (m, 2H), 3.57-3.65 (m, 4H), 4.57-4.85 (m, 4H), 4.77 (dd, 2.4 Hz, 8.0 Hz, 1H), 6.84-7.00 (m, 4H), 7.70-7.77 (m, 1H), 7.80-7.86 (m, 3H), 8.07 (s, 1H), 8.60 (d, 4.4 Hz, 2H). Single peak by HPLC.

### Example 34

### 2-(2,3-dihydrobenzo[b][1,4]dioxin-2-yl)-1-(2-methoxyethyl)-6-(pyridin-4-yl)-1H-benzo[d]imidazole

### 34A. 5-bromo-N1-(2-methoxyethyl)benzene-1,2-diamine

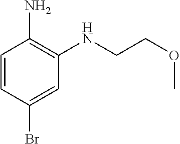

The desired product was prepared by substituting 2-methoxyethylamine (1.82 mmol) for aniline in Example 29A, and scaling appropriately, the substitution reaction run for 2 hours to give 481 mg of the desired product as a yellow solid (97% yield). ¹H-NMR (DMSO-d₆, 400 MHz) δ 3.30 (s, 3H), 3.48-3.52 (m, 3H), 6.83 (dd, 2.0 Hz, 9.2 Hz, 1H), 7.30 (d, 2.0 Hz, 1H), 7.97 (d, 8.8 Hz, 1H), 8.20 (t, 4.8 Hz, 1H). Single peak by HPLC. The nitro intermediate was treated with SnCl₂.2H₂O as in Example 9 to give the desired diamine product, which was used without further purification.

### 34B. 6-bromo-2-(2,3-dihydrobenzo[b][1,4]dioxin-2-yl)-1-(2-methoxyethyl)-1H-benzo[d]imidazole

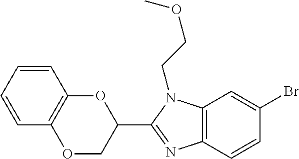

The desired product was prepared by substituting Example 34A (1.63 mmol) for Example 29A in Example 29B, and scaling appropriately. Purification by silica gel chromatography (DCM:EtOAc gradient) gave 445 mg of the desired product as a colorless solid (70% yield). ¹H-NMR (DMSO-d₆, 400 MHz) δ 3.21 (s, 3H), 3.63-3.73 (m, 2H), 4.54-4.65 (m, 3H), 4.71 (dd, 2.8 Hz, 7.6 Hz, 1H), 5.70 (dd, 2.6 Hz, 7.6 Hz, 1H), 6.83-6.97 (m, 4H), 7.37 (dd, 2.0 Hz, 8.4 Hz, 1H), 7.62 (d, 8.4 Hz, 1H), 7.76 (d, 1.6 Hz, 1H). Single peak by HPLC.

### 34C. 2-(2,3-dihydrobenzo[b][1,4]dioxin-2-yl)-1-(2-methoxyethyl)-6-(pyridin-4-yl)-1H-benzo[d]imidazole

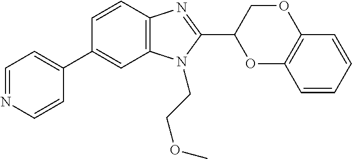

The desired product was prepared by substituting Example 34B (0.147 mmol) for Example 29B in Example 29C, and scaling appropriately. Purification by silica gel chromatography (DCM:MeOH gradient) gave 25 mg of the desired product as a colorless solid (44% yield). LCMS (found 388.2, MH+ calculated for C₂₃H₂₂N₃O₃: 388.2). Single peak by HPLC.

### Example 35

### 2-(2,3-dihydrobenzo[b][1,4]dioxin-2-yl)-1-(2-methoxyethyl)-6-(1H-pyrazol-4-yl)-1H-benzo[d]imidazole

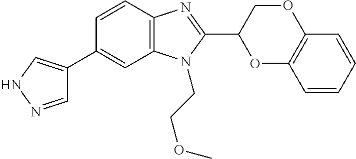

The desired product was prepared by substituting 4-(4,4,5,5-tetramethyl-1,3,2-dioxaborolan-2-yl)-1H-pyrazole for 4-pyridineboronic acid in Example 34C using Example 34B (0.129 mmol). Purification by silica gel chromatography (DCM:MeOH gradient) gave 35 mg of the product as a colorless solid (71% yield). LCMS (found 377.1, MH+ calculated for C₂₁H₂₁N₄O₃: 377.1). Single peak by HPLC.

### Example 36

### 1-(cyclopropylmethyl)-2-(2,3-dihydrobenzo[b][1,4]dioxin-2-yl)-6-(pyridin-4-yl)-1H-benzo[d]imidazole

### 36A. 5-bromo-N1-(cyclopropylmethyl)benzene-1,2-diamine

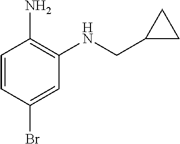

The desired product was prepared by substituting cyclopropylmethylamine (1.82 mmol) for aniline in Example 29A, and scaling appropriately, the substitution reaction run for 2 hours to give 450 mg of the desired product as an orange solid (91% yield). ¹H-NMR (DMSO-d₆, 400 MHz) δ 0.25-0.35 (m, 2H), 0.47-0.58 (m, 2H), 1.08-1.20 (m, 1H), 3.22 (dd, 5.6 Hz, 7.2 Hz, 2H), 6.82 (dd, 2.0 Hz, 9.2 Hz, 1H), 7.24 (d, 2.0 Hz, 1H), 7.97 (d, 9.2 Hz, 1H), 8.18 (t, 5.6 Hz, 1H). Single peak by HPLC. The nitro intermediate was treated with SnCl₂.2H₂O as in Example 9 to give the desired diamine product, which was used without further purification.

### 36B. 6-bromo-1-(cyclopropylmethyl)-2-(2,3-dihydrobenzo[b][1,4]dioxin-2-yl)-1H-benzo[d]imidazole

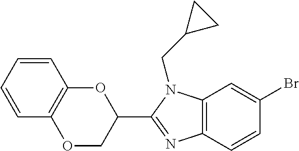

The desired product was prepared by substituting Example 36A (1.53 mmol) for Example 29A in Example 29B, and scaling appropriately. Purification by silica gel chromatography (DCM:EtOAc gradient) gave 436 mg of the desired product as a colorless solid (74% yield). ¹H-NMR (DMSO-d₆, 400 MHz) δ 0.43-0.57 (m, 4H), 1.32-1.43 (m, 1H), 4.25-4.41 (m, 2H), 4.60 (dd, 8.0 Hz, 11.6 Hz, 1H), 4.76 (dd, 2.4 Hz, 7.6 Hz, 1H), 5.72 (dd, 2.4 Hz, 8.0 Hz, 1H), 6.82-6.99 (m, 4H), 7.38 (dd, 2.8 Hz, 8.4 Hz, 1H), 7.64 (d, 8.4 Hz, 1H), 8.04 (d, 1.6 Hz, 1H). Single peak by HPLC.

### 36C. 1-(cyclopropylmethyl)-2-(2,3-dihydrobenzo[b][1,4]dioxin-2-yl)-6-(pyridin-4-yl)-1H-benzo[d]imidazole

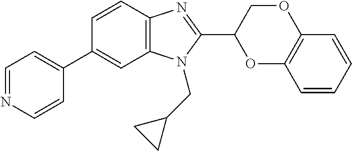

The desired product was prepared by substituting Example 36B (0.166 mmol) for Example 29B in Example 29C, and scaling appropriately. Purification by silica gel chromatography (DCM:MeOH gradient) gave 15 mg of the desired product as a colorless solid (23% yield). LC-MS (found 384.2, MH+ calculated for C₂₄H₂₂N₃O₂: 384.2). Single peak by HPLC.

### Example 37

### 2-(2,3-dihydrobenzo[b][1,4]dioxin-2-yl)-6-(pyridin-4-yl)-1-(tetrahydro-2H-pyran-4-yl)-1H-benzo[d]imidazole

### 37A. 5-bromo-N1-(tetrahydro-2H-pyran-4-yl)benzene-1,2-diamine

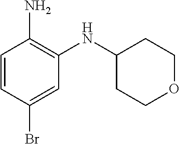

The desired product was prepared by substituting tetrahydro-2H-pyran-4-amine (1.82 mmol) for aniline in Example 29A, and scaling appropriately, the substitution reaction run for 2 hours 505 mg of the desired product as an orange solid (93% yield). ¹H-NMR (DMSO-d₆, 400 MHz) δ 1.49-1.62 (m, 2H), 1.85-1.97 (m, 2H), 3.48 (td, 2.0 Hz, 7.6 Hz, 2H), 3.80-3.98 (m, 3H), 6.83 (dd, 2.0 Hz, 9.2 Hz, 1H), 7.41 (d, 2.0 Hz, 1H), 7.93 (d, 7.6 Hz, 1H), 7.98 (d, 8.8 Hz, 1H). Single peak by HPLC. The nitro intermediate was treated with SnCl₂.2H₂O as in Example 9 to give the desired diamine product, which was used without further purification.

### 37B. 6-bromo-2-(2,3-dihydrobenzo[b][1,4]dioxin-2-yl)-1-(tetrahydro-2H-pyran-4-yl)-1H-benzo[d]imidazole

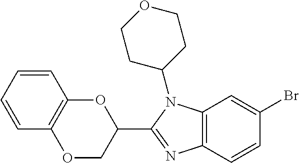

The desired product was prepared by substituting Example 37A (1.10 mmol) for Example 29A in Example 29B, and scaling appropriately. Purification by silica gel chromatography (DCM:EtOAc gradient) gave 101 mg of the desired product as a colorless solid (22% yield). LC-MS (found 415.0, 417.0, MH+ calculated for C₂₀H₂₀BrN₂O₃: 415.0, 417.0). Single peak by HPLC.

### 37C. 2-(2,3-dihydrobenzo[b][1,4]dioxin-2-yl)-6-(pyridin-4-yl)-1-(tetrahydro-2H-pyran-4-yl)-1H-benzo[d]imidazole

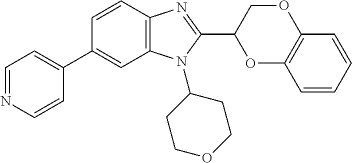

The desired product was prepared by substituting Example 37B (0.113 mmol) for Example 29B in Example 29C, and scaling appropriately. Purification by silica gel chromatography (DCM:MeOH gradient) gave 25 mg of the desired product as a colorless solid (53% yield). LC-MS (found 414.2, MH+ calculated for C₂₅H₂₄N₃O₃: 414.2). Single peak by HPLC.

### Example 38

### 2-(2,3-dihydrobenzo[b][1,4]dioxin-2-yl)-4-fluoro-(1H-pyrazol-4-yl)-1H-benzo[d]imidazole

### 38A. 6-bromo-2-(2,3-dihydrobenzo[b][1,4]dioxin-2-yl)-4-fluoro-1H-benzo[d]imidazole

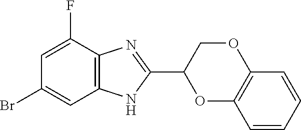

A solution of 5-bromo-3-fluorobenzene-1,2-diamine (0.200 g, 0.976 mmol) and 1,4-benzodioxane-2-carboxylic acid (0.176 g, 1.0 equiv.) in 3 mL DMF was treated with HATU (0.408 g, 1.1 equiv.) followed by DIEA (0.187 mL, 1.1 equiv.). The reaction mixture was stirred for 1 hour and then poured into 10 mL distilled water. The aqueous phase was extracted 2× with DCM. The organic layers were combined, washed 3× with saturated sodium bicarbonate solution, dried with sodium sulfate, and concentrated to give an oil. Presence of the amide intermediate was confirmed by LC-MS. This crude product was dissolved in 10 mL glacial acetic acid and heated at 60-65° C. for two hours. Disappearance of amide was indicated by LC-MS and the reaction mixture was concentrated in vacuo. The residue was taken up in 20 mL water which was neutralized with saturated sodium bicarbonate. The aqueous solution was then extracted 3× with DCM. The organic layers were combined, dried with sodium sulfate, and concentrated to give the desired benzimidazole product (269 mg, 79% yield). LCMS (found 349.0, 351.0, MH+ calculated for C₁₅H₁₁BrFN₂O₂: 349.0, 351.0). Single peak by HPLC.

### 38B. 2-(2,3-dihydrobenzo[b][1,4]dioxin-2-yl)-4-fluoro-6-(1H-pyrazol-4-yl)-1H-benzo[d]imidazole

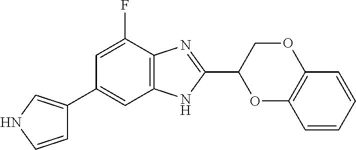

Example 38A (60 mg, 0.172 mmol) in 2 mL of a degassed 2:1 1,2-dimethoxyethane:water solution was treated with 4-(4,4,5,5-tetramethyl-1,3,2-dioxaborolan-2-yl)-1H-pyrazole (pyrazole-4-pinacolboronate) (40 mg, 1.2 equiv.), sodium carbonate (55 mg, 3.0 equiv.), and a catalytic amount of tetrakis(triphenylphosphino) palladium(0). The reaction was heated at 120° C. for 30 min. in a microwave. The solvent was removed and the residue was purified by preparative HPLC to give 9 mg of the desired product as a colorless solid. LC-MS (found 337.1, MH+ calculated for C₁₈H₁₄₁FN₄O₂: 337.1). Single peak by HPLC.

### Example 39

### 2-(chroman-3-yl)-5-(pyridin-4-yl)-1H-benzo[d]imidazole

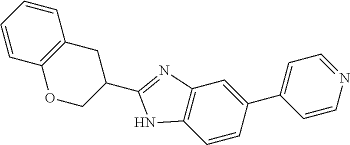

Example 23C (0.324 mmol) was added to a room temperature solution of chroman-3-carboxylic acid (0.324 mmol), HATU (0.356 mmol), and Et₃N (0.648 mmol) in DMF (1.2 mL). The resulting mixture was stirred at room temperature for 60 minutes. At this time the solution was sealed in a microwave pressure tube and heated to 160° C. in a microwave for 50 minutes. After cooling, the reaction was diluted with water and the resulting precipitate was collected by filtration to give 20 mg of the desired cyclized product. LC-MS: single peak at 254 nm, MH⁺ calcd. for C₂₁H₁₇N₃O: 328, obtained 328. HPLC: single peak by analytical HPLC. ¹H-NMR (MeOD- d₄, 400 MHz): 8.59 (2H, m), 7.95 (1H, s), 7.77 (2H, m), 7.68 (2H, m), 7.13 (2H, m), 6.87 (2H, m), 4.61 (1H, m), 4.34 (1H, dd, J=9.5 Hz, 10.7 Hz), 3.66 (1H, m), 3.34 (2H, m).

### Example 40

### 2-(chroman-3-yl)-5-(1H-pyrazol-4-yl)-1H-benzo[d]imidazole, trifluoroacetic acid salt

### 40A. 5-bromo-2-(chroman-3-yl)-1H-benzo[d]imidazole

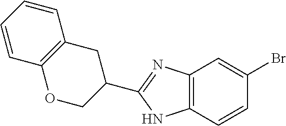

4-bromobenzene-1,2-diamine (420 mg, 1.0 equiv.) was added to a room temperature solution of chroman-3-carboxylic acid (400 mg, 1.0 equiv.), HATU (1.2 equiv), and Et₃N (2.0 equiv.) in DMF (3.0 mL/mmol). The resulting mixture was stirred at room temperature for 60 minutes and then concentrated in vacuo. The residue was dissolved in AcOH (3.0 mL/mmol) and warmed to 65° C. until the cyclodehydration was complete. The material was then concentrated in vacuo and purified on silica gel (CH₂Cl₂/EtOAc) to give the desired arylbromide (80%). LC-MS: single peak at 254 nm, MH⁺ calcd. for C₁₆H₁₃BrN₂O: 329, obtained 329.

### 40B. 2-(chroman-3-yl)-5-(1H-pyrazol-4-yl)-1H-benzo[d]imidazole, trifluoroacetic acid salt

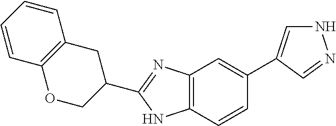

Example 40A (65.0 mg, 1.0 equiv) was combined with 4-(4,4,5,5-tetramethyl-1,3,2-dioxaborolan-2-yl)-1H-pyrazole (pyrazole-4-pinacolboronate) (1.3 equiv.), Na₂CO₃ (3.0 equiv.) and PdCl₂(PPh₃)₂ (0.1 equiv.) under streaming argon. Aqueous dioxane (20%, 10 mL/mmol) was then added and the solution was sparged with argon for 10 minutes. The solution was then heated to 120° C. in a microwave until complete. Upon completion, the material was purified via preparative HPLC (gradient; mobile phase: solvent A: 0.1% TFA in water, solvent B: CH₃CN) to obtain the desired product as the TFA salt (7.47 mg). LC-MS: single peak at 254 nm, MH⁺ calcd. for C₁₉H₁₆N₄O: 317, obtained 317. HPLC: single peak by analytical HPLC. ¹H-NMR (MeOD- d₄, 400 MHz): 8.16 (2H, s), 7.85 (1H, s), 7.69 (2H, s), 7.21 (1H, d, J=7.4 Hz), 7.13 (1H, t, J=7.8 Hz), 6.93 (1H, t, J=7.5 Hz), 6.84 (1H, d, J=8.2 Hz), 4.62 (1H, m), 4.41 (1H, m), 3.85 (1H, m), 3.35 (2H, d, J=7.0 Hz).

### Example 41

### 2-(chroman-3-yl)-5-(5-methyl-1H-pyrazol-4-yl)-1H-benzo[d]imidazole, trifluoroacetic acid salt

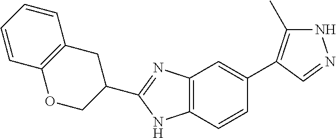

The desired product was prepared by substituting 5-methyl-4-(4,4,5,5-tetramethyl-1,3,2-dioxaborolan-2-yl)-1H-pyrazole (available from Focus Synthesis LLC, San Diego, Calif.) for 4-(4,4,5,5-tetramethyl-1,3,2-dioxaborolan-2-yl)-1H-pyrazole (pyrazole-4-pinacolboronate in Example 40B (4.27 mg). LC-MS: single peak at 254 nm, MH⁺ calcd. for C₂₀H₁₈N₄O: 331, obtained 331. HPLC: single peak by analytical HPLC.

### Example 42

### 4-(2-chroman-3-yl)-1H-benzo[d]imidazol-5-yl)-7H-pyrrolo[2,3-d]pyrimidine trifluoroacetic acid salt

### 42A. 2-(chroman-3-yl)-5-(4,4,5,5-tetramethyl-1,3,2-dioxaborolan-2-yl)-1H-benzo[d]imidazole

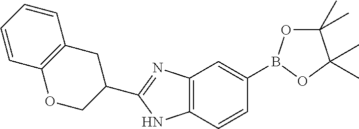

Example 40A (360 mg, 1.0 equiv.) was combined with bis(pinacolato)diboron (2.5 equiv.), KOAc (5.0 equiv.) and PdCl₂(dppf) (0.1 equiv.) under streaming argon in a microwave pressure tube. Dioxane (10 mL/mmol) was then added and the solution was sparged with argon for 10 minutes. The solution was then heated to 100° C. in a microwave until the conversion was complete. Upon completion, the reaction mixture was diluted with EtOAc and washed with brine. The aqueous fraction was extracted with additional EtOAc and the combined organic portions were dried over MgSO₄ and concentrated to give the desired product arylboronate (85%). LC-MS: single peak at 254 nm, MH⁺ calcd. for C₂₂H₂₅BN₂O₃: 377, obtained 377.

### 42B. 4-(2-(chroman-3-yl)-1H-benzo[d]imidazol-5-yl)-7H-pyrrolo[2,3-d]pyrimidine, trifluoroacetic acid salt

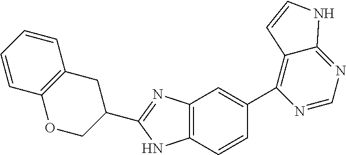

Example 42A (67.0 mg, 1.0 equiv.) was then combined with 4-chloro-7H-pyrrolo[2,3-d]pyrimidine (1.0 equiv.), Na₂CO₃ (3.0 equiv.) and PdCl₂(PPh₃)₂ (0.1 equiv.) under streaming argon. Aqueous dioxane (20%, 10 mL/mmol) was then added and the solution was sparged with argon for 10 minutes. The solution was then heated to 120° C. in a microwave until complete. Upon completion, the solution was purified via preparative HPLC (gradient; mobile phase: solvent A: 0.1% TFA in water, solvent B: CH₃CN) to obtain the desired product as the TFA salt (17.9 mg). LC-MS: single peak at 254 nm, MH⁺ calcd. for C₂₂H₁₇N₅O: 368, obtained 368. HPLC: single peak by analytical HPLC.

### Example 43

### 2-(chroman-3-yl)-5-(1H-pyrrolo[2,3-b]pyridin-4-yl)-1H-benzo[d]imidazole, trifluoroacetic acid salt

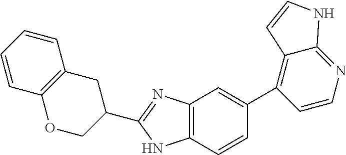

The desired product was prepared by substituting 4-chloro-1H-pyrrolo[2,3-b]pyridine for 4-chloro-7H-pyrrolo[2,3-d]pyrimidine in Example 42B (16.8 mg). LC-MS: single peak at 254 nm, MH⁺ calcd. for C₂₃H₁₈N₄O: 367, obtained 367. HPLC: single peak by analytical HPLC. ¹H-NMR (DMSO-d₆, 400 MHz): 8.34 (1H, d, J=5.0 Hz), 8.01 (1H, s), 7.84 (1H, d, J=8.6 Hz), 7.77 (1H, d, J=8.1 Hz), 7.60 (1H, m), 7.29 (1H, d, J=5.1 Hz), 7.15 (1H, m), 6.93 (1H, m), 6.85 (1H, d, J=8.1 Hz), 6.67 (1H, m), 4.64 (1H, m), 4.41 (1H, m), 3.83 (1H, m), 3.36 (2H, m).

### Example 44

### 4-(2-(chroman-3-yl)-1H-benzo[d]imidazol-5-yl)pyrimidin-2-amine

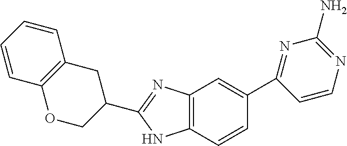

The desired product was prepared by substituting 4-chloropyrimidin-2-amine for 4-chloro-7H-pyrrolo[2,3-d]pyrimidine in Example 42B (13.3 mg). LC-MS: single peak at 254 MH⁺ calcd. for C₂₀H₁₇N₅O: 344, obtained 344. HPLC: single peak by analytical HPLC.

### Example 45

### 2-(2,3-dihydrobenzo[b][1,4]dioxin-2-yl)-6-(pyridin-4-yl)-3H-imidazo[4,5-b]pyridine

### 45A. 5-(pyridin-4-yl)pyridine-2,3-diamine

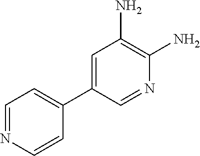

A mixture of 2,3-diamino-5-bromopyridine (470 mg, 1.0 equiv), 4-pyridine boronic acid (1.1 equiv.) and Pd(PPh₃)₄ (0.03 equiv.) in THF (10 mL) was treated with 2M Na₂CO₃ (3 equiv.) solution dropwise at 25° C. After stirring overnight at 120° C. in a sealed tube, the resulting mixture was cooled to room temperature. Solvent was removed in vacuo and water was added. The solution was extracted with EtOAc three times. The organic layers were combined, dried and concentrated in vacuo. The residue was purified by flash chromatography to give the desired product as a yellow solid (45%). LC-MS: single peak at 254 nm, MH⁺ calcd. for C₁₀H₁₀N₄: 187, obtained: 187. ¹H-NMR (DMSO-d₆, 400 MHz), δ 8.51 (dd, J=4.6, 1.6 Hz, 2H), 7.80 (d, J=2.2 Hz, 1H), 7.51 (dd, J=4.6, 1.6 Hz, 2H), 7.10 (d, J=2.2 Hz, 1H), 5.83 (s, 2H), 4.88 (s, 2H).

### 45B. 2-(2,3-dihydrobenzo[b][1,4]dioxin-2-yl)-6-(pyridin-4-yl)-3H-imidazo[4,5-b]pyridine

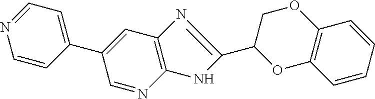

A solution of Example 45A (1 equiv.) and 1,4-benzodioxan-2-carboxylic acid (1 equiv.) in DMF (10 mL/mmol) was treated with HATU (1 equiv.) and DIEA (3 equiv.) sequentially. The resulting mixture was stirred at room temperature for 1 hour. The solution was diluted with EtOAc and washed with saturated NaHCO₃ solution. The organic layer was dried over sodium sulfate and concentrated in vacuo. The residue was dissolved in glacial acetic acid and heated to 110° C. for 5 hours. Acetic acid was evaporated and the residue was purified by preparative HPLC to give the desired product (54%). LC-MS: single peak at 254 nm, MH⁺ calcd. for C₁₉H₁₄N₄O₂: 331, obtained: 331. ¹H-NMR (DMSO-d₆, 400 MHz), δ 8.93 (d, J=2.2 Hz, 1H), 8.82 (dd, J=5.3, 1.4 Hz, 2H), 8.56 (s, 1H), 8.26 (d, J=6.6 Hz, 2H), 7.00 (m, 1H), 6.85 (m, 3H), 5.67 (dd, J=6.5, 2.7 Hz, 1H), 4.62 (dd, J=11.6, 2.7 Hz, 1H), 4.51 (dd, J=11.6, 6.6 Hz, 1H).

### Example 46

### (S)-2-(2,3-dihydrobenzo[b][1,4]dioxin-2-yl)-6-(pyridin-4-yl)-3H-imidazo[4,5-b]pyridine

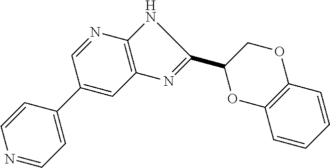

The desired product was prepared by using Example 45A (100 mg), and substituting (R)-1,4-benzodioxan-2-carboxylic acid for 1,4-benzodioxan-2-carboxylic acid in Example 45B, and scaling appropriately. Preparative HPLC gave 118 mg of the title compound (67%). LC-MS: single peak at 254 nm, MH⁺ calcd. for C₁₉H₁₄N₄O₂: 331, obtained: 331. ¹H-NMR (DMSO-d₆, 400 MHz), δ 8.93 (d, J=2.2 Hz, 1H), 8.82 (dd, J=5.3, 1.4 Hz, 2H), 8.56 (s, 1H), 7.83 (d, J=6.6 Hz, 2H), 7.00 (m, 1H), 6.85 (m, 3H), 5.67 (dd, J=6.5, 2.7 Hz, 1H), 4.62 (dd, J=11.6, 2.7 Hz, 1H), 4.51 (dd, J=11.6, 6.6 Hz, 1H).

### Example 47

### 2-(2,3-dihydronaphtho[2,3-b][1,4]dioxin-2-yl)-6-(pyridin-4-yl)-3H-imidazo[4,5-b]pyridine

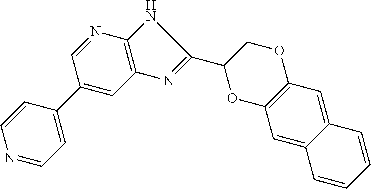

The desired product was prepared by using Example 45A (100 mg), and substituting 2,3-dihydronaphtho[2,3-b][1,4]dioxine-2-carboxylic acid for 1,4-benzodioxan-2-carboxylic acid in Example 45B, and scaling appropriately. Preparative HPLC gave 55 mg of the title compound (27%). LC-MS: single peak at 254 nm, MH⁺ calcd. for C₂₃H₁₆N₄O₂: 381, obtained: 381. ¹H-NMR (DMSO-d₆, 400 MHz), δ 8.92 (d, J=1.8 Hz, 1H), 8.78 (d, J=5.4 Hz, 2H), 8.54 (m, 1H), 8.09 (m, 2H), 7.76 (m, 2H), 7.53 (s, 1H), 7.42 (s, 1H), 7.34 (m, 2H), 5.89 (dd, J=6.3, 2.7 Hz, 1H), 4.79 (dd, J=11.7, 2.7 Hz, 1H), 4.71 (dd, J=11.7, 6.3 Hz, 1H).

### Example 48

### 2-(2,3-dihydrobenzo[b][1,4]dioxin-2-yl)-6-(1H-pyrazol-4-yl)-3H-imidazo[4,5-b]pyridine

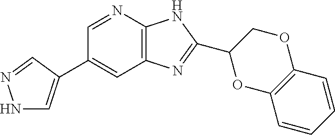

A solution of 2,3-diamino-5-bromopyridine (1 equiv.) and 1,4-benzodioxan-2-carboxylic acid (1 equiv.) in DMF (10 mL/mmol) was treated with HATU (1 equiv.) and DIEA (3 equiv.) sequentially. The resulting mixture was stirred at room temperature for 1 hour. The solution was diluted with EtOAc and washed with saturated NaHCO₃ solution. The organic layer was dried over sodium sulfate and concentrated in vacuo. The residue was dissolved in glacial acetic acid and heated to 110° C. for 8 hours. Acetic acid was evaporated and the residue was used for the next reaction without further purification (85%). Thus the crude arylbromide (1 equiv.) and N1-BOC-4-(4,4,5,5-tetramethyl-1,3,2-dioxaborolan-2-yl)-1H-pyrazole (1.5 equiv.) were dissolved in THF in a sealed tube. Pd(PPh₃)₄ (0.03 equiv.) and 2M solution of Na₂CO₃ (3 equiv.) were added sequentially. The resulting mixture was heated to 100° C. for one hour in a microwave reactor. After cooling to room temperature, the mixture was diluted with water and extracted with ethyl acetate. The organic layers were combined, dried over sodium sulfate and concentrated in vacuo. The residue thus produced was purified by preparative HPLC to give the desired product as solid (32%). LC-MS: single peak at 254 nm, MH⁺ calcd. for C₁₇H₁₃N₅O₂: 320, obtained: 320. ¹H-NMR (DMSO-d₆, 400 MHz), δ 8.64 (s, 1H), 8.12 (s, 3H), 6.96 (m, 1H), 6.83 (m, 3H), 5.58 (dd, J=6.9, 2.6 Hz, 1H), 4.61 (dd, J=11.6, 2.6 Hz, 1H), 4.46 (dd, J=11.6, 6.9 Hz, 1H).

### Example 49

### 6-bromo-2-(2,3-dihydrobenzo[b][1,4]dioxin-2-yl)-1H-imidazo[4,5-b]pyridine

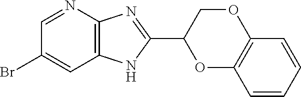

A solution of 1,4-benzodioxan-2-carboxylic acid (4.0 mmol), HATU (4.4 mmol) and Et₃N (8.0 mmol) in anhydrous DMF (11.0 mL) was stirred for 5 minutes at room temperature. To this solution was then added 5-bromo-2,3-diaminopyridine (4.0 mmol), and the resulting mixture was stirred until the full consumption of the starting material by LC-MS (1 hour). Upon completion, the solution was transferred to a microwave pressure tube containing p-TsOH (0.4 mmol) and this solution was then subjected to microwave heating at 160° C. for 90 minutes. The DMF solvent was then partially removed in vacuo, and H₂O was added to the residual solution to induce precipitation of the desired product. Filtration of the precipitate yielded 723 mg (55%) of the desired product. LC-MS: single peak at 254 nm, MH⁺ calcd. for C₁₄H₁₀BrN₃O₂: 332, obtained 332. ¹H-NMR (DMSO-d₆, 400 MHz): 8.47 (1H, s), 8.30 (1H, s), 7.04 (1H, m), 6.92 (31-1, m), 5.68 (1H, m), 4.67 (1H, dd, J=2.6 Hz, 11.7 Hz), 4.52 (1H, dd, J=6.8 Hz, 11.7 Hz).

### Example 50

### General Procedures for Alkylation of 6-bromo-2-(2,3-dihydrobenzo[b][1,4]dioxin-2-yl)-1H-imidazo[4,5-b]pyridine followed by Suzuki Coupling

### 50A. 1-alkyl-6-bromo-2-(2,3-dihydrobenzo[b][1,4]dioxin-2-yl)-1H-imidazo[4,5-b]pyridine

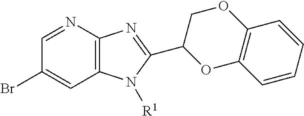

Example 49 (1.0 equiv.) is combined with Cs₂CO₃ (1.1 equiv.) in anhydrous DMF (8.0 mL/mmol) at room temperature. To this solution is added an alkylbromide R¹—Br (1.0 equiv.). The resulting solution is stirred until alkylation is complete (temperatures ranging from 25° C. to 125° C. depending on R¹—Br) as determined by LC-MS. Upon completion of the reaction the solution is diluted with EtOAc and washed with brine. The aqueous layer is twice back-extracted with additional EtOAc and the combined organic portions dried over MgSO₄. Purification on silica gel (hexane/EtOAc) gives the desired product (note: both possible N-alkylated regioisomers can be formed, but are easily separable).

### 50B. 1-alkyl-2-(2,3-dihydrobenzo[b][1,4]dioxin-2-yl)-6-(pyridin-4-yl)-1H-imidazo[4,5-b]pyridine

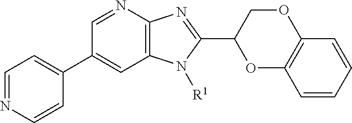

An aryl bromide prepared according to Example 50A (1.0 equiv.) is combined with 4-pyridylboronic acid (1.30 equiv.) and K₂CO₃ (1.50 equiv.) in 10% aqueous dioxane (5.0 mL/mmol) The reaction mixture is stirred in a microwave pressure tube at room temperature. To this solution is added PdCl₂(PPh₃)₂ (0.05 equiv.) and the solution is briefly sparged with argon. The reaction is subsequently heated in a microwave at 125° C. until the reaction is complete (60-240 minutes) as determined by LC-MS. The solution is then diluted with EtOAc and washed with brine. The aqueous layer is twice back-extracted with additional EtOAc and the combined organic portions are dried over MgSO₄. Purification on silica gel (hexane/EtOAc) gives the desired product.

### Example 51

### 1-benzyl-2-(2,3-dihydrobenzo[b][1,4]dioxin-2-yl)-6-(pyridin-4-yl)-1H-imidazo[4,5-b]pyridine

### 51A. 1-benzyl-6-bromo-2-(2,3-dihydrobenzo[b][1,4]dioxin-2-yl)-1H-imidazo[4,5-b]pyridine

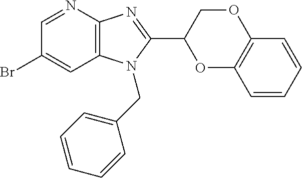

The desired product was prepared by using Example 49 (210 mg) and benzyl bromide in Example 50A to give the desired product (35.0 mg, 13%). LC-MS: single peak at 254 nm, MH⁺ calcd. for C₂₁H₁₆BrN₃O₂: 422, obtained 422. ¹H-NMR (CDCl₃, 400 MHz): 8.62 (1H, d, J=2.1 Hz), 7.74 (1H, d, J=2.1 Hz), 7.35 (3H, m), 7.14 (2H, m), 6.88 (4H, m), 5.41 (1H, dd, J=2.5 Hz, 8.4 Hz), 4.90 (1H, dd, J=2.5 Hz, 11.9 Hz), 4.71 (1H, dd, J=8.4 Hz, 11.9 Hz). The other regioisomer was also isolated as a by-product (69.0 mg, 26%).

### 51B. 1-benzyl-2-(2,3-dihydrobenzo[b][1,4]dioxin-2-yl)-6-(pyridin-4-yl)-1H-imidazo[4,5-b]pyridine

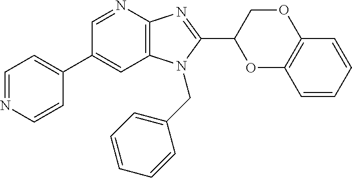

The desired product was prepared by using Example 51A (35.0 mg) in Example 50B to give the desired product (16.8 mg, 48%). LC-MS: single peak at 254 nm, MH⁺ calcd. for C₂₆H₂₀N₄O₂: 421, obtained 421. HPLC: single peak by analytical HPLC. ¹H-NMR (MeOD-d₄, 400 MHz): 8.89 (1H, d, J=2.1 Hz), 8.64 (2H, m), 8.34 (1H, d, J=2.0 Hz), 7.78 (2H, m), 7.36 (3H, m), 7.28 (2H, m), 6.88 (3H, m), 6.77 (1H, m), 5.91 (1H, d, J=16.4 Hz), 5.81 (1H, d, J=16.4 Hz), 5.66 (1H, dd, J=2.7 Hz, 7.5 Hz), 4.73 (1H, dd, J=2.7 Hz, 11.7 Hz), 4.65 (1H, dd, J=7.5 Hz, 11.7 Hz).

### Example 52

### 2-(2,3-dihydrobenzo[b][1,4]dioxin-2-yl)-1-isopropyl-6-(pyridin-4-yl)-1H-imidazo[4,5-b]pyridine

### 52A. 6-bromo-2-(2,3-dihydrobenzo[b][1,4]dioxin-2-yl)-1-isopropyl-1H-imidazo[4,5-b]pyridine

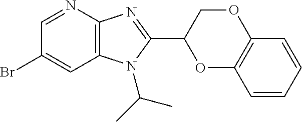

The desired product was prepared by using Example 49 (210 mg) and isopropyl bromide in Example 50A to give the desired product (28.0 mg, 12%). LC-MS: single peak at 254 nm, MH⁺ calcd. for C₁₇H₁₆BrN₃O₂: 374, obtained 374. ¹H-NMR (CDCl₃, 400 MHz): 8.60 (1H, d, J=2.1 Hz), 8.06 (1H, d, J=2.1 Hz), 6.93 (4H, m), 5.44 (1H, dd, J=2.4 Hz, 8.5 Hz), 5.07 (1H, p, J=6.9 Hz), 4.94 (1H, dd, J=2.4 Hz, 12.0 Hz), 4.75 (1H, dd, J=8.4 Hz, 12.0 Hz), 1.75 (3H, d, J=6.9 Hz), 1.68 (3H, d, J=7.0 Hz). The other regioisomer was also isolated as a by-product (105 mg, 45%).

### 52B. 2-(2,3-dihydrobenzo[b][1,4]dioxin-2-yl)-1-isopropyl-6-(pyridin-4-yl)-1H-imidazo[4,5-b]pyridine

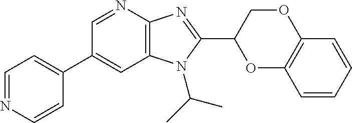

The desired product was prepared by using Example 52A (26.0 mg) in Example 50B to give the desired product (5.23 mg, 20%). LC-MS: single peak at 254 nm, MH⁺ calcd. for C₂₂H₂₀N₄O₂: 373, obtained 373. HPLC: single peak by analytical HPLC. ¹H-NMR (MeOD-d₄, 400 MHz): 8.86 (1H, d, J=2.0 Hz), 8.68 (2H, m), 8.58 (1H, d, J=2.1 Hz), 7.89 (2H, m), 6.92 (4H, m), 5.75 (1H, dd, J=2.5 Hz, 7.7 Hz), 5.26 (1H, p, J=6.9 Hz), 4.86 (1H, dd, J=2.5 Hz, 11.8 Hz), 4.73 (1H, dd, J=7.7 Hz, 11.7 Hz), 1.84 (3H, d, J=6.9 Hz), 1.81 (3H, d, J=7.0 Hz).

### Example 53

### 1-allyl-2-(2,3-dihydrobenzo[b][1,4]dioxin-2-yl)-6-(pyridin-4-yl)-1H-imidazo[4,5-b]pyridine

### 53A. 1-allyl-6-bromo-2-(2,3-dihydrobenzo[b][1,4]dioxin-2-yl)-1H-imidazo[4,5-b]pyridine

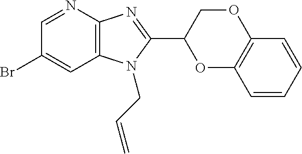

The desired product was prepared by using Example 49 (200 mg) and allyl bromide in Example 50A to give the desired product (38.0 mg, 16%). LC-MS: single peak at 254 nm, MH⁺ calcd. for C₁₇H₁₄BrN₃O₂: 372, obtained 372. ¹H-NMR (CDCl₃, 400 MHz): 8.62 (1H, d, J=2.1 Hz), 7.87 (1H, d, J=2.1 Hz), 6.92 (4H, m), 6.02 (1H, m), 5.45 (1H, dd, J=2.5 Hz, 8.4 Hz), 5.33 (1H, d, J=10.3 Hz), 5.13 (1H, d, J=17.1 Hz), 5.01 (2H, m), 4.91 (1H, dd, J=2.5 Hz, 11.9 Hz), 4.72 (1H, dd, J=8.4 Hz, 11.9 Hz). The other regioisomer was also isolated as a by-product (91.0 mg, 40%).

### 53B. 1-allyl-2-(2,3-dihydrobenzo[b][1,4]dioxin-2-yl)-6-(pyridin-4-yl)-1H-imidazo[4,5-b]pyridine

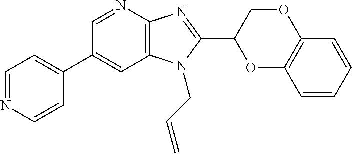

The desired product was prepared by using Example 53A (36.0 mg) in Example 50B to give the desired product (4.14 mg, 12%). LC-MS: single peak at 254 nm, MH⁺ calcd. for C₂₂H₁₈N₄O₂: 371, obtained 371. HPLC: single peak by analytical HPLC. ¹H-NMR (MeOD-d₄, 400 MHz): 8.89 (1H, s), 8.67 (2H, d, J=4.7 Hz), 8.45 (1H, s), 7.86 (2H, d, J=4.7 Hz), 6.92 (4H, m), 6.18 (1H, m), 5.70 (1H, m), 5.34 (1H, d, J=10.3 Hz), 5.26 (2H, m), 5.20 (1H, d, J=16.8 Hz), 4.81 (1H, d, J=11.6 Hz), 4.70 (1H, dd, J=7.7 Hz, 11.7 Hz).

### Example 54

### 2-(2,3-dihydrobenzo[b][1,4]dioxin-2-yl)-1-(4-fluorophenethyl)-6-(pyridin-4-yl)-1H-imidazo[4,5-b]pyridine

### 54A. 6-bromo-2-(2,3-dihydrobenzo[b][1,4]dioxin-2-yl)-1-(4-fluorophenethyl)-1H-imidazo[4,5-b]pyridine

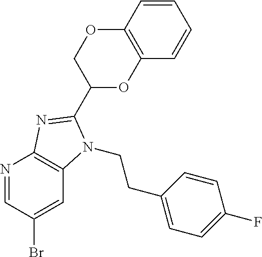

The desired product was prepared by using Example 49 (200 mg) and 1-(2-bromoethyl)-4-fluorobenzene in Example 50A to give the desired product (49.0 mg, 18%). LC-MS: single peak at 254 nm, MH⁺ calcd. for C₂₂H₁₇BrFN₃O₂: 454, obtained 454. ¹H-NMR (CDCl₃, 400 MHz): 8.58 (1H, d, J=2.1 Hz), 7.59 (1H, d, J=2.2 Hz), 6.95 (8H, m), 5.11 (1H, dd, J=2.6 Hz, 8.2 Hz), 4.74 (1H, dd, J=2.6 Hz, 11.9 Hz), 4.65 (1H, dd, J=8.3 Hz, 11.9 Hz), 4.55 (2H, t, J=7.1 Hz), 3.20 (2H, t, J=7.1 Hz).

### 54B. 2-(2,3-dihydrobenzo[b][1,4]dioxin-2-yl)-1-(4-fluorophenethyl)-6-(pyridin-4-yl)-1H-imidazo[4,5-b]pyridine

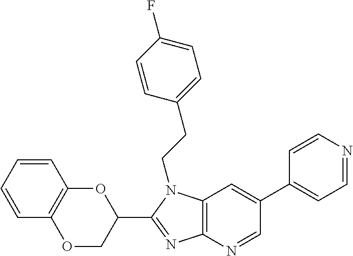

The desired product was prepared by using Example 54A (49.0 mg) in Example 50B to give the desired product (10.9 mg, 22%). LC-MS: single peak at 254 nm, MH⁺ calcd. for C₂₇H₂₁FN₄O₂: 453, obtained 453. HPLC: single peak by analytical HPLC. ¹H-NMR (MeOD-d₄, 400 MHz): 8.82 (1H, d, J=2.0 Hz), 8.67 (2H, m), 8.10 (1H, d, J=2.0 Hz), 7.78 (2H, m), 7.11 (2H, m), 6.94 (6H, m), 5.44 (1H, dd, J=3.3 Hz, 7.2 Hz), 4.83 (2H, m), 4.62 (2H, m), 3.32 (2H, m).

### Example 55

### 2-(2,3-dihydrobenzo[b][1,4]dioxin-2-yl)-1-isobutyl-6-(pyridin-4-yl)-1H-imidazo[4,5-b]pyridine

### 55A. 6-bromo-2-(2,3-dihydrobenzo[b][1,4]dioxin-2-yl)-1-isobutyl-1H-imidazo[4,5-b]pyridine

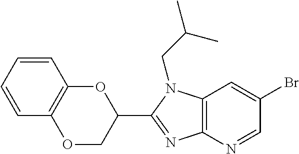

The desired product was prepared by using Example 49 (200 mg) and 1-bromo-2-methylpropane in Example 50A to give the desired product (33.0 mg). LC-MS: single peak at 254 nm, MH⁺ calcd. for C₁₈H₁₈BrN₃O₂: 388, obtained 388. ¹H-NMR (CDCl₃, 400 MHz): 8.33 (1H, d, J=1.5 Hz), 7.82 (1H, d, J=1.5 Hz), 7.10 (1H, m), 6.91 (3H, m), 5.54 (1H, dd, J=2.4 Hz, 8.4 Hz), 4.71 (1H, dd, J=2.3 Hz, 11.3 Hz), 4.48 (3H, m), 2.51 (1H, m, J=7.5 Hz), 1.00 (3H, d, J=6.7 Hz), 0.99 (3H, d, J=6.7 Hz).

### 55B. 2-(2,3-dihydrobenzo[b][1,4]dioxin-2-yl)-1-isobutyl-6-(pyridin-4-yl)-1H-imidazo[4,5-b]pyridine

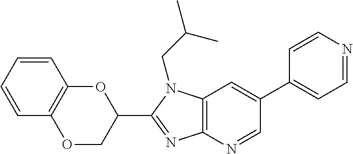

The desired product was prepared by using Example 55A (30.0 mg) in Example 50B to give the desired product (24.8 mg, 83%). LC-MS: single peak at 254 nm, MH⁺ calcd. for C₂₃H₂₂N₄O₂: 387, obtained 387. HPLC: single peak by analytical HPLC.

### Example 56

### 1-sec-butyl-2-(2,3-dihydrobenzo[b][1,4]dioxin-2-yl)-6-(pyridin-4-yl)-1H-imidazo[4,5-b]pyridine

### 56A. 6-bromo-1-sec-butyl-2-(2,3-dihydrobenzo[b][1,4]dioxin-2-yl)-1H-imidazo[4,5-b]pyridine

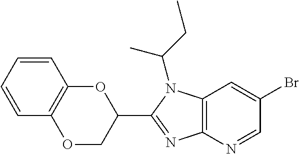

The desired product was prepared by using Example 49 (200 mg) and 2-bromobutane in Example 50A to give the desired product (48.0 mg). LC-MS: single peak at 254 nm, MH⁺ calcd. for C₁₈H₁₈BrN₃O₂: 388, obtained 388.

### 56B. 1-sec-butyl-2-(2,3-dihydrobenzo[b][1,4]dioxin-2-yl)-6-(pyridin-4-yl)-1H-imidazo[4,5-b]pyridine

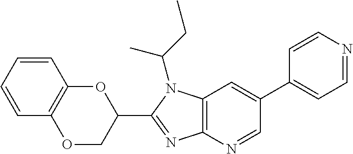

The desired product was prepared by using Example 56A (48.0 mg) in Example 50B to give the desired product (31.3 mg, 65%) as an approximately 4:1 mixture of diastereomers. LC-MS: single peak at 254 nm, MH⁺ calcd. for C₂₃H₂₂N₄O₂: 387, obtained 387. HPLC: two peaks by analytical HPLC.

### Example 57

### 1-(but-3-en-2-yl)-2-(2,3-dihydrobenzo[b][1,4]dioxin-2-yl)-6-(pyridin-4-yl)-1H-imidazo[4,5-b]pyridine

### 57A. 6-bromo-1-(but-3-en-2-yl)-2-(2,3-dihydrobenzo[b][1,4]dioxin-2-yl)-1H-imidazo[4,5-b]pyridine

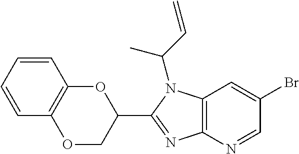

The desired product was prepared by using Example 49 (200 mg) and crotyl bromide in Example 50A to give the desired product (33.0 mg). LC-MS: single peak at 254 nm, MH⁺ calcd. for C₁₈H₁₆BrN₃O₂: 386, obtained 386.

### 57B. 1-(but-3-en-2-yl)-2-(2,3-dihydrobenzo[b][1,4]dioxin-2-yl)-6-(pyridin-4-yl)-1H-imidazo[4,5-b]pyridine

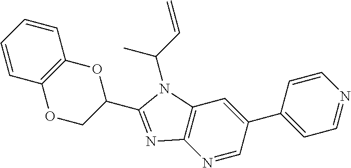

The desired product was prepared by using Example 57A (33.0 mg) in Example 50B to give the desired product (7.94 mg, 24%) as an approximately 3:1 mixture of diastereomers. LC-MS: single peak at 254 nm, MH⁺ calcd. for C₂₃H₂₀N₄O₂: 385, obtained 385. HPLC: two peaks by analytical HPLC.

### Example 58

### 2-(2,3-dihydrobenzo[b][1,4]dioxin-2-yl)-1-(3-methylbut-2-enyl)-6-(pyridin-4-yl)-1H-imidazo[4,5-b]pyridine

### 58A. 6-bromo-2-(2,3-dihydrobenzo[b][1,4]dioxin-2-yl)-1-(3-methylbut-2-enyl)-1H-imidazo[4,5-b]pyridine

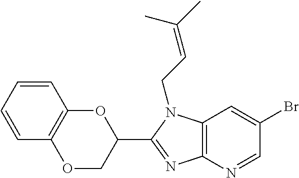

The desired product was prepared by using Example 49 (200 mg) and 1-bromo-3-methylbut-2-ene in Example 50A to give the desired product (58.0 mg). LC-MS: single peak at 254 nm, MH⁺ calcd. for C₁₉H₁₈BrN₃O₂: 400, obtained 400.

### 58B. 2-(2,3-dihydrobenzo[b][1,4]dioxin-2-yl)-1-(3-methylbut-2-enyl)-6-(pyridin-4-yl)-1H-imidazo[4,5-b]pyridine

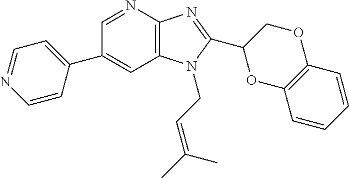

The desired product was prepared by using Example 58A (58.0 mg) in Example 50B to give the desired product (1.56 mg). LC-MS: single peak at 254 nm, MH⁺ calcd. for C₂₄H₂₂N₄O₂: 399, obtained 399. HPLC: single peak by analytical HPLC.

### Example 59

### (R)-2-phenyl-1-(5-(pyridin-4-yl)-1H-benzo[d]imidazol-2-yl)ethanamine

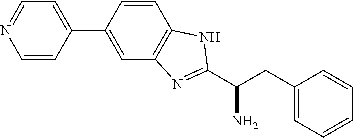

A mixture of above Example 23C (37 mg, 1.0 equiv.), Boc-D-Phe-OH (55 mg, 1.0 equiv.), HATU (1.5 equiv.), and DIEA (1.5 equiv.) in DMF (2 mL) was stirred at room temperature for 40 minutes. The reaction mixture was diluted with EtOAc, washed with saturated NaHCO₃ and brine, dried with Na₂SO₄, filtered, and concentrated. The residue was dissolved in glacial HOAc and heated to 60° C. for 2 hours. After removing the solvent, the residue was treated with 40% TFA in DCM for 30 minutes. The reaction mixture was concentrated and purified by HPLC to afford the title compound (36%). ¹H NMR (CDCl₃, 400 MHz) δ 3.39-3.50 (m, 2H), 4.95 (m, 1H), 7.17-7.19 (m, 2H), 7.24-7.33 (m, 3H), 7.83-7.86 (m, 1H), 7.91-7.93 (m, 1H), 8.30-8.36 (complex, 3H), 8.88-8.93 (complex, 5H); LC/MS: C₂₀H₁₉N₄ (M+1) 315.10.

### Example 60

### (S)-2-(4-chlorophenyl)-1-(5-(pyridin-4-yl)-1H-benzo[d]imidazol-2-yl)ethanamine

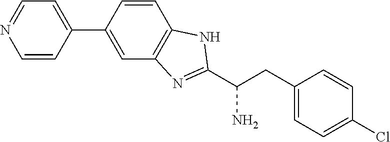

The desired product was prepared by substituting Boc-p-chloro-Phe-OH for Boc-D-Phe-OH in Example 59. ¹H NMR (CDCl₃, 400 MHz) δ 3.38-3.49 (m, 2H), 4.96 (m, 1H), 7.19-7.22 (m, 2H), 7.37-7.41 (m, 2H), 7.84-7.86 (m, 1H), 7.90-7.93 (m, 1H), 8.29-8.31 (complex, 3H), 8.86-8.91 (complex, 5H); LC/MS: Cₒ₂H₁₈ClN₄ (M+1) 349.07.

### Example 61

### (S)-2-(3,4-difluorophenyl)-1-(5-(pyridin-4-yl)-1H-benzo[d]imidazol-2-yl)ethanamine

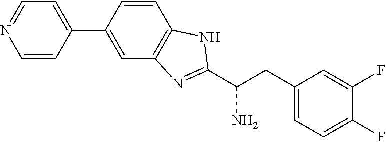

The desired product was prepared by substituting Boc-3,4-difluoro-Phe-OH for Boc-D-Phe-OH in Example 59. ¹H NMR (CDCl₃, 400 MHz) δ 3.19-3.35 (m, 2H), 4.85 (m, 1H), 6.87-6.88 (m, 1H), 7.17-7.30 (m, 2H), 7.70-7.75 (m, 2H), 8.00-8.02 (d, J=6.0 Hz, 2H), 8.09 (s, 1H), 8.69-8.71 (complex, 5H); LC/MS: Cₒ₂H₁₇F₂N₄ (M+1) 351.09.

### Example 62

### (S)-3-(2-amino-2-(5-(pyridin-4-yl)-1H-benzo[d]imidazol-2-yl)ethyl)benzonitrile

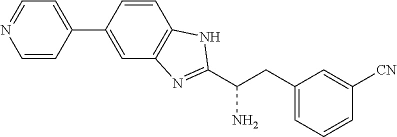

The desired product was prepared by substituting Boc-m-cyano-Phe-OH for Boc-D-Phe-OH in Example 59. ¹H NMR (CDCl₃, 400 MHz) δ 3.31-3.43 (m, 2H), 4.94 (m, 1H), 7.35-7.42 (m, 2H), 7.60 (s, 1H), 7.64-7.67 (m, 1H), 7.72-7.75 (m, 1H), 7.79-7.82 (m, 1H), 8.19 (s, 1H), 8.22-8.24 (d, J=6.0 Hz, 2H), 8.74-8.80 (complex, 5H); LC/MS: C₂₁H₁₈H₅ (M+1) 340.09.

### Example 63

### (S)-1-(5-(pyridin-4-yl)-1H-benzo[d]imidazol-2-yl)-2-m-tolylethanamine

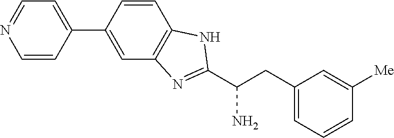

The desired product was prepared by substituting Boc-m-methyl-Phe-OH for Boc-D-Phe-OH in Example 59. ¹H NMR (CDCl₃, 400 MHz) δ 2.14 (s, 3H), 3.24-3.29 (m, 2H), 4.80 (m, 1H), 6.79-6.81 (m, 1H), 6.93-7.06 (complex, 3H), 7.71-7.73 (m, 1H), 7.77-7.80 (m, 1H), 8.16-8.20 (complex, 3H), 8.69-8.78 (complex, 5H); LC/MS: C₂, H₂₁N₄ (M+1) 329.06.

### Example 64

### (R)-1-(5-(pyridin-4-yl)-1H-benzo[d]imidazol-2-yl)-2-(4-(trifluoromethyl)phenyl)ethanamine

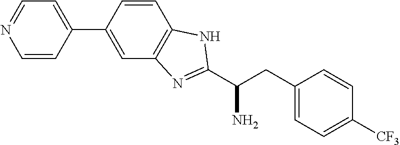

The desired product was prepared by substituting Boc-p-trifluoromethyl-D-Phe-OH for Boc-D-Phe-OH in Example 59. ¹H NMR (CDCl₃, 400 MHz) δ 3.31-3.47 (m, 2H), 4.90 (m, 1H), 7.42-7.44 (m, 1H), 7.57-7.59 (m, 2H), 7.64-7.68 (m, 1H), 7.72-7.80 (m, 2H), 8.16-8.20 (complex, 3H), 8.76-8.78 (complex, 5H); LC/MS: C₂, H₁₈F₃N₄ (M+1) 383.08.

### Example 65

### (R)-2-(naphthalen-2-yl)-1-(5-(pyridin-4-yl)-1H-benzo[d]imidazol-2-yl)ethanamine

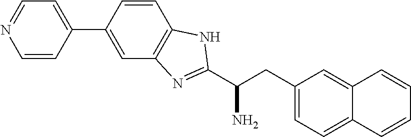

The desired product was prepared by substituting Boc-D-2-Nal-OH for Boc-D-Phe-OH in Example 59. ¹H NMR (CDCl₃, 400 MHz) δ 3.31-3.54 (m, 2H), 4.94 (m, 1H), 7.42-7.44 (dd, J=2, 8.4 Hz, 1H), 7.39-7.44 (m, 2H), 7.65 (s, 1H), 7.69-7.80 (complex, 5H), 8.14-8.16 (complex, 3H), 8.73-8.76 (complex, 5H); LC/MS: C₂₄H₂₁N₄ (M+1) 383.08.

### Example 66

### (R)-2-(3,4-difluorophenyl)-1-(5-(pyridin-4-yl)-1H-benzo[d]imidazol-2-yl)ethanamine

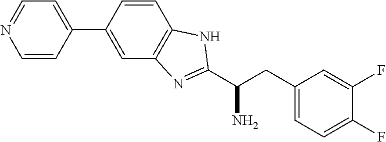

The desired product was prepared by substituting Boc-3,4-difluoro-D-Phe-OH for Boc-D-Phe-OH in Example 59. ¹H NMR (CDCl₃, 400 MHz) δ 3.31-3.43 (m, 2H), 4.95 (m, 1H), 6.95-6.97 (m, 1H), 7.24-7.37 (m, 1H), 7.80-7.82 (m, 1H), 7.86-7.89 (m, 1H), 8.25-8.30 (complex, 3H), 8.79-8.87 (complex, 5H); LC/MS: C₂₀H₁₇F₂N₄ (M+1) 351.11.

### Example 67

### (R)-2-(3,4-dichlorophenyl)-1-(5-(pyridin-4-yl)-1H-benzo[d]imidazol-2-yl)ethanamine

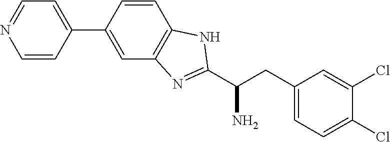

The desired product was prepared by substituting Boc-3,4-dichloro-D-Phe-OH for Boc-D-Phe-OH in Example 59. ¹H NMR (CDCl₃, 400 MHz) δ 3.31-3.43 (m, 2H), 4.95 (m, 1H), 6.95-6.97 (m, 1H), 7.24-7.37 (m, 1H), 7.80-7.82 (m, 1H), 7.86-7.89 (m, 1H), 8.25-8.30 (complex, 3H), 8.79-8.87 (complex, 5H); LC/MS: C₂₀H₁₇Cl₂N₄ (M+1) 383.03.

### Example 68

### (R)-2-(4-methoxyphenyl)-1-(5-(pyridin-4-yl)-1H-benzo[d]imidazol-2-yl)ethanamine

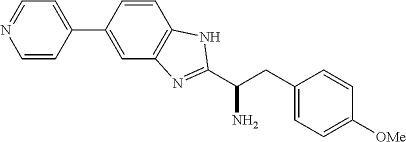

The desired product was prepared by substituting Boc-p-methoxy-D-Phe-OH for Boc-D-Phe-OH in Example 59. ¹H NMR (CDCl₃, 400 MHz) δ 3.22-3.27 (m, 2H), 3.61 (s, 3H), 4.75 (m, 1H), 6.73-6.75 (m, 2H), 6.95-6.97 (m, 2H), 7.71-7.81 (m, 2H), 8.18-8.23 (complex, 3H), 8.67-8.80 (complex, 5H); LC/MS: C₂₁H₂₁N₄O (M+1) 345.11.

### Example 69

### (R)-(2,4-difluorophenyl)-(5-(pyridin-4-yl)-1H-benzo[d]imidazol-2-yl)methanamine

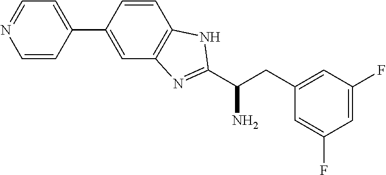

The desired product was prepared by substituting Boc-2,4-difluoro-D-Phg-OH for Boc-D-Phe-OH in Example 59. ¹H NMR (CDCl₃, 400 MHz) δ 6.09 (m, 1H), 7.14-7.19 (m, 1H), 7.39-7.47 (m, 2H), 7.72-7.73 (m, 2H), 8.06-8.08 (m, 2H), 8.68-8.69 (m, 2H), 9.16 (complex, 3H); LC/MS: C₁₉H₁₅F₂N₄ (M+1) 337.04.

### Example 70

### (R)-2-(2-fluorophenyl)-1-(5-(pyridin-4-yl)-1H-benzo[d]imidazol-2-yl)ethanamine

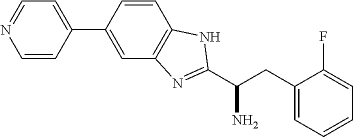

The desired product was prepared by substituting Boc-o-fluoro-D-Phe-OH for Boc-D-Phe-OH in Example 59. ¹H NMR (CDCl₃, 400 MHz) δ 3.34-3.45 (m, 2H), 4.79 (m, 1H), 6.96-7.08 (complex, 4H), 7.70-7.73 (m, 1H), 7.79-7.81 (m, 1H), 8.18 (s, 1H), 8.25-8.27 (m, 2H), 8.80-8.82 (complex, 5H); LC/MS: C₂₀H₁₈FN₄ (M+1) 333.14.

### Example 71

### (R)-2-(2-chlorophenyl)-1-(5-(pyridin-4-yl)-1H-benzo[d]imidazol-2-yl)ethanamine

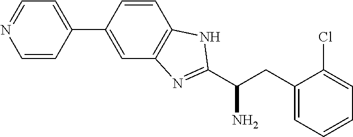

The desired product was prepared by substituting Boc-o-chloro-D-Phe-OH for Boc-D-Phe-OH in Example 59. ¹H NMR (CDCl₃, 400 MHz) δ 3.37-3.47 (m, 2H), 4.80 (m, 1H), 7.03-7.05 (m, 1H), 7.08-7.13 (m, 1H), 7.18-7.22 (m, 1H), 7.37-7.39 (m, 1H), 7.71-7.73 (m, 1H), 7.79-7.82 (m, 1H), 8.18 (s, 1H), 8.24-8.26 (m, 2H), 8.79-8.84 (complex, 5H); LC/MS: C₂₀H₁₈ClN₄ (M+1) 349.08.

### Example 72

### (3-fluorophenyl)-(5-(pyridin-4-yl)-1H-benzo[d]imidazol-2-yl)methanamine

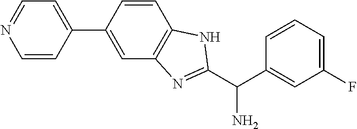

The desired product was prepared by substituting Boc-m-fluoro-D,L-Phg-OH for Boc-D-Phe-OH in Example 59. ¹H NMR (CDCl₃, 400 MHz) δ 5.97 (m, 1H), 7.14-7.39 (complex, 3H), 7.45-7.51 (m, 1H), 7.70-7.77 (m, 2H), 8.06-8.08 (complex, 3H), 8.72-8.74 (m, 2H), 9.20 (complex, 3H); LC/MS: C₁₉H₁₆FN₄ (M+1) 319.04.

### Example 73

### biphenyl-4-yl-(5-(pyridin-4-yl)-1H-benzo[d]imidazol-2-yl)methanamine

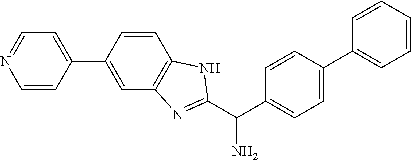

The desired product was prepared by substituting Boc-4-phenyl-D,L-Phg-OH for Boc-D-Phe-OH in Example 59. ¹H NMR (CDCl₃, 400 MHz) δ 5.97 (m, 1H), 7.30-7.35 (m, 1H), 7.39-7.43 (m, 2H), 7.52-7.64 (complex, 5H), 7.69-7.83 (complex, 4H), 8.11-8.12 (m, 3H), 8.74-8.76 (m, 2H), 9.47 (br s, 3H); LC/MS: C₂₅H₂₁N₄ (M+1) 377.01.

### Example 74

### (S)-1,2,3,4-tetrahydro-3-(5-(pyridin-4-yl)-1H-benzo[d]imidazol-2-yl)isoquinoline

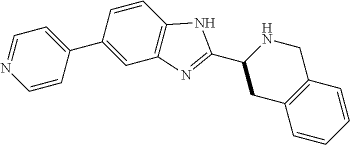

The desired product was prepared by substituting (S)—N-Boc-1,2,3,4-tetrahydroisoquinoline-3-carboxylic acid for Boc-D-Phe-OH in Example 59. ¹H NMR (CDCl₃, 400 MHz) δ 3.30-3.40 (m, 1H), 3.55-3.61 (m, 1H), 4.56-4.59 (m, 2H), 5.19-5.21 (m, 1H), 7.32-7.34 (complex, 4H), 7.83-7.94 (m, 2H), 8.29-8.31 (m, 3H), 8.87-8.88 (m, 2H), 10.24 (br s, 2H); LC/MS: C₂₁H₁₉N₄ (M+1) 327.14.

### Example 75

### 2-(3,4-dimethoxyphenyl)-1-(5-(pyridin-4-yl)-1H-benzo[d]imidazol-2-yl)ethanamine

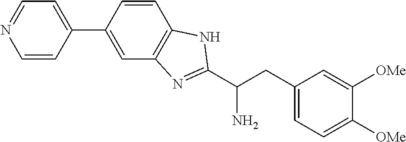

The desired product was prepared by substituting Boc-3,4-methoxy-D,L-Phe-OH for Boc-D-Phe-OH in Example 59. ¹H NMR (CDCl₃, 400 MHz) δ 3.20-3.24 (m, 2H), 3.43 (s, 3H), 3.61 (s, 3H), 4.75-4.80 (m, 1H), 6.53-6.59 (m, 2H), 6.73-6.75 (m, 1H), 7.72-7.75 (m, 2H), 8.07-8.12 (complex, 3H), 8.63-8.74 (complex, 5H); LC/MS: C₂₂H₂₃N₄O₂ (M+1) 375.10.

### Example 76

### (4-methoxyphenyl)-(5-(pyridin-4-yl)-1H-benzo[d]imidazol-2-yl)methanamine

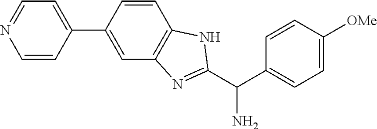

The desired product was prepared by substituting Boc-p-methoxy-D,L-Phg-OH for Boc-D-Phe-OH in Example 59. ¹H NMR (CDCl₃, 400 MHz) δ 3.70 (s, 3H), 5.85 (m, 1H), 6.95-6.97 (m, 2H), 7.37-7.39 (m, 2H), 7.74-7.77 (m, 2H), 8.09-8.11 (m, 3H), 8.73-8.75 (m, 2H), 9.03 (br s, 3H); LC/MS: C₂₀H₁₉N₄O (M+1) 330.93.

### Example 77

### (4-bromophenyl)-(5-(pyridin-4-yl)-1H-benzo[d]imidazol-2-yl)methanamine

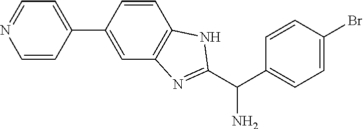

The desired product was prepared by substituting Boc-p-bromo-D,L-Phg-OH for Boc-D-Phe-OH in Example 59. ¹H NMR (CDCl₃, 400 MHz) δ 5.94 (m, 1H), 7.39-7.43 (m, 2H), 7.63-7.78 (complex, 4H), 8.08-8.09 (complex, 3H), 8.73-8.75 (m, 2H), 9.15 (br s, 3H); LC/MS: C₁₉H₁₆BrN₄ (M+1) 378.93.

### Example 78

### (4-chlorophenyl)-5-(pyridin-4-yl)-1H-benzo[d]imidazol-2-yl)methanamine

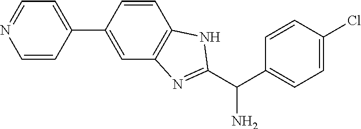

The desired product was prepared by substituting Boc-p-chloro-D,L-Phg-OH for Boc-D-Phe-OH in Example 59. ¹H NMR (CDCl₃, 400 MHz) δ 5.97 (m, 1H), 7.47-7.53 (complex, 4H), 7.71-7.79 (complex, 2H), 8.13-8.15 (complex, 3H), 8.76-8.77 (m, 2H), 9.17 (br s, 3H); LC/MS: C₁₉H₁ClN₄ (M+1) 335.00.

### Example 79

### (R)-2-(4-chlorophenyl)-1-(5-(pyridin-4-yl)-1H-benzo[d]imidazol-2-yl)ethanamine

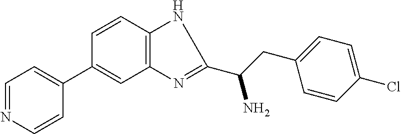

The desired product was prepared by substituting Boc-p-chloro-D-Phe-OH for Boc-D-Phe-OH in Example 59. After removal of the DCM and TFA, the residue was further purified by preparative HPLC to give the product (12%). LC-MS: single peak at 254 nm, MH⁺ calcd. for C₂₀H₁₇ClN₄: 349, obtained: 349. ¹H-NMR (DMSO-d₆, 400 MHz), δ 8.78 (d, J=6.7 Hz, 2H), 8.72 (m, 3H), 8.19 (d, J=6.7 Hz, 2H), 8.16 (m, 1H), 7.78 (dd, J=8.5, 1.7 Hz 1H), 7.72 (d, J=8.5 Hz, 1H), 7.26 (d, J=8.4 Hz, 2H), 7.08 (d, J=8.4 Hz, 2H), 4.83 (m, 1H), 3.31 (m, 1H).

### Example 80

### (S)-2-(naphthalen-1-yl)-1-(5-(pyridin-4-yl)-1H-benzo[d]imidazol-2-yl)ethanamine

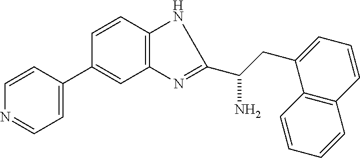

The desired product was prepared by substituting Boc-1-Nal-OH for Boc-D-Phe-OH in Example 59. LC-MS: single peak at 254 nm, MH⁺ calcd. for C₂₄H₂₀N₄: 365, obtained: 365. ¹H-NMR (DMSO-d₆, 400 MHz), δ 8.74 (m, 2H), 8.69 (d, J=6.0 Hz, 2H), 8.20 (d, J=8.8 Hz, 1H), 7.99 (m, 1H), 7.92 (d, J=8.8 Hz 1H), 7.77 (d, J=8.4 Hz, 1H), 7.73 (m, 2H), 7.54 (m, 2H), 7.25 (m, 1H), 7.09 (d, J=7.2 Hz, 1H), 4.81 (m, 1H), 4.40-3.30 (br, 2H), 3.75 (m, 2H).

### Example 81

### (R)-2-(3-bromophenyl)-1-(5-(pyridin-4-yl)-1H-benzo[d]imidazol-2-yl)ethanamine

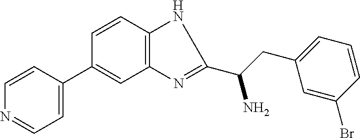

The desired product was prepared by substituting Boc-m-bromo-D-Phe-OH for Boc-D-Phe-OH in Example 59. LC-MS: single peak at 254 nm, MH⁺ calcd. for C₂₀H₁₇BrN₄: 393, obtained: 393. ¹H-NMR (DMSO-d₆, 400 MHz), δ 8.74 (m, 3H), 8.20 (m, 3H), 7.75 (ddd, J=17.6, 8.4, 1.6 Hz, 1H), 7.44 (m, 1H), 7.38 (m, 1H), 7.20 (m, 3H), 7.03 (d, J=7.6 Hz, 1H), 4.86 (m, 1H), 4.19 (m, 1H), 3.30 (m, 1H), 3.00 (m, 1H).

### Example 82

### (R)-2-(5-bromo-2-methoxyphenyl)-1-(5-(pyridin-4-yl)-1H-benzo[d]imidazol-2-yl)ethanamine

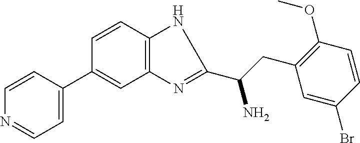

The desired product was prepared by substituting Boc-5-bromo-2-methoxy-D-Phe-OH for Boc-D-Phe-OH in Example 59. LC-MS: single peak at 254 nm, MH⁺ calcd. for C₂₁H₁₉BrN₄O: 424, obtained: 424. ¹H-NMR (DMSO-d₆, 400 MHz), δ 8.70 (d, J=7.6 Hz, 2H), 8.64 (m, 2H), 8.10 (m, 1H), 8.03 (m, 2H), 7.72 (m, 2H), 7.34 (dd, J=8.8, 2.5 Hz, 1H), 7.14 (d, J=8.9 Hz, 1H), 4.76 (m, 2H), 3.58 (s, 3H), 3.23 (m, 2H).

### Example 83

### (S)-4-(2-amino-2-(5-(pyridin-4-yl)-1H-benzo[d]imidazol-2-yl)ethyl)benzonitrile

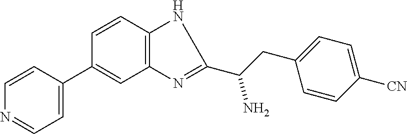

The desired product was prepared by substituting Boc-p-cyano-Phe-OH for Boc-D-Phe-OH in Example 59. LC-MS: single peak at 254 nm, MH⁺ calcd. for C₂₁H₁₇N₅: 340, obtained: 340. ¹H-NMR (DMSO-d₆, 400 MHz), δ 8.75 (m, 4H), 8.12 (m, 3H), 7.72 (m, 4H), 7.29 (d, J=6.7 Hz, 2H), 4.90 (m, 1H), 3.43 (dd, J=13.7, 7.7 Hz, 1H), 3.36 (dd, J=13.7, 6.4 Hz, 1H).

### Example 84

### (S)-2-(4-tert-butylphenyl)-1-(5-(pyridin-4-yl)-1H-benzo[d]imidazol-2-yl)ethanamine

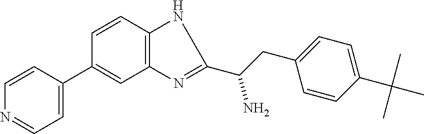

The desired product was prepared by substituting Boc-p-t-butyl-Phe-OH for Boc-D-Phe-OH in Example 59. LC-MS: single peak at 254 nm, MH⁺ calcd. for C₂₄H₂₆N₄: 371, obtained: 371. ¹H-NMR (DMSO-d₆, 400 MHz), δ 8.72 (d, J=6.4 Hz, 2H), 8.62 (m, 2H), 8.11 (m, 1H), 8.03 (m, 2H), 7.73 (m, 2H), 7.21 (d, J=8.3 Hz, 2H), 6.99 (d, J=8.3 Hz, 2H), 4.77 (m, 2H), 3.33 (dd, J=13.9, 8.0 Hz, 1H), 3.21 (dd, J=13.9, 6.1 Hz, 1H), 1.15 (s, 9H).

### Example 85

### 2-phenyl-1-(6-(pyridin-4-yl)-1H-benzo[d]imidazol-2-yl)ethanamine, trifluoroacetic acid salt

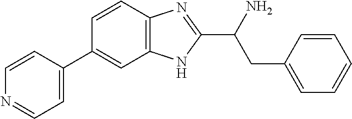

4-bromo-1,2-diaminobenzene (1 equiv.) was added to a mixture of Boc-D,L-Phe-OH (1.2 equiv.), DIEA (3 equiv.), and HATU (1.5 equiv.) in DMF (10 mL/mmol). After stirring at room temperature overnight, the DMF was removed under reduced pressure. The resulting residue was suspended in ethyl acetate, and washed with brine (2×), saturated NaHCO₃ (3×), brine (2×), 1N HCl (2×), and brine again (2×). The organic phase was dried under anhydrous Na₂SO₄, and the solvent was evaporated to give a crude mixture of two regioisomer amides. This crude product was used directly in the next step without subjecting to chromatography. Thus, a solution of the amide mixture in acetic acid was heated to and stirred at 60° C. for 2 hours. After removing the acetic acid under reduced pressure, the residue was suspended in ethyl acetate and subjected to routine washing, and drying (over Na₂SO₄). The solvent was removed to give the crude benzimidazole product. Next, the Suzuki reaction was carried out by heating a degassed and sealed solution of benzimidazole (1 equiv.), pyridine-4-boronic acid (1.5 equiv.), Pd[P(Ph)₃]₄ (10% by weight), and K₂CO₃ (5 equiv.) in water/Dioxane (1:4 by volume) at 100° C. for 20-48 hours. After removing the solvents, the residue was subjected directly to preparative reverse phase HPLC to obtain the Boc-protected product. The Boc group was then removed by treating with a solution of 30% TFA in DCM for 30 minutes. After the solvents were evaporated under reduced pressure, the residue was suspended (dissolved) in water and lyophilized to give the desired product as a powder (TFA salt, 35% from the bromodiamine). LC-MS: single peak at 254 nm, MH⁺ calcd. for C₂₀H₁₈H₄: 315, obtained: 315.

### Example 86

### 1-(6-(isoquinolin-4-yl)-1H-benzo[d]imidazol-2-yl)-2-phenylethanamine, trifluoroacetic acid salt

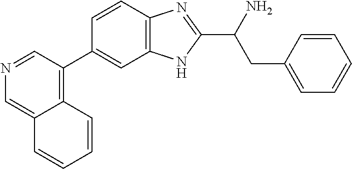

The desired product was prepared by substituting isoquinoline-4-boronic acid for pyridine-4-boronic acid in Example 85. Preparative HPLC was used to obtain the final compound as the TFA salt (2% from the bromodiamine). LC-MS: single peak at 254 nm, MH⁺ calcd. for C₂₄H₂₀N₄: 365, obtained: 365.

### Example 87

### 1-(6-(isoquinolin-5-yl)-1H-benzo[d]imidazol-2-yl)-2-phenylethanamine, trifluoroacetic acid salt

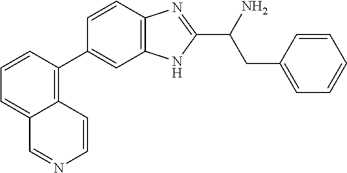

The desired product was prepared by substituting isoquinoline-5-boronic acid for pyridine-4-boronic acid in Example 85. Preparative HPLC was used to obtain the final compound as the TFA salt (34% from the bromodiamine). LC-MS: single peak at 254 nm, MH⁺ calcd. for C₂₄H₂₀N₄: 365, obtained: 365.

### Example 88

### 1-(6-(3,5-dimethyl-1H-pyrazol-4-yl)-1H-benzo[d]imidazol-2-yl)-2-phenylethanamine, trifluoroacetic acid salt

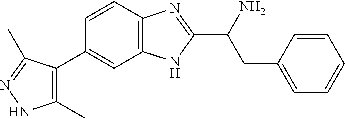

The desired product was prepared by substituting 3,5-dimethylpyrazole-4-boronic acid, pinacol ester for pyridine-4-boronic acid in Example 85. Preparative HPLC was used to obtain the final compound as the TFA salt (26% from the bromodiamine). LC-MS: single peak at 254 nm, MH⁺ calcd. for C₂₀H₂₁N₅: 332, obtained: 332.

### Example 89

### 2-phenyl-1-(6-(quinolin-4-yl)-1H-benzo[d]imidazol-2-yl)ethanamine, trifluoroacetic acid salt

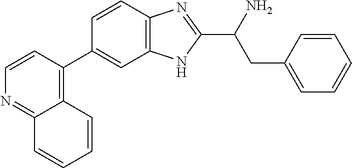

The desired product was prepared by substituting quinoline-4-boronic acid for pyridine-4-boronic acid in Example 85. Preparative HPLC was used to obtain the final compound as the TFA salt (12% from the bromodiamine). LC-MS: single peak at 254 nm, MH⁺ calcd. for C₂₄H₂₀N₄: 365, obtained: 365.

### Example 90

### 1-(6-(pyridin-4-yl)-1H-benzo[d]imidazol-2-yl)-2-(4-(pyridin-4-yl)phenyl)ethanamine, trifluoroacetic acid salt

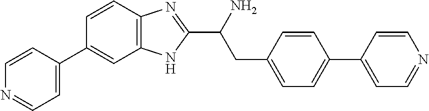

The desired product was prepared by substituting Boc-4-bromo-D,L-Phe-OH for Boc-D,L-Phe-OH in Example 85. Preparative HPLC was used to obtain the final compound as the TFA salt (30% from the bromodiamine). LC-MS: single peak at 254 nm, MH⁺ calcd. for C₂₅H₂₁N₅: 392, obtained: 392.

### Example 91

### 1-(6-(1H-pyrazol-4-yl)-1H-benzo[d]imidazol-2-yl)-2-(4-(1H-pyrazol-4-yl)phenyl)ethanamine, trifluoroacetic acid salt

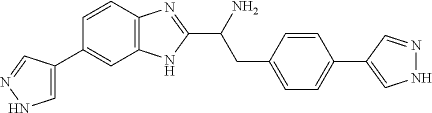

The desired product was prepared by substituting Boc-4-bromo-D,L-Phe-OH for Boc-D,L-Phe-OH and N1-BOC-4-(4,4,5,5-tetramethyl-1,3,2-dioxaborolan-2-yl)-1H-pyrazole for pyridine-4-boronic acid in Example 85. Preparative HPLC was used to obtain the final compound as the TFA salt (10% from the bromodiamine). LC-MS: single peak at 254 nm, MH⁺ calcd. for C₂₁H₁₉N₇: 370, obtained: 370.

### Example 92

### 1-(6-(3,5-dimethyl-1H-pyrazol-4-yl)-1H-benzo[d]imidazol-2-yl)-2-(4-(3,5-dimethyl-1H-pyrazol-4-yl)phenyl)ethanamine, trifluoroacetic acid salt

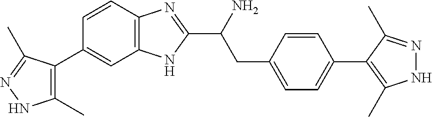

The desired product was prepared by substituting Boc-4-bromo-D,L-Phe-OH for Boc-D,L-Phe-OH and 3,5-dimethylpyrazole-4-boronic acid, pinacol ester for pyridine-4-boronic acid in Example 85. Preparative HPLC was used to obtain the final compound as the TFA salt (32% from the bromodiamine). LC-MS: single peak at 254 nm, MH⁺ calcd. for C₂₅H₂₇N₇: 426, obtained: 426.

### Example 93

### (R)-1-(6-(1H-pyrazol-4-yl)-1H-benzo[d]imidazol-2-yl)-2-(3-fluorophenyl)ethanamine, trifluoroacetic acid salt

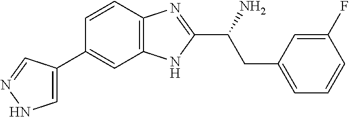

The desired product was prepared by substituting Boc-m-fluoro-D-Phe-OH for Boc-D,L-Phe-OH and N1-BOC-4-(4,4,5,5-tetramethyl-1,3,2-dioxaborolan-2-yl)-1H-pyrazole for pyridine-4-boronic acid in Example 85. Preparative HPLC was used to obtain the final compound as the TFA salt (20% from the bromodiamine). LC-MS: single peak at 254 nm, MH⁺ calcd. for C₁₈H₁₆N₅: 322, obtained: 322.

### Example 94

### (R)-2-(3-fluorophenyl)-1-(6-(pyridin-4-yl)-1H-benzo[d]imidazol-2-yl)ethanamine, trifluoroacetic acid salt

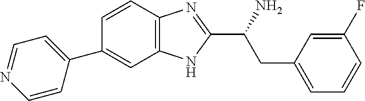

The desired product was prepared by substituting Boc-m-fluoro-D-Phe-OH for Boc-D,L-Phe-OH in Example 85. Preparative HPLC was used to obtain the final compound as the TFA salt (22% from the bromodiamine). LC-MS: single peak at 254 nm, MH⁺ calcd. for C₂₀H₁₇FN₄: 333, obtained: 333.

### Example 95

### (R)-2-(3-fluorophenyl)-1-(6-(3-fluoropyridin-4-yl)-1H-benzo[d]imidazol-2-yl)ethanamine, trifluoroacetic acid salt

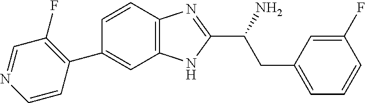

The desired product was prepared by substituting Boc-m-fluoro-D-Phe-OH (available from Chem-Impex International, Inc., Wood Dale, Ill.) for Boc-D,L-Phe-OH and 3-fluoropyridine-4-boronic acid for pyridine-4-boronic acid in Example 85. Preparative HPLC was used to obtain the final compound as the TFA salt (22% from the bromodiamine). LC-MS: single peak at 254 nm, MH⁺ calcd. for C₂₀H₁₆F₂N₄: 351, obtained: 351.

### Example 96

### (R)-1-(6-(3-chloropyridin-4-yl)-1H-benzo[d]imidazol-2-yl)-2-(3-fluorophenyl)ethanamine, trifluoroacetic acid salt

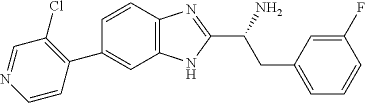

The desired product was prepared by substituting Boc-m-fluoro-D-Phe-OH for Boc-D,L-Phe-OH and 3-chloropyridine-4-boronic acid (available from Medinoah, Wallingford, Conn.) for pyridine-4-boronic acid in Example 85. Preparative HPLC was used to obtain the final compound as the TFA salt (20% from the bromodiamine). LC-MS: single peak at 254 nm, MH⁺ calcd. for C₁₈H₁₆ClFN₄: 367, obtained: 367.

### Example 97

### (R)-2-(3-fluorophenyl)-1-(6-(2-methoxypyridin-4-yl)-1H-benzo[d]imidazol-2-yl)ethanamine, trifluoroacetic acid salt

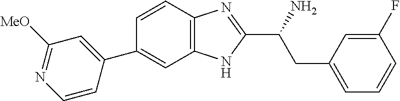

The desired product was prepared by substituting Boc-m-fluoro-D-Phe-OH for Boc-D,L-Phe-OH and 2-methoxypyridine-4-boronic acid (available from Combi-Blocks, San Diego, Calif.) for pyridine-4-boronic acid in Example 85. Preparative HPLC was used to obtain the final compound as the TFA salt (15% from the bromodiamine). LC-MS: single peak at 254 nm, MH⁺ calcd. for C₂₁H₁₉FN₄O: 363, obtained: 363.

### Example 98

### (R)-2-(3-fluorophenyl)-1-(6-(2-methylpyridin-4-yl)-1H-benzo[d]imidazol-2-yl)ethanamine, trifluoroacetic acid salt

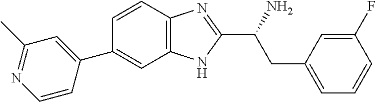

The desired product was prepared by substituting Boc-m-fluoro-D-Phe-OH for Boc-D,L-Phe-OH and 2-methylpyridine-4-boronic acid (available from Combi-Blocks, San Diego, Calif.) for pyridine-4-boronic acid in Example 85. Preparative HPLC was used to obtain the final compound as the TFA salt (7% from the bromodiamine). LC-MS: single peak at 254 nm, MH⁺ calcd. for C₂₁H₁₉FN₄: 347, obtained: 347.

### Example 99

### (R)-1-(6-(2-chloropyridin-4-yl)-1H-benzo[d]imidazol-2-yl)-2-(3-fluorophenyl)ethanamine, trifluoroacetic acid salt

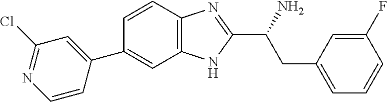

The desired product was prepared by substituting Boc-m-fluoro-D-Phe-OH for Boc-D,L-Phe-OH and 2-chloropyridine-4-boronic acid (available from Aldrich Chemical Co., Milwaukee, Wis.) for pyridine-4-boronic acid in Example 85. Preparative HPLC was used to obtain the final compound as the TFA salt (12% from the bromodiamine). LC-MS: single peak at 254 nm, MH⁺ calcd. for C₂₀H₁₆ClFN₄: 367, obtained: 367.

### Example 100

### (R)-2-(3-fluorophenyl)-1-(6-(2-fluoropyridin-4-yl)-1H-benzo[d]imidazol-2-yl)ethanamine, trifluoroacetic acid salt

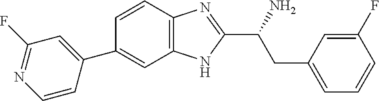

The desired product was prepared by substituting Boc-m-fluoro-D-Phe-OH for Boc-D,L-Phe-OH and 2-fluoropyridine-4-boronic acid (available from Frontier Scientific, Logan, Utah) for pyridine-4-boronic acid in Example 85. Preparative HPLC was used to obtain the final compound as the TFA salt (10% from the bromodiamine). LC-MS: single peak at 254 nm, MH⁺ calcd. for C₂₀H₁₆F₂N₄: 351, obtained: 351.

### Example 101

### (R)-2-(3-fluorophenyl)-1-(6-(2-(4-methylpiperazin-1-yl)pyridin-4-yl)-1H-benzo[d]imidazol-2-yl)ethanamine, trifluoroacetic acid salt

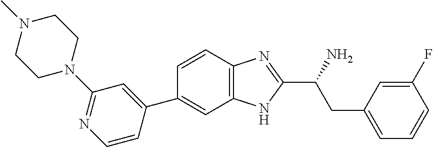

The desired product was prepared by substituting Boc-m-fluoro-D-Phe-OH for Boc-D,L-Phe-OH and 2-(4-methylpiperazin-1-yl)pyridine-4-boronic acid, pinacol ester (available from Boron Molecular Inc., Research Triangle Park, N.C.) for pyridine-4-boronic acid in Example 85. Preparative HPLC was used to obtain the final compound as the TFA salt (62% from the bromodiamine). LC-MS: single peak at 254 nm, MH⁺ calcd. for C₂₅H₂₁FN₆: 431, obtained: 431.

### Example 102

### (R)-2-(3-fluorophenyl)-1-(6-(2-(4-methylpiperazin-1-yl)pyridin-3-yl)-1H-benzo[d]imidazol-2-yl)ethanamine, trifluoroacetic acid salt

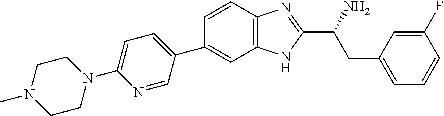

The desired product was prepared by substituting Boc-m-fluoro-D-Phe-OH for Boc-D,L-Phe-OH and 2-(4-methylpiperazin-1-yl)pyridine-3-boronic acid, pinacol ester (available from Boron Molecular Inc., Research Triangle Park, N.C.) for pyridine-4-boronic acid in Example 85. Preparative HPLC was used to obtain the final compound as the TFA salt (35% from the bromodiamine). LC-MS: single peak at 254 nm, MH⁺ calcd. for C₂₅H₂₁FN₆: 431, obtained: 431.

### Example 103

### (R)-1-(6-(1H-pyrazol-4-yl)-3H-imidazo[4,5-b]pyridin-2-yl)-2-(4-chlorophenyl)ethanamine

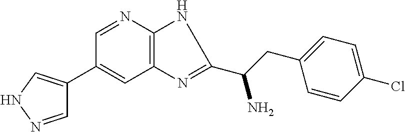

A solution of 2,3-diamino-5-bromopyridine (1 equiv.) and Ac-p-chloro-D-Phe-OH (1 equiv.) in DMF (10 mL/mmol) was treated with HATU (1 equiv.) and DIEA (3 equiv.) sequentially. The resulting mixture was stirred at room temperature for 1 hour. The solution was diluted with EtOAc and washed with saturated NaHCO₃ solution. The organic layer was dried over sodium sulfate and concentrated in vacuo. The residue was dissolved in glacial acetic acid and heated to 110° C. for 8 hours. Acetic acid was evaporated and the residue was used for the next reaction without further purification. Thus the crude arylbromide (1 equiv.) and N1-BOC-4-(4,4,5,5-tetramethyl-1,3,2-dioxaborolan-2-yl)-1H-pyrazole (1.5 equiv.) were dissolved in THF in a sealed tube. Pd(PPh₃)₄ (0.03 equiv.) and 2M solution of Na₂CO₃ (3 equiv.) were added sequentially. The resulting mixture was heated to 100° C. for one hour in a microwave reactor. After cooling to room temperature, the mixture was diluted with water and extracted with ethyl acetate. The organic layers were combined, dried over sodium sulfate and concentrated in vacuo. The residue was dissolved in MeOH and was treated with 1M HCl at 50° C. for 1 hour to remove the acetyl group. The residue thus produced was purified by preparative HPLC to give the desired product as solid (31%). LC-MS: single peak at 254 nm, MH⁺ calcd. for C₁₇H₁₅ClN₆: 339, obtained: 339. ¹H-NMR (DMSO-d₆, 400 MHz), δ 8.63 (s, 5H), 8.12 (s, 3H), 7.27 (d, J=8.4 Hz, 2H), 7.06 (d, J=8.4 Hz, 2H), 4.75 (m, 1H), 3.28 (m, 2H).

### Example 104

### (R)-2-(4-chlorophenyl)-1-(6-(pyridin-4-yl)-3H-imidazo[4,5-b]pyridin-2-yl)ethanamine

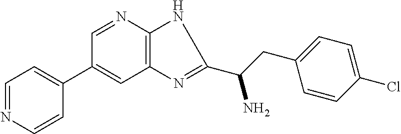

The arylbromide prepared according to Example 103 (1 equiv.) and pyridine-4-boronic acid (1.5 equiv) were dissolved in THF (15 mL/mmol) in a sealed tube. Pd(PPh₃)₄ (0.03 equiv.) and 2M solution of Na₂CO₃ (3 equiv.) were added sequentially. The resulting mixture was heated to 100° C. for one hour in a microwave reactor. After cooling to room temperature, the mixture was diluted with water and extracted with ethyl acetate. The organic layers were combined, dried over sodium sulfate and concentrated in vacuo. The residue was dissolved in MeOH and was treated with 1M HCl at 50° C. for 1 hour to remove the acetyl group. The residue thus produced was purified by preparative HPLC to give the desired product as a solid (12%). LC-MS: single peak at 254 nm, MH⁺ calcd. for C₁₉H₁₆ClN₅: 350, obtained: 350. ¹H-NMR (DMSO-d₆, 400 MHz), δ 8.86 (d, J=2.0 Hz, 1H), 8.73 (m, 5H), 8.49 (s, 1H), 8.04 (d, J=6.1 Hz, 2H), 7.27 (d, J=8.5 Hz, 2H), 7.08 (d, J=8.5 Hz, 2H), 4.83 (m, 1H), 3.26 (m, 2H).

### Example 105

### (2,4-difluorophenyl)-(6-(pyridin-4-yl)-3H-imidazo[4,5-b]pyridin-2-yl)methanamine

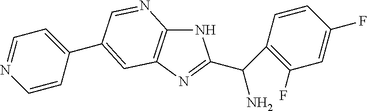

A solution of Example 45A (1 equiv.) and Ac-2,4-difluoro-D,L-Phg-OH (1 equiv.) in DMF (10 mL/mmol) was treated with HATU (1 equiv.) and DIEA (3 equiv.). The resulting mixture was stirred at room temperature for one hour and LC/MS showed the starting material was completely consumed. Saturated aqueous NaHCO₃ was added and the mixture was extracted three times with EtOAc. The organic layers were combined and dried over anhydrous Na₂SO₄. The ethyl acetate was removed and the residue was dried overnight under high vacuum to give amide product. This amide mixture was used directly in the next step without further purification and characterizations. The amide residue was dissolved in HOAc and stirred at 110° C. for 2 hours to give the cyclized product. The residue was dissolved in MeOH and was treated with 1M HCl at 50° C. for 1 hour to remove the acetyl group. The residue thus produced was purified by preparative HPLC to give the desired product as a solid (11%). LC-MS: single peak at 254 nm, MH⁺ calcd. for C₁₈H₁₃F₂N₅: 337, obtained: 337. ¹H-NMR (DMSO-d₆, 400 MHz), δ 9.01 (d, J=2.2 Hz, 1H), 8.69 (d, J=6.2 Hz, 3H), 8.22 (m, 1H), 7.96 (d, J=5.1 Hz, 2H), 7.45 (m, 1H), 7.29 (m, 1H), 3.49 (m, 4H).

### Example 106

### (R)-N-(2-(4-chlorophenyl)-1-(6-(pyridin-4-yl)-3H-imidazo[4,5-b]pyridin-2-yl)ethyl)acetamide

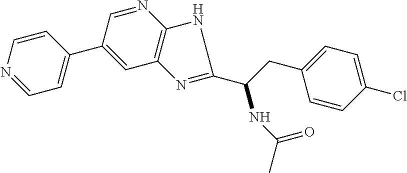

The desired product was prepared by using Example 45A (40 mg, 1 equiv.) and substituting Ac-p-chloro-D-Phe-OH for Ac-2,4-difluoro-D,L-Phg-OH in Example 105, but omitting the deprotection step. Preparative HPLC gave 13 mg of the title compound (16%). LC-MS: single peak at 254 nm, MH⁺ calcd. for C₂₁H₁₈N₅O: 392, obtained: 392. ¹H-NMR (DMSO-d₆, 400 MHz), δ 8.85 (t, J=2.1 Hz, 1H), 8.79 (d, J=6.6 Hz, 2H), 8.57 (d, J=8.2 Hz, 1H), 8.46 (m, 1H), 8.20 (d, J=6.3 Hz, 2H), 7.26 (d, J=8.5 Hz, 2H), 7.20 (d, J=8.5 Hz, 2H), 5.24 (td, J=8.5, 5.8 Hz, 1H), 3.31 (dd, J=13.6, 5.8 Hz, 1H), 3.04 (dd, J=13.6, 9.2 Hz, 1H), 1.77 (s, 3H).

### Example 107

### (S)-N-(2-(3,4-difluorophenyl)-1-(6-(pyridin-4-yl)-3H-imidazo[4,5-b]pyridin-2-yl)ethyl)acetamide

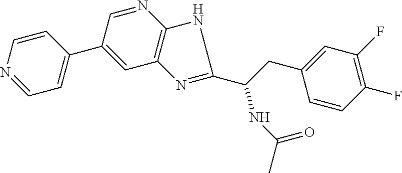

The desired product was prepared by using Example 45A (100 mg, 1 equiv.) and substituting Ac-3,4-difluoro-Phe-OH for Ac-2,4-difluoro-D,L-Phg-OH in Example 105, but omitting the deprotection step. Preparative HPLC gave 21 mg of the title compound (10%). LC-MS: single peak at 254 nm, MH⁺ calcd. for C₂₁H₁₇F₂N₅O: 394, obtained: 394. ¹H-NMR (DMSO-d₆, 400 MHz), δ 8.90 (s, 1H), 8.83 (d, J=6.7 Hz, 2H), 8.58 (d, J=8.2 Hz, 1H), 8.50 (m, 1H), 8.29 (d, J=6.7 Hz, 2H), 7.29 (m, 2H), 7.01 (m, 2H), 5.25 (m, 1H), 3.34 (dt, J=13.5, 5.8 Hz, 1H), 3.07 (td, J=13.5, 9.3 Hz, 1H), 1.77 (s, 3H).

### Example 108

### (S)-N-(2-(3-cyanophenyl)-1-(6-(pyridin-4-yl)-3H-imidazo[4,5-b]pyridin-2-yl)ethyl)acetamide

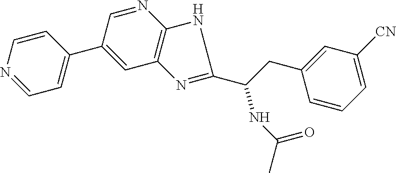

The desired product was prepared by using Example 45A (100 mg, 1 equiv.) and substituting Ac-3-cyano-Phe-OH for Ac-2,4-difluoro-D,L-Phg-OH in Example 105, but omitting the deprotection step. Preparative HPLC gave 59 mg of the title compound (29%). LC-MS: single peak at 254 nm, MH⁺ calcd. for C₂₂H₁₈N₆O: 383, obtained: 383. ¹H-NMR (DMSO-d₆, 400 MHz), δ 8.90 (t, J=2.1 Hz, 1H), 8.83 (d, J=6.8 Hz, 2H), 8.60 (d, J=8.3 Hz, 2H), 8.51 (d, J=2.1 Hz, 1H), 8.30 (d, J=6.7 Hz, 2H), 7.68 (s, 1H), 7.62 (dt, J=7.7, 1.3 Hz, 1H), 7.53 (d, J=7.9 Hz, 1H), 7.42 (t, J=7.7 Hz, 1H), 5.30 (m, 1H), 3.40 (dd, J=13.7, 5.7 Hz, 1H), 3.12 (dd, J=13.7, 9.4 Hz, 1H), 1.77 (s, 3H).

### Example 109

### (S)-N-(1-(6-(pyridin-4-yl)-3H-imidazo[4,5-b]pyridin-2-yl)-2-m-tolylethyl)acetamide

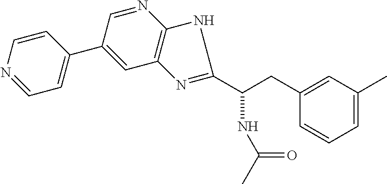

The desired product was prepared by using Example 45A (40 mg, 1 equiv.) and substituting Ac-3-methyl-Phe-OH for Ac-2,4-difluoro-D,L-Phg-OH in Example 105, but omitting the deprotection step. Preparative HPLC gave 31 mg of the title compound (16%). LC-MS: single peak at 254 nm, MH⁺ calcd. for C₂₂H₂₁N₅O: 372, obtained: 372. ¹H-NMR (DMSO-d₆, 400 MHz), δ 8.88 (t, J=2.1 Hz, 1H), 8.82 (d, J=6.7 Hz, 2H), 8.56 (d, J=8.1 Hz, 1H), 8.49 (m, 1H), 8.28 (d, J=6.7 Hz, 2H), 7.07 (m, 2H), 6.94 (m, 2H), 5.23 (dd, J=8.7, 6.1 Hz, 1H), 3.27 (dd, J=13.6, 6.1 Hz, 1H), 3.01 (dd, J=13.6, 8.7 Hz, 1H), 2.18 (s, 3H), 1.77 (s, 3H).

### Example 110

### (R)-2-(3-fluorophenyl)-1-(6-(pyridin-4-yl)-3H-imidazo[4,5-b]pyridin-2-yl)ethanamine

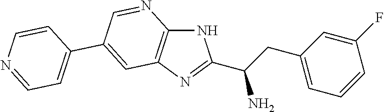

The desired product was prepared by substituting Boc-3-fluoro-D-Phe-OH for Ac-p-chloro-D-Phe-OH in Example 104. Preparative HPLC was utilized to obtain the title compound (36%). LC-MS: single peak at 254 nm, MH⁺ calcd. for C₁₉H₁₆FN₅: 334, obtained: 334.

### Example 111

### (R)-N-(2-(3-fluorophenyl)-1-(6-(pyridin-4-yl)-3H-imidazo[4,5-b]pyridin-2-yl)ethyl)acetamide

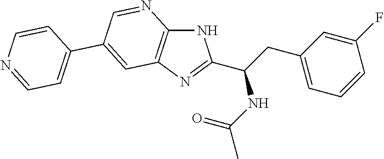

The desired product was prepared by substituting Ac-3-fluoro-D-Phe-OH for Ac-p-chloro-D-Phe-OH in Example 104, but omitting the deprotection step. Preparative HPLC was utilized to obtain the title compound (36%). LC-MS: single peak at 254 nm, MH⁺ calcd. for C₂₁H₁₈FN₅O: 376, obtained: 376.

### Example 112

### (R)-1-(6-(1H-pyrazol-4-yl)-3H-imidazo[4,5-b]pyridin-2-yl)-2-(3-fluorophenyl)ethanamine

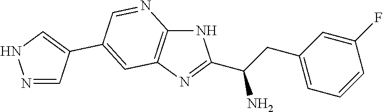

The desired product was prepared by substituting Boc-3-fluoro-D-Phe-OH for Ac-p-chloro-D-Phe-OH in Example 103. Preparative HPLC was utilized to obtain the title compound (36%). LC-MS: single peak at 254 nm, MH⁺ calcd. for C₁₇H₁₅FN₆: 323, obtained: 323.

### Example 113

### (R)-N-(1-(6-(1H-pyrazol-4-yl)-3H-imidazo[4,5-b]pyridin-2-yl)-2-(3-fluorophenyl)ethyl)acetamide

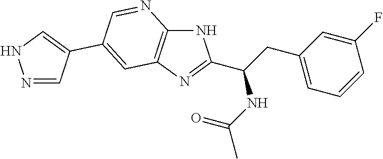

The desired product was prepared by substituting Ac-3-fluoro-D-Phe-OH for Ac-p-chloro-D-Phe-OH in Example 103, but omitting the deprotection step. Preparative HPLC was utilized to obtain the title compound (36%). LC-MS: single peak at 254 nm, MH⁺ calcd. for C₁₉H₁₇FN₆O: 365, obtained: 365.

### Example 114

### (s)-6-(pyridin-4-yl)-2-(pyrolidin-2-yl)-1H-benzo[d]imidazole

### 114A. bis(tert-butyl) 4-bromo-1,2-phenylenedicarbamate

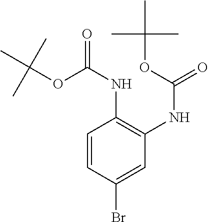

A solution of 1,2-diamino-4-bromobenzene (1.87 g, 10 mmol) in EtOH (20 mL) was added Boc₂O (4.36 g, 20 mmol). The reaction mixture was stirred at 30° C. overnight and the solvent was removed by rotary evaporation. The residue was purified by flash column chromatography (5 to 15% EtOAc in hexanes) to afford the desired product (3.27 g, 84%) as an off-white solid. LC-MS: single peak at 254 nm, MH⁺ calcd. for C₂₁H₂₈N₃O₄: 388, obtained: 388.

### 114B. bis(tert-butyl) 4-(pyridin-4-yl)-1,2-phenylenedicarbamate

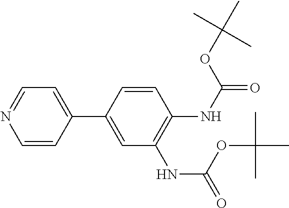

A solution of Example 114A (0.600 g, 1.55 mmol) in DME (5 mL) was treated with 4-pyridine boronic acid (0.247 g, 2.01 mmol), an aqueous 2 M K₂CO₃ solution (2.3 mL), and tetrakis(triphenylphosphine)palladium(0) (0.09 g, 0.08 mmol). The mixture was stirred vigorously at 90° C. overnight, cooled, and the organic solvent was removed by rotary evaporation. After addition of EtOAc (10 mL) and water (10 mL), the layers were separated and the aqueous layer was further extracted with EtOAc (2×10 mL). The combined organic layers were washed with brine, dried over sodium sulfate, filtered, and concentrated to dryness. The residue was purified by flash column chromatography (15 to 60% EtOAc in hexanes) to yield the desired product (0.41 g, 69%) as colorless foam. LC-MS: single peak at 254 nm, MH⁺ calcd. for C₂₁H₂₈N₃O₄: 386, obtained: 386.

### 114C. 4-(pyridin-4-yl)benzene-1,2-diamine

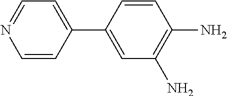

The starting material (270 mg, 0.698 mmol) was dissolved in a solution of 50% TFA in methylene chloride (5 mL) and the mixture was stirred at room temperature for 1 hour. The solvent was removed under reduced pressure and excess acid was removed by repeated evaporation from toluene in vacuo. The crude amine was used without further purification.

### 114D. (S)-tert-butyl 2-(6-(pyridin-4-yl)-1H-benzo[c]imidazol-2-yl)pyrrolidine-1-carboxylate

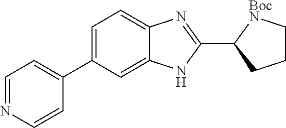

Example 114C was treated with enough DMF (0.5 mL) to dissolve the residue. The solution was treated with N-Boc L-proline (151 mg, 1 equiv.), HOBt (1.1 equiv.), NMM (2 equiv.), and EDC (1.1 equiv.). The resulting mixture was stirred at 0° C., then allowed to warm up to room temperature overnight. After removal of solvent by rotary evaporation, the residue was dissolved in ethyl acetate (20 mL) and a saturated aqueous solution of NaHCO₃ (10 mL). The layers were separated and the organic layer was washed with brine (10 mL), dried over Na₂SO₄, filtered, and the solvent was removed in vacuo. Without further purification, the residue was dissolved in acetic acid and heated by microwave irradiation at 80° C. for 65 min. The solvent was removed by rotary evaporation and the residue was purified by HPLC to yield desired benzimidazole (180 mg, 54%). LC-MS: single peak at 254 nm, MH⁺ calcd. for C₂₁H₂₅N₄O₂: 365, obtained: 365.

### 114E. (S)-6-(pyridin-4-yl)-2-(pyrrolidin-2-yl)-1H-benzo[d]imidazole

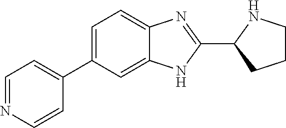

Example 114D (20 mg, 0.042 mmol) was dissolved in a solution of 50% TFA in methylene chloride and the mixture was stirred at 0° C. for 1 hour. The solvent and excess acid was removed by repeated evaporation from toluene in vacuo and the crude product purified by HPLC to yield the desired amine product (78%). LC-MS: single peak at 254 nm, MH⁺ calcd. for C₁₆H₁₇N₄: 265, obtained: 265.

### Example 115

### (S)-2-(1-(3-fluorobenzyl)pyrrolidin-2-yl)-6-(pyridin-4-yl)-1H-benzo[d]imidazole, trifluoroacetic acid salt

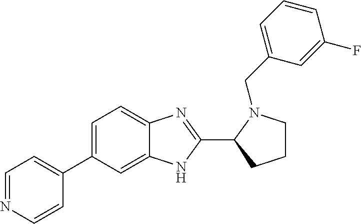

A solution of Example 114E (40 mg) in acetonitrile (1 mL) was treated with 3-fluorobenzyl bromide (1.2 eq) and triethylamine (1.2 eq). The reaction was stirred at 70° C. overnight and then the solvent was removed by rotary evaporation. This crude product was purified by preparative HPLC (gradient; mobile phase: solvent A: 0.1% TFA in water, solvent B: CH₃CN) to give 17 mg of the desired compound (34%) as the TFA salt. LC-MS: single peak at 254 nm, MH⁺ calcd. for C₂₃H₂₂FN₄: 373, obtained: 373.

### Example 116

### (S)-6-(pyridin-4-yl)-2-(1-(3-(trifluoromethyl)benzyl)pyrrolidin-2-yl)-1H-benzo[d]imidazole, trifluoroacetic acid salt

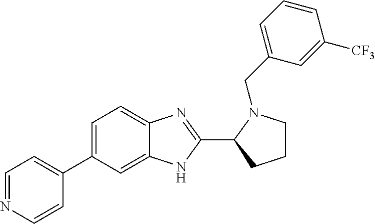

The desired product was prepared by substituting 3-(trifluoromethyl)benzyl bromide for 3-fluorobenzyl bromide in Example 115. Preparative HPLC gave 12 mg of the title compound (21%) as the TFA salt. LC-MS: single peak at 254 nm, MH⁺ calcd. for C₂₄H₂₂F₃N₄: 423, obtained: 423.

### Example 117

### (S)-2-(1-phenethylpyrrolidin-2-yl)-6-(pyridin-4-yl)-1H-benzo[d]imidazole, trifluoroacetic acid salt

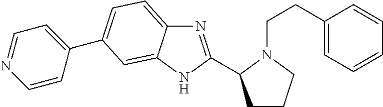

The desired product was prepared by substituting 1-(2-bromoethyl)benzene for 3-fluorobenzyl bromide in Example 115. Preparative HPLC gave 14 mg of the title compound (28%) as the TFA salt. LC-MS: single peak at 254 nm, MH⁺ calcd. for C₂₄H₂₅N₄: 369, obtained: 369.

### Example 118

### (S)-2-(1-(4-bromobenzyl)pyrrolidin-2-yl)-6-(pyridin-4-yl)-1H-benzo[d]imidazole, trifluoroacetic acid salt

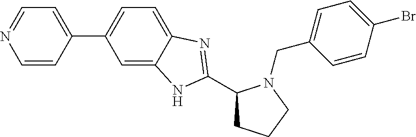

The desired product was prepared by using Example 114E (33 mg) and substituting 4-bromobenzyl bromide for 3-fluorobenzyl bromide in Example 115, and scaling appropriately. Preparative HPLC gave 9 mg of the title compound (20%) as the TFA salt. LC-MS: single peak at 254 nm, MH⁺ calcd. for C₂₃H₂₂BrN₄: 433, obtained: 433.

### Example 119

### (S)-2-(1-benzylpyrrolidin-2-yl)-6-(pyridin-4-yl)-1H-benzo[d]imidazole, trifluoroacetic acid salt

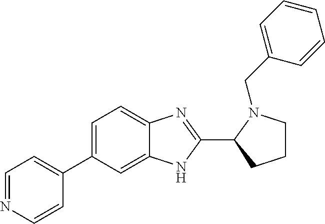

The desired product was prepared by using Example 114E (29 mg) and substituting benzyl bromide for 3-fluorobenzyl bromide in Example 115, and scaling appropriately. Preparative HPLC gave 13 mg of the title compound (39%) as the TFA salt. LC-MS: single peak at 254 nm, MH⁺ calcd. for C₂₃H₂₃N₄: 355, obtained: 355. ¹H NMR (DMSO-d₆, 400 MHz) δ 9.84 (br s, 1H), 9.30 (br s, 1H), 9.17 (d, J=7.1 Hz, 2H), 8.59 (d, J=7.1 Hz, 2H), 8.38 (s, 1H), 7.97 (dd, J=8.6, 1.7 Hz, 1H), 7.83 (d, J=8.4 Hz, 1H), 7.58 (dd, J=7.9, 1.6 Hz, 2H), 7.46 (m, 3H), 5.82 (s, 2H), 5.07 (br s, 1H), 3.40 (br s, 3H), 2.25-2.16 (m, 1H), 2.12-2.05 (m, 2H).

### Example 120

### (S)-2-(1-(2,4-difluorobenzyl)pyrrolidin-2-yl)-6-(pyridin-4-yl)-1H-benzo[d]imidazole, trifluoroacetic acid salt

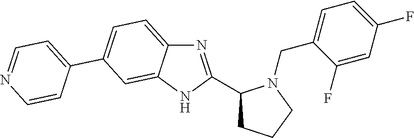

The desired product was prepared by using Example 114E (29 mg) and substituting 2,4-difluorobenzyl bromide for 3-fluorobenzyl bromide in Example 115, and scaling appropriately. Preparative HPLC gave 16 mg of the title compound (43%) as the TFA salt. LC-MS: single peak at 254 nm, MH⁺ calcd. for C₂₃H₂₁F₂N₄: 391, obtained: 391. ¹H NMR (DMSO-d₆, 400 MHz) δ 9.86 (br s, 1H), 9.31 (br s, 1H), 9.08 (d, J=6.9 Hz, 2H), 8.58 (d, J=7.1 Hz, 2H), 8.39 (s, 1H), 7.97 (dd, J=8.6, 1.7 Hz, 1H), 7.84 (d, J=8.6 Hz, 1H), 7.76 (dt, J=8.8, 6.6 Hz, 2H), 7.44 (ddd, J=10.6, 9.3, 2.5 Hz, 3H), 7.26 (dt, J=5.89 (s, 2H), 5.04 (br s, 1H), 3.40 (br s, 3H), 2.25-2.16 (m, 1H), 2.12-2.05 (m, 2H).

### Example 121

### (S)-2-(1-(4-chlorobenzyl)pyrrolidin-2-yl)-6-(pyridin-4-yl)-1H-benzo[d]imidazole, trifluoroacetic acid salt

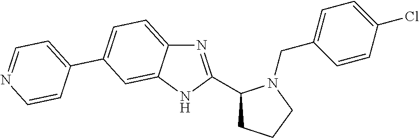

The desired product was prepared by using Example 114E (29 mg) and substituting 4-chlorobenzyl bromide for 3-fluorobenzyl bromide in Example 115, and scaling appropriately. Preparative HPLC gave 15 mg of the title compound (42%) as the TFA salt. LC-MS: single peak at 254 nm, MH⁺ calcd. for C₂₃H₂₁ClN₄: 389, obtained: 389. ¹H NMR (DMSO-d₆, 400 MHz) δ 9.83 (br s, 1H), 9.31 (br s, 1H), 9.15 (d, J=7.2 Hz, 2H), 8.59 (d, J=7.1 Hz, 2H), 8.39 (s, 1H), 7.97 (dd, J=8.6, 1.6 Hz, 1H), 7.84 (d, J=8.4 Hz, 1H), 7.62 (d, J=8.6 Hz, 2 H), 7.55 (d, J=8.6 Hz, 2H), 5.81 (s, 2H), 5.02 (br s, 1H), 3.40 (br s, 3H), 2.25-2.16 (m, 1H), 2.13-2.05 (m, 2H).

### Example 122

### (S)-2-(1-(4-methoxybenzyl)pyrrolidin-2-yl)-6-(pyridin-4-yl)-1H-benzo[d]imidazole, trifluoroacetic acid salt

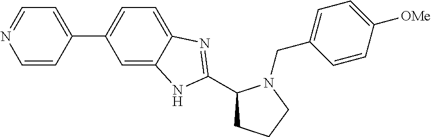

The desired product was prepared by substituting 4-methoxybenzyl bromide for 3-fluorobenzyl bromide in Example 115. Preparative HPLC gave 19 mg of the title compound (53%) as the TFA salt. LC-MS: single peak at 254 nm, MH⁺ calcd. for C₂₄H₂₅N₄O: 385, obtained: 385. ¹H NMR (DMSO-d₆, 400 MHz) δ 9.84 (br s, 1H), 9.31 (br s, 1H), 9.14 (d, J=7.0 Hz, 2H), 8.56 (d, J=7.1 Hz, 2H), 8.37 (s, 1H), 7.95 (dd, J=8.6, 1.8 Hz, 1H), 7.83 (d, J=8.6 Hz, 1H), 7.57 (d, J=8.8 Hz, 2H), 7.02 (d, J=8.8 Hz, 2H), 5.73 (s, 2H), 5.03 (br s, 1H), 3.39 (br s, 2H), 2.25-2.15 (m, 1H), 2.14-2.02 (m, 2H).

### Example 123

### 2-(piperidin-3-yl)-6-(pyridin-4-yl)-1H-benzo[d]imidazole

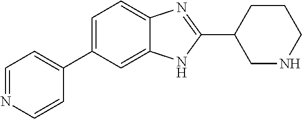

Example 114B (0.39 g, 1 mmol) was dissolved in a solution of 50% TFA in methylene chloride (2 mL) and the mixture was stirred at room temperature for 1 hour. The solvent was removed under reduced pressure and excess acid was removed by repeated evaporation from toluene in vacuo. Without further purification, the crude diamine was treated with enough DMF (10 mL) to dissolve the residue. To this solution was added N-Boc nipecotic acid (1 equiv), HOBt (1.1 equiv), NMM (2 equiv), and EDC (1.1 equiv). The resulting mixture was stirred at 0° C., then allowed to warm up to room temperature overnight. After removal of solvent by rotary evaporation, the residue was dissolved in ethyl acetate (20 mL) and a saturated aqueous solution of NaHCO₃ (10 mL). The layers were separated and the organic layer was washed with brine (10 mL), dried over Na₂SO₄, filtered, and the solvent was removed in vacuo. Without further purification, the residue was dissolved in acetic acid and heated by microwave irradiation at 80° C. for 65 min. The solvent was removed by rotary evaporation and the residue was dissolved in a solution of 50% TFA in methylene chloride (2 mL). After stirring at room temperature for 1 h, the solvent and excess acid was removed by repeated evaporation from toluene in vacuo and purified by HPLC to yield the desired amine product (0.28 g, 56%).

### Example 124

### 2-(1-(3-fluorobenzyl)piperidin-3-yl)-6-(pyridin-4-yl)-1H-benzo[d]imidazole, trifluoroacetic acid salt

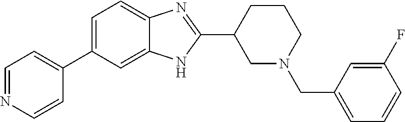

A solution of the Example 123 (21 mg) in acetonitrile (2 mL) was treated with 3-fluorobenzyl bromide (1.2 equiv.) and triethylamine (1.2 equiv.). The reaction was stirred at 70° C. overnight and then the solvent was removed by rotary evaporation. This crude product was purified by preparative HPLC (gradient; mobile phase: solvent A: 0.1% TFA in water, solvent B: CH₃CN) to give 5 mg of the desired compound (20%) as the TFA salt. LC-MS: single peak at 254 nm, MH⁺ calcd. for C₂₄H₂₄FN₄: 387, obtained: 387.

### Example 125

### 2-(1-(3-methoxybenzyl)piperidin-3-yl)-6-(pyridin-4-yl)-1H-benzo[d]imidazole, trifluoroacetic acid salt

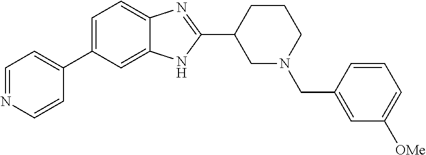

The desired product was prepared by using Example 123 (30 mg) and substituting 3-methoxybenzyl bromide for 3-fluorobenzyl bromide in Example 124, and scaling appropriately. Preparative HPLC gave 10 mg of the title compound (28%) as the TFA salt. LC-MS: single peak at 254 nm, MH⁺ calcd. for C₂₅H₂₇N₄O: 399, obtained: 399. ¹H NMR (DMSO-d₆, 400 MHz) δ 9.14 (d, J=7.1 Hz, 2H), 8.77 (br s, 1H), 8.55 (d, J=7.1 Hz, 2H), 8.32 (s, 1H), 7.91 (dd, J=8.6, 1.8, 1H), 7.75 (d, J=8.6 Hz, 1H), 7.38 (t, J=8.0 Hz, 1H), 7.21 (t, J=2 Hz, 1H), 7.13 (d, J=7.4 Hz, 1H), 7.01 (dd, J=8.0, 2.2 Hz, 1H), 5.76 (s, 2H), 3.78 (s, 3H), 3.64 (d, J=11.0 Hz, 1H), 3.41-3.31 (m, 3H), 2.96 (m, 1H), 2.21 (m, 1H), 1.91-1.80 (m, 3H).

### Example 126

### 6-(pyridin-4-yl)-2-(1-(3-(trifluoromethyl)benzyl)piperidin-3-yl)-1H-benzo[d]imidazole, trifluoroacetic acid salt

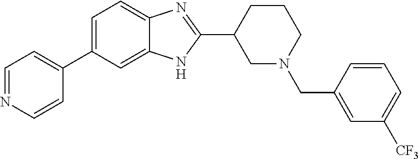

The desired product was prepared by using Example 123 (30 mg) and substituting 3-(trifluoromethyl)benzyl bromide for 3-fluorobenzyl bromide in Example 124, and scaling appropriately. Preparative HPLC gave 10 mg of the title compound (25%) as the TFA salt. LC-MS: single peak at 254 nm, MH⁺ calcd. for C₂₅H₂₄F₃N₄: 437, obtained: 437. ¹H NMR (DMSO-d₆, 400 MHz) δ 9.19 (d, J=7.1 Hz, 2H), 8.75 (br s, 1H), 8.58 (d, J=7.2 Hz, 2H), 8.33 (s, 1H), 8.09 (s, 1H), 7.92 (dd, J=8.6, 1.8, 1H), 7.89 (d, J=7.9 Hz, 1H), 7.82 (d, J=7.6 Hz, 1H), 7.74 (d, J=8.7 Hz, 1H), 7.71 (t, J=7.8 Hz, 1H), 5.89 (s, 2H), 3.63 (d, J=11.3 Hz, 1H), 3.44-3.31 (m, 3H), 3.01-2.93 (m, 1H), 2.21 (m, 1H), 1.91 (m, 1H), 1.76 (m, 2H).

### Example 127

### 2-(1-(4-bromobenzyl)piperidin-3-yl)-6-(pyridin-4-yl)-1H-benzo[d]imidazole, trifluoroacetic acid salt

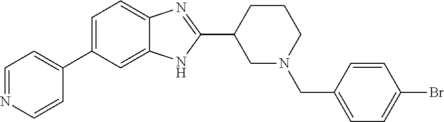

The desired product was prepared by using Example 123 (30 mg) and substituting 4-bromobenzyl bromide for 3-fluorobenzyl bromide in Example 124, and scaling appropriately. Preparative HPLC gave 12 mg of the title compound (29%) as the TFA salt. LC-MS: single peak at 254 nm, MH⁺ calcd. for C₂₄H₂₄BrN₄: 447, obtained: 447. ¹H NMR (DMSO-d₆, 400 MHz) δ 9.13 (d, J=7.1 Hz, 2H), 8.76 (br s, 1H), 8.56 (d, J=7.1 Hz, 2H), 8.33 (s, 1H), 7.91 (dd, J=8.6, 1.8, 1H), 7.75 (d, J=8.5 Hz, 1H), 7.69 (d, J=8.5 Hz, 1H), 7.54 (d, J=8.5 Hz, 2H), 5.78 (s, 2H), 3.63 (d, J=11.8 Hz, 1H), 3.44-3.36 (m, 1H), 3.32 (d, J=10.6 Hz, 2H), 2.97 (m, 1H), 2.21 (m, 1H), 1.92-1.85 (m, 1H), 1.81-1.76 (m, 2H).

### Example 128

### 2-(1-allylpiperidin-3-yl)-6-(pyridin-4-yl)-1H-benzo[d]imidazole, trifluoroacetic acid salt

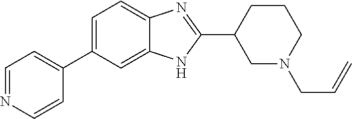

The desired product was prepared by using Example 123 (40 mg) and substituting allyl bromide for 3-fluorobenzyl bromide in Example 124, and scaling appropriately. Preparative HPLC gave 26 mg of the title compound (60%) as the TFA salt. LC-MS: single peak at 254 nm, MH⁺ calcd. for C₂₀H₂₃H₄: 319, obtained: 319. ¹H NMR (DMSO-d₆, 400 MHz) δ 8.98 (d, J=7.1 Hz, 2H), 8.85-8.74 (m, 2H), 8.57 (d, J=7.1 Hz, 2H), 8.34 (s, 1H), 7.93 (dd, J=8.6, 1.8, 1H), 7.76 (d, J=6.7 Hz, 1H), 6.25-6.15 (m, 1H), 5.46 (dd, J=10.2, 1.0 Hz, 1H), 5.42 (dd, J=17.0, 1.2 Hz, 1H), 5.23 (d, J=6.2 Hz, 2H), 3.64 (d, J=11.7 Hz, 1H), 3.45-3.32 (m, 3H), 3.10-2.91 (m, 1H), 2.22 (m, 1H), 1.93-1.86 (m, 1H), 1.84-1.75 (m, 2H).

### Example 129

### 2-(1-(2,4-difluorobenzyl)piperidin-3-yl)-6-(pyridin-4-yl)-1H-benzo[d]imidazole, trifluoroacetic acid salt

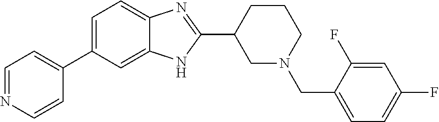

The desired product was prepared by using Example 123 (40 mg) and substituting 2,4-difluorobenzyl bromide for 3-fluorobenzyl bromide in Example 124, and scaling appropriately. Preparative HPLC gave 3 mg of the title compound (7%) as the TFA salt. LC-MS: single peak at 254 nm, MH⁺ calcd. for C₂₄H₂₃F₂N₄: 405, obtained: 405.

### Example 130

### 2-(1-(4-chlorobenzyl)piperidin-3-yl)-6-(pyridin-4-yl)-1H-benzo[d]imidazole, trifluoroacetic acid salt

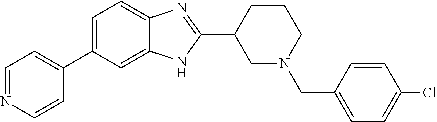

The desired product was prepared by using Example 123 (40 mg) and substituting 4-chlorobenzyl bromide for 3-fluorobenzyl bromide in Example 124, and scaling appropriately. Preparative HPLC gave 17 mg of the title compound (34%) as the TFA salt. LC-MS: single peak at 254 nm, MH⁺ calcd. for C₂₄H₂₄ClN₄: 403, obtained: 403. ¹H NMR (DMSO-d₆, 400 MHz) δ 9.13 (d, J=7.1 Hz, 2H), 8.74 (br s, 1H), 8.56 (d, J=7.1 Hz, 2H), 8.33 (s, 1H), 7.91 (dd, J=8.5, 1.7, 1H), 7.74 (d, J=8.6 Hz, 1H), 7.62 (d, J=8.6 Hz, 1H), 7.55 (d, J=8.6 Hz, 2H), 5.80 (s, 2H), 3.64 (d, J=10.9 Hz, 1H), 3.43-3.28 (m, 3H), 3.02-2.92 (m, 1H), 2.21 (m, 1H), 1.93-1.86 (m, 1H), 1.83-1.75 (m, 2H).

### Example 131

### 2-(1-(4-methoxybenzyl)piperidin-3-yl)-6-(pyridin-4-yl)-1H-benzo[d]imidazole, trifluoroacetic acid salt

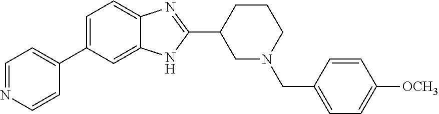

The desired product was prepared by using Example 123 (40 mg) and substituting 4-methoxybenzyl bromide for 3-fluorobenzyl bromide in Example 124, and scaling appropriately. Preparative HPLC gave 10 mg of the title compound (19%) as the TFA salt. LC-MS: single peak at 254 nm, MH⁺ calcd. for C₂₅H₂₇N₄O: 399, obtained: 399. ¹H NMR (DMSO-d₆, 400 MHz), δ 9.11 (d, J=7.1 Hz, 2H), 8.72 (br s, 2H), 8.53 (d, J=7.1 Hz, 2H), 8.31 (s, 1H), 7.89 (dd, J=8.5, 1.8, 1H), 7.74 (d, J=8.6 Hz, 1H), 7.56 (d, J=8.8 Hz, 1H), 7.02 (d, J=8.8 Hz, 1H), 5.72 (s, 2H), 3.64 (d, J=11.3 Hz, 1H), 3.43-3.29 (m, 3H), 3.06-2.88 (m, 1H), 2.21 (m, 1H), 1.92-1.85 (m, 1H), 1.83-1.75 (m, 2H).

### Example 132

### tert-butyl 4-(6-(pyridin-4-yl)-1H-benzo[d]imidazol-2-yl)piperidine-1-carboxylate

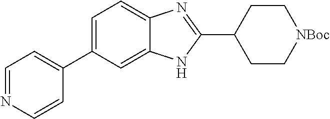

Example 114B (120 mg) was dissolved in a solution of 50% TFA in methylene chloride (2 mL) and the mixture was stirred at room temperature for 1 hour. The solvent was removed under reduced pressure and excess acid was removed by repeated evaporation from toluene in vacuo. Without further purification, the crude diamine was treated with enough DMF (0.5 mL) to dissolve the residue. The solution was treated with N-Boc-isonipecotic acid (71 mg, 0.311 mmol, 1 equiv.), HOBt (1.1 equiv.), NMM (2 equiv.), and EDC (1.1 equiv.). The resulting mixture was stirred at 0° C., then allowed to warm up to room temperature overnight. After removal of solvent by rotary evaporation, the residue was dissolved in ethyl acetate (15 mL) and a saturated aqueous solution of NaHCO₃ (5 mL). The layers were separated and the organic layer was washed with brine (5 mL), dried over Na₂SO₄, filtered, and the solvent was removed in vacuo. Without further purification, the residue was dissolved in acetic acid and heated by microwave irradiation at 100° C. for 65 min. The solvent was removed by rotary evaporation and the residue was purified by HPLC. Preparative HPLC gave 10 mg of the title compound (7%). LC-MS: single peak at 254 nm, MH⁺ calcd. for C₂₂H₂₇N₄O₂: 379, obtained: 379.

### Example 133

### 2-(1-benzylpiperidin-4-yl)-6-(pyridin-4-yl)-1H-benzo[d]imidazole

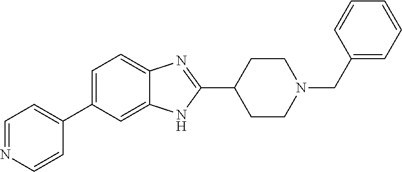

The desired product was prepared using by substituting N-benzylisonipecotic acid (130 mg) for N-Boc-isonipecotic acid, and scaling appropriately. Preparative HPLC gave 20 mg of the title compound (10%). LC-MS: single peak at 254 nm, MH⁺ calcd. for C₂₄H₂₅N₄: 369, obtained: 369.

### Example 134

### 2-(1-(3-fluorobenzyl)piperidin-4-yl)-6-(pyridin-4-yl)-1H-benzo[d]imidazole

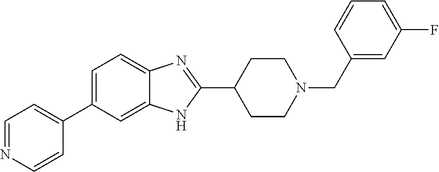

The desired product was prepared using by substituting N-(3-fluorobenzyl)isonipecotic acid (127 mg) for N-Boc-isonipecotic acid, and scaling appropriately. Preparative HPLC gave 43 mg of the title compound (21%). LC-MS: single peak at 254 nm, MH⁺ calcd. for C₂₄H₂₄FN₄: 387, obtained: 387.

### Example 135

### 2-(1-allylpiperidin-4-yl)-6-(pyridin-4-yl)-1H-benzo[d]imidazole

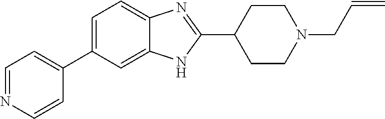

The desired product was prepared using by substituting N-allylisonipecotic acid (120 mg) for N-Boc-isonipecotic acid, and scaling appropriately. Preparative HPLC gave 51 mg of the title compound (30%). LC-MS: single peak at 254 nm, MH⁺ calcd. for C₂₀H₂₃N₄: 319, obtained: 319.

### Example 136

### 2-(1-phenethylpiperidin-4-yl)-6-(pyridin-4-yl)-1H-benzo[d]imidazole

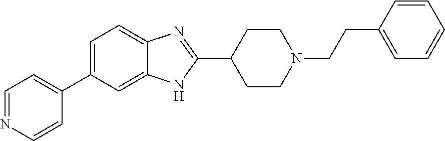

The desired product was prepared using by substituting N-phenethylisonipecotic acid (121 mg) for N-Boc-isonipecotic acid, and scaling appropriately. Preparative HPLC gave 46 mg of the title compound (24%). LC-MS: single peak at 254 nm, MH⁺ calcd. for C₂₅H₂₇N₄: 383, obtained: 383.

### Example 137

### 2-((3R,4S)-4-phenylpyrrolidin-3-yl)-5-(pyridin-4-yl)-1H-benzo[d]imidazole

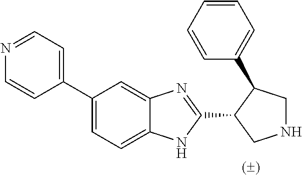

A mixture of 4-bromobenzene-1,2-diamine (187 mg, 1 equiv.), (±)-trans-1-(tert-butoxycarbonyl)-4-phenylpyrrolidine-3-carboxylic acid (291 mg, 1 equiv., available from PepTech Corporation, Burlington, Mass.), HATU (1.5 equiv.), DIEA (3 equiv.) in a minimal amount of DMF (2 mL solvent per mmol of the diamine) was maintained at 25° C. for 2 hours (or until complete as determined by LC-MS). This solution was directly placed on dry silica gel which was then eluted with an appropriate gradient of ethyl acetate and hexane to obtain the pure amide products. The mixture of regioisomeric amides was dissolved in a minimal amount of glacial acetic acid. The solution was heated at 60° C. for 1 hour (or until complete as determined by LC-MS), concentrated in vacuo, then the residue was dissolved in dichloromethane and this solution was applied to a silica gel column which was then eluted with an appropriate gradient of ethyl acetate and hexane to obtain the pure benzimidazole arylbromide product. A mixture of the arylbromide (1 equiv.), 4-(4,4,5,5-tetramethyl-1,3,2-dioxaborolan-2-yl)pyridine (1.2 equiv.), tetrakistriphenylphosphine palladium (0.03 equiv.), and sodium bicarbonate (3.4 equiv.) was suspended in a 2:1 mixture of dimethoxyethane and water (3 mL total, when 0.2 mmol of bromide was used) in a microwave pressure vessel. The sealed vessel was heated for 20 minutes at 120° C. using 300W of microwave energy. The solution was cooled, poured into water, and extracted with chloroform. The organic extracts were dried, filtered, concentrated in vacuo, then the residue was dissolved in a minimal amount of dichloromethane and this solution was applied to a silica gel column which was then eluted with an appropriate gradient of ethyl acetate and hexane to obtain the desired Boc-protected product. This product was dissolved in a 3:1 (v/v) mixture of dichloromethane and TFA and the reaction was maintained at room temperature for 30 minutes (or until complete as determined by LC-MS). The solution was concentrated in vacuo, then the residue was dissolved in a minimal amount of dichloromethane and this solution was applied to a silica gel column which was then eluted with an appropriate gradient of methanol in dichloromethane containing 1% ammonia, to obtain the desired product. LC-MS: single peak at 254 nm, retention time 1.22 minutes using Agilent LC-MS general method 1, MH⁺ calcd. for C₂₄H₂₀N₄: 341.2, obtained: 341.2.

### Example 138

### 2-(5-(pyridin-4-yl)-1H-benzo[d]imidazol-2-yl)-3,4-dihydro-2H-benzo[b][1,4]oxazine

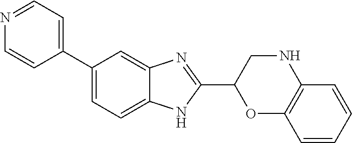

The desired product was prepared by substituting 3,4-dihydro-2H-benzo[b][1,4]oxazine-2-carboxylic acid (available from Fisher Scientific, Pittsburgh, Pa.) for (±)-trans-1-(tert-butoxycarbonyl)-4-phenylpyrrolidine-3-carboxylic acid in Example 137, but omitting the deprotection step. LC-MS: single peak at 254 nm, retention time 1.51 minutes using Agilent LC-MS general method 1, MH⁺ calcd. for C₂₀H₁₆N₄O: 329.1, obtained: 329.1.

### Example 139

### 2-(5-(pyridin-4-yl)-1H-benzo[d]imidazol-2-yl)-2,3-dihydro-1H-inden-2-amine

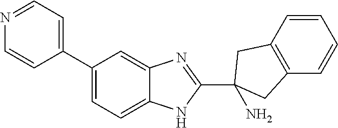

The desired product was prepared by substituting 2-(tert-butoxycarbonylamino)-2,3-dihydro-1H-indene-2-carboxylic acid (available from Acros Organics USA, Morris Plains, N.J.) for (±)-trans-1-(tert-butoxycarbonyl)-4-phenylpyrrolidine-3-carboxylic acid in Example 137. LC-MS: single peak at 254 nm, retention time 1.24 minutes using Agilent LC-MS general method 1, MH⁺ calcd. for C₂₁H₁₈N₄: 327.2, obtained: 327.2.

### Example 140

### (1R,3S)-3-(5-(pyridin-4-yl)-1H-benzo[d]imidazol-2-yl)cyclopentanamine

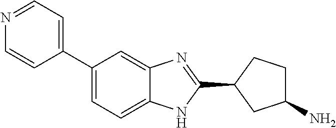

The desired product was prepared by substituting (1S,3R)-3-(tert-butoxycarbonylamino)cyclopentanecarboxylic acid (available from Acros Organics USA, Morris Plains, N.J.) for (±)-trans-1-(tert-butoxycarbonyl)-4-phenylpyrrolidine-3-carboxylic acid in Example 137. LC-MS: single peak at 254 nm, retention time 0.44 minutes using Agilent LC-MS general method 1, MH⁺ calcd. for C₁₇H₁₈N₄: 279.2, obtained: 279.2.

### Example 141

### (1S,3R)-3-(5-(pyridin-4-yl)-1H-benzo[d]imidazol-2-yl)cyclopentanamine

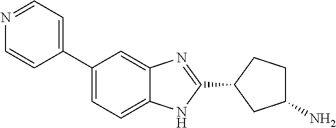

The desired product was prepared by substituting (1R,3S)-3-(tert-butoxycarbonylamino)cyclopentanecarboxylic acid (available from Acros Organics USA, Morris Plains, N.J.) for (±)-trans-1-(tert-butoxycarbonyl)-4-phenylpyrrolidine-3-carboxylic acid in Example 137. LC-MS: single peak at 254 nm, retention time 0.44 minutes using Agilent LC-MS general method 1, MH⁺ calcd. for C₁₇H₁₈N₄: 279.2, obtained: 279.2.

### Example 142

### General Procedure for the Preparation of Substituted Benzimidazoles No. 1

A primary amine (1.0 equiv) is added to a solution of a 4-bromo-2-fluoro-1-nitrobenzene derivative (1.0 equiv) and K₂CO₃ (1.1 equiv) in DMSO (20 mL/mmol). The mixture is stirred at room temperature until the reaction was complete. Upon completion, the solution is diluted with water and the resulting precipitate is collected by filtration to give an arylamine. The arylamine (1.0 equiv) is then dissolved in an EtOAc/EtOH solution (2:1 by volume, 20 mL/mmol) and SnCl₂′2H₂O (5.0 equiv) is subsequently added. The resulting mixture is warmed to 70° C. and stirred until reduction is complete. Upon completion, the solution is portioned between saturated NaHCO₃ and EtOAc. The aqueous portion is then extracted twice more with additional EtOAc. The combined organic portions are dried over MgSO₄ and concentrated to give a diamine. The diamine (1.0 equiv) is added to a solution of a carboxylic acid (1.0 equiv), HATU (1.2 equiv) and Et₃N (2.0 equiv) in DMF (30 mL/mmol). The solution is then stirred for 60 minutes at room temperature. At this time the solution is concentrated in vacuo to give an arylamide intermediate. This unpurified material is dissolved in AcOH (20 mL/mmol) and warmed to 60° C. until the cyclodehyration is complete (1-72 hours). Upon completion, the solution is concentrated in vacuo and the residue is diluted with EtOAc and washed with saturated NaHCO₃. The aqueous portion is then extracted twice more with additional EtOAc. The combined organic portions were dried over MgSO₄ and concentrated. Purification on silica gel (CH₂Cl₂/EtOAc gradient) gives an N-substituted benzimidazole bromide (arylbromide). This arylbromide can then participate in a Suzuki reaction with an aryl or heteroaryl boronate (or boronic acid) to give compound the desired product directly. Thus, arylbromide (1.0 equiv.) is combined with an arylboronate (1.3 equiv.), Na₂CO₃ (3.0 equiv.) and PdCl₂(PPh₃)₂ (0.1 equiv.) under streaming argon in a pressure tube. Aqueous dioxane (5:1 dioxane:H₂O by volume, 40 mL/mmol) is then added and the solution is sparged with argon for 10 minutes. The solution is then heated in a microwave at 120° C. until the reaction is complete. The solution is then purified, for example via preparative HPLC to give the desired product as the TFA salt. Alternatively, the arylbromide can be converted into an arylboronate prior to the Suzuki coupling. This is done by mixing arylbromide (1.0 equiv.), bis(pinicalato)diboron (2.5 equiv.), KOAc (5.0 equiv.) and PdCl₂(dppf) (0.1 equiv.) under an argon atmosphere in a pressure tube. Dioxane (20 mL/mmol) is then added and the solution is sparged for 10 minutes with argon. The reaction is then warmed to 100° C. in a microwave for 60 minutes. The solution is then partitioned between brine and EtOAc. The aqueous portion is then extracted twice more with additional EtOAc. The combined organic portions are dried over MgSO₄ and concentrated. Purification on silica gel (CH₂Cl₂/EtOAc gradient) gives the arylboronate. This arylboronate can then participate in a Suzuki reaction to give compound the desired product. Thus, the arylboronate (1.0 equiv.) is combined with an aryl or heteroaryl halide (1.0 equiv.), Na₂CO₃ (3.0 equiv.) and PdCl₂(PPh₃)₂ (0.1 equiv.) under streaming argon in a pressure tube. Aqueous dioxane (5:1 dioxane:H₂O by volume, 20 mL/mmol) is then added and the solution is sparged with argon for 10 minutes. The solution is then heated in a microwave at 120° C. until the reaction is complete. The solution is then purified, for example via preparative HPLC to give the desired product as the TFA salt.

### Example 143

### 2-(2-(chroman-3-yl)-5-fluoro-6-(pyridin-4-yl)-1H-benzo[d]imidazol-1-yl)-N,N-dimethylethanamine

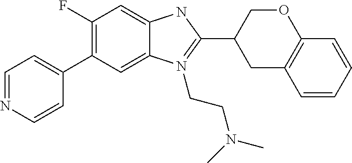

The desired product was prepared by using 1-bromo-2,5-difluoro-4-nitrobenzene (0.7 mmol), N,N-dimethylethylenediamine, chroman-3-carboxylic acid, and a pyridine-4-boronate in Example 142. 23.3 mg desired product was obtained from 48.0 mg of the arylbromide. LC-MS: single peak at 254 nm, MH⁺ calcd. for C₂₅H₂₅FN₄O: 417, obtained 417. HPLC: single peak by analytical HPLC. ¹H-NMR (MeOD- d₄, 400 MHz): 8.77 (2H, s), 8.11 (3H, m), 7.57 (1H, m), 7.15 (2H, m), 6.90 (2H, m), 4.89 (2H?, obscured by solvent peak), 4.58 (1H, m), 4.29 (1H, m), 3.79 (1H, m), 3.66 (2H, m), 3.34 (1H, m), 3.26 (1H, m), 3.04 (6H, s).

### Example 144

### 2-(2-(chroman-3-yl)-5-fluoro-6-(1H-pyrazol-4-yl)-1H-benzo[d]imidazol-1-yl)-N,N-dimethylethanamine

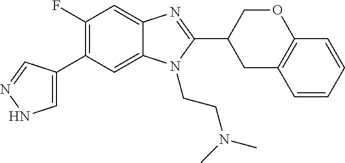

The desired product was prepared by using 1-bromo-2,5-difluoro-4-nitrobenzene (0.7 mmol), N,N-dimethylethylenediamine, chroman-3-carboxylic acid, and a pyrazole-4-boronate in Example 142. 8.28 mg desired product was obtained from 48.0 mg of the arylbromide. LC-MS: single peak at 254 nm, MH⁺ calcd. for C₂₃H₂₄FN₅O: 406, obtained 406. HPLC: single peak by analytical HPLC. ¹H-NMR (MeOD- d₄, 400 MHz): 8.10 (2H, s), 7.85 (1H, m), 7.39 (1H, m), 7.14 (2H, m), 6.88 (2H, m), 4.50 (3H, m), 4.23 (1H, m), 3.69 (1H, m), 3.38 (2H, m), 3.17 (1H, m), 2.76 (2H, bs), 2.32 (6H, s).

### Example 145

### 2-(2-(chroman-3-yl)-5-fluoro-6-(5-methyl-1H-pyrazol-4-yl)-1H-benzo[d]imidazol-1-yl)-N,N-dimethylethanamine

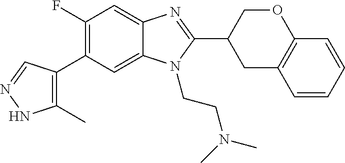

The desired product was prepared by using 1-bromo-2,5-difluoro-4-nitrobenzene (0.7 mmol), N,N-dimethylethylenediamine, chroman-3-carboxylic acid, and a 5-methylpyrazole-4-boronate in Example 142. 6.77 mg desired product was obtained from 48.0 mg of the arylbromide. LC-MS: single peak at 254 nm, MH⁺ calcd. for C₂₄H₂₆FN₅O: 420, obtained 420. HPLC: single peak by analytical HPLC. ¹H-NMR (MeOD- d₄, 400 MHz): 7.70 (1H, b), 7.54 (1H, dd, J=1.5 Hz, 6.3 Hz), 7.41 (1H, dd, J=2.1 Hz, 10.5 Hz), 7.15 (2H, m), 6.89 (2H, m), 4.55 (1H, dd, J=2.3 Hz, 10.6 Hz), 4.47 (1H, t, J=6.3 Hz), 4.26 (1H, dt, J=2.1 Hz, 10.6 Hz), 3.72 (1H, m), 3.36 (1H, m), 3.17 (1H, m), 2.75 (2H, m), 2.38 (3H, s), 2.31 (6H, s).

### Example 146

### 2-(2,3-dihydrobenzo[b][1,4]dioxin-2-yl)-1-(1-methylpiperidin-4-yl)-6-(pyridin-4-yl)-1H-benzo[d]imidazole, trifluoroacetic acid salt

### 146A. N-(5-bromo-2-nitrophenyl)-1-methylpiperidin-4-amine

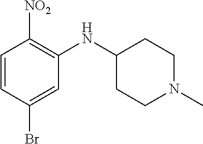

The desired product was prepared by using 4-bromo-2-fluoro-1-nitrobenzene (3.55 mmol) and 1-methylpiperidin-4-amine (3.55 mmol) in Example 142. The reaction was run for 2 hours to give 985 mg of the desired product as an yellow solid (88% yield). Single peak by HPLC.

### 146B. 5-bromo-N1-(1-methylpiperidin-4-yl)benzene-1,2-diamine

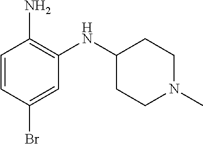

The desired product was prepared according to Example 142. The reaction was run for 24 hours on 1.84 mmol scale to give 474 mg of the desired product as a colorless oil (90% yield). Single peak by HPLC.

### 146C. 6-bromo-2-(2,3-dihydrobenzo[b][1,4]dioxin-2-yl)-1-(1-methylpiperidin-4-yl)-1H-benzo[d]imidazole

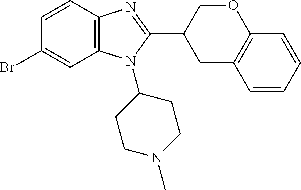

The desired product was prepared by using 1,4-benzodioxan-2-carboxylic acid (1.67 mmol) in Example 142. Purification by extraction alone gave 516 mg of the product as a colorless solid (72% yield). LCMS (found 428.1, 430.1, MH+ calculated for C₂₁H₂₃BrN₃O₂: 428.1, 430.1). Single peak by HPLC.

### 146D. 2-(2,3-dihydrobenzo[b][1,4]dioxin-2-yl)-1-(1-methylpiperidin-4-yl)-6-(pyridin-4-yl)-1H-benzo[d]imidazole, trifluoroacetic acid salt

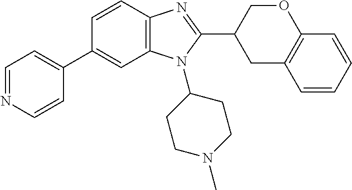

The desired product was prepared by using a pyridine-4-boronate in Example 142. The reaction was run on a 0.154 mmol scale. Purification by preparative HPLC gave 54 mg of the product (TFA salt) as a tan solid (64% yield). LCMS (found 427.2, MH+ calculated for C₂₆H₂₇N₄O₂: 427.2). ¹H-NMR (MeOH-d₄, 400 MHz) δ 2.18-2.27 (m, 2H), 2.75-2.94 (m, 5H), 3.23-3.36 (m, 2H), 3.59-3.72 (m, 2H), 4.65 (dd, 8.0 Hz, 12.0 Hz, 1H), 4.87 (dd, 2.4 Hz, 7.6 Hz, 1H), 5.05-5.14 (m, 1H), 5.84 (dd, 2.4 Hz, 7.6 Hz, 1H), 6.85-7.05 (m, 4H), 7.76 (d, 8.8 Hz, 1H), 7.90 (d, 8.8 Hz, 1H), 8.13 (d, 5.8 Hz, 2H), 8.26 (s, 1H), 8.83 (d, 5.8 Hz, 2H), 9.93 (bs, 1H). Single peak by HPLC.

### Example 147

### General Procedure for the Preparation of Substituted Benzimidazoles No. 2

A primary amine R¹NH₂ (1.1 equiv.) is added to a solution of a 4-bromo-2-fluoro-1-nitrobenzene derivative (1.0 equiv.) and K₂CO₃ (2 equiv.) in DMF (10 mL/mmol). After the mixture is stirred at 23° C. overnight, the DMF is removed under reduced pressure. The resulting residue is suspended in ethyl acetate, washed with brine (2×), saturated NaHCO₃ (2×), brine (2×), and dried over Na₂SO₄. The organic solvents are evaporated by a Rotovapor, and the resulting residue is subjected to flash chromatography (gradient methanol in dichloromethane) to give an arylamine (characterized by LC-MS). Suzuki coupling reaction: Pd[P(Ph)₃]₄ (10% by weight) is added to a degassed (under argon) solution of the arylamine (1.0 equiv), an aryl or heteroaryl boronic acid (or its ester derivative) Ar¹-B(V)₂ (2 equiv.), and K₂CO₃ (4 equiv.) in dioxane/water (4:1 by volume, 15 mL/mmol) in a high pressure reactor. After the reactor is sealed, the mixture is heated to and stirred at 100° C. for 5-40 hours. The solvents are evaporated in vacuo, and the residue is subjected to flash chromatography (gradient methanol in dichloromethane) to give the biaryl product. The SnCl₂ method is used to reduce the nitro group. Thus, dihydrated SnCl₂ (4.0 equiv.) is added to a solution of the biaryl product (1.0 equiv.) in 5% isopropanol/dioxane (10 mL/mmol), and the mixture is stirred at 23° C. overnight. The solvents are evaporated in vacuo, the residue is suspended in water with KOH (15 equiv based on compound the biaryl product), and the aqueous solution is extracted with dichloromethane (5×). The organic phases are combined, dried over Na₂SO₄, evaporated to a residue, that is subjected to flash chromatography (gradient methanol in dichloromethane) to give a diamine. Next, a general amide coupling method using HATU is utilized to synthesize an arylamide intermediate. Thus, HATU (1.4 equiv.) is added to a stirring solution of the diamine (1.0 equiv.), a carboxylic acid E-COOH (1.3 equiv.), and DIEA (3 equiv.) in DMF (10 mL/mmol). After the reaction is finished (monitored by LC-MS, 1 hour to overnight), the DMF is removed under reduced pressure. The residue is suspended in ethyl acetate, washed consecutively with brine (2×), saturated NaHCO₃ (3×), brine (2×), and dried over Na₂SO₄. The solvent is evaporated in vacuo to obtain a crude product arylamide intermediate. This crude material was used directly in the next step without further purification. Thus, the crude arylamide intermediate is suspended in acetic acid (15 mL/mmol), and the mixture is heated at 60° C. for several hours (the reaction was monitored by LC-MS). After the removal of the acetic acid by evaporation under reduced pressure, the residue is subjected to reverse phase preparative HPLC to give the desired final product as a TFA salt.

### Example 148

### 3-(2-(6-chlorochroman-3-yl)-5-fluoro-6-(1H-pyrazol-4-yl)-1H-benzo[d]imidazol-1-yl)-N,N-dimethylpropan-1-amine, bis-trifluoroacetic acid salt

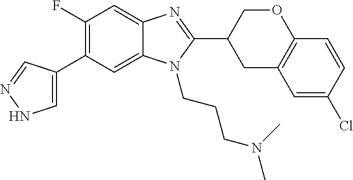

The desired product was prepared by using 1-bromo-2,5-difluoro-4-nitrobenzene, 3-dimethylamino-1-propylamine, 6-chlorochroman-3-carboxylic acid, and a pyrazole-4-boronate in Example 147. 60 mg desired product (bis-TFA salt) were obtained from 0.3 mmol of the diamine intermediate. LC-MS: single peak at 254 nm, MH⁺ calcd. for C₂₄H₂₅FClN₅O: 454, obtained 454. HPLC: single peak by analytical HPLC. ¹H-NMR (DMSO-d₆, 400 MHz): 9.60 (1H, b), 8.07 (2H, d, J=2.0 Hz), 7.94 (1H, d, J=6.8 Hz), 7.50 (1H, d, J=12 Hz), 7.27 (1H, d, J=2.4 Hz), 7.19 (1H, dd, J=2.8 Hz, 8.8 Hz), 6.89 (1H, d, J=8.8 Hz), 4.53 (1H, m), 4.40 (2H, t, J=7.6 Hz), 4.18 (1H, t, J=6.4 Hz), 3.68 (1H, m), 3.18 (4H, m), 2.78 (3H, s), 2.77 (3H, s), 2.14 (2H, m).

### Example 149

### 3-(2-(6-chlorochroman-3-yl)-5-fluoro-6-(5-methyl-1H-pyrazol-4-yl)-1H-benzo[d]imidazol-1-yl)-N,N-dimethylpropan-1-amine, bis trifluoroacetic acid salt

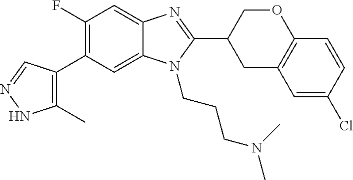

The desired product was prepared by using 1-bromo-2,5-difluoro-4-nitrobenzene, 3-dimethylamino-1-propylamine, 6-chlorochroman-3-carboxylic acid, and a 5-methylpyrazole-4-boronate in Example 147. 55 mg desired product (bis-TFA salt) were obtained from 0.3 mmol of the diamine intermediate. LC-MS: single peak at 254 nm, MH⁺ calcd. for C₂₅H₂₇FClN₅O: 468, obtained 468. HPLC: single peak by analytical HPLC.

### Example 150

### 3-(2-(6-chlorochroman-3-yl)-5-fluoro-6-(pyridin-4-yl)-1H-benzo[d]imidazol-1-yl)-N,N-dimethylpropan-1-amine, bis trifluoroacetic acid salt

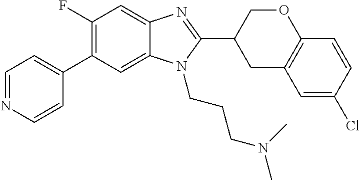

The desired product was prepared by using 1-bromo-2,5-difluoro-4-nitrobenzene, 3-dimethylamino-1-propylamine, 6-chlorochroman-3-carboxylic acid, and a pyridine-4-boronate in Example 147. 82 mg desired product (bis-TFA salt) were obtained from 0.3 mmol of the diamine intermediate. LC-MS: single peak at 254 nm, MH⁺ calcd. for C₂₆H₂₆FClN₄O: 465, obtained 465. HPLC: single peak by analytical HPLC. ¹H-NMR (DMSO-d₆, 400 MHz): 8.89 (2H, dd, J=1.2 Hz, 5.2 Hz), 8.08 (1H, d, J=7.2 Hz), 8.03 (2H, d, J=5.2 Hz), 7.70 (1H, d, J=12 Hz), 7.28 (1H, d, J=2.4 Hz), 7.19 (1H, dd, J=2.8 Hz, 8.8 Hz), 6.90 (1H, d, J=8.8 Hz), 4.56 (1H, dt, J=2.4 Hz, 8.4 Hz), 4.46 (2H, t, J=7.6 Hz), 4.21 (1H, t, J=6.4 Hz), 3.74 (1H, m), 3.19 (4H, m), 2.78 (3H, s), 2.77 (3H, s), 2.14 (2H, m).

### Example 151

### (R)-2-(2-(2,3-dihydrobenzo[b][1,4]dioxin-2-yl)-6-(1H-pyrazol-4-yl)-1H-benzo[d]imidazol-1-yl)-N,N-dimethylethanamine, bis trifluoroacetic acid salt

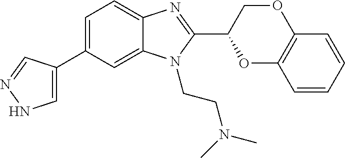

The desired product was prepared by using 4-bromo-2-fluoro-1-nitrobenzene, N,N-dimethylethylenediamine, (S)-1,4-benzodioxan-2-carboxylic acid, and a pyrazole-4-boronate in Example 147. 90 mg desired product (bis-TFA salt) were obtained from 0.4 mmol of the diamine intermediate. LC-MS: single peak at 254 nm, MH⁺ calcd. for C₂₂H₂₃N₅O₂: 390, obtained 390. HPLC: single peak by analytical HPLC.

### Example 152

### 2-(6-chlorochroman-3-yl)-5-(pyridin-4-yl)-1H-benzo[d]imidazole

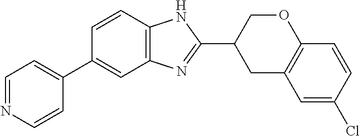

The desired product was prepared by substituting Example 23C (1.0 mmol) for 4-bromobenzene-1,2-diamine and 6-chlorochroman-3-carboxylic acid for chroman-3-carboxylic acid in Example 40A (11.04 mg). LC-MS: single peak at 254 nm, MH⁺ calcd. for C₂₁H₁₆ClN₃O: 362, obtained 362. HPLC: single peak by analytical HPLC.

### Example 153

### 2-(6-methoxychroman-3-yl)-5-(pyridin-4-yl)-1H-benzo[d]imidazole

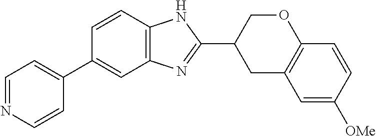

The desired product was prepared by substituting 6-methoxychroman-3-carboxylic acid for 6-chlorochroman-3-carboxylic acid in Example 152 (2.10 mg). LC-MS: single peak at 254 nm, MH⁺ calcd. for C₂₂H₁₉N₃O₂: 358, obtained 358. HPLC: single peak by analytical HPLC.

### Example 154

### (R)-2-(2,3-dihydrobenzo[b][1,4]dioxin-2-yl)-5-(pyridin-4-yl)-1H-benzo[d]imidazole

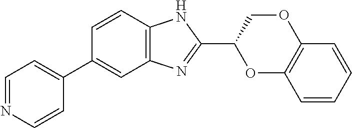

The desired product was prepared by substituting (S)-1,4-benzodioxan-2-carboxylic acid for 6-chlorochroman-3-carboxylic acid in Example 152 (17.60 mg). LC-MS: single peak at 254 nm, MH⁺ calcd. for C₂₀H₁₅N₃O₂: 330, obtained 330. HPLC: single peak by analytical HPLC. ¹H-NMR (MeOD- d₄, 400 MHz): 8.81 (2H, b), 8.36 (2H, d, J=6.6 Hz), 8.24 (1H, d, J=1.4 Hz), 7.90 (1H, dd, J=1.8 Hz, 8.6 Hz), 7.82 (1H, d, J=8.6 Hz), 7.09 (1H, m), 6.92 (3H, m), 5.64 (1H, dd, J=7.1 Hz, 2.6 Hz), 4.70 (1H, dd, J=11.6 Hz, 2.6 Hz), 4.49 (1H, dd, J=7.1 Hz, 11.6 Hz).

### Example 155

### 2-(6-chlorochroman-3-yl)-6-(pyridin-4-yl)-3H-imidazo[4,5-b]pyridine

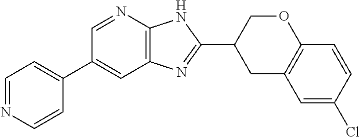

The desired product was prepared by substituting Example 45A (1.0 mmol) for Example 23C and 6-chlorochroman-3-carboxylic acid for chroman-3-carboxylic acid in Example 39 (5.31 mg). LC-MS: single peak at 254 nm, MH⁺ calcd. for C₂₀H₁₅ClN₄O: 363, obtained 363. HPLC: single peak by analytical HPLC.

### Example 156

### 2-(6-methoxychroman-3-yl)-6-(pyridin-4-yl)-3H-imidazo[4,5-b]pyridine

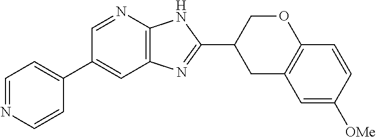

The desired product was prepared by substituting 6-methoxychroman-3-carboxylic acid for 6-chlorochroman-3-carboxylic acid in Example 155 (12.94 mg). LC-MS: single peak at 254 nm, MH⁺ calcd. for C₂₁H₁₈N₄O₂: 359, obtained 359. HPLC: single peak by analytical HPLC. ¹H-NMR (DMSO-d₆, 400 MHz): 8.95 (1H, d, J=2.1 Hz), 8.89 (2H, d, J=6.3 Hz), 8.60 (1H, d, J=1.5 Hz), 8.33 (2H, d, J=6.2 Hz), 6.74 (3H, m), 4.54 (1H, m), 4.30 (1H, m), 3.71 (3H, s), 3.65 (1H, m), 3.36 (1H, m), 3.25 (1H, m).

### Example 157

### (R)-2-(2,3-dihydrobenzo[b][1,4]dioxin-2-yl)-6-(pyridin-4-yl)-3H-imidazo[4,5-b]pyridine

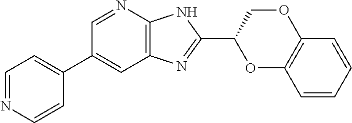

The desired product was prepared by substituting (S)-1,4-benzodioxan-2-carboxylic acid for 6-chlorochroman-3-carboxylic acid in Example 155 (15.28 mg). LC-MS: single peak at 254 nm, MH⁺ calcd. for C₁₉H₁₄H₄O₂: 331, obtained 331. HPLC: single peak by analytical HPLC. ¹H-NMR (DMSO-d₆, 400 MHz): 8.95 (1H, s), 8.83 (2H, d, J=5.3 Hz), 8.57 (1H, b), 8.19 (2H, m), 7.06 (1H, m), 6.91 (3H, m), 5.74 (1H, m), 4.70 (1H, dd, J=2.7 Hz, 11.7 Hz), 4.58 (1H, dd, J=6.9 Hz, 11.8 Hz).

### Example 158

### 2-(6-chlorochroman-3-yl)-7-fluoro-5-(pyridin-4-yl)-1H-benzo[d]imidazole

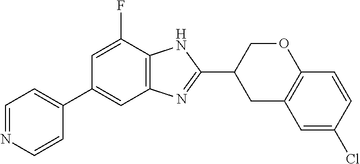

The desired product was prepared by substituting 5-bromo-3-fluorobenzene-1,2-diamine for 4-bromobenzene-1,2-diamine, 6-chlorochroman-3-carboxylic acid for chroman-3-carboxylic acid, and pyridine-4-pinacolboronate for pyrazole-4-pinacolboronate in Example 40. 35.0 mg desired product were synthesized according to the general procedures above from 1.0 mmol of the diamine intermediate. LC-MS: single peak at 254 nm, MH⁺ calcd. for C₂₁H₁₅ClFN₃O: 380, obtained 380. HPLC: single peak by analytical HPLC. ¹H-NMR (DMSO-d₆, 400 MHz): 8.63 (2H, d, J=5.6 Hz), 7.78 (2H, d, J=6.0 Hz), 7.77 (1H, bs), 7.50 (1H, bs), 7.29 (1H, d, J=2.2 Hz), 7.16 (1H, dd, J=2.4 Hz, 8.6 Hz), 6.84 (1H, d, J=8.7 Hz), 4.60 (1H, m), 4.33 (1H, dd, J=9.1 Hz, 10.4 Hz), 3.64 (1H, m), 3.35 (1H, m), 3.25 (1H, dd, J=6.0 Hz, 6.5 Hz).

### Example 159

### 2-(6-chlorochroman-3-yl)-7-fluoro-5-(1H-pyrazol-4-yl)-1H-benzo[d]imidazole

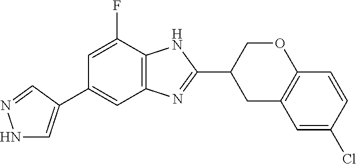

The desired product was prepared by substituting pyrazole-4-pinacolboronate for pyridine-4-pinacolboronate in Example 158. 32.0 mg desired product were synthesized according to the general procedures above from 1.0 mmol of the diamine intermediate. LC-MS: single peak at 254 nm, MH⁺ calcd. for C₁₉H₁₄ClFN₄O: 369, obtained 369.

### Example 160

### 4-(2-(6-chlorochroman-3-yl)-7-fluoro-1H-benzo[d]imidazol-5-yl)pyrimidin-2-amine

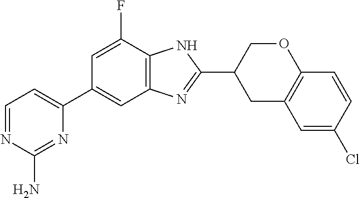

The desired product was prepared by substituting 5-bromo-3-fluorobenzene-1,2-diamine for 4-bromobenzene-1,2-diamine and 6-chlorochroman-3-carboxylic acid for chroman-3-carboxylic acid in Example 40A to give an arylbromide, which was treated as in Example 42A to give an arylboronate, which was treated by substituting 4-chloropyrimidin-2-amine for 4-chloro-7H-pyrrolo[2,3-d]pyrimidine in Example 42B. Preparative HPLC was utilized to give 30.0 mg (27% yield) of the desired product. LC-MS: single peak at 254 nm, MH⁺ calcd. for C₂₀H₁₅ClFN₅O: 396, obtained 396. HPLC: single peak by analytical HPLC. ¹H-NMR (DMSO-d₆, 400 MHz): 8.30 (1H, d, J=5.2 Hz), 8.09 (1H, b), 7.75 (1H, b), 7.29 (1H, d, J=2.4 Hz), 7.21 (1H, d, J=5.3 Hz), 7.15 (1H, dd, J=2.5 Hz, 8.7 Hz), 6.84 (1H, d, J=8.7 Hz), 6.67 (2H, bs), 4.61 (1H, m), 4.32 (1H, m), 3.63 (1H, m), 3.34 (1H, m), 3.27 (1H, dd, J=5.8 Hz, 7.0 Hz).

### Example 161

### 2-(chroman-3-yl)-4-methoxy-5-(pyridin-4-yl)-1H-benzo[d]imidazole

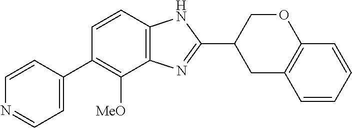

The desired product was prepared by substituting 4-bromo-3-methoxybenzene-1,2-diamine for 4-bromobenzene-1,2-diamine, and pyridine-4-pinacolboronate for pyrazole-4-pinacolboronate in Example 40. Using 1.0 mmol of the diamine intermediate, 2.02 mg of the desired product was obtained via preparative HPLC. LC-MS: single peak at 254 nm, MH⁺ calcd. for C₂₂H₁₉N₃O₂: 358, obtained 358. HPLC: single peak by analytical HPLC.

### Example 162

### tert-butyl (4-chlorophenyl)-(1-(2-(dimethylamino)ethyl)-5-fluoro-6-(1H-pyrazol-4-yl)-1H-benzo[d]imidazol-2-yl)methylcarbamate

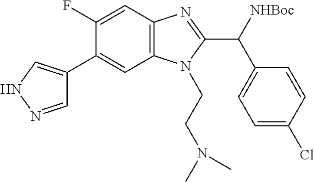

The desired product was prepared by using 1-bromo-2,5-difluoro-4-nitrobenzene, N,N-dimethylethylenediamine, Boc-p-chloro-D,L-Phg-OH and a pyrazole-4-boronate in Example 147. Preparative HPLC was utilized to give the product as white solid (82%). LC/MS: calcd. for C₂₆H₃₀ClFN₆O₂ (M+1) 513, obsd: 513. ¹H-NMR (DMSO-d₆, 400 MHz), δ: 9.87 (br, 1H), 8.14-8.16 (m, 1H), 8.05-8.06 (m, 2H), 7.88-7.89 (m, 1H), 7.44-7.54 (m, 5H), 6.23 (d, J=8.4 Hz, 1H), 4.57-4.68 (m, 2H), 3.38-3.47 (m, 2H), 2.91 (s, 6H), 1.39 (s, 9H).

### Example 163

### 1-allyl-3-(6-(pyridin-4-yl)-1H-benzo[d]imidazol-2-yl)-1,2,3,4-tetrahydroquinoline

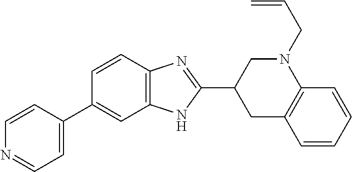

The desired product was prepared by substituting Example 23C for 4-bromobenzene-1,2-diamine and 1-allyl-1,2,3,4-tetrahydroquinoline-3-carboxylic acid for chroman-3-carboxylic acid in Example 40A (31 mg). LC-MS: single peak at 254 nm, MH⁺ calcd. for C₂₄H₂₂N₄: 367, obtained 367. HPLC: single peak by analytical HPLC. ¹H-NMR (MeOD- d₄, 400 MHz): 8.90 (2H, d, J=7.0 Hz), 8.42 (2H, d, J=6.9 Hz), 8.34 (1H, m), 8.11 (1H, dd, J=1.7 Hz, 8.7 Hz), 7.97 (1H, dd, J=8.6 Hz, 0.5 Hz), 7.09 (2H, m), 6.76 (1H, d, J=8.0 Hz), 6.69 (1H, t, J=7.4 Hz), 5.90 (1H, m), 5.19 (2H, m), 4.03 (2H, m), 3.94 (1H, m), 3.81 (1H, ddd, J=1.4 Hz, 3.5 Hz, 11.7 Hz), 3.75 (1H, dd, J=8.0 Hz, 11.8 Hz), 3.40 (2H, m).

### Example 164

### 6-methoxy-1-methyl-3-(6-(pyridin-4-yl)-1H-benzo[d]imidazol-2-yl)-1,2,3,4-tetrahydroquinoline

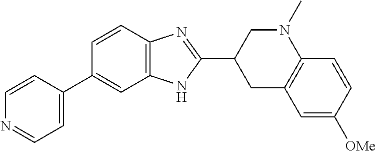

The desired product was prepared by substituting Example 23C for 4-bromobenzene-1,2-diamine and 1,2,3,4-tetrahydro-6-methoxy-1-methylquinoline-3-carboxylic acid for chroman-3-carboxylic acid in Example 40A (7.94 mg). LC-MS: single peak at 254 nm, MH⁺ calcd. for C₂₃H₂₂N₄O: 371, obtained 371. HPLC: single peak by analytical HPLC.

### Example 165

### 2-(6-methylchroman-3-yl)-6-(pyridin-4-yl)-1H-benzo[d]imidazole

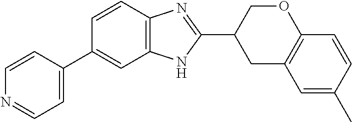

The desired product was prepared by substituting Example 23C for 4-bromobenzene-1,2-diamine and 6-methylchroman-3-carboxylic acid for chroman-3-carboxylic acid in Example 40A (10.13 mg). LC-MS: single peak at 254 nm, MH⁺ calcd. for C₂₂H₁₉N₃O: 342, obtained 342. HPLC: single peak by analytical HPLC. ¹H-NMR (MeOD- d₄, 400 MHz): 8.57 (2H, d, J=6.3 Hz), 7.94 (1H, s), 7.76 (2H, d, J=6.3 Hz), 7.67 (2H, m), 6.97 (1H, s), 6.92 (1H, d, J=8.4 Hz), 6.72 (1H, d, J=8.3 Hz), 4.56 (1H, ddd, J=1.8 Hz, 3.3 Hz, 10.7 Hz), 4.30 (1H, dd, J=9.4 Hz, 10.7 Hz), 3.63 (1H, m), 3.32 (1H, dd, J=9.9 Hz, 16.2 Hz), 3.22 (1H, dd, J=5.3 Hz, 15.9 Hz), 2.26 (3H, s).

### Example 166

### 1-allyl-3-(6-(pyridin-4-yl)-3H-imidazo[4,5-b]pyridin-2-yl)-1,2,3,4-tetrahydroquinoline

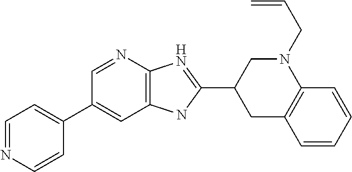

The desired product was prepared by substituting Example 45A for 4-bromobenzene-1,2-diamine and 1-allyl-1,2,3,4-tetrahydroquinoline-3-carboxylic acid for chroman-3-carboxylic acid in Example 40A (4.30 mg). LC-MS: single peak at 254 nm, MH⁺ calcd. for C₂₃H₂₁N₅: 368, obtained 368. HPLC: single peak by analytical HPLC. ¹H-NMR (MeOD- d₄, 400 MHz): 9.00 (1H, d, J=2.1 Hz), 8.89 (2H, d, J=6.9 Hz), 8.57 (1H, d, J=2.1 Hz), 8.48 (2H, d, J=6.9 Hz), 7.06 (2H, m), 6.72 (1H, d, J=8.8 Hz), 6.64 (1H, dt, J=7.4 Hz, 1.0 Hz), 5.92 (1H, m), 5.20 (2H, m), 4.03 (2H, dq, J=5.1 Hz, 9.6 Hz), 3.72 (3H, m), 3.32 (2H, m).

### Example 167

### 1-propyl-3-(6-(pyridin-4-yl)-1H-benzo[d]imidazol-2-yl)-1,2,3,4-tetrahydroquinoline

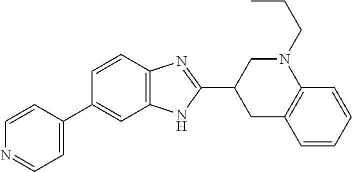

The desired product was prepared by substituting Example 23C for 4-bromobenzene-1,2-diamine and 1-propyl-1,2,3,4-tetrahydroquinoline-3-carboxylic acid for chroman-3-carboxylic acid in Example 40A (5.63 mg). LC-MS: single peak at 254 nm, MH⁺ calcd. for C₂₄H₂₄N₄: 369, obtained 369. HPLC: single peak by analytical HPLC.

### Example 168

### 1-propyl-3-(6-(pyridin-4-yl)-1H-imidazo[4,5-b]pyridin-2-yl)-1,2,3,4-tetrahydroquinoline

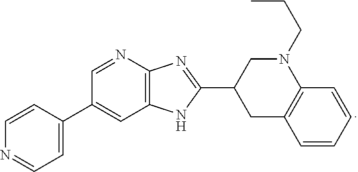

The desired product was prepared by using Example 45A for 4-bromobenzene-1,2-diamine and 1-propyl-1,2,3,4-tetrahydroquinoline-3-carboxylic acid for chroman-3-carboxylic acid in Example 40A (2.54 mg). LC-MS: single peak at 254 nm, MH⁺ calcd. for C₂₃H₂₃N₅: 370, obtained 370. HPLC: single peak by analytical HPLC.

### Example 169

### 7-fluoro-2-(6-methoxychroman-3-yl)-5-(pyridin-4-yl)-1H-benzo[d]imidazole

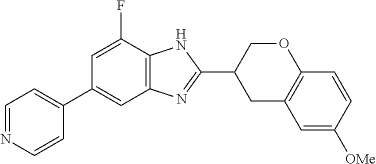

The desired product was prepared by using 5-bromo-3-fluorobenzene-1,2-diamine for 4-bromobenzene-1,2-diamine, 6-methoxychroman-3-carboxylic acid for chroman-3-carboxylic acid, and pyridine-4-pinacolboronate for pyrazole-4-pinacolboronate in Example 40 (34.22 mg). LC-MS: single peak at 254 nm, MH⁺ calcd. for C₂₂H₁₈FN₃O₂: 376, obtained 376.

### Example 170

### 7-fluoro-2-(6-methoxychroman-3-yl)-5-(1H-pyrazol-4-yl)-1H-benzo[d]imidazole

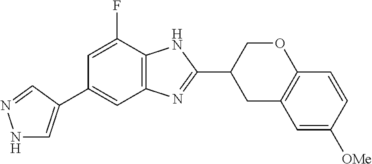

The desired product was prepared by substituting 5-bromo-3-fluorobenzene-1,2-diamine for 4-bromobenzene-1,2-diamine and 6-methoxychroman-3-carboxylic acid for chroman-3-carboxylic acid in Example 40 (3.75 mg). LC-MS: single peak at 254 nm, MH⁺ calcd. for C₂₀H₁₇FN₄O₂: 365, obtained 365. HPLC: single peak by analytical HPLC. ¹H-NMR (MeOD- d₄, 400 MHz): 7.99 (2H, bs), 7.51 (1H, bs), 7.25 (1H, d, J=11.8 Hz), 6.75 (3H, m), 4.53 (1H, ddd, J=1.8 Hz, 3.3 Hz, 10.7 Hz), 4.26 (1H, dd, J=10.5 Hz, 10.8 Hz), 3.76 (3H, s), 3.61 (1H, m), 3.34 (1H, m), 3.22 (1H, dd, J=1.2 Hz, 16.5 Hz).

### Example 171

### 4-(7-fluoro-2-(6-methoxychroman-3-yl)-1H-benzo[d]imidazol-5-yl)pyrimidin-2-amine

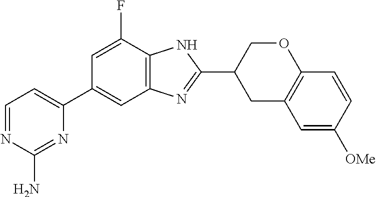

The desired product was prepared by substituting 6-methoxychroman-3-carboxylic acid for 6-chlorochroman-3-carboxylic acid in Example 160. Preparative HPLC was used to give 2.92 mg of the desired product. LC-MS: single peak at 254 nm, MH⁺ calcd. for C₂₁H₁₈FN₅O₂: 392, obtained 392. HPLC: single peak by analytical HPLC.

### Example 172

### methyl 3-(5-(2-aminopyrimidin-4-yl)-1H-benzo[d]imidazol-2-yl)-chroman-6-carboxylate

### 172A. 6-(methoxycarbonyl)chroman-3-carboxylic acid

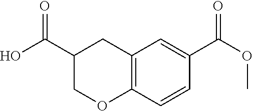

### Step 1: Methyl 3-formyl-4-hydroxybenzoate (Aldrich; 2.69 g, 14.93 mmol) was placed in a 20 mL microwave tube. 1,4-diazabicyclo[2.2.2]octane (Aldrich; 0.25 equiv., 1.7 mmol, 419 mg) was added, followed by benzyl acrylate (Alfa Aesar; 2 equiv., 29.9 mmol, 4.84 g). The mixture was heated in a Biotage Initiator Microwave reactor at 160° C. for 30 minutes and cooled to room temperature. The viscous liquid was placed on a dry 80 g silica cartridge. The tube was rinsed with minimal dichloromethane, and the rinses were added to the column. The column was purged with air and then eluted with a slow gradient of 0 to 60% dichloromethane in hexanes. Fractions containing the purified product of this step, 3-benzyl 6-methyl 2H-chromene-3,6-dicarboxylate, as determined by analytical HPLC, were concentrated in vacuo to provide 2.1 g of material (43%).

Step 2: The product of step 1,3-benzyl 6-methyl 2H-chromene-3,6-dicarboxylate, was dissolved in 100 mL of 1:1 ethyl acetate and hexane and placed in a 1L pressure hydrogenation bottle. 200 mg of 10% palladium on carbon (Aldrich) was added, and the solution was pressurized in a Parr apparatus with 50 psi hydrogen gas and shaken 12 h at room temperature. Venting of the excess hydrogen, filtration through Celite, and concentration in vacuo gave the crude chroman acid. This white solid was dissolved in minimal hot dichloromethane and placed on a dry 80 g silica cartridge. The flask was rinsed with minimal dichloromethane, and the rinses were added to the column. The column was purged with air and then eluted with a slow gradient of 0 to 60% hexane in ethyl acetate, except that the ethyl acetate solution also contained 1% acetic acid. Fractions containing the pure product were concentrated in vacuo to provide 1.16 g of the desired product (76%). These pure fractions showed a single peak by analytical HPLC; LC-MS (negative mode detection): C₁₂H₁₁O₅(M−1) calc 235.1, found 235.1.

### 172B. methyl 3-(5-(2-aminopyrimidin-4-yl)-1H-benzo[d]imidazol-2-yl)chroman-6-carboxylate

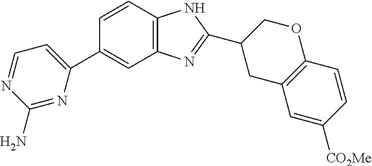

The desired product was prepared in three steps: 1) by substituting 6-(methoxycarbonyl)chroman-3-carboxylic acid for chroman-3-carboxylic acid in Example 40A to give an arylbromide; 2) by treating the arylbromide according to Example 42A to give an arylboronate; and 3) by treating the arylboronate according to Example 44. Preparative HPLC was used to give 23 mg of the desired product. LC-MS: single peak at 254 nm, MH⁺ calcd. for C₂₂H₁₉N₅O₃: 402, obtained 402. HPLC: single peak by analytical HPLC.

### Example 173

### 3-(5-(2-aminopyrimidin-4-yl)-1H-benzo[d]imidazol-2-yl)-N,N-dimethylchroman-6-carboxamide

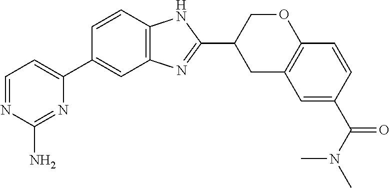

The desired product was prepared by substituting 6-(dimethylcarbamoyl)chroman-3-carboxylic acid for 6-(methoxycarbonyl)chroman-3-carboxylic acid in Example 172B. Preparative HPLC provided 6.91 mg of the desired amide: LC-MS: single peak at 254 nm, MH⁺ calcd. for C₂₃H₂₂N₆O₂: 415, obtained 415. HPLC: single peak by analytical HPLC. ¹H-NMR (MeOD- d₄, 400 MHz): 8.33 (1H, m), 8.28 (1H, d, J=5.5 Hz), 8.01 (1H, dd, J=1.7 Hz, 8.5 Hz), 7.65 (1H, d, J=8.5 Hz), 7.32 (1H, m), 7.25 (1H, dd, J=2.2 Hz, 8.4 Hz), 7.22 (1H, d, J=5.6 Hz), 6.92 (1H, d, J=8.4 Hz), 4.67 (1H, ddd, J=1.6 Hz, 3.4 Hz, 10.9 Hz), 4.43 (1H, dd, J=9.2 Hz, 10.9 Hz), 3.70 (1H, m), 3.34 (2H, m), 3.09 (6H, bs).

### Example 174

### 2-(2-amino(4-chlorophenyl)methyl)-5-fluoro-6-(1H-pyrazol-4-yl)-1H-benzo[d]imidazol-1-yl)-N,N-dimethylethanamine

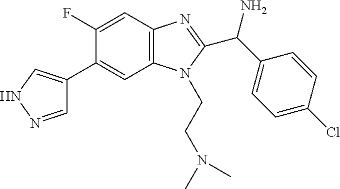

Example 162 was treated with 30% TFA in DCM at room temperature for 30 minutes, then the solvent was removed. Preparative HPLC was used to give the desired product as a white solid (82%). ¹H-NMR (DMSO-d₆, 400 MHz), δ: 9.27 (br, 3H), 8.09-8.10 (m, 2H), 7.97-7.98 (m, 1H), 7.51-7.71 (m, 5H), 6.22 (s, 1H), 4.5-4.72 (m, 2H), 4.30-4.38 (m, 2H), 2.85 (s, 6H); LC/MS: C₂₁H₂₂ClFN₆ (M+1) 412.99.

### Example 175

### 2-(2-(amino(4-chlorophenyl)methyl)-5-fluoro-6-(1H-pyrrolo[2,3-b]pyridin-4-yl)-1H-benzo[d]imidazol-1-yl)-N,N-dimethylethanamine

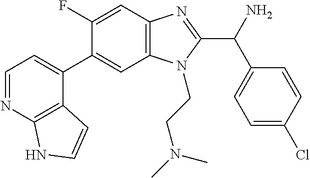

The desired product was prepared in two steps: 1) by using 1-bromo-2,5-difluoro-4-nitrobenzene, N,N-dimethylethylenediamine, Boc-p-chloro-D,L-Phg-OH, and 4-chloro-1H-pyrrolo[2,3-b]pyridine via the alternative route in Example 142 (arylbromide converted into arylboronate); 2) by removal of the Boc-group according to Example 174. The residue was purified by preparative HPLC to give the desired product as a white solid (96%). ¹H-NMR (DMSO-d₆, 400 MHz), δ: 9.24 (br, 3H), 8.30-8.34 (m, 1H), 7.94-7.96 (m, 1H), 7.83-7.86 (m, 1H), 7.56-7.61 (m, 6H), 7.21-7.23 (m, 1H), 6.40 (s, 1H), 4.52-4.59 (m, 2H), 4.18-4.25 (m, 2H), 2.85 (s, 6H); LC/MS: C₂₅H₂₄ClFN₆ (M+1) 463.08.

### Example 176

### (4-chlorophenyl)(1-ethyl-5-fluoro-6-(1H-pyrazol-4-yl)-1H-benzo[d]imidazol-2-yl)methanamine

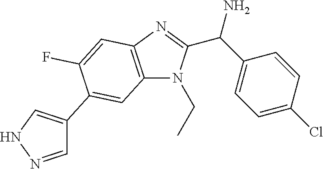

The desired product was prepared in two steps: 1) by using 1-bromo-2,5-difluoro-4-nitrobenzene, ethylamine, Boc-p-chloro-D,L-Phg-OH and a pyrazole-4-boronate in Example 147; 2) by removal of the Boc-group according to Example 174. ¹H-NMR (DMSO-d₆, 400 MHz), δ: 9.16 (br, 3H), 8.10 (s, 2H), 7.94-7.96 (m, 1H), 7.55-7.66 (m, 5H), 6.20 (s, 1H), 4.27 (q, J=7.2 Hz, 1H), 4.12 (q, J=7.2 Hz, 1H), 0.93 (t, J=7.2 Hz, 3H); LC/MS: C₁₉H₁₇ClFN₅ (M+1) 369.98.

### Example 177

### (4-chlorophenyl)-(1-ethyl-5-fluoro-6-(1H-pyrrolo[2,3-b]pyridin-4-yl)-1H-benzo[c]imidazol-2-yl)methanamine

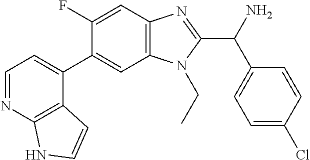

The desired product was prepared by substituting ethylamine for N,N-dimethylethylenediamine in Example 175. ¹H-NMR (DMSO-d₆, 400 MHz), δ: 9.24 (br, 3H), 8.32-8.34 (m, 1H), 7.79-7.87 (m, 2H), 7.55-7.56 (m, 6H), 7.20-7.22 (m, 1H), 6.40 (s, 1H), 4.30 (q, J=6.8 Hz, 1H), 4.21 (q, J=6.8 Hz, 1H), 0.92 (t, J=6.8 Hz, 3H); LC/MS: C₂₃H₁₉ClFN₅ (M+1) 420.07.

### Example 178

### 4-(2-(amino(4-chlorophenyl)methyl)-1-ethyl-5-fluoro-1H-benzo[d]imidazol-6-yl)pyrimidin-2-amine

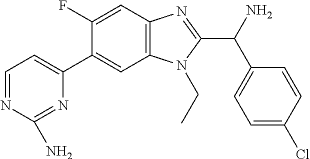

The desired product was prepared by substituting 4-chloropyrimidin-2-amine for 4-chloro-1H-pyrrolo[2,3-b]pyridine in Example 177. ¹H-NMR (DMSO-d₆, 400 MHz), δ: 9.20 (br, 3H, 8.34-8.35 (m, 1H), 8.09-8.11 (m, 1H), 7.73-7.76 (m, 1H), 7.56-7.60 (m, 4H), 7.00-7.02 (m, 1H), 6.79 (s, 1H), 6.26 (s, 1H), 4.29 (q, J=7.2 Hz, 1H), 4.16 (q, J=7.2 Hz, 1H), 0.94 (t, J=7.2 Hz, 3H); LC/MS: C₂₀H₁₈ClFN₆ (M+1) 397.01.

### Example 179

### (1-ethyl-5-fluoro-6-(1H-pyrazol-4-yl)-1H-benzo[d]imidazol-2-yl)-(4-methoxyphenyl)methanamine

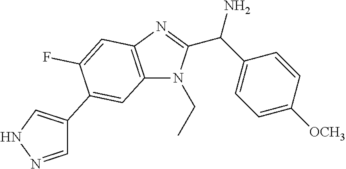

The desired product was prepared by substituting Boc-p-methoxy-D,L-Phg-OH for Boc-p-chloro-D,L-Phg-OH in Example 176. ¹H-NMR (DMSO-d₆, 400 MHz), δ: 9.07 (br, 3H), 7.92-8.01 (m, 2H), 7.60-7.65 (m, 2H), 7.45 (d, J=8.8 Hz, 2H), 7.02 (d, J=8.8 Hz, 2H), 6.09 (s, 1H), 4.24 (q, J=7.8 Hz, 1H), 4.10 (q, J=7.8 Hz, 1H), 0.89 (t, J=7.8 Hz, 3H); LC/MS: C₂₀H₂₀FN₅O (M+1) 365.96.

### Example 180

### (1-ethyl-5-fluoro-6-(1H-pyrazol-4-yl)-1H-benzo[d]imidazol-2-yl)-(3,4,5-trimethoxyphenyl)methanamine

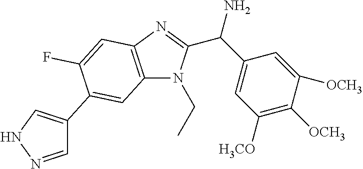

The desired product was prepared by substituting Boc-3,4,5-trimethoxy-D,L-Phg-OH for Boc-p-chloro-D,L-Phg-OH in Example 176. ¹H-NMR (DMSO-d₆, 400 MHz), δ: 9.13 (br, 3H), 8.09-8.10 (m, 2H), 7.93-7.95 (m, 1H), 7.63-7.66 (m, 1H), 6.96-6.97 (m, 2H), 6.02 (s, 1H), 4.34 (q, J=7.2 Hz, 1H), 4.19 (q, J=7.2 Hz, 1H), 3.76 (s, 6H), 3.66 (s, 3H), 0.98 (t, J=7.2 Hz, 3H); LC/MS: C₂₂H₂₄FN₅O₃ (M+1) 425.96.

### Example 181

### 2-(chroman-2-yl)-1-ethyl-5-fluoro-6-(1H-pyrazol-4-yl)-1H-benzo[d]imidazole

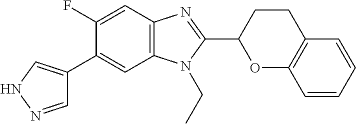

The desired product was prepared by using 1-bromo-2,5-difluoro-4-nitrobenzene, ethylamine, chroman-2-carboxylic acid, and a pyrazole-4-boronate in Example 147. ¹H-NMR (DMSO-d₆, 400 MHz), δ: 8.11-8.13 (m, 3H), 7.63-7.66 (m, 1H), 7.17-7.24 (m, 2H), 6.89-6.70 (m, 2H), 5.71 (t, J=0.4 Hz, 1H), 4.55 (q, J=7.2 Hz, 2H), 3.07-3.11 (m, 2H), 2.48-2.52 (m, 2H), 1.54 (t, J=7.2 Hz, 3H); LC/MS: C₂₁H₁₉FN₄O (M+1) 363.17.

### Example 182

### 2-(chroman-2-yl)-1-ethyl-5-fluoro-6-(1H-pyrrolo[2,3-b]pyridin-4-yl)-1H-benzo[d]imidazole

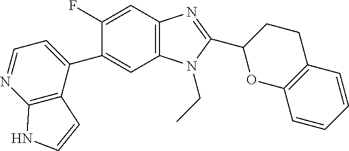

The desired product was prepared by using 1-bromo-2,5-difluoro-4-nitrobenzene, ethylamine, chroman-2-carboxylic acid, and 4-chloro-1H-pyrrolo[2,3-b]pyridine via the alternative route in Example 142 (arylbromide converted into arylboronate). ¹H-NMR (DMSO-d₆, 400 MHz), δ: 8.36-8.37 (s, 1H), 7.59-7.92 (m, 4H), 0.82-7.28 (m, 5H), 6.45-6.46 (m, 1H), 5.66-5.68 (m, 1H), 5.67 (t, J=6.8 Hz, 1H), 4.50 (q, J=6.8 Hz, 2H), 3.04-3.05 (m, 2H), 2.50-2.51 (m, 2H), 1.46 (t, J=6.8 Hz, 3H); LC/MS: C₂₅H₂₁FN₄O (M+1) 413.19.

### Example 183

### 4-(2-(amino(4-methoxyphenyl)methyl)-1-ethyl-5-fluoro-1H-benzo[d]imidazol-6-yl)pyrimidin-2-amine

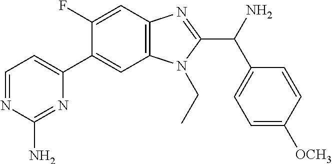

The desired product was prepared in two steps: 1) by using 1-bromo-2,5-difluoro-4-nitrobenzene, ethylamine, Boc-p-methoxy-D,L-Phg-OH, and 4-chloropyrimidin-2-amine via the alternative route in Example 142 (arylbromide converted into arylboronate); 2) by removal of the Boc-group according to Example 174. ¹H-NMR (DMSO-d₆, 400 MHz), δ: 9.13 (br, 3H), 8.39-8.40 (m, 1H), 8.11-8.13 (m, 1H), 7.76-7.79 (m, 1H), 7.48-7.50 (m, 2H), 7.04-7.11 (m, 4H), 6.16 (s, 1H), 4.15-4.35 (m, 2H), 3.75 (s, 3H), 0.98 (t, J=7.2 Hz, 3H); LC/MS: C₂₁H₂₁FN₆O (M+1) 392.96.

### Example 184

### 4-(2-(amino(3,4,5-trimethoxyphenyl)methyl)-1-ethyl-5-fluoro-1H-benzo[d]imidazol-6-yl)pyrimidin-2-amine

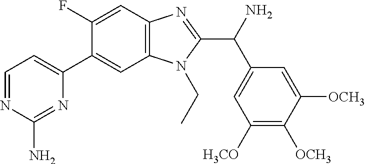

The desired product was prepared by substituting Boc-3,4,5-trimethoxy-D,L-Phg-OH for Boc-p-methoxy-D,L-Phg-OH in Example 183. ¹H-NMR (DMSO-d₆, 400 MHz), δ: 9.22 (br, 3H), 8.36-8.42 (m, 2H), 8.10-8.12 (m, 1H), 7.74-7.76 (m, 1H), 6.99-7.07 (m, 3H), 0.09 (s, 1H), 4.38 (q, J=7.2 Hz, 1H), 4.22 (q, J=7.2 Hz, 1H), 3.76 (s, 6H), 3.66 (s, 3H), 0.99 (t, J=7.2 Hz, 3H); LC/MS: C₂₃H₂₅FN₆O₃ (M+1) 452.98.

### Example 185

### 4-(2-(chroman-2-yl)-1-ethyl-5-fluoro-1H-benzo[d]imidazol-6-yl)pyrimidin-2-amine

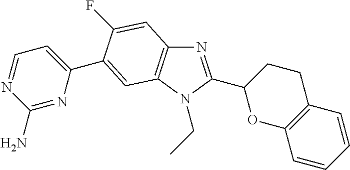

The desired product was prepared by substituting 4-chloropyrimidin-2-amine for 4-chloro-1H-pyrrolo[2,3-b]pyridine in Example 182. ¹H-NMR (DMSO-d₆, 400 MHz), δ: 8.31-8.33 (m, 1H), 8.12-8.13 (m, 1H), 7.59-7.62 (m, 1H), 7.02-7.10 (m, 5H), 6.73-6.84 (m, 2H), 5.58 (t, J=5.2 Hz, 1H), 4.37-4.46 (m, 2H), 2.93-2.96 (m, 2H), 2.37-2.40 (m, 2H), 1.40 (t, J=7.2 Hz, 3H); LC/MS: C₂₂H₂₀FN₅O (M+1) 390.17.

### Example 186

### 4-(1-ethyl-5-fluoro-2-(6-methoxy-1,2,3,4-tetrahydroisoquinolin-1-yl)-1H-benzo[d]imidazol-6-yl)pyrimidin-2-amine

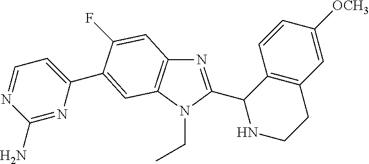

The Boc-protected product was prepared by using 1-bromo-2,5-difluoro-4-nitrobenzene, ethylamine, N-Boc-6-methoxy-1,2,3,4-tetrahydroisoquinoline-1-carboxylic acid (available from Anichem LLC, North Brunswick, N.J.), and 4-chloropyrimidin-2-amine via the alternative route in Example 142 (arylbromide converted into arylboronate). The Boc-protecting group was removed by treatment with 30% TFA in DCM at room temperature for 30 minutes, then the solvent was removed. Purification by preparative HPLC gave the desired product. ¹H-NMR (DMSO-d₆, 400 MHz), S: 8.26-8.38 (m, 2H), 7.62-7.64 (m, 1H), 6.60-7.08 (m, 5H), 6.23 (s, 1H), 4.50-4.75 (m, 2H), 3.78 (s, 3H), 3.01-3.35 (m, 5H), 1.50 (t, J=7.2 Hz, 3H); LC/MS: C₂₃H₂₃FN₆O (M+1) 418.95.

### Example 187

### 4-(2-(6,7-dimethoxy-1,2,3,4-tetrahydroisoquinolin-1-yl)-1-ethyl-5-fluoro-1H-benzo[d]imidazol-6-yl)pyrimidin-2-amine

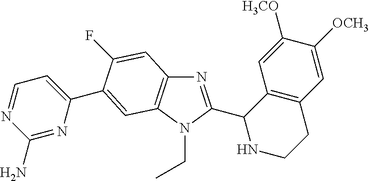

The desired product was prepared by substituting N-Boc-6,7-dimethoxy-1,2,3,4-tetrahydroisoquinoline-1-carboxylic acid for N-Boc-6-methoxy-1,2,3,4-tetrahydroisoquinoline-1-carboxylic acid in Example 186. ¹H-NMR (DMSO-d₆, 400 MHz), δ: 8.26-8.38 (m, 2H), 7.62-7.65 (m, 1H), 6.94-7.09 (m, 5H), 6.22 (s, 1H), 4.63-4.75 (m, 1H), 4.49-4.59 (m, 1H), 3.78 (s, 3H), 3.52 (s, 3H), 3.00-3.35 (m, 5H), 1.50 (t, J=7.2 Hz, 3H); LC/MS: C₂₄H₂₅FN₆O₂ (M+1) 448.95.

### Example 188

### 5-fluoro-2-(7-methoxychroman-3-yl)-6-(1H-pyrazol-4-yl)-1H-benzo[d]imidazole

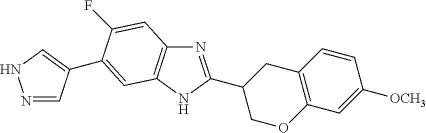

The desired product was prepared according to Example 4 using an arylbromide prepared by substituting 4-bromo-5-fluorobenzene-1,2-diamine for 1,2-diamino-4-bromobenzene and 7-methoxychroman-3-carboxylic acid for 1,4-benzodioxan-2-carboxylic acid in Example 1. LC/MS: C₂₀H₁₇FN₄O₂ (M+1) 365.16, obsd: 365.

### Example 189

### (5-fluoro-6-(1H-pyrazol-4-yl)-1H-benzo[d]imidazol-2-yl)-(4-methoxyphenyl)methanamine

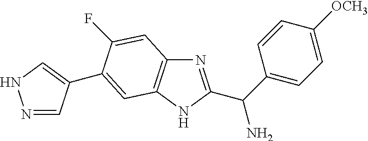

The desired product was prepared in two steps: 1) by using 1-bromo-2,5-difluoro-4-nitrobenzene, Boc-p-methoxy-D,L-Phg-OH and a pyrazole-4-boronate in Example 147; 2) by removal of the Boc-group according to Example 93. LC/MS: C₁₈H₁₆FN₅O (M+1) 337.88, obsd.: 337.

### Example 190

### 4-(2-(amino(4-methoxyphenyl)methyl)-5-fluoro-1H-benzo[d]imidazol-6-yl)pyrimidin-2-amine

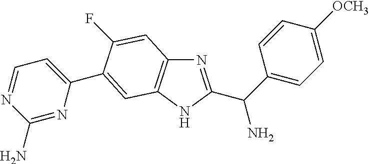

The desired product was prepared in two steps: 1) by using 1-bromo-2,5-difluoro-4-nitrobenzene, Boc-p-methoxy-D,L-Phg-OH, and 4-chloropyrimidin-2-amine via the alternative route in Example 142 (arylbromide converted into arylboronate); 2) by removal of the Boc-group according to Example 93. LC/MS: C₁₉H₁₇FN₆O (M+1) 364.91, obsd.: 364.

### Example 191

### 4-(5-fluoro-2-(6-methoxychroman-3-yl)-1H-benzo[d]imidazol-6-yl)pyrimidin-2-amine

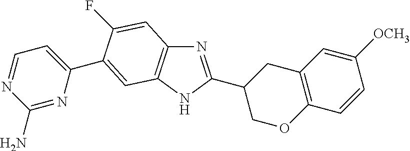

The desired product was prepared by using 1-bromo-2,5-difluoro-4-nitrobenzene, 6-methoxychroman-3-carboxylic acid, and 4-chloropyrimidin-2-amine via the alternative route in Example 142 (arylbromide converted into arylboronate). LC/MS: C21H₁₈FN₅O₂ (M+1) calcd.: 392.20, obsd: 392.

### Example 192

### 5-fluoro-2-(6-methoxychroman-3-yl)-6-(1H-pyrrolo[2,3-b]pyridin-4-yl)-1H-benzo[d]imidazole

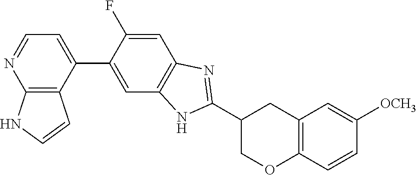

The desired product was prepared by substituting 4-chloro-1H-pyrrolo[2,3-b]pyridine for 4-chloropyrimidin-2-amine in Example 191. LC/MS: calcd. For C₂₄H₁₉FN₄O₂ (M+1) 415.22, obsd: 415.

### Example 193

### 2-(2,3-dihydrobenzo[b]oxepin-4-yl)-6-(pyridin-4-yl)-1H-benzo[d]imidazole

### 193A. (E)-2,3-dihydrobenzo[b]oxepine-4-carboxylic acid

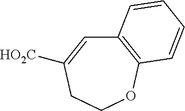

Salicylaldehyde (10.0 g, 81.9 mmol), ethyl 4-bromobutyrate (19.2 g, 1.2 eq), and potassium carbonate (22.6 g, 2.0 eq) were suspended in 20 mL of anhydrous DMF. The reaction was heated in a 90° C. bath for 2 hours before the heat was removed and the reaction was allowed to stir overnight. The reaction was diluted with ethyl acetate and acidified with a combination of 5N and 1N HCl. The organic layer was separated and washed 2× with 1N HCl and 3× with brine. The organic layer was dried with sodium sulfate and concentrated to give 18.2 g of an intermediate aldehyde as a brownish oil. ¹H-NMR (CDCl₃-d₄, 400 MHz) δ 1.23 (t, 7.2 Hz, 3H), 2.17 (pentet, 6.8 Hz, 2H), 2.49 (t, 6.8 Hz, 2H), 4.06-4.16 (m, 2H), 6.95 (d, 8.4 Hz, 1H), 6.70 (t, 7.2 Hz, 1H), 7.48-7.54 (m, 1H), 7.80 (dd, 1.7 Hz, 7.6 Hz, 1H), 10.46 (s, 1H). Single peak by HPLC.

The above aldehyde (9.50 g, 40.3 mmol) was put under an argon atmosphere before being dissolved in 30 mL ethyl carbonate. To this solution was added sodium ethoxide (13.6 mL, 21% solution in ethanol) before being heated at 90° C. for 2 hours and allowed to cool back to room temperature for 4 hours. The reaction was diluted with ethyl acetate and washed 3× with 1N HCl and 3× with brine. The organic layer was dried with sodium sulfate and concentrated. The residue was purified by silica gel chromatography (80 g×2, hexanes:EtOAc) to give 3.27 g of homochroman ester as a colorless oil. ¹H-NMR (CDCl₃-d₄, 400 MHz) δ1.32 (t, 7.0 Hz, 3H), 2.95 (t, 4.8 Hz, 2H), 4.21-4.28 (m, 4H), 6.95 (dd, 1.2 Hz, 8.4 Hz, 1H), 6.99 (td, 1.2 Hz, 8.4 Hz, 1H), 7.21 (td, 1.2 Hz, 8.4 Hz, 1H), 7.31 (dd, 1.6 Hz, 7.6 Hz, 1H), 7.56 (s, 1H). Single peak by HPLC.

The above unsaturated homochroman ester (3.27 g, 15.0 mmol) was dissolved in 20 mL anhydrous THF. Lithium hydroxide monohydrate (786 mg, 1.25 eq) was added and the reaction was heated at 60 C for 48 hours. The solvent was removed in vacuo and the residue taken up in water. The solution was acidified with 1N HCl and the resulting precipitate was filtered off. After drying by high vacuum, 2.74 g of the desired product was obtained as a white solid. ¹H-NMR (DMSO-d₄, 400 MHz) δ 2.83 (t, 4.9 Hz, 2H), 4.20 (t, 4.9 Hz, 2H), 6.95 (dd, 1.2 Hz, 8.0 Hz, 1H), 7.03 (td, 1.2 Hz, 8.0 Hz, 1H), 7.27 (td, 1.6 hz, 8.0 Hz, 1H), 7.44 (dd, 1.2 Hz, 8.0 Hz, 1H), 7.49 (s, 1H), 12.5 (s, 1H). Single peak by HPLC.

### 193B. 6-bromo-2-(2,3-dihydrobenzo[b]oxepin-4-yl)-1H-benzo[d]imidazole

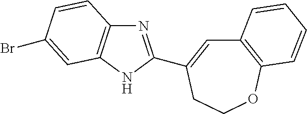

The desired product was prepared by substituting Example 193A for chroman-3-carboxylic acid in Example 40A.

### 193C. 2-(2,3-dihydrobenzo[b]oxepin-4-yl)-6-(pyridin-4-yl)-1H-benzo[d]imidazole

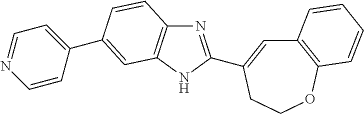

The desired product was prepared by substituting Example 193B for Example 40A and pyridine-4-boronic acid for 4-(4,4,5,5-tetramethyl-1,3,2-dioxaborolan-2-yl)-1H-pyrazole (pyrazole-4-pinacolboronate) in Example 40B. The crude product was purified by silica gel chromatography (4 g column, DCM:MeOH) to give 47 mg of the desired product. LCMS (found 339.8 MH+1 calculated for C₂₂H₁₇N₂O: 339.1). Single peak by HPLC. ¹H-NMR (CD₃OH-d₄, 400 MHz) δ 3.27 (t, 4.0 Hz, 2H), 4.40 (t, 2.4 Hz, 2H), 6.99 (dd, 1.0 Hz, 8.0 Hz, 1H), 7.05 (td, 1.0 Hz, 8.0 Hz, 1H), 7.23 (td, 1.0 Hz, 8.0 Hz, 1H), 7.41 (dd, 1.6 Hz, 8.0 Hz, 1H), 7.46 (s, 1H), 7.62-7.70 (m, 2H), 7.75 (dd, 1.6 Hz, 4.4 Hz, 2H), 7.92 (bs, 1H), 8.56 (dd, 1.6 Hz, 4.4 Hz, 2H).

### Example 194

### 2-(2,3-dihydrobenzo[b]oxepin-4-yl)-6-(1H-pyrazol-4-yl)-1H-benzo[d]imidazole

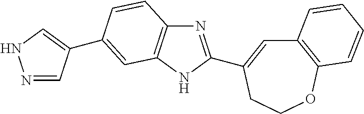

The desired product was prepared by substituting Example 193B for Example 40A in Example 40B. As an additional change, pyrazole-4-pinacolateboronic ester (3.0 equiv.) was used with methanol as the solvent for the reaction run on 0.462 mmol scale. The crude product was purified by silica gel chromatography (4 g column, DCM:MeOH) to give 58 mg of the desired product. LCMS (found 328.8 MH+1 calculated for C₂₀H₁₆N₄O: 328.1). Single peak by analytical HPLC.

### Example 195

### 6-(pyridin-4-yl)-2-(2,3,4,5-tetrahydrobenzo[b]oxepin-4-yl)-1H-benzo[d]imidazole, trifluoroacetic acid salt

### 193A. 2,3,4,5-tetrahydrobenzo[b]oxepine-4-carboxylic acid

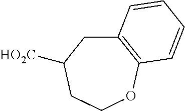

Example 193A was dissolved in 10 mL methanol. A catalytic amount of 10% palladium on carbon was added and the reaction was put under a 55 psi hydrogen atmosphere and shaken for 18 hours. The reaction was filtered and concentrated to give the desired product as a colorless solid. ¹H-NMR (CDCl₃-d₄, 400 MHz) δ 2.14-2.21 (m, 2H), 2.56-2.64 (m, 1H), 2.97-3.11 (m, 2H), 3.77 (pentet, 6.0 Hz, 1H), 4.27 (dt, 4.4 Hz, 12.0 Hz, 1H), 6.92 (d, 7.4 Hz, 1H), 6.97 (td, 1.2 Hz, 7.4 Hz, 1H), 7.12 (dt, 1.6 Hz, 7.4 Hz, 1H), 7.17 (dd, 1.2 Hz, 7.4 Hz, 1H). Single peak by HPLC.

### 195B. 6-bromo-2-(2,3,4,5-tetrahydrobenzo[b]oxepin-4-yl)-1H-benzo[d]imidazole

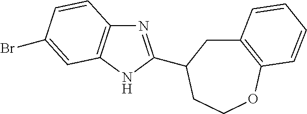

The desired product was prepared by substituting 2,3,4,5-tetrahydrobenzo[b]oxepine-4-carboxylic acid for (E)-2,3-dihydrobenzo[b]oxepine-4-carboxylic acid in Example 193B.

### 195C. 6-(pyridin-4-yl)-2-(2,3,4,5-tetrahydrobenzo[b]oxepin-4-yl)-1H-benzo[d]imidazole, trifluoroacetic acid salt

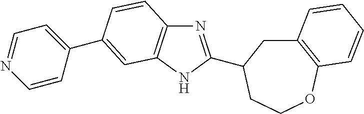

The desired product was prepared by substituting Example 195B for Example 193B in Example 193C. As an additional change, 4-pyridine boronic acid (1.5 equiv.) was used for the reaction run on 0.332 mmol scale. The product was purified by prep HPLC to give the desired product as the TFA salt. LCMS (found 342.3 MH+1 calculated for C₂₂H₁₉N₂O: 342.3). Single peak by HPLC.

### Example 196

### 6-(1H-pyrazol-4-yl)-2-(2,3,4,5-tetrahydrobenzo[b]oxepin-4-yl)-1H-benzo[d]imidazole, trifluoroacetic acid salt

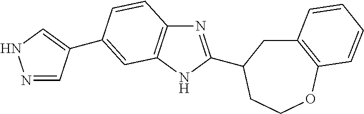

The desired product was prepared by substituting Example 195B for Example 193B in Example 194, run on 0.461 mmol scale. The product was purified by prep HPLC to give the desired product as the TFA salt. LCMS (found 331.3 MH+1 calculated for C₂₀H₁₈N₄O: 331.2). Single peak by HPLC.

### Example 197

### General Procedure for the Preparation of Substituted Benzimidazoles No. 3

HATU (1.2 equiv), Et₃N (2.0 equiv) and carboxylic acid (1.0 equiv) are combined in anhydrous DMF (10 mL/mmol) at room temperature. To this solution is added 4-bromobenzene-1,2-diamine (1.0 equiv) and the resulting mixture is stirred at room temperature until complete by LC-MS analysis. Upon completion, the solution is concentrated in vacuo and then diluted with glacial AcOH (15 mL/mmol). This solution is warmed to 65° C. and stirred until the cyclodehydration is complete by LC-MS. The reaction is then concentrated in vacuo and diluted with EtOAc. The solution is then washed with concentrated aqueous NaHCO₃ and back extracted twice with additional EtOAc. The combined organic fractions are dried over MgSO₄ and purified on SiO₂ (Hexane/EtOAc) to give an arylbromide product. The aryl bromide (1.0 equiv) can then be combined with an arylboronate species (1.30 equiv) and Na₂CO₃ (3.0 equiv) in 20% aqueous dioxane (10 mL/mmol) in a microwave pressure tube at room temperature. To this solution is added PdCl₂(PPh₃)₂ (0.10 equiv) and the solution is sparged with argon for 10 minutes. The reaction is subsequently heated in a microwave at 120° C. until the reaction is complete (60-240 minutes). The product is then purified via preparative HPLC. Alternatively, the above arylbromide (1.0 equiv) can be combined with bis(pinacolato)boron (2.5 equiv), KOAc (5.0 equiv) and PdCl₂(dppf) (0.1 equiv) under streaming argon in a microwave pressure tube. Dioxane (10 mL/mmol) is then added and the solution sparged with argon for 10 minutes. The solution is then heated to 100° C. in a microwave until the conversion is complete (30 min). Upon completion, the solution is diluted with EtOAc and washed with brine. The aqueous fraction is extracted with additional EtOAc and the combined organic portions are dried over MgSO₄ and concentrated. Purification on silica gel (hexane/EtOAc) gave the desired arylboronate product. The arylboronate (1.0 equiv) is then combined with an aryl halide (1.0 equiv), Na₂CO₃ (3.0 equiv) and PdCl₂(PPh₃)₂ (0.1 equiv) under streaming argon. Aqueous dioxane (20%, 10 mL/mmol) is then added and the solution sparged with argon for 10 minutes. The solution is then heated to 120° C. in a microwave until complete (30 min). Upon completion, the solution is purified via preparative HPLC to give the desired product.

### Example 198

### 4-(2-(6-(trifluoromethoxy)chroman-3-yl)-1H-benzo[d]imidazol-6-yl)pyrimidin-2-amine

### 198A. 6-(trifluoromethoxy)chroman-3-carboxylic acid

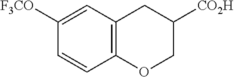

5-(trifluoromethoxy)salicylaldehyde (1.0 equiv) was combined with benzyl acrylate (1.2 equiv) and DABCO (0.2 equiv) in a microwave pressure tube. The mixture was then heated to 170° C. for 60 minutes. The solution was then diluted with CH₂Cl₂ and washed with saturated NH₄Cl. The product was then concentrated and purified on silica gel (hexane/EtOAc) to give the chromene product (59% yield). This material was then dissolved in MeOH containing Pd/C (0.05 equiv) and stirred under a hydrogen balloon atmosphere until complete. The solution was then filtered through a plug of Celite and concentrated to give the desired chromane which was used directly in the HATU coupling.

### 198B. 4-(2-(6-(trifluoromethoxy)chroman-3-yl)-1H-benzo[d]imidazol-6-yl)pyrimidin-2-amine

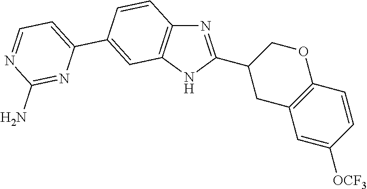

The desired product was prepared by using Example 198A in Example 197, and employing the alternative method to obtain the arylboronate, which was treated with 4-chloropyrimidin-2-amine (4.71 mg). LC-MS: single peak at 254 nm, MH⁺ calcd. for C₂₁H₁₆F₃N₅O₂: 428, obtained 428. HPLC: single peak by analytical HPLC. ¹H-NMR (MeOD- d₄, 400 MHz): 8.57 (1H, d, J=1.5 Hz), 8.31 (1H, d, J=6.6 Hz), 8.28 (1H, dd, J=8.7 Hz, 1.7 Hz), 7.79 (1H, d, J=8.6 Hz), 7.58 (1H, d, J=6.6 Hz), 7.16 (1H, s), 7.08 (1H, d, J=8.9 Hz), 6.94 (1H, d, J=8.9 Hz), 4.66 (1H, dd, J=3.2 Hz, 11.0 Hz), 4.48 (1H, dd, J=8.2 Hz, 11.0 Hz), 3.84 (1H, m), 3.42 (2H, d, J=7.3 Hz).

### Example 199

### 4-(2-(6-methoxychroman-3-yl)-1H-benzo[d]imidazol-6-yl)pyrimidin-2-amine

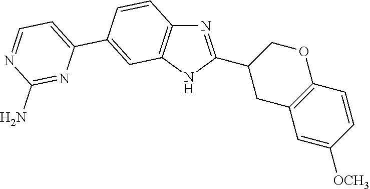

The desired product was prepared by using 6-methoxychroman-3-carboxylic acid in Example 197, and employing the alternative method to obtain the arylboronate, which was treated with 4-chloropyrimidin-2-amine (15.5 mg). LC-MS: single peak at 254 nm, MH⁺ calcd. for C₂₁H₁₉N₅O₂: 374, obtained 374. HPLC: single peak by analytical HPLC. ¹H-NMR (MeOD- d₄, 400 MHz): 8.53 (1H, s), 8.31 (1H, d, J=6.4 Hz), 8.25 (1H, dd, J=1.6 Hz, 8.6 Hz), 7.77 (1H, d, J=8.5 Hz), 7.52 (1H, d, J=6.4 Hz), 6.78 (3H, m), 4.56 (1H, m), 4.40 (1H, dd, J=8.3 Hz, 11.1 Hz), 3.78 (1H, m), 3.76 (3H, s), 3.42 (2H, m).

### Example 200

### 2-(6-methoxychroman-3-yl)-6-(1H-pyrazol-4-yl)-1H-benzo[d]imidazole

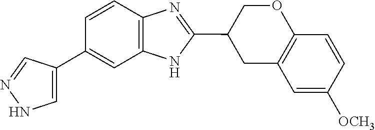

The desired product was prepared by using 6-methoxychroman-3-carboxylic acid and 4-(4,4,5,5-tetramethyl-1,3,2-dioxaborolan-2-yl)-1H-pyrazole in Example 197 (5.3 mg). LC-MS: single peak at 254 nm, MH⁺ calcd. for C₂₀H₁₈N₄O₂: 347, obtained 347. HPLC: single peak by analytical HPLC. ¹H-NMR (MeOD- d₄, 400 MHz): 8.09 (2H, s), 7.91 (1H, s), 7.85 (1H, d, J=8.6 Hz), 7.75 (1H, d, J=8.6 Hz), 6.80 (3H, m), 4.56 (2H, d, J=4.4 Hz), 4.00 (1H, m), 3.76 (3H, s), 3.52 (1H, dd, J=6.1 Hz, 6.9 Hz), 3.37 (1H, m).

### Example 201

### 2-(2,3-dihydrobenzofuran-2-yl)-6-(pyridin-4-yl)-1H-benzo[d]imidazole

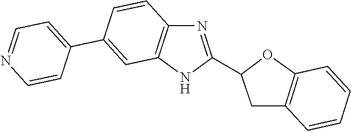

The desired product was prepared by using 2,3-dihydrobenzofuran-2-carboxylic acid and 4-(4,4,5,5-tetramethyl-1,3,2-dioxaborolan-2-yl)pyridine in Example 197 (22.0 mg). LC-MS: single peak at 254 nm, MH⁺ calcd. for C₂₀H₁₅N₃O: 314, obtained 314. HPLC: single peak by analytical HPLC.

### Example 202

### 2-(2,3-dihydrobenzofuran-2-yl)-6-(1H-pyrazol-4-yl)-1H-benzo[d]imidazole

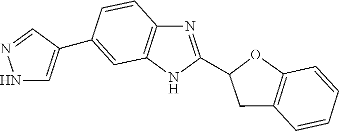

The desired product was prepared by using 2,3-dihydrobenzofuran-2-carboxylic acid and 4-(4,4,5,5-tetramethyl-1,3,2-dioxaborolan-2-yl)-1H-pyrazole in Example 197 (30.0 mg). LC-MS: single peak at 254 nm, MH⁺ calcd. for C₁₈H₁₄N₄O: 303, obtained 303. HPLC: single peak by analytical HPLC. ¹H-NMR (MeOD- d₄, 400 MHz): 7.97 (2H, br), 7.73 (1H, br), 7.58 (1H, br), 7.51 (1H, d, J=7.1 Hz), 7.28 (1H, d, J=7.2 Hz), 7.18 (1H, t, J=7.8 Hz), 6.92 (2H, m), 6.00 (1H, dd, J=7.5 Hz, 9.8 Hz), 3.78 (1H, dd, J=9.8 Hz, 15.8 Hz), 3.64 (1H, dd, J=7.4 Hz, 15.8 Hz).

### Example 203

### 4-(2-(2,3-dihydrobenzofuran-2-yl)-1H-benzo[d]imidazol-6-yl)pyrimidin-2-amine

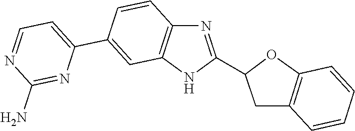

The desired product was prepared by using 2,3-dihydrobenzofuran-2-carboxylic acid in Example 197, and employing the alternative method to obtain the arylboronate, which was treated with 4-chloropyrimidin-2-amine (23.3 mg). LC-MS: single peak at 254 nm, MH⁺ calcd. for C₁₉H₁₅N₅O: 330, obtained 330. HPLC: single peak by analytical HPLC. ¹H-NMR (MeOD- d₄, 400 MHz): 8.59 (1H, s), 8.30 (1H, d, J=6.7 Hz), 8.27 (1H, dd, J=1.4 Hz, 8.8 Hz), 7.78 (1H, d, J=8.7 Hz), 7.61 (1H, d, J=6.7 Hz), 7.31 (1H, d, J=7.6 Hz), 7.21 (1H, t, J=7.6 Hz), 6.99 (2H, d, J=7.6 Hz), 6.13 (1H, dd, J=7.1 Hz, 10.1 Hz), 3.88 (1H, dd, J=9.8 Hz, 15.9 Hz), 3.66 (1H, dd, J=7.3 Hz, 16.0 Hz).

### Example 204

### 6-(2-(2,3-dihydrobenzofuran-2-yl)-1H-benzo[d]imidazol-6-yl)pyrimidin-4-amine

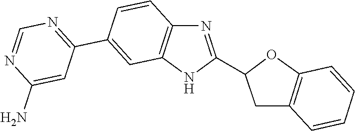

The desired product was prepared by using 2,3-dihydrobenzofuran-2-carboxylic acid in Example 197, and employing the alternative method to obtain the arylboronate, which was treated with 6-chloropyrimidin-4-amine (13.6 mg). LC-MS: single peak at 254 nm, MH⁺ calcd. for C₁₉H₁₅N₅O: 330, obtained 330. HPLC: single peak by analytical HPLC. ¹H-NMR (MeOD- d₄, 400 MHz): 8.64 (1H, s), 8.12 (1H, s), 7.81 (1H, d, J=8.5 Hz), 7.75 (1H, d, J=8.5 Hz), 7.31 (1H, d, J=7.5 Hz), 7.20 (1H, t, J=7.4 Hz), 7.04 (1H, s), 6.96 (1H, m), 6.11 (1H, dd, J=7.3 Hz, 9.7 Hz), 3.87 (1H, d, J=9.8 Hz, 15.9 Hz), 3.67 (1H, dd, J=15.7 Hz, 7.0 Hz).

### Example 205

### 2-(2,3-dihydrobenzofuran-2-yl)-6-(1H-pyrrolo[2,3-b]pyridin-4-yl)-1H-benzo[d]imidazole

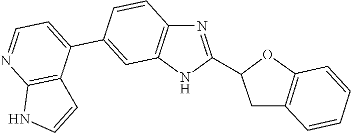

The desired product was prepared by using 2,3-dihydrobenzofuran-2-carboxylic acid in Example 197, and employing the alternative method to obtain the arylboronate, which was treated with 4-chloro-1H-pyrrolo[2,3-b]pyridine (16.7 mg). LC-MS: single peak at 254 nm, MH⁺ calcd. for C₂₂H₁₆N₄O: 353, obtained 353. HPLC: single peak by analytical HPLC.

### Example 206

### 2-(6-(methylsulfonyl)chroman-3-yl)-6-(pyridin-4-yl)-1H-benzo[d]imidazole

### 206A. 6-(methylsulfonyl)chroman-3-carboxylic acid

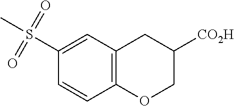

In a round bottom flask equipped with a condenser 4-(methylsulfonyl)phenol (500 mg, 2.90 mmol) was dissolved in 5 mL TFA. Hexamethylaminetetramine (427 mg, 1.05 equiv.) was added and the reaction mixture was heated at 100° C. overnight. The solvent was removed in vacuo. The residue was taken up in 0.5 M HCl which was washed 2× with DCM. The organic layers were combined, dried with sodium sulfate, and concentrated. The residue was purified by silica gel chromatography (12 g column DCM:EtOAc gradient) to give 289 mg (50% yield) of 2-hydroxy-5-methylsulfonylbenzaldehyde as a white solid. ¹H-NMR (CDCl₃-d₄, 400 MHz) δ 3.06 (s, 3H), 7.15 (d, 8.8 Hz, 1H), 8.02 (dd, 2.8 Hz, 8.8 Hz, 1H), 8.21 (d, 2.8 HZ, 1H), 9.95 (s, 1H), 11.48 (s, 1H). Single peak by HPLC.

In sealed microwave vial 2-hydroxy-5-methylsulfonylbenzaldehyde (655 mg, 3.28 mmol) was combined with benzyl acrylate (531 mg, 3.0 equiv.) and DABCO (531 mg, 0.2 equiv.). The vial was heated on the microwave reactor at 150° C. for 30 min. The residue was dissolved in DCM and adsorbed onto silica gel. Silica gel chromatography (12 g column, hexanes:EtOAc gradient) gave benzyl 6-(methylsulfonyl)-2H-chromene-3-carboxylate (49 0 mg, 43% yield) as a colorless solid. ¹H-NMR (CDCl₃-d₄, 400 MHz) δ 3.00 (s, 3H), 5.11 (d, 1.6 Hz, 2H), 5.25 (s, 2H), 6.93 (d, 8.4 Hz, 1H), 7.32-7.40 (m, 5H), 7.43 (s, 1H), 7.67 (d, 2.4 Hz, 1H), 7.75 (dd, 2.4 Hz, 8.6 Hz, 1H). Single peak by HPLC.

The unsaturated benzyl ester (86 mg, 0.247 mmol) was dissolved in 10 mL methanol along with a catalytic amount of 10% Pd/C. The reaction was placed under a 45-55 psi hydrogen atmosphere and shaken for 24 hours. The reaction was filtered through a syringe filter and concentrated to give the desired product as a colorless oil (54 mg, 85% yield). ¹H-NMR (CD₃OD-d₄, 400 MHz) δ 3.00-3.14 (m, 6H), 4.30 (dd, 7.6 Hz, 10.8 Hz, 1H), 4.45 (dd, 3.2 Hz, 10.9 Hz, 1H), 6.94 (d, 8.4 Hz, 1H), 7.63 (dd, 2.4 Hz, 8.4 Hz, 1H), 7.70-7.73 (m, 1H). Single peak by HPLC.

### 206B. 2-(6-(methylsulfonyl)chroman-3-yl)-6-(pyridin-4-yl)-1H-benzo[d]imidazole

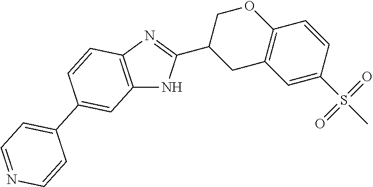

The desired product was prepared by using Example 206A and 4-(4,4,5,5-tetramethyl-1,3,2-dioxaborolan-2-yl)pyridine in Example 197 (8.0 mg). LC-MS: single peak at 254 nm, MH⁺ calcd. for C₂₂H₁₉N₃O₃S: 406, obtained 406. HPLC: single peak by analytical HPLC. ¹H-NMR (MeOD- d₄, 400 MHz): 8.85 (2H, d, J=6.5 Hz), 8.42 (2H, d, J=6.4 Hz), 8.28 (1H, s), 7.99 (1H, d, J=8.6 Hz), 7.85 (2H, m), 7.73 (1H, dd, J=2.1 Hz, 8.7 Hz), 7.08 (1H, d, J=8.7 Hz), 4.76 (1H, dd, J=3.2 Hz, 11.1 Hz), 4.61 (1H, dd, J=11.0 Hz, 7.9 Hz), 3.92 (1H, m), 3.50 (2H, m), 3.10 (3H, s).

### Example 207

### 2-(6-(methylsulfonyl)chroman-3-yl)-6-(1H-pyrazol-4-yl)-1H-benzo[d]imidazole

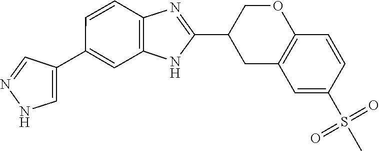

The desired product was prepared by using Example 206A and 4-(4,4,5,5-tetramethyl-1,3,2-dioxaborolan-2-yl)-1H-pyrazole in Example 197 (7.8 mg). LC-MS: single peak at 254 nm, MH⁺ calcd. for C₂₀H₁₈N₄O₃S: 395, obtained 395. HPLC: single peak by analytical HPLC.

### Example 208

### 4-(2-(6-(methylsulfonyl)chroman-3-yl)-1H-benzo[d]imidazol-6-yl)pyrimidin-2-amine

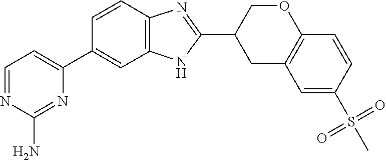

The desired product was prepared by using Example 206A in Example 197, and employing the alternative method to obtain the arylboronate, which was treated with 4-chloropyrimidin-2-amine (8.4 mg). LC-MS: single peak at 254 nm, MH⁺ calcd. for C₂₁H₁₉N₅O₃S: 422, obtained 422. HPLC: single peak by analytical HPLC. ¹H-NMR (MeOD-d₄, 400 MHz): 8.61 (1H, s), 8.32 (2H, m), 7.83 (2H, m), 7.73 (1H, dd, J=1.9 Hz, 8.7 Hz), 7.61 (1H, d, J=6.7 Hz), 7.08 (1H, d, 8.7 Hz), 4.75 (1H, dd, J=3.1 Hz, 11.1 Hz), 4.61 (1H, dd, J=8.0 Hz, 11.1 Hz), 3.93 (1H, m), 3.50 (2H, m), 3.11 (3H, s).

### Example 209

### 6-(2-(6-(methylsulfonyl)chroman-3-yl)-1H-benzo[d]imidazol-6-yl)pyrimidin-4-amine

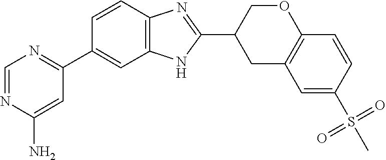

The desired product was prepared by using Example 206A in Example 197, and employing the alternative method to obtain the arylboronate, which was treated with 6-chloropyrimidin-4-amine (3.9 mg). LC-MS: single peak at 254 nm, MH⁺ calcd. for C₂₁H₁₉N₅O₃S: 422, obtained 422. HPLC: single peak by analytical HPLC.

### Example 210

### N,N-dimethyl-3-(6-(pyridin-4-yl)-1H-benzo[d]imidazol-2-yl)chroman-6-sulfonamide

### 210A. 6-(N,N-dimethylsulfamoyl)chroman-3-carboxylic acid

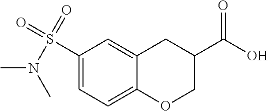

Methyl chroman-3-carboxylate (480 mg, 2.50 mmol) was slowly added to 3 mL ice cold chlorosulfonic acid. The reaction mixture was stirred for 30 minutes, then was slowly quenched by dripping onto crushed ice. This aqueous solution was then washed 3× with ethyl ether. The organic layers were combined, dried with sodium sulfate, and concentrated. The residue was taken up in a 2M solution of dimethylamine in methanol (10 mL) and allowed to stir overnight. The reaction was concentrated and the residue was taken up in DCM and adsorbed onto silica gel. Silica gel chromatography (12 g column, hexanes:EtOAc gradient) gave methyl 6-(N,N-dimethylsulfamoyl)chroman-3-carboxylate as a colorless oil (250 mg, 33% yield). ¹H-NMR (CDCl₃-d₄, 400 MHz) δ 2.66 (s, 6H), 3.00-3.16 (m, 3H), 3.74 (s, 3H), 4.20 (dd, 7.8 Hz, 11.0 Hz, 1H), 4.43-4.24 (m, 1H), 6.90 (d, 8.8 Hz, 1H), 7.46-7.52 (m, 2H). Single peak by HPLC.

The methyl ester (249 mg, 0.833 mmol) was dissolved in 5 mL THF along with lithium hydroxide monohydrate (38.5 mg, 1.0 eq). The reaction was heated to 40° C. and monitored by HPLC. Upon disappearance of starting material (about 72 hours) the solvent was removed. The residue was suspended in water and slowly acidified with 1N HCl. A white precipitate formed which was isolated by filtration and dried in vacuo to give the desired product (216 mg, 91% yield). Single peak by HPLC.

### 210B. N,N-dimethyl-3-(6-(pyridin-4-yl)-1H-benzo[d]imidazol-2-yl)chroman-6-sulfonamide

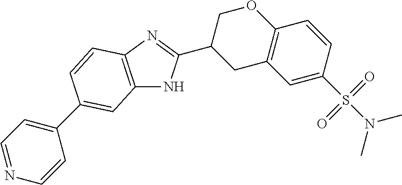

The desired product was prepared by using Example 210A and 4-(4,4,5,5-tetramethyl-1,3,2-dioxaborolan-2-yl)pyridine in Example 197 (4.6 mg). LC-MS: single peak at 254 nm, MH⁺ calcd. for C₂₃H₂₂N₄O₃S: 435, obtained 435. HPLC: single peak by analytical HPLC. ¹H-NMR (MeOD- d₄, 400 MHz): 8.84 (2H, m), 8.40 (2H, m), 8.25 (1H, d, J=1.7 Hz), 7.97 (1H, dd, J=1.8 Hz, 8.6 Hz), 7.84 (1H, d, J=8.6 Hz), 7.68 (1H, m), 7.58 (1H, dd, J=2.3 Hz, 8.6 Hz), 7.07 (1H, d, J=8.7 Hz), 4.74 (1H, dd, J=3.2 Hz, 11.0 Hz), 4.57 (1H, dd, J=8.4 Hz, 11.0 Hz), 3.87 (1H, m), 3.48 (2H, m), 2.70 (6H, s).

### Example 211

### 3-(6-(1H-pyrazol-4-yl)-1H-benzo[d]imidazol-2-yl)-N,N-dimethylchroman-6-sulfonamide

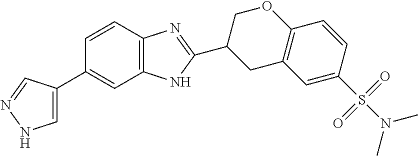

The desired product was prepared by using Example 210A and 4-(4,4,5,5-tetramethyl-1,3,2-dioxaborolan-2-yl)-1H-pyrazole in Example 197 (8.0 mg). LC-MS: single peak at 254 nm, MH⁺ calcd. for C₂₁H₂₁N₅O₃S: 424, obtained 424. HPLC: single peak by analytical HPLC. ¹H-NMR (MeOD- d₄, 400 MHz): 8.11 (2H, s), 7.93 (1H, m), 7.87 (1H, dd, J=1.5 Hz, 8.6 Hz), 7.77 (1H, d, J=8.6 Hz), 7.70 (1H, m), 7.61 (1H, dd, J=2.3 Hz, 8.6 Hz), 7.12 (1H, d, J=8.6 Hz), 4.76 (1H, dd, J=3.1 Hz, 11.3 Hz), 4.68 (1H, dd, J=6.8 Hz, 11.4 Hz), 4.11 (1H, m), 3.63 (1H, dd, J=5.8 Hz, 16.7 Hz), 3.48 (1H, dd, J=7.5 Hz, 16.8 Hz), 2.69 (6H, s).

### Example 212

### 3-(6-(2-aminopyrimidin-4-yl)-1H-benzo[d]imidazol-2-yl)-N,N-dimethylchroman-6-sulfonamide

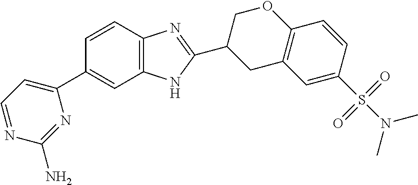

The desired product was prepared by using Example 210A in Example 197, and employing the alternative method to obtain the arylboronate, which was treated with 4-chloropyrimidin-2-amine (4.7 mg). LC-MS: single peak at 254 nm, MH⁺ calcd. for C₂₂H₂₂N₆O₃S: 451, obtained 451. HPLC: single peak by analytical HPLC. ¹H-NMR (MeOD-d₄, 400 MHz): 8.57 (1H, s), 8.31 (1H, d, J=6.6 Hz), 8.27 (1H, dd, J=1.6 Hz, 8.6 Hz), 7.78 (1H, d, J=8.6 Hz), 7.67 (1H, m), 7.58 (2H, m), 7.07 (1H, d, J=8.6 Hz), 4.74 (1H, dd, J=3.2 Hz, 11.0 Hz), 4.56 (1H, dd, J=8.5 Hz, 11.0 Hz), 3.86 (1H, m), 3.47 (2H, m), 2.70 (6H, s).

### Example 213

### 4-(2-(6-isopropoxychroman-3-yl)-1H-benzo[d]imidazol-6-yl)pyrimidin-2-amine

### 213A. 6-isopropoxychroman-3-carboxylic acid

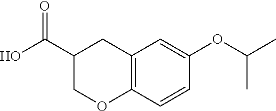

6-methoxychromane-3-carboxylic acid (1.0 equiv.) was combined with pyridinium chloride (3.0 equiv.) in a microwave pressure tube. The mixture was then heated at 175° C. in a microwave for 60 minutes to cleave the methyl ether. Upon completion, the reaction mixture was diluted with DMF (10 mL) and combined with Cs₂CO₃ (4.0 equiv.) and iodoethane (5.0 equiv.). The resulting solution was stirred at room temperature until alkylation of the acid was complete. The solution was then poured into water and thrice extracted with EtOAc. The combined organic portions were dried over MgSO₄ and purified on silica gel (hexane/EtOAc) to give the desired ester (85% yield). The ester (1.0 equiv) was dissolved in DMF containing Cs₂CO₃ (2.0 equiv.) and 2-bromopropane (3.0 equiv.). The solution was heated to 60° C. until alkylation of the phenolic hydroxy group was complete. The reaction mixture was poured into water and thrice extracted with EtOAc. The combined organic portions were dried over MgSO₄ and concentrated to give the desired isopropyl ether. This product was then stirred with LiOH (5.0 equiv.) in 1:1 dioxane:water to saponify the ester. Once saponification was complete, the solution was quenched with HCl and concentrated. The resulting acid was used directly in the HATU coupling.

### 213B. 4-(2-(6-isopropoxychroman-3-yl)-1H-benzo[d]imidazol-6-yl)pyrimidin-2-amine

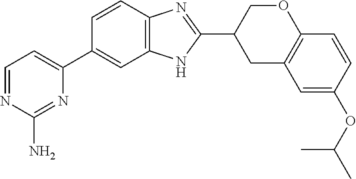

The desired product was prepared by using Example 213A in Example 197, and employing the alternative method to obtain the arylboronate, which was treated with 4-chloropyrimidin-2-amine (6.0 mg). LC-MS: single peak at 254 nm, MH⁺ calcd. for C₂₃H₂₃N₅O₂: 402, obtained 402.

### Example 214

### 2-(6-ethoxychroman-3-yl)-6-(pyridin-4-yl)-1H-benzo[d]imidazole

### 214A. 6-ethoxychroman-3-carboxylic acid

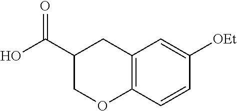

6-methoxychromane-3-carboxylic acid (0.5 g, 1.0 equiv.) was combined with pyridinium chloride (0.83 g, 3.0 equiv.) in a microwave pressure tube. The mixture was then heated at 175° C. in a microwave for 60 minutes to cleave the methyl ether. Upon completion, the reaction mixture was diluted with DMF (10 mL) and combined with Cs₂CO₃ (3.1 g, 4.0 equiv.) and iodoethane (0.97 mL, 5.0 equiv.). The resulting solution was warmed to 55° C. and stirred for 24 hours. The reaction mixture was then poured into water and thrice extracted with EtOAc. The combined organic portions were dried over MgSO₄ and purified on silica gel (hexane/EtOAc) to give the desired ester (140 mg). The ester was then stirred with LiOH (5.0 equiv.) in 1:1 dioxane:water to saponify the ester. Once saponification was complete, the solution was quenched with HCl and concentrated. The resulting acid was used directly in the HATU coupling.

### 214B. 2-(6-ethoxychroman-3-yl)-6-(pyridin-4-yl)-1H-benzo[d]imidazole

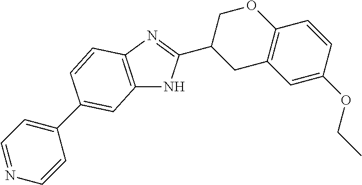

The desired product was prepared by using Example 214A and 4-(4,4,5,5-tetramethyl-1,3,2-dioxaborolan-2-yl)pyridine in Example 197 (6.3 mg). LC-MS: single peak at 254 nm, MH⁺ calcd. for C₂₃H₂₁N₃O₂: 372, obtained 372. HPLC: single peak by analytical HPLC. ¹H-NMR (MeOD- d₄, 400 MHz): 8.84 (2H, m), 8.37 (2H, m), 8.26 (1H, m), 8.01 (1H, dd, J=1.7 Hz, 8.6 Hz), 7.88 (1H, d, J=8.6 Hz), 6.76 (3H, m), 4.57 (1H, dd, J=3.0 Hz, 10.9 Hz), 4.46 (1H, dd, J=7.5 Hz, 11.1 Hz), 3.99 (2H, q, J=7.0 Hz), 3.86 (1H, m), 3.39 (2H, m), 1.37 (3H, t, J=7.0 Hz).

### Example 215

### 4-(2-(6-fluorochroman-3-yl)-1H-benzo[d]imidazol-6-yl)pyrimidin-2-amine

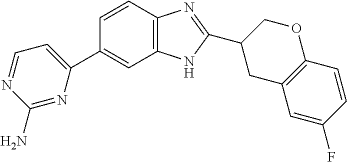

The desired product was prepared by using 6-fluorochroman-3-carboxylic acid in Example 197, and employing the alternative method to obtain the arylboronate, which was treated with 4-chloropyrimidin-2-amine (22.0 mg). LC-MS: single peak at 254 nm, MH⁺ calcd. for C₂₀H₁₆FN₅O: 362, obtained 362. HPLC: single peak by analytical HPLC. ¹H-NMR (MeOD- d₄, 400 MHz): 8.50 (1H, d, J=1.1 Hz), 8.22 (2H, m), 7.72 (1H, d, J=8.8 Hz), 7.49 (1H, d, J=6.7 Hz), 6.80 (3H, m), 4.50 (1H, dd, J=3.1 Hz, 11.0 Hz), 4.38 (1H, dd, J=7.6 Hz, 11.1 Hz), 3.78 (1H, m), 3.30 (2H, m).

### Example 216

### 3(6-(pyridin-4-yl)-1H-benzo[d]imidazol-2-yl)chroman-6-carbonitrile

### 216A. 6-cyanochroman-3-carboxylic acid

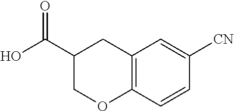

5-bromosalicylaldehyde (1.0 equiv.), ethyl acrylate (2.0 equiv.) and DABCO (0.2 equiv.) were combined in a microwave pressure tube. The mixture was then heated to 160° C. for 60 minutes. The solution was then diluted with CH₂Cl₂ and washed with saturated NH₄Cl. The product was then concentrated and purified on silica gel (hexane/EtOAc) to give the bromochromene product (23% yield). The chromene was then dissolved in MeOH containing Rh/Al₂O₃ (0.05 equiv) and stirred under a hydrogen balloon atmosphere until olefin reduction was complete. The solution was filtered through a plug of Celite and concentrated to give the desired bromochromane (96% yield). The bromochromane (1.0 equiv.) was then combined with Zn(CN)₂ (5.0 equiv.) and PdCl₂(PPh₃)₂ (0.05 equiv.) in DMF. The solution was degassed with argon and then heated to 180° C. for 30 minutes in a microwave. The solution was then poured into water and thrice extracted with EtOAc. The combined organic portions were dried over MgSO₄ and concentrated to give the nitrile product. This product was then stirred with LiOH (5.0 equiv.) in 1:1 dioxane:water to saponify the ester. Once saponification was complete, the solution was quenched with HCl and concentrated. The resulting acid was used directly in the HATU coupling.

### 216B. 3-(6-(pyridin-4-yl)-1H-benzo[d]imidazol-2-yl)chroman-6-carbonitrile

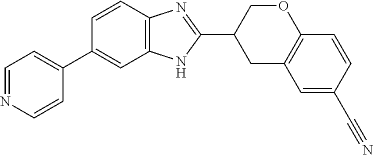

The desired product was prepared by using Example 216A and 4-(4,4,5,5-tetramethyl-1,3,2-dioxaborolan-2-yl)pyridine in Example 197 (7.8 mg). LC-MS: single peak at 254 nm, MH⁺ calcd. for C₂₂H₁₆N₄O: 353, obtained 353. HPLC: single peak by analytical HPLC. ¹H-NMR (MeOD- d₄, 400 MHz): 8.84 (2H, m), 8.41 (2H, m), 8.25 (1H, d, J=1.8 Hz), 7.97 (1H, dd, J=1.8 Hz, 8.6 Hz), 7.85 (1H, d, J=8.6 Hz), 7.63 (1H, m), 7.52 (1H, dd, J=2.1 Hz, 8.5 Hz), 7.01 (1H, d, J=8.5 Hz), 4.73 (1H, dd, J=3.2 Hz, 11.1 Hz), 4.58 (1H, dd, J=8.1 Hz, 11.1 Hz), 3.86 (1H, m), 3.44 (2H, m).

### Example 217

### 3-(6-(2-aminopyrimidin-4-yl)-1H-benzo[d]imidazol-2-yl)chroman-6-carbonitrile

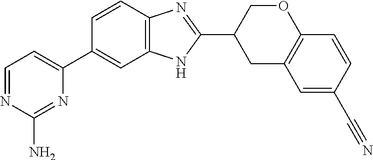

The desired product was prepared by using Example 216A in Example 197, and employing the alternative method to obtain the arylboronate, which was treated with 4-chloropyrimidin-2-amine (13.6 mg). LC-MS: single peak at 254 nm, MH⁺ calcd. for C₂₁H₁₆N₆O: 369, obtained 369. HPLC: single peak by analytical HPLC. ¹H-NMR (MeOD-d₄, 400 MHz): 8.59 (1H, d, J=1.2 Hz), 8.32 (1H, d, J=6.7 Hz), 8.29 (1H, dd, J=1.7 Hz, 8.7 Hz), 7.80 (1H, d, J=8.6 Hz), 7.61 (2H, m), 7.51 (1H, dd, J=2.1 Hz, 8.5 Hz), 7.01 (1H, d, J=8.5 Hz), 4.73 (1H, dd, J=3.0 Hz, 11.0 Hz), 4.58 (1H, dd, J=8.0 Hz, 11.1 Hz), 3.88 (1H, m), 3.44 (2H, m).

### Example 218

### 2-(6-(1-propoxy)chroman-3-yl)-6-(pyridin-4-yl)-1H-benzo[d]imidazole

### 218A. 6-(1-propoxy)chroman-3-carboxylic acid

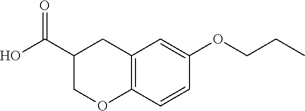

Ethyl 6-hydroxychroman-3-carboxylate (1.0 equiv.) was dissolved in DMF containing Cs₂CO₃ (2.0 equiv.) and allyl bromide (5.0 equiv.). The solution was stirred at room temperature until alkylation was complete. Upon completion, the reaction mixture was poured into water and thrice extracted with EtOAc. The combined organic portions were dried over MgSO₄ and concentrated to give the allylated product as an oil (93% yield). This material was then dissolved in EtOAc containing Pd/C (0.05 equiv.) and stirred under a hydrogen balloon atmosphere for 1 hour. The solution was then filtered through a plug of Celite and concentrated to give the propyl ether product in near quantitative yield. This product was then stirred with LiOH (5.0 equiv.) in 1:1 dioxane:water to saponify the ester. Once saponification was complete, the solution was quenched with HCl and concentrated. The resulting acid was used directly in the HATU coupling.

### 218B. 2-(6-(1-propoxy)chroman-3-yl)-6-(pyridin-4-yl)-1H-benzo[d]imidazole

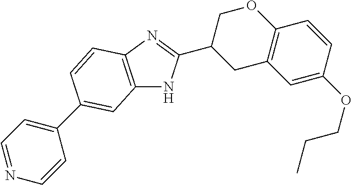

The desired product was prepared by using Example 218A and 4-(4,4,5,5-tetramethyl-1,3,2-dioxaborolan-2-yl)pyridine in Example 197 (6.23 mg). LC-MS: single peak at 254 nm, MH⁺ calcd. for C₂₄H₂₃N₃O₂: 386, obtained 386. HPLC: single peak by analytical HPLC. ¹H-NMR (MeOD- d₄, 400 MHz): 8.78 (2H, d, J=5.7 Hz), 8.32 (2H, d, J=5.7 Hz), 8.21 (1H, s), 7.96 (1H, d, J=8.6 Hz), 7.84 (1H, d, J=8.6 Hz), 6.65 (3H, m), 4.43 (2H, m), 3.85 (1H, m), 3.76 (2H, t, J=6.4 Hz), 3.32 (2H, m), 1.65 (2H, m), 0.92 (3H, t, J=7.4 Hz).

### Example 219

### 2-(6-(1-propoxy)chroman-3-yl)-6-(1H-pyrazol-4-yl)-1H-benzo[d]imidazole

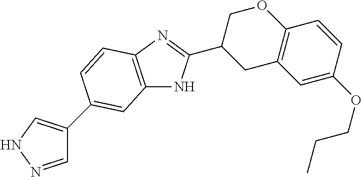

The desired product was prepared by using Example 218A and 4-(4,4,5,5-tetramethyl-1,3,2-dioxaborolan-2-yl)-1H-pyrazole in Example 197 (9.64 mg). LC-MS: single peak at 254 nm, MH⁺ calcd. for C₂₂H₂₂N₄O₂: 375, obtained 375. HPLC: single peak by analytical HPLC.

### Example 220

### 4-(2-(6-(1-propoxy)chroman-3-yl)-1H-benzo[d]imidazol-6-yl)pyrimidin-2-amine

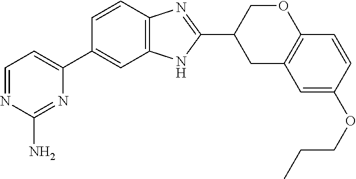

The desired product was prepared by using Example 218A in Example 197, and employing the alternative method to obtain the arylboronate, which was treated with 4-chloropyrimidin-2-amine (4.39). LC-MS: single peak at 254 nm, MH⁺ calcd. for C₂₃H₂₂N₅O₂: 402, obtained 402. HPLC: single peak by analytical HPLC. ¹H-NMR (MeOD- d₄, 400 MHz): 8.48 (1H, s), 8.21 (2H, m), 7.71 (1H, d, J=8.6 Hz), 7.46 (1H, d, J=6.6 Hz), 6.64 (3H, m), 4.45 (1H, dd, J=2.9 Hz, 11.1 Hz), 4.34 (1H, dd, J=7.7 Hz, 11.1 Hz), 3.76 (3H, m), 3.26 (2H, m), 1.66 (2H, m), 0.92 (3H, t, J=7.4 Hz).

### Example 221

### General Procedure for Amidation of Example 172 for Preparation of 3-(5-(2-aminopyrimidin-4-yl)-1H-benzo[d]imidazol-2-yl)-chroman-6-carboxamide

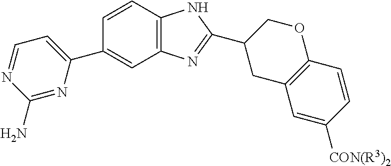

Example 172 (1.0 equiv.) is dissolved in a 1:1 dioxane:water solution at room temperature. To this solution is added LiOH (5.0 equiv.) and the resulting mixture is stirred at room temperature until saponification is complete. Upon completion, the solution is quenched with HCl (4.0 M in dioxane, 5.0 equiv.) and concentrated to give the carboxylic acid. This material is then dissolved in DMF containing Et₃N (10.0 equiv.). To this mixture is sequentially added an amine (3.0 equiv.) and HATU (3.0 equiv.). This mixture is stirred for 60 minutes at which time the reaction is monitored for completion (LC-MS). Preparative HPLC is utilized to obtain the final product as a TFA salt.

### Example 222

### (3-(5-(2-aminopyrimidin-4-yl)-1H-benzo[d]imidazol-2-yl)chroman-6-yl)(morpholino)methanone, trifluoroacetic acid salt

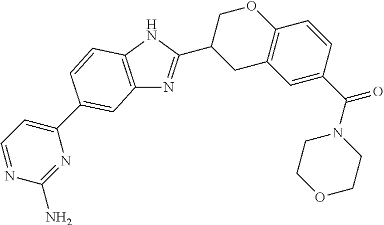

The desired product was prepared by using morpholine in Example 221 (10.2 mg). LC-MS: single peak at 254 nm, MH⁺calcd. for C₂₅H₂₄N₆O₃: 457, obtained 457. HPLC: single peak by analytical HPLC. ¹H-NMR (MeOD- d₄, 400 MHz): 8.61 (1H, s), 8.33 (2H, m), 7.83 (1H, d, J=8.7 Hz), 7.60 (1H, d, J=6.7 Hz), 7.33 (1H, s), 7.26 (1H, d, J=8.4 Hz), 6.95 (1H, d, J=8.4 Hz), 4.68 (1H, dd, J=3.1 Hz, 11.1 Hz), 4.57 (1H, dd, J=7.6 Hz, 11.1 Hz), 3.92 (1H, m), 3.70 (8H, m), 3.45 (2H, m).

### Example 223

### (3-(5-(2-aminopyrimidin-4-yl)-1H-benzo[d]imidazol-2-yl)chroman-6-yl)(4-methylpiperazin-1-yl)methanone, trifluoroacetic acid salt

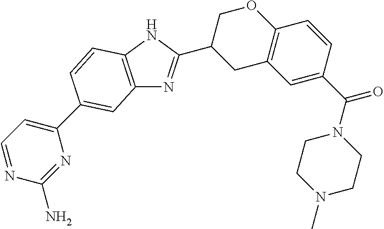

The desired product was prepared by using N-methylpiperazine in Example 221 (10.6 mg). LC-MS: single peak at 254 nm, MH⁺ calcd. for C₂₆H₂₇N₇O₂: 470, obtained 470. HPLC: single peak by analytical HPLC.

### Example 224

### 3-(5-(2-aminopyrimidin-4-yl)-1H-benzo[d]imidazol-2-yl)-N-(2-methoxyethyl)chroman-6-carboxamide, trifluoroacetic acid salt

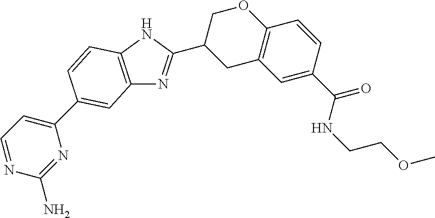

The desired product was prepared by using 2-methoxyethylamine in Example 221 (3.8 mg). LC-MS: single peak at 254 nm, MH⁺ calcd. for C₂₄H₂₄N₆O₃: 445, obtained 445. HPLC: single peak by analytical HPLC.

### Example 225

### 3-(5-(2-aminopyrimidin-4-yl)-1H-benzo[d]imidazol-2-yl)-N-(2-(dimethylamino)ethyl)chroman-6-carboxamide, trifluoroacetic acid salt

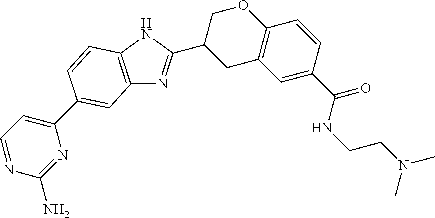

The desired product was prepared by using N,N-dimethylethylenediamine in Example 221 (7.7 mg). LC-MS: single peak at 254 nm, MH⁺ calcd. for C₂₅H₂₇N₇O₂: 458, obtained 458. HPLC: single peak by analytical HPLC. ¹H-NMR (MeOD- d₄, 400 MHz): 8.56 (1H, d, J=1.4 Hz), 8.31 (1H, d, J=6.6 Hz), 8.26 (1H, dd, J=1.6 Hz, 8.7 Hz), 7.79 (1H, d, J=2.1 Hz), 7.77 (1H, d, J=8.7 Hz), 7.70 (1H, dd, J=2.3 Hz, 8.7 Hz), 7.58 (1H, d, J=6.6 Hz), 6.96 (1H, d, J=8.6 Hz), 4.71 (1H, dd, J=2.7 Hz, 10.9 Hz), 4.51 (1H, dd, J=8.5 Hz, 10.9 Hz), 3.83 (1H, m), 3.75 (2H, t, J=5.8 Hz), 3.44 (2H, m), 3.38 (2H, t, J=5.8 Hz), 3.00 (6H, s).

### Example 226

### 3-(5-(2-aminopyrimidin-4-yl)-1H-benzo[d]imidazol-2-yl)-N-(2-morpholinoethyl)chroman-6-carboxamide, trifluoroacetic acid salt

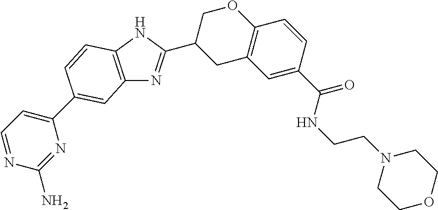

The desired product was prepared by using 2-morpholinoethylamine in Example 221 (8.5 mg). LC-MS: single peak at 254 nm, MH⁺ calcd. for C₂₇H₂₉N₇O₃: 500, obtained 500. HPLC: single peak by analytical HPLC. ¹H-NMR (MeOD- d₄, 400 MHz): 8.30 (1H, s), 8.26 (1H, d, J=5.4 Hz), 7.98 (1H, dd, J=1.6 Hz, 8.5 Hz), 7.72 (1H, s), 7.63 (1H, d, J=8.5 Hz), 7.18 (1H, d, J=5.4 Hz), 6.90 (1H, d, J=8.5 Hz), 4.66 (1H, m), 4.41 (1H, dd, J=9.4 Hz, 10.8 Hz), 3.73 (4H, t, J=4.7 Hz), 3.68 (1H, m), 3.55 (2H, t, J=6.8 Hz), 3.36 (2H, m), 2.60 (6H, m).

### Example 227

### 3-(5-(2-aminopyrimidin-4-yl)-1H-benzo[d]imidazol-2-yl)-N-isopropylchroman-6-carboxamide, trifluoroacetic acid salt

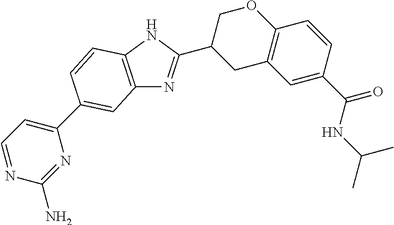

The desired product was prepared by using isopropylamine in Example 221 (4.0 mg). LC-MS: single peak at 254 nm, MH⁺ calcd. for C₂₄H₂₄N₆O₂: 429, obtained 429. HPLC: single peak by analytical HPLC.

### Example 228

### 3-(5-(2-aminopyrimidin-4-yl)-1H-benzo[d]imidazol-2-yl)-N-cyclopropylchroman-6-carboxamide, trifluoroacetic acid salt

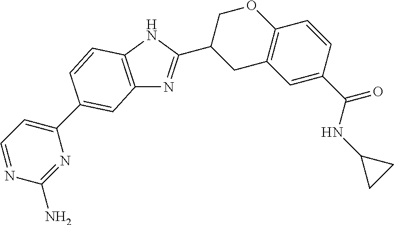

The desired product was prepared by using cyclopropylamine in Example 221 (6.4 mg). LC-MS: single peak at 254 nm, MH⁺ calcd. for C₂₄H₂₂N₆O₂: 427, obtained 427. HPLC: single peak by analytical HPLC.

### Example 229

### 3-(5-(2-aminopyrimidin-4-yl)-1H-benzo[d]imidazol-2-yl)-N-benzylchroman-6-carboxamide, trifluoroacetic acid salt

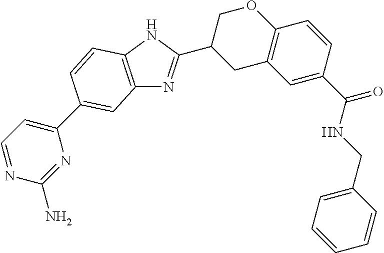

The desired product was prepared by using benzylamine in Example 221 (5.0 mg). LC-MS: single peak at 254 nm, MH⁺ calcd. for C₂₈H₂₄N₆O₂: 477, obtained 477. HPLC: single peak by analytical HPLC.

### Example 230

### 3-(5-(2-aminopyrimidin-4-yl)-1H-benzo[d]imidazol-2-yl)-N-(4-methoxybenzyl)chroman-6-carboxamide, trifluoroacetic acid salt

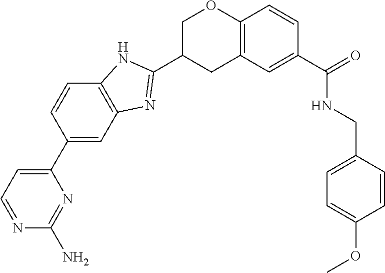

The desired product was prepared by using 4-methoxybenzylamine in Example 221 (19.2 mg). LC-MS: single peak at 254 nm, MH⁺ calcd. for C₂₉H₂₆N₆O₃: 507, obtained 507. HPLC: single peak by analytical HPLC. ¹H-NMR (MeOD- d₄, 400 MHz): 8.60 (1H, s), 8.32 (2H, m), 7.82 (1H, d, J=8.8 Hz), 7.76 (1H, s), 7.67 (1H, dd, J=2.3 Hz, 8.6 Hz), 7.59 (1H, d, J=6.7 Hz), 7.28 (2H, m), 6.93 (1H, d, J=8.6 Hz), 6.88 (2H, m), 4.69 (1H, dd, J=2.8 Hz, 11.1 Hz), 4.56 (1H, dd, J=7.7 Hz, 11.1 Hz), 4.50 (2H, s), 3.92 (1H, m), 3.78 (3H, s), 3.46 (2H, m).

### Example 231

### 3-(5-(2-aminopyrimidin-4-yl)-1H-benzo[d]imidazol-2-yl)-N-((thiophen-2-yl)methyl)chroman-6-carboxamide, trifluoroacetic acid salt

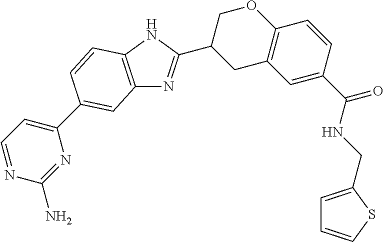

The desired product was prepared by using 2-thiophenemethylamine in Example 221 (19.8 mg). LC-MS: single peak at 254 nm, MH⁺ calcd. for C₂₆H₂₂N₆O₂S: 483, obtained 483. HPLC: single peak by analytical HPLC.

### Example 232

### 3-(5-(2-aminopyrimidin-4-yl)-1H-benzo[d]imidazol-2-yl)-N-phenethylchroman-6-carboxamide, trifluoroacetic acid salt

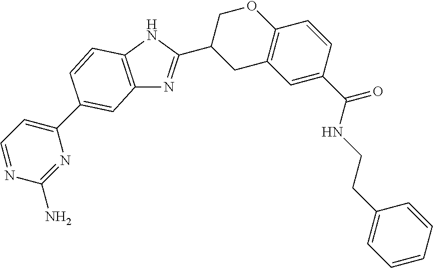

The desired product was prepared by using phenethylamine in Example 221 (29.7 mg). LC-MS: single peak at 254 nm, MH⁺ calcd. for C₂₉H₂₆N₆O₂: 491, obtained 491. HPLC: single peak by analytical HPLC.

### Example 233

### 3-(5-(2-aminopyrimidin-4-yl)-1H-benzo[d]imidazol-2-yl)-N-(1-(thiophen-2-yl)propan-2-yl)chroman-6-carboxamide, trifluoroacetic acid salt

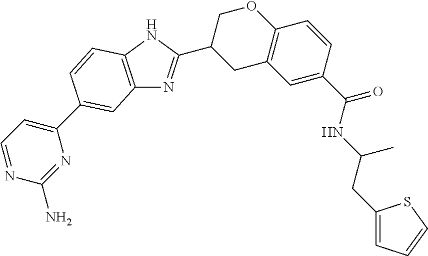

The desired product was prepared by using 1-(thiophen-2-yl)propan-2-amine in Example 221 (19.2 mg). LC-MS: single peak at 254 nm, MH⁺ calcd. for C₂₈H₂₆N₆O₂S: 511, obtained 511. HPLC: single peak by analytical HPLC.

### Example 234

### 2-(6-(1H-pyrazol-4-yl)chroman-3-yl)-5-(1H-pyrazol-4-yl)-1H-benzo[d]imidazole

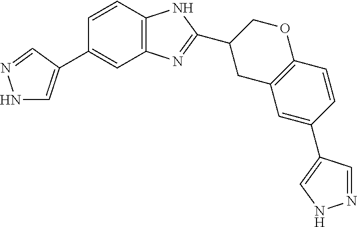

HATU (1.2 equiv.), Et₃N (2.0 equiv.) and 6-bromochroman-3-carboxylic acid (1.0 equiv.) were combined in anhydrous DMF at room temperature. To this solution was then added 4-bromobenzene-1,2-diamine (1.0 equiv.) and the resulting mixture was stirred at room temperature until complete by LC-MS analysis. Upon completion, the solution was concentrated in vacuo and then diluted with glacial AcOH (5 mL). This solution was warmed to 65° C. and stirred until the cyclodehydration was complete by LC-MS. The reaction was then concentrated in vacuo and diluted with EtOAc. The solution was then washed with concentrated aqueous NaHCO₃ and back extracted twice with additional EtOAc. The combined organic fractions were dried over MgSO₄ and purified on SiO₂ (Hexane/EtOAc) to give the bis-arylbromide product (74%). The bis-aryl bromide (1.0 equiv.) was then combined with 4-(4,4,5,5-tetramethyl-1,3,2-dioxaborolan-2-yl)-1H-pyrazole (1.30 equiv.) and Na₂CO₃ (3.0 equiv.) in MeOH in a microwave pressure tube at room temperature. To this solution was added PdCl₂(PPh₃)₂ (0.10 equiv.) and the solution was sparged with argon for 10 minutes. The reaction was subsequently heated in a microwave at 120° C. until the reaction was complete. The product was then purified via preparative HPLC. LC-MS: single peak at 254 nm, MH⁺ calcd. for C₂₂H₁₈N₆O: 383, obtained 383. 1-HPLC: single peak by analytical HPLC. ¹H-NMR (MeOD- d₄, 400 MHz): 8.11 (2H, s), 7.96 (2H, s), 7.92 (1H, m), 7.86 (1H, dd, J=1.6 Hz, 8.6 Hz), 7.76 (1H, d, J=8.6 Hz), 7.45 (1H, s), 7.40 (1H, dd, J=2.2 Hz, 8.5 Hz), 6.92 (1H, d, J=8.5 Hz), 4.64 (2H, m), 4.06 (1H, m), 3.58 (1H, dd, J=6.0 Hz, 16.8 Hz), 3.42 (1H, dd, J=6.7 Hz, 16.8 Hz).

### Example 235

### (R)-2-phenyl-1-(5-(pyridin-4-yl)-1H-benzo[d]imidazol-2-yl)ethanol

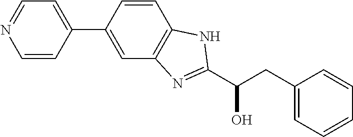

The desired product was prepared by substituting (R)-2-hydroxy-3-phenylpropanoic acid for Boc-D-Phe-OH in Example 59. ¹H NMR (CDCl₃, 400 MHz) δ 3.04-3.10 (m, 1H), 3.23-3.27 (m, 1H), 5.15-5.19 (m, 1H), 7.11-7.22 (m, 5H), 7.72-7.74 (m, 2H), 7.84-7.86 (m, 2H), 8.13-8.15 (complex, 3H), 8.76-8.79 (m, 5H); LC/MS: C₂₀H₁₈N₃O (M+1) 316.19.

### Example 236

### 2-(6-ethoxychroman-3-yl)-7-fluoro-5-(pyridin-4-yl)-1H-benzo[d]imidazole

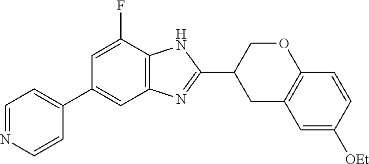

Procedures in Scheme 11 were utilized to synthesize this compound. ¹H-NMR (MeOD-d₄, 400 MHz) δ 8.81 (2H, d, J=6.1 Hz), 8.34 (2H, d, J=6.1 Hz), 8.0 (1H, s), 7.67 (1H, d, J=11.5 Hz), 6.74 (3H, m), 4.55 (1H, m), 4.32 (1H, m), 3.98 (2H, q, J=7.0 Hz), 3.68 (1H, m), 3.37 (1H, m), 3.26 (1H, dd, J=5.6 Hz, 16.4 Hz), 1.36 (3H, t, J=7.0 Hz). LC/MS: C₂₃H₂₀FN₃O₂ (M+1) 390. Single peak at both 215 nm and 254 nm in analytical HPLC traces.

### Example 237

### 2-(6-ethoxychroman-3-yl)-7-fluoro-5-(1H-pyrazol-4-yl)-1H-benzo[d]imidazole

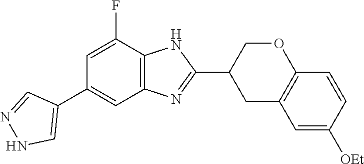

Procedures in Scheme 11 were utilized to synthesize this compound. ¹H-NMR (MeOD-d₄, 400 MHz) δ 8.04 (2H, s), 7.61 (1H, s), 7.44 (1H, d, J=11.8 Hz), 6.75 (3H, m), 4.54 (1H, dd, J=1.9 Hz, 10.6 Hz), 4.37 (1H, dd, J=9.7 Hz, 10.4 Hz), 3.97 (2H, q, J=7.0 Hz), 3.77 (1H, m), 3.34 (2H, m), 1.36 (3H, t, J=7.0 Hz). LC/MS: C₂₁H₁₉FN₄O₂ (M+1) 379. Single peak at both 215 nm and 254 nm in analytical HPLC traces.

### Example 238

### 4-(2-(6-ethoxychroman-3-yl)-7-fluoro-1H-benzo[d]imidazol-5-yl)pyrimidin-2-amine

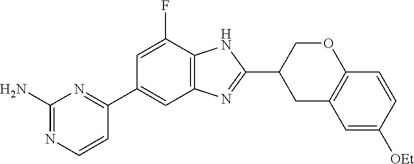

Procedures in Scheme 11 were utilized to synthesize this compound. ¹H-NMR (MeOD-d₄, 400 MHz) δ 8.29 (2H, m), 7.96 (1H, dd, J=1.4 Hz, 11.8 Hz), 7.56 (1H, d, J=6.8 Hz), 6.72 (3H, m), 4.53 (1H, m), 4.31 (1H, dd, J=9.1 Hz, 10.8 Hz), 3.97 (2H, q, J=7.0 Hz), 3.67 (1H, m), 3.35 (1H, m), 3.24 (1H, dd, J=5.8 Hz, 16.1 Hz), 1.35 (3H, t, J=7.0 Hz). LC/MS: C₂₂H₂₀FN₅O₂ (M+1) 406. Single peak at both 215 nm and 254 nm in analytical HPLC traces.

### Example 239

### methyl 3-(5-(2-aminopyrimidin-4-yl)-7-fluoro-1H-benzo[d]imidazol-2-yl)chroman-6-carboxylate

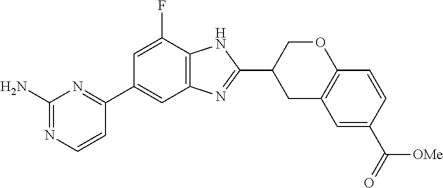

Procedures in Scheme 11 were utilized to synthesize this compound. ¹H-NMR (DMSO-d₆, 400 MHz) δ 8.30 (1H, d, J=5.0 Hz), 8.07 (1H, m), 7.84 (1H, s), 7.72 (2H, m), 7.20 (1H, d, J=5.0 Hz), 6.92 (1H, d, J=8.5 Hz), 6.65 (2H, bs), 4.67 (1H, m), 4.42 (1H, dd, J=9.0 Hz, 11.0 Hz), 3.82 (3H, s), 3.66 (1H, m), 3.34 (2H, m). LC/MS: C₂₂H₁₈FN₅O₃ (M+1) 420. Single peak at both 215 nm and 254 nm in analytical HPLC traces

### Example 240

### 3-(5-(2-aminopyrimidin-4-yl)-7-fluoro-1H-benzo[d]imidazol-2-yl)chroman-6-carboxylic acid

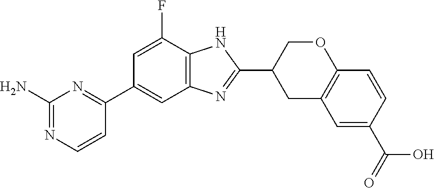

The methyl ester Example 239 (1.0 equiv) was dissolved in a 1:1 dioxane:water solution at room temperature. To this solution was added LiOH (5.0 equiv) and the resulting mixture was stirred at room temperature until saponification was complete. Upon completion, the solution was quenched with HCl (4.0 M in dioxane, 5.0 equiv) and concentrated to give the carboxylic acid. ¹H-NMR (MeOD-d₄, 400 MHz) δ 8.32 (1H, d, J=1.4 Hz), 8.28 (1H, d, J=6.7 Hz), 7.96 (1H, dd, J=1.4 Hz, 11.8 Hz), 7.90 (1H, m), 7.80 (1H, dd, J=2.1 Hz, 8.5 Hz), 7.57 (1H, d, J=6.7 Hz), 6.90 (1H, d, J=8.6 Hz), 4.69 (1H, m), 4.46 (1H, dd, J=9.3 Hz, 10.9 Hz), 3.73 (1H, m), 3.37 (2H, m). LC/MS: C₂₁H₁₆FN₅O₃ (M+1) 406. Single peak at both 215 nm and 254 nm in analytical HPLC traces.

### Example 241

### (3-(5-(2-aminopyrimidin-4-yl)-7-fluoro-1H-benzo[d]imidazol-2-yl)chroman-6-yl)methanol

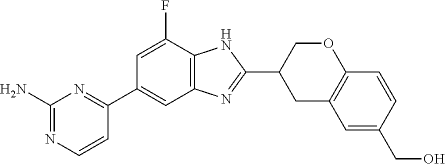

This compound was synthesized by treating the corresponding carboxylic acid (1.0 equiv) (from Example 239) with BH₃ THF (4.0 equiv) in THF under argon. Upon completion of the reaction by LC-MS, the solution was quenched with MeOH and concentrated to give the primary alcohol. LC/MS: C₂₁H₁₈FN₅O₂ (M+1) 392. Single peak at both 215 nm and 254 nm in analytical HPLC traces.

### Example 242

### 3-(5-(2-aminopyrimidin-4-yl)-7-fluoro-1H-benzo[d]imidazol-2-yl)-N-isobutylchroman-6-carboxamide

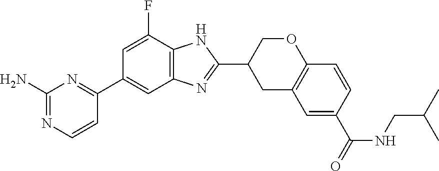

The free acid Example 240 was dissolved in DMF containing Et₃N (10.0 equiv). To this mixture was sequentially added an amine (3.0 equiv) and HATU (3.0 equiv). This mixture was stirred for 60 minutes at which time the reaction was complete. Purified via preparative HPLC. LC/MS: C₂₅H₂₅FN₆O₂ (M+1) 461. Single peak at both 215 nm and 254 nm in analytical HPLC traces.

### Example 243

### 3-(5-(2-aminopyrimidin-4-yl)-7-fluoro-1H-benzo[d]imidazol-2-yl)-N-cyclopropylchroman-6-carboxamide

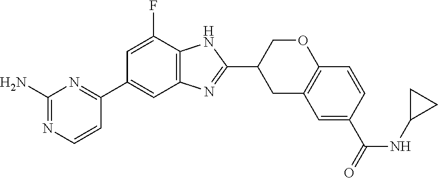

Procedures in Example 242 were utilized to synthesize this compound. LC/MS: C₂₄H₂₁FN₆O₂ (M+1) 445. Single peak at both 215 nm and 254 nm in analytical HPLC traces.

### Example 244

### 3-(5-(2-aminopyrimidin-4-yl)-7-fluoro-1H-benzo[d]imidazol-2-yl)-N-cyclobutylchroman-6-carboxamide

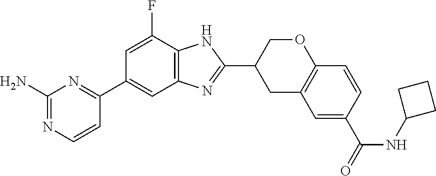

Procedures in Example 242 were utilized to synthesize this compound. LC/MS: C₂₅H₂₃FN₆O₂ (M+1) 459. Single peak at both 215 nm and 254 nm in analytical HPLC traces.

### Example 245

### 3-(5-(2-aminopyrimidin-4-yl)-7-fluoro-1H-benzo[d]imidazol-2-yl)-N-(2-(thiophen-2-yl)ethyl)chroman-6-carboxamide

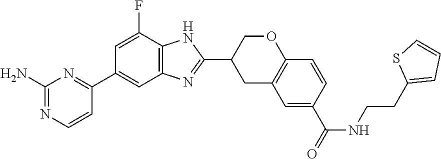

Procedures in Example 242 were utilized to synthesize this compound. LC/MS: C₂₇H₂₃FN₆O₂S (M+1) 515. Single peak at 254 nm in analytical HPLC traces.

### Example 246

### 3-(5-(2-aminopyrimidin-4-yl)-7-fluoro-1H-benzo[d]imidazol-2-yl)-N-(4-methoxyphenethyl)chroman-6-carboxamide

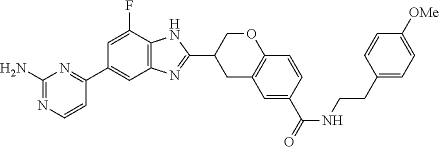

Procedures in Example 242 were utilized to synthesize this compound. LC/MS: C₃₀H₂₇FN₆O₃ (M+1) 539. Single peak at 254 nm in analytical HPLC traces.

### Example 247

### 3-(5-(2-aminopyrimidin-4-yl)-7-fluoro-1H-benzo[d]imidazol-2-yl)-N-(2-(dimethylamino)ethyl)chroman-6-carboxamide

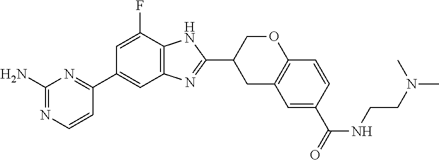

Procedures in Example 242 were utilized to synthesize this compound. ¹H-NMR (MeOD-d₄, 400 MHz) δ 8.31 (1H, d, J=1.3 Hz), 8.29 (1H, d, J=6.6 Hz), 7.95 (1H, dd, J=1.3 Hz, 11.9 Hz), 7.76 (1H, d, J=2.1 Hz), 7.67 (1H, dd, J=2.3 Hz, 8.6 Hz), 7.55 (1H, d, J=6.6 Hz), 6.93 (1H, d, J=8.6 Hz), 4.68 (1H, m), 4.45 (1H, dd, J=9.2 Hz, 10.8 Hz), 3.74 (3H, m), 3.45 (1H, m), 3.37 (3H, m), 2.98 (6H, s). LC/MS: C₂₅H₂₆FN₇O₂ (M+1) 476. Single peak at both 215 nm and 254 nm in analytical HPLC traces.

### Example 248

### 3-(5-(2-aminopyrimidin-4-yl)-7-fluoro-1H-benzo[d]imidazol-2-yl)-N-cyclopentylchroman-6-carboxamide

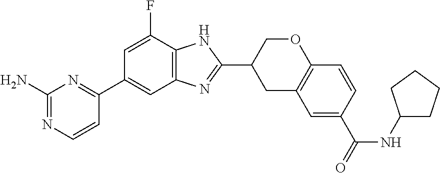

Procedures in Example 242 were utilized to synthesize this compound. LC/MS: C₂₆H₂₅FN₆O₂ (M+1) 473. Single peak at both 215 nm and 254 nm in analytical HPLC traces.

### Example 249

### 3-(5-(2-aminopyrimidin-4-yl)-7-fluoro-1H-benzo[d]imidazol-2-yl)-N-methoxy-N-methylchroman-6-carboxamide

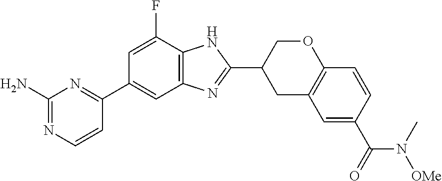

Procedures in Example 242 were utilized to synthesize this compound. ¹H-NMR (MeOD-d₄, 400 MHz) δ 8.28 (1H, d, J=5.4 Hz), 8.10 (1H, bs), 7.77 (1H, m), 7.57 (1H, m), 7.50 (1H, dd, J=2.2 Hz, 8.5 Hz), 7.18 (1H, d, J=5.4 Hz), 6.90 (1H, d, J=8.5 Hz), 4.67 (1H, m), 4.42 (1H, dd, J=9.5 Hz, 10.8 Hz), 3.69 (1H, m), 3.61 (3H, s), 3.39 (2H, m), 3.35 (3H, s). LC/MS: C₂₃H₂₁FN₆O₃ (M+1) 449. Single peak at both 215 nm and 254 nm in analytical HPLC traces.

### Example 250

### 3-(6-(2-aminopyrimidin-4-yl)-3H-imidazo[4,5-h]pyridin-2-yl)-N-isobutylchroman-6-carboxamide

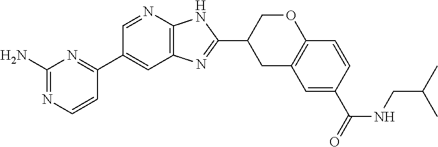

Similar procedures as in the preparation from Examples 240& 242 were utilized to synthesize this compound. ¹H-NMR (MeOD-d₄, 400 MHz) δ 9.23 (1H, d, J=2.0 Hz), 8.78 (1H, d, J=2.0 Hz), 8.38 (1H, d, J=6.5 Hz), 7.71 (1H, m), 7.61 (2H, m), 6.89 (1H, d, J=8.6 Hz), 4.68 (1H, m), 4.48 (1H, dd, J=8.7 Hz, 10.9 Hz), 3.76 (1H, m), 3.40 (2H, m), 3.17 (2H, d, J=7.0 Hz), 1.92 (1H, m), 0.96 (6H, d, J=6.7 Hz). LC/MS: C₂₄H₂₅N₇O₂ (M+1) 444. Single peak at both 215 nm and 254 nm in analytical HPLC traces.

### Example 251

### 3-(6-(2-aminopyrimidin-4-yl)-3H-imidazo[4,5-b]pyridin-2-yl)-N-(2-methoxyethyl)chroman-6-carboxamide

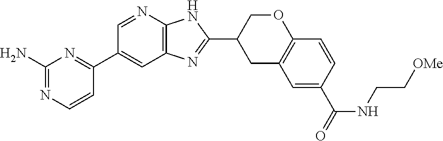

Similar procedures as in the preparation from Examples 240& 242 were utilized to synthesize this compound. ¹H-NMR (MeOD-d₄, 400 MHz) δ 9.25 (1H, d, J=1.9 Hz), 8.80 (1H, d, J=1.9 Hz), 8.33 (1H, d, J=6.7 Hz), 7.71 (1H, s), 7.63 (2H, m), 6.89 (1H, d, J=8.6 Hz), 4.68 (1H, m), 4.48 (1H, dd, J=8.9 Hz, 10.8 Hz), 3.76 (1H, m), 3.55 (4H, m), 3.44 (5H, m). LC/MS: C₂₃H₂₃N₇O₃ (M+1) 446. Single peak at both 215 nm and 254 nm in analytical HPLC traces.

### Example 252

### 3-(6-(2-aminopyrimidin-4-yl)-3H-imidazo[4,5-b]pyridin-2-yl)-N-(2-(dimethylamino)ethyl)chroman-6-carboxamide

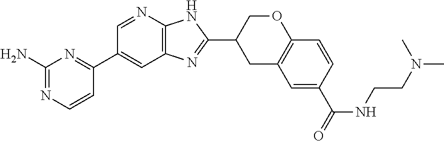

Similar procedures as in the preparation from Examples 240& 242 were utilized to synthesize this compound. LC/MS: C₂₄H₂₆N₈O₂ (M+1) 459. Single peak at both 215 nm and 254 nm in analytical HPLC traces.

### Example 253

### N-(2-(1H-imidazol-5-yl)ethyl)-3-(6-(2-aminopyrimidin-4-yl)-3H-imidazo[4,5-b]pyridin-2-yl)chroman-6-carboxamide

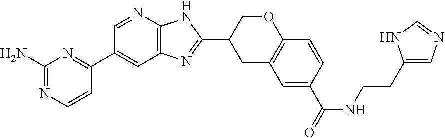

Similar procedures as in the preparation from Examples 240& 242 were utilized to synthesize this compound. LC/MS: C₂₅H₂₃N₉O₂ (M+1) 482. Single peak at both 215 nm and 254 nm in analytical HPLC traces.

### Example 254

### 3-(6-(2-aminopyrimidin-4-yl)-3H-imidazo[4,5-b]pyridin-2-yl)-N-(2-(pyridin-3-yl)ethyl)chroman-6-carboxamide

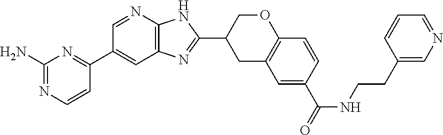

Similar procedures as in the preparation from Examples 240& 242 were utilized to synthesize this compound. ¹H-NMR (MeOD-d₄, 400 MHz) δ 9.25 (1H, d, J=1.9 Hz), 8.79 (2H, m), 8.71 (1H, d, J=5.7 Hz), 8.52 (1H, d, J=8.0 Hz), 8.33 (1H, d, J=6.6 Hz), 7.99 (1H, dd, J=5.8 Hz, 8.0 Hz), 7.64 (2H, m), 7.55 (1H, dd, J=2.0 Hz, 6.6 Hz), 6.88 (1H, d, J=8.6 Hz), 4.68 (1H, m), 4.47 (1H, dd, J=9.0 Hz, 10.9 Hz), 3.73 (3H, m), 3.39 (2H, m), 3.16 (2H, t, J=6.7 Hz). LC/MS: C₂₇H₂₄N₈O₂ (M+1) 493. Single peak at both 215 nm and 254 nm in analytical HPLC traces.

### Example 255

### 3-(6-(2-aminopyrimidin-4-yl)-3H-imidazo[4,5-b]pyridin-2-yl)-N-cyclopropylchroman-6-carboxamide

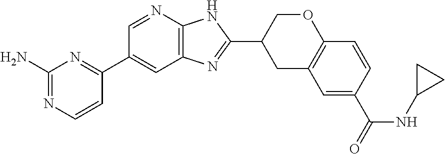

Similar procedures as in the preparation from Examples 240& 242 were utilized to synthesize this compound. LC/MS: C₂₃H₂₁N₇O₂ (M+1) 428. Single peak at both 215 nm and 254 nm in analytical HPLC traces.

### Example 256

### 3-(5-(2-aminopyrimidin-4-yl)-1H-benzo[d]imidazol-2-yl)-N-ethylchroman-6-carboxamide

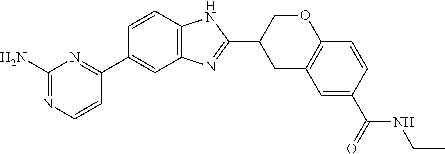

Procedures in Example 242 were utilized to synthesize this compound. ¹H-NMR (MeOD-d₄, 400 MHz) δ 8.59 (1H, s), 8.31 (2H, m), 7.81 (1H, d, J=8.7 Hz), 7.72 (1H, m), 7.62 (1H, dd, J=2.3 Hz, 8.6 Hz), 7.59 (1H, d, J=6.7 Hz), 6.91 (1H, d, J=8.5 Hz), 4.68 (1H, m), 4.55 (1H, dd, J=7.8 Hz, 11.1 Hz), 3.92 (1H, m), 3.45 (2H, m), 3.39 (2H, q, J=7.2 Hz), 1.21 (3H, t, J=7.2 Hz). LC/MS: C₂₃H₂₂N₆O₂ (M+1) 415. Single peak at both 215 nm and 254 nm in analytical HPLC traces.

### Example 257

### 3-(5-(2-aminopyrimidin-4-yl)-1H-benzo[d]imidazol-2-yl)-N-isobutylchroman-6-carboxamide

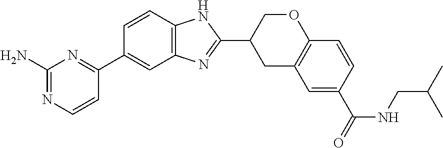

Procedures in Example 242 were utilized to synthesize this compound. LC/MS: C₂₅H₂₆N₆O₂ (M+1) 443. Single peak at both 215 nm and 254 nm in analytical HPLC traces.

### Example 258

### N-allyl-3-(5-(2-aminopyrimidin-4-yl)-1H-benzo[d]imidazol-2-yl)chroman-6-carboxamide

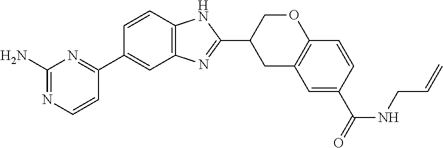

Procedures in Example 242 were utilized to synthesize this compound. LC/MS: C₂₄H₂₂N₆O₂ (M+1) 427. Single peak at both 215 nm and 254 nm in analytical HPLC traces.

### Example 259

### 3-(5-(2-aminopyrimidin-4-yl)-1H-benzo[d]imidazol-2-yl)-N-cyclohexylchroman-6-carboxamide

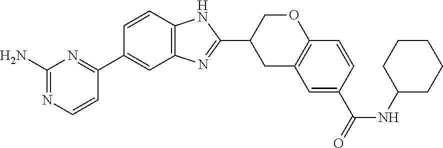

Procedures in Example 242 were utilized to synthesize this compound. ¹H-NMR (MeOD-d₄, 400 MHz) δ 8.59 (1H, m), 8.31 (2H, m), 7.81 (1H, d, J=8.6 Hz), 7.71 (1H, d, J=2.2 Hz), 7.62 (1H, dd, J=2.3 Hz, 8.6 Hz), 7.59 (1H, d, J=6.7 Hz), 6.91 (1H, d, J=8.6 Hz), 4.68 (1H, m), 4.55 (1H, dd, J=7.7 Hz, 11.1 Hz), 3.91 (1H, m), 3.84 (1H, m), 3.45 (2H, m), 1.93 (2H, m), 1.80 (2H, m), 1.68 (1H, m), 1.36 (4H, m), 1.22 (1H, m). LC/MS: C₂₇H₂₈N₆O₂ (M+1) 469. Single peak at both 215 nm and 254 nm in analytical HPLC traces.

### Example 260

### 3-(5-(2-aminopyrimidin-4-yl)-1H-benzo[d]imidazol-2-yl)-N-cyclopentylchroman-6-carboxamide

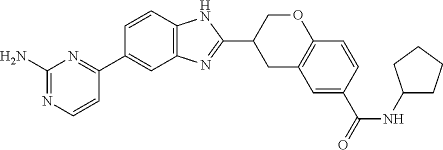

Procedures in Example 242 were utilized to synthesize this compound. LC/MS: C₂₆H₂₆N₆O₂ (M+1) 455. Single peak at both 215 nm and 254 nm in analytical HPLC traces.

### Example 261

### 3-(5-(2-aminopyrimidin-4-yl)-1H-benzo[d]imidazol-2-yl)-N-cyclobutylchroman-6-carboxamide

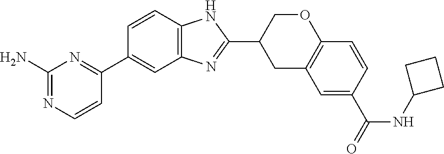

Procedures in Example 242 were utilized to synthesize this compound. LC/MS: C₂₅H₂₄N₆O₂ (M+1) 441. Single peak at both 215 nm and 254 nm in analytical HPLC traces.

### Example 262

### N-(2-(1H-imidazol-5-yl)ethyl)-3-(5-(2-aminopyrimidin-4-yl)-1H-benzo[d]imidazol-2-yl)chroman-6-carboxamide

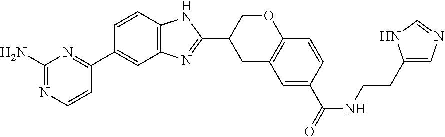

Procedures in Example 242 were utilized to synthesize this compound. ¹H-NMR (MeOD-d₄, 400 MHz) δ 8.80 (1H, d, J=1.4 Hz), 8.61 (1H, d, J=1.5 Hz), 8.31 (2H, m), 7.81 (1H, d, J=8.7 Hz), 7.69 (1H, m), 7.59 (2H, m), 7.35 (1H, s), 6.92 (1H, d, J=8.7 Hz), 4.68 (1H, dd, J=3.1 Hz, 11.1 Hz), 4.54 (1H, dd, J=8.0 Hz, 11.1 Hz), 3.91 (1H, m), 3.68 (2H, t, J=6.8 Hz), 3.44 (2H, m), 3.02 (2H, t, J=6.8 Hz). LC/MS: C₂₄H₂₆N₈O₂ (M+1) 481. Single peak at both 215 nm and 254 nm in analytical HPLC traces.

### Example 263

### 3-(5-(2-aminopyrimidin-4-yl)-1H-benzo[d]imidazol-2-yl)-N-(2-hydroxyethyl)chroman-6-carboxamide

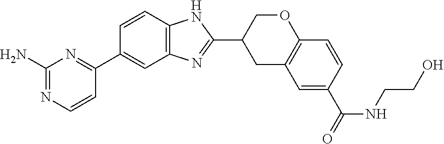

Procedures in Example 242 were utilized to synthesize this compound. LC/MS: C₂₃H₂₂N₆O₃ (M+1) 431. Single peak at both 215 nm and 254 nm in analytical HPLC traces.

### Example 264

### 3-(5-(2-aminopyrimidin-4-yl)-1H-benzo[d]imidazol-2-yl)-N-methyl-N-phenethylchroman-6-carboxamide

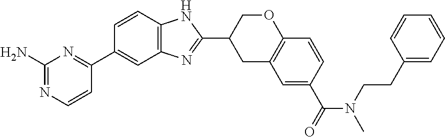

Procedures in Example 242 were utilized to synthesize this compound. LC/MS: C₃₀H₂₈N₆O₂ (M+1) 505. Single peak at both 215 nm and 254 nm in analytical HPLC traces.

### Example 265

### 3-(5-(2-aminopyrimidin-4-yl)-1H-benzo[d]imidazol-2-yl)-N-(2-(thiophen-2-yl)ethyl)chroman-6-carboxamide

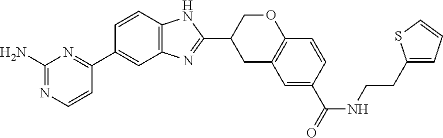

Procedures in Example 242 were utilized to synthesize this compound. ¹H-NMR (MeOD-d₄, 400 MHz) δ 8.56 (1H, d, J=1.2 Hz), 8.29 (1H, d, J=6.6 Hz), 8.26 (1H, dd, J=1.7 Hz, 8.7 Hz), 7.77 (1H, d, J=8.8 Hz), 7.70 (1H, m), 7.60 (1H, dd, J=2.3 Hz, 8.6 Hz), 7.56 (1H, d, J=6.6 Hz), 7.20 (1H, dd, J=1.3 Hz, 5.1 Hz), 6.91 (3H, m), 4.68 (1H, dd, J=3.1 Hz, 11.1 Hz), 4.51 (1H, dd, J=8.1 Hz, 11.1 Hz), 3.85 (1H, m), 3.60 (2H, t, J=7.1 Hz), 3.44 (2H, m), 3.13 (2H, t, J=7.1 Hz). LC/MS: C₂₇H₂₄N₆O₂S (M+1) 497. Single peak at both 215 nm and 254 nm in analytical HPLC traces.

### Example 266

### 3-(5-(2-aminopyrimidin-4-yl)-1H-benzo[d]imidazol-2-yl)-N-(2-(thiophen-2-yl)propyl)chroman-6-carboxamide

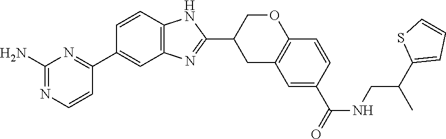

Procedures in Example 242 were utilized to synthesize this compound. LC/MS: C₂₈H₂₆N₆O₂S (M+1) 511. Single peak at both 215 nm and 254 nm in analytical HPLC traces.

### Example 267

### 3-(5-(2-aminopyrimidin-4-yl)-1H-benzo[d]imidazol-2-yl)-N-(2-phenylpropyl)chroman-6-carboxamide

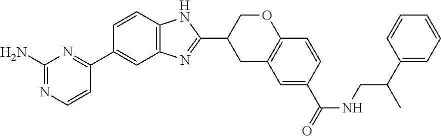

Procedures in Example 242 were utilized to synthesize this compound. LC/MS: C₃₀H₂₈N₆O₂ (M+1) 505. Single peak at both 215 nm and 254 nm in analytical HPLC traces.

### Example 268

### 3-(5-(2-aminopyrimidin-4-yl)-1H-benzo[d]imidazol-2-yl)-N-(2-methoxyphenethyl)chroman-6-carboxamide

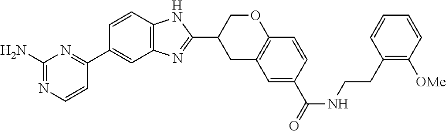

Procedures in Example 242 were utilized to synthesize this compound. LC/MS: C₃₀H₂₈N₆O₃ (M+1) 521. Single peak at both 215 nm and 254 nm in analytical HPLC traces.

### Example 269

### 3-(5-(2-aminopyrimidin-4-yl)-1H-benzo[d]imidazol-2-yl)-N-(3-methoxyphenethyl)chroman-6-carboxamide

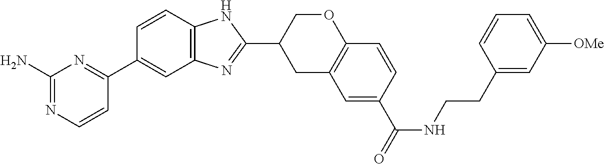

Procedures in Example 242 were utilized to synthesize this compound. LC/MS: C₃₀H₂₈N₆O₃ (M+1) 521. Single peak at both 215 nm and 254 nm in analytical HPLC traces.

### Example 270

### 3-(5-(2-aminopyrimidin-4-yl)-1H-benzo[d]imidazol-2-yl)-N-(4-methoxyphenethyl)chroman-6-carboxamide

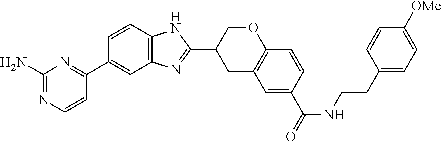

Procedures in Example 242 were utilized to synthesize this compound. ¹H-NMR (MeOD-d₄, 400 MHz) δ 8.55 (1H, d, J=1.1 Hz), 8.28 (1H, d, J=6.6 Hz), 8.24 (1H, dd, J=1.7 Hz, 8.7 Hz), 7.76 (1H, d, J=8.8 Hz), 7.67 (1H, d, J=2.1 Hz), 7.57 (1H, dd, J=2.3 Hz, 8.6 Hz), 7.54 (1H, d, J=6.6 Hz), 7.15 (2H, m), 6.89 (1H, m), 6.83 (2H, m), 4.67 (1H, dd, J=3.1 Hz, 11.0 Hz), 4.50 (1H, dd, J=8.2 Hz, 11.0 Hz), 3.83 (1H, m), 3.75 (3H, s), 3.53 (2H, t, J=7.3 Hz), 3.40 (2H, m), 2.83 (2H, t, J=7.1 Hz). LC/MS: C₃₀H₂₈N₆O₃ (M+1) 521. Single peak at both 215 nm and 254 nm in analytical HPLC traces.

### Example 271

### 3-(5-(2-aminopyrimidin-4-yl)-1H-benzo[d]imidazol-2-yl)-N-(2-(pyridin-3-yl)ethyl)chroman-6-carboxamide

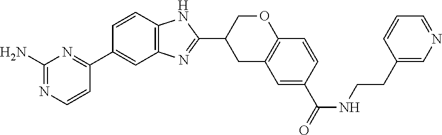

Procedures in Example 242 were utilized to synthesize this compound. LC/MS: C₂₈H₂₅N₇O₂ (M+1) 492. Single peak at both 215 nm and 254 nm in analytical HPLC traces.

### Example 272

### 3-(5-(2-aminopyrimidin-4-yl)-1H-benzo[d]imidazol-2-yl)-N-(2-(pyridin-2-yl)ethyl)chroman-6-carboxamide

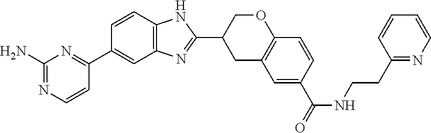

Procedures in Example 242 were utilized to synthesize this compound. LC/MS: C₂₈H₂₅N₇O₂ (M+1) 492. Single peak at both 215 nm and 254 nm in analytical HPLC traces.

### Example 273

### 3-(5-(2-aminopyrimidin-4-yl)-1H-benzo[d]imidazol-2-yl)-N-(pyridin-3-ylmethyl)chroman-6-carboxamide

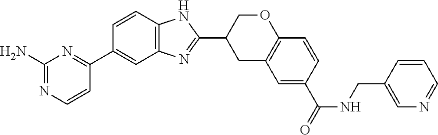

Procedures in Example 242 were utilized to synthesize this compound. LC/MS: C₂₇H₂₃N₇O₂ (M+1) 478. Single peak at both 215 nm and 254 nm in analytical HPLC traces.

### Example 274

### 3-(5-(2-aminopyrimidin-4-yl)-1H-benzo[d]imidazol-2-yl)-N-(3-(dimethylamino)propyl)chroman-6-carboxamide

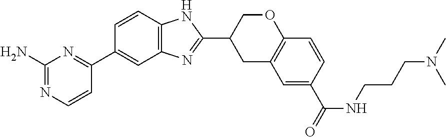

Procedures in Example 242 were utilized to synthesize this compound. LC/MS: C₂₆H₂₉N₇O₂ (M+1) 472. Single peak at both 215 nm and 254 nm in analytical HPLC traces.

### Example 275

### 3-(5-(2-aminopyrimidin-4-yl)-1H-benzo[d]imidazol-2-yl)chroman-6-carboxamide

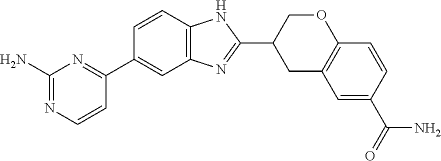

Procedures in Example 242 were utilized to synthesize this compound. ¹H-NMR (MeOD-d₄, 400 MHz) δ 8.55 (1H, s), 8.29 (1H, d, J=6.5 Hz), 8.25 (1H, dd, J=1.4 Hz, 8.7 Hz), 7.76 (2H, m), 7.68 (1H, dd, J=2.0 Hz, 8.5 Hz), 7.55 (1H, d, J=6.6 Hz), 6.91 (1H, d, J=8.6 Hz), 4.68 (1H, dd, J=3.3 Hz, 11.2 Hz), 4.51 (1H, dd, J=8.3 Hz, 11.0 Hz), 3.83 (1H, m), 3.42 (2H, m). LC/MS: C₂₁H₁₈N₆O₂ (M+1) 387. Single peak at both 215 nm and 254 nm in analytical HPLC traces.

### Example 276

### 3-(5-(2-aminopyrimidin-4-yl)-1H-benzo[d]imidazol-2-yl)-N-methoxy-N-methylchroman-6-carboxamide

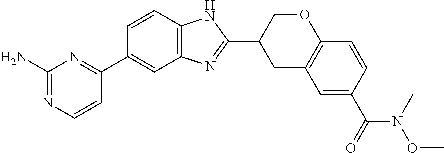

Procedures in Example 242 were utilized to synthesize this compound. LC/MS: C₂₃H₂₂N₆O₃ (M+1) 431. Single peak at both 215 nm and 254 nm in analytical HPLC traces.

### Example 277

### (3-(5-(2-aminopyrimidin-4-yl)-7-fluoro-1H-benzo[d]imidazol-2-yl)chroman-6-yl)(cyclopropyl)methanone

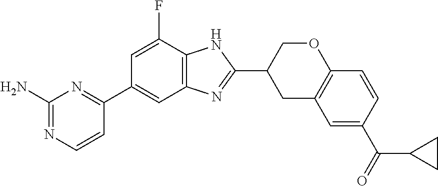

The Weinreb amide Example 276 (1.0 equiv) was dissolved in anhydrous THF under an atmosphere of argon. To this solution was added the Grignard reagent (4.0 equiv) and the resulting mixture was stirred at room temperature until complete by LC-MS. Upon completion, the reaction was poured into aqueous NH₄Cl and thrice extracted with EtOAc. The combined organic fractions were then concentrated and purified via preparative HPLC (MeCN/H₂O+TFA). ¹H-NMR (MeOD-d₄, 400 MHz) δ 8.29 (2H, m), 7.95 (2H, m), 7.87 (1H, dd, J=2.2 Hz, 8.6 Hz), 7.53 (1H, d, J=6.6 Hz), 6.95 (1H, d, J=8.6 Hz), 4.70 (1H, m), 4.48 (1H, dd, J=9.2 Hz, 10.9 Hz), 3.74 (1H, m), 3.41 (2H, m), 2.81 (1H, m), 1.11 (2H, m), 1.06 (2H, m). LC/MS: C₂₄H₂₀FN₅O₂ (M+1) 430. Single peak at both 215 nm and 254 nm in analytical HPLC traces.

### Example 278

### 1-(3-(5-(2-aminopyrimidin-4-yl)-7-fluoro-1H-benzo[d]imidazol-2-yl)chroman-6-yl)-3-phenylpropan-1-one

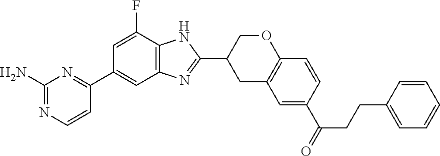

Procedures in Example 277 were utilized to synthesize this compound. ¹H-NMR (MeOD-d₄, 400 MHz) δ 8.32 (1H, d, J=1.4 Hz), 8.28 (1H, d, J=6.7 Hz), 7.97 (1H, dd, J=1.3 Hz, 11.8 Hz), 7.87 (1H, m), 7.80 (1H, dd, J=1.2 Hz, 8.6 Hz), 7.57 (1H, d, J=6.7 Hz), 7.24 (4H, m), 7.15 (1H, m), 6.91 (1H, d, J=8.6 Hz), 4.68 (1H, m), 4.47 (1H, dd, J=9.1 Hz, 10.9 Hz), 3.73 (1H, m), 3.37 (2H, m), 3.29 (2H, t, J=7.8 Hz), 3.00 (2H, t, J=7.6 Hz). LC/MS: C₂₉H₂₄FN₅O₂ (M+1) 494. Single peak at both 215 nm and 254 nm in analytical HPLC traces.

### Example 279

### 1-(3-(5-(2-aminopyrimidin-4-yl)-7-fluoro-1H-benzo[d]imidazol-2-yl)chroman-6-yl)pentan-1-one

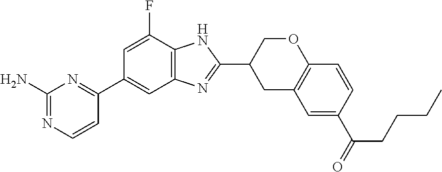

Procedures in Example 277 were utilized to synthesize this compound. LC/MS: C₂₅H₂₄FN₅O₂ (M+1) 446. Single peak at both 215 nm and 254 nm in analytical HPLC traces.

### Example 280

### 1-(3-(5-(2-aminopyrimidin-4-yl)-7-fluoro-1H-benzo[d]imidazol-2-yl)chroman-6-yl)ethanone

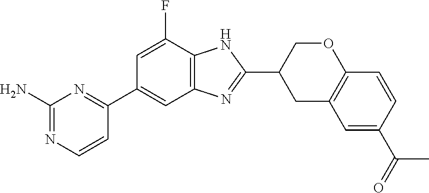

Procedures in Example 277 were utilized to synthesize this compound. ¹H-NMR (MeOD-d₄, 400 MHz) δ 8.32 (1H, d, J=1.4 Hz), 8.28 (1H, d, J=6.6 Hz), 7.96 (1H, dd, J=1.4 Hz, 11.9 Hz), 7.89 (1H, m), 7.81 (1H, dd, J=2.2 Hz, 8.6 Hz), 7.57 (1H, d, J=6.7 Hz), 6.93 (1H, d, J=8.6 Hz), 4.70 (1H, m), 4.49 (1H, dd, J=9.1 Hz, 10.9 Hz), 3.74 (1H, m), 3.40 (2H, m), 2.56 (3H, s). LC/MS: C₂₂H₁₈FN₅O₂ (M+1) 404. Single peak at both 215 nm and 254 nm in analytical HPLC traces.

### Example 281

### 1-(3-(6-(2-aminopyrimidin-4-yl)-3H-imidazo[4,5-b]pyridin-2-yl)chroman-6-yl)ethanone

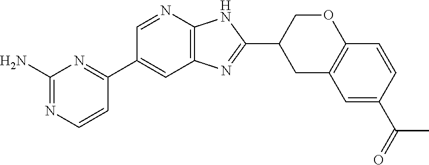

Similar procedures as in Example 277 were utilized to synthesize this compound. LC/MS: C₂₁H₁₈N₆O₂ (M+1) 387. Single peak at both 215 nm and 254 nm in analytical HPLC traces.

### Example 282

### 1-(3-(5-(2-aminopyrimidin-4-yl)-1H-benzo[d]imidazol-2-yl)chroman-6-yl)-2-phenylethanone

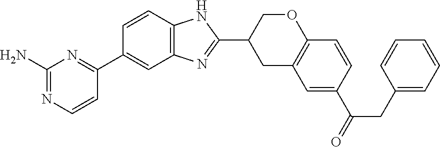

Similar procedures as in Example 277 were utilized to synthesize this compound. ¹H-NMR (MeOD-d₄, 400 MHz) δ 8.29 (1H, bs), 8.25 (1H, d, J=5.4 Hz), 7.97 (1H, d, J=8.6 Hz), 7.94 (1H, s), 7.86 (1H, d, J=8.7 Hz), 7.62 (1H, m), 7.26 (5H, m), 7.17 (1H, d, J=5.4 Hz), 6.91 (1H, d, J=8.5 Hz), 4.67 (1H, m), 4.42 (1H, m), 4.28 (2H, s), 3.66 (1H, m), 3.35 (2H, m). LC/MS: C₂₈H₂₃N₅O₂ (M+1) 462. Single peak at both 215 nm and 254 nm in analytical HPLC traces.

### Example 283

### 1-(3-(5-(2-aminopyrimidin-4-yl)-1H-benzo[d]imidazol-2-yl)chroman-6-yl)-3-methylbutan-1-one

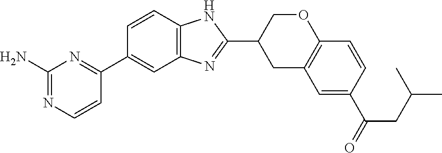

Similar procedures as in Example 277 were utilized to synthesize this compound. LC/MS: C₂₅H₂₅N₅O₂ (M+1) 428. Single peak at both 215 nm and 254 nm in analytical HPLC traces.

### Example 284

### 1-(3-(5-(2-aminopyrimidin-4-yl)-1H-benzo[d]imidazol-2-yl)chroman-6-yl)pentan-1-one

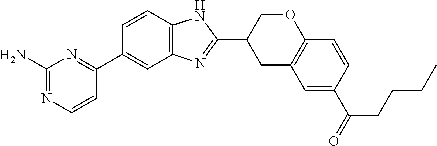

Similar procedures as in Example 277 were utilized to synthesize this compound. LC/MS: C₂₅H₂₅N₅O₂ (M+1) 428. Single peak at both 215 nm and 254 nm in analytical HPLC traces.

### Example 285

### 1-(3-(5-(2-aminopyrimidin-4-yl)-1H-benzo[d]imidazol-2-yl)chroman-6-yl)ethanone

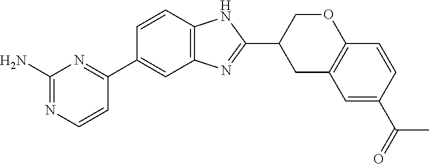

Similar procedures as in Example 277 were utilized to synthesize this compound. ¹H-NMR (MeOD-d₄, 400 MHz) δ 8.64 (1H, s), 8.35 (2H, m), 7.85 (3H, m), 7.62 (1H, d, J=6.7 Hz), 6.96 (1H, d, J=8.6 Hz), 4.72 (1H, ddd, J=1.0 Hz, 3.1 Hz, 11.1 Hz), 4.62 (1H, dd, J=7.4 Hz, 11.2 Hz), 4.00 (1H, m), 3.49 (2H, m), 2.55 (3H, s). LC/MS: C₂₂H₁₉N₅O₂ (M+1) 386. Single peak at both 215 nm and 254 nm in analytical HPLC traces.

### Example 286

### 1-(3-(5-(2-aminopyrimidin-4-yl)-1H-benzo[d]imidazol-2-yl)chroman-6-yl)ethanol

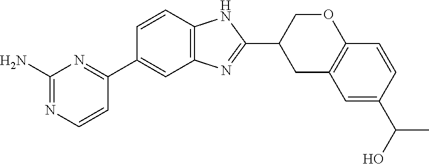

This compound was synthesized via the treatment of the corresponding methyl ketone with NaBH₄ in MeOH. Upon consumption of the ketone, the solution was diluted with THF and washed with aqueous NH₄Cl. The organic fraction was dried over MgSO₄ and purified via preparatory HPLC (MeCN/H₂O+TFA) to give the clean product. LC/MS: C₂₂H₂₁N₅O₂ (M+1) 388. Single peak at both 215 nm and 254 nm in analytical HPLC traces.

### Example 287

### 4-(2-(6-methoxychroman-3-yl)-1H-benzo[d]imidazol-5-yl)pyridin-2-amine

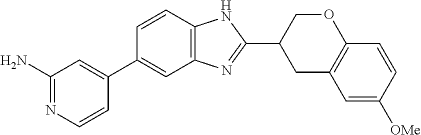

Procedures in Scheme 6 were utilized to synthesize this compound. ¹H-NMR (MeOD-d₄, 400 MHz) δ 8.07 (1H, m), 7.92 (1H, m), 7.83 (1H, d, J=1.2 Hz), 7.28 (2H, m), 6.75 (3H, m), 4.54 (1H, ddd, J=1.0 Hz, 3.1 Hz, 11.0 Hz), 4.45 (1H, dd, J=7.3 Hz, 11.1 Hz), 3.86 (1H, m), 3.74 (3H, s), 3.38 (2H, m). LC/MS: C₂₂H₂₀N₄O₂ (M+1) 373. Single peak at both 215 nm and 254 nm in analytical HPLC traces.

### Example 288

### 4-(2-(6-methylchroman-3-yl)-1H-benzo[d]imidazol-5-yl)pyridin-2-amine

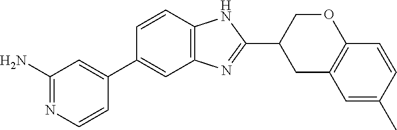

Procedures in Scheme 6 were utilized to synthesize this compound. ¹H-NMR (MeOD-d₄, 400 MHz) δ 8.07 (1H, m), 7.91 (1H, m), 7.83 (1H, d, J=1.2 Hz), 7.28 (2H, m), 6.99 (1H, s), 6.94 (1H, dd, J=1.6 Hz, 8.3 Hz), 6.74 (1H, d, J=8.3 Hz), 4.57 (1H, ddd, J=1.0 Hz, 3.1 Hz, 11.0 Hz), 4.46 (1H, dd, J=7.5 Hz, 11.1 Hz), 3.86 (1H, m), 3.35 (2H, m), 2.25 (3H, s). LC/MS: C₂₂H₂₀N₄O (M+1) 357. Single peak at both 215 nm and 254 nm in analytical HPLC traces.

### Example 289

### methyl 3-(5-(2-aminopyridin-4-yl)-1H-benzo[d]imidazol-2-yl)chroman-6-carboxylate

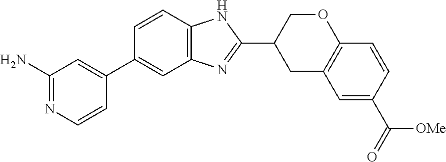

Procedures in Scheme 6 were utilized to synthesize this compound. LC/MS: C₂₃H₂₀N₄O₃ (M+1) 401. Single peak at both 215 nm and 254 nm in analytical HPLC traces.

### Example 290

### 3-(5-(2-aminopyridin-4-yl)-1H-benzo[d]imidazol-2-yl)-N-isobutylchroman-6-carboxamide

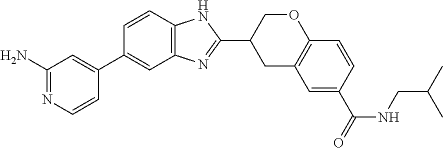

Procedures in Scheme 6 were utilized to synthesize this compound. ¹H-NMR (MeOD-d₄, 400 MHz) δ 8.09 (1H, s), 7.92 (1H, dd, J=2.0 Hz, 6.5 Hz), 7.85 (2H, s), 7.72 (1H, s), 7.63 (1H, d, J=8.4 Hz), 7.28 (2H, m), 6.93 (1H, dd, J=2.6 Hz, 8.5 Hz), 4.68 (1H, m), 4.59 (1H, m), 3.96 (1H, m), 3.47 (2H, m), 3.17 (2H, d, J=6.9 Hz), 1.91 (1H, m), 0.96 (6H, d, J=6.6 Hz). LC/MS: C₂₆H₂₇N₅O₂ (M+1) 442. Single peak at both 215 nm and 254 nm in analytical HPLC traces.

### Example 291

### 3-(5-(2-aminopyridin-4-yl)-1H-benzo[d]imidazol-2-yl)-N-cyclopropylchroman-6-carboxamide

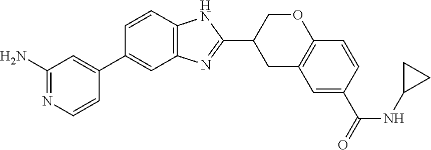

Procedures in Scheme 6 were utilized to synthesize this compound. LC/MS: C₂₅H₂₃N₅O₂ (M+1) 426. Single peak at both 215 nm and 254 nm in analytical HPLC traces.

### Example 292

### 4-(2-(8-methoxychroman-3-yl)-1H-benzo[d]imidazol-5-yl)pyrimidin-2-amine

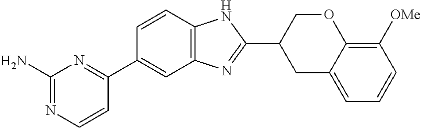

Procedures in Scheme 6 were utilized to synthesize this compound. ¹H-NMR (MeOD-d₄, 400 MHz) δ 8.55 (1H, d, J=1.4 Hz), 8.30 (1H, d, J=6.5 Hz), 8.27 (1H, dd, J=1.6 Hz, 8.7 Hz), 7.78 (1H, d, J=8.6 Hz), 7.54 (1H, d, J=6.5 Hz), 6.84 (3H, m), 4.65 (1H, dd, J=3.0 Hz, 11.0 Hz), 4.46 (1H, dd, J=8.1 Hz, 10.9 Hz), 3.84 (1H, m), 3.82 (3H, s), 3.38 (2H, m). LC/MS: C₂₁H₁₉N₅O₂ (M+1) 374. Single peak at both 215 nm and 254 nm in analytical HPLC traces.

### Example 293

### 4-(2-(5-methoxychroman-3-yl)-1H-benzo[d]imidazol-5-yl)pyrimidin-2-amine

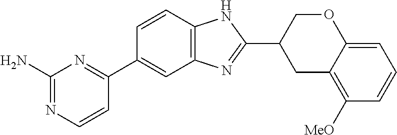

Procedures in Scheme 6 were utilized to synthesize this compound. ¹H-NMR (MeOD-d₄, 400 MHz) δ 8.58 (1H, m), 8.31 (2H, m), 7.81 (1H, d, J=8.7 Hz), 7.56 (1H, d, J=6.5 Hz), 7.11 (1H, t, J=8.2 Hz), 6.57 (1H, d, J=8.2 Hz), 6.51 (1H, d, J=8.3 Hz), 4.57 (1H, m), 4.42 (1H, dd, J=7.8 Hz, 10.9 Hz), 3.85 (4H, m), 3.34 (1H, m), 3.17 (1H, dd, J=8.3 Hz, 17.2 Hz). LC/MS: C₂₁H₁₉N₅O₂ (M+1) 374. Single peak at both 215 nm and 254 nm in analytical HPLC traces.

### Example 294

### 4-(2-(7-methoxychroman-3-yl)-1H-benzo[d]imidazol-5-yl)pyrimidin-2-amine

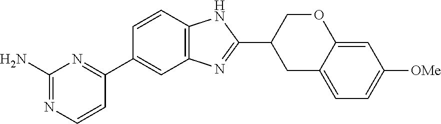

Procedures in Scheme 6 were utilized to synthesize this compound. LC/MS: C₂₁H₁₉N₅O₂ (M+1) 374. Single peak at both 215 nm and 254 nm in analytical HPLC traces.

### Example 295

### 4-(2-(8-fluorochroman-3-yl)-1H-benzo[d]imidazol-5-yl)pyrimidin-2-amine

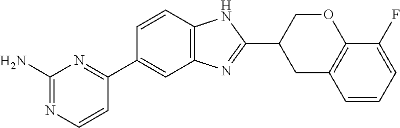

Procedures in Scheme 6 were utilized to synthesize this compound. ¹H-NMR (MeOD-d₄, 400 MHz) δ 8.62 (1H, s), 8.34 (2H, m), 7.85 (1H, d, J=8.7 Hz), 7.61 (1H, d, J=6.7 Hz), 6.98 (2H, m), 6.88 (1H, m), 4.70 (1H, m), 4.57 (1H, dd, J=7.6 Hz, 11.1 Hz), 3.96 (1H, m), 3.45 (2H, m). LC/MS: C₂₀H₁₆FN₅O (M+1) 362. Single peak at both 215 nm and 254 nm in analytical HPLC traces.

### Example 296

### 4-(2-(5-(3-(benzyloxy)propoxy)chroman-3-yl)-1H-benzo[d]imidazol-5-yl)pyrimidin-2-amine

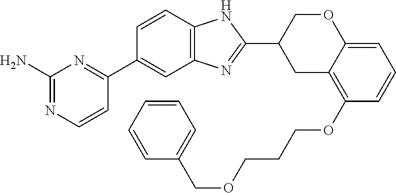

Procedures in Scheme 6 were utilized to synthesize this compound. ¹H-NMR (MeOD-d₄, 400 MHz) δ 8.54 (1H, d, J=1.1 Hz), 8.30 (1H, d, J=6.4 Hz), 8.25 (1H, dd, J=1.7 Hz, 8.7 Hz), 7.77 (1H, d, J=8.8 Hz), 7.51 (1H, d, J=6.4 Hz), 7.26 (2H, m), 7.19 (2H, m), 7.11 (2H, m), 6.55 (1H, d, J=8.2 Hz), 6.50 (1H, d, J=8.3 Hz), 4.55 (1H, m), 4.50 (2H, s), 4.33 (1H, dd, J=8.4 Hz, 10.8 Hz), 4.12 (2H, d, J=6.0 Hz), 3.73 (1H, m), 3.68 (2H, t, J=6.2 Hz), 3.22 (1H, m), 3.05 (1H, dd, J=8.8 Hz, 17.1 Hz), 2.08 (2H, m). LC/MS: C₃₀H₂₉N₅O₃ (M+1) 508. Single peak at both 215 nm and 254 nm in analytical HPLC traces.

### Example 297

### 4-(2-(5-(2-morpholinoethoxy)chroman-3-yl)-1H-benzo[d]imidazol-5-yl)pyrimidin-2-amine

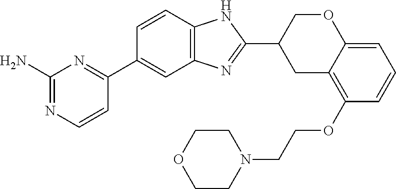

Procedures in Scheme 6 were utilized to synthesize this compound. LC/MS: C₂₆H₂₈N₆O₃ (M+1) 473. Single peak at both 215 nm and 254 nm in analytical HPLC traces.

### Example 298

### 4-(2-(5-(2-(diethylamino)ethoxy)chroman-3-yl)-1H-benzo[d]imidazol-5-yl)pyrimidin-2-amine

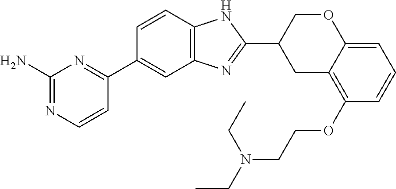

Procedures in Scheme 6 were utilized to synthesize this compound. ¹H-NMR (MeOD-d₄, 400 MHz) δ 8.63 (1H, m), 8.34 (2H, m), 7.84 (1H, d, J=8.7 Hz), 7.61 (1H, d, J=6.7 Hz), 7.15 (1H, t, J=8.3 Hz), 6.64 (1H, d, J=8.3 Hz), 6.60 (1H, d, J=8.3 Hz), 4.55 (1H, dd, J=3.2 Hz, 11.0 Hz), 4.49 (1H, dd, J=6.8 Hz, 11.0 Hz), 4.40 (2H, t, J=4.8 Hz), 3.91 (1H, m), 3.67 (2H, m), 3.37 (6H, m), 1.38 (6H, t, J=7.3 Hz). LC/MS: C₂₆H₃₀N₆O₂ (M+1) 459. Single peak at both 215 nm and 254 nm in analytical HPLC traces.

### Example 299

### 4-(2-(6-bromochroman-3-yl)-1H-benzo[d]imidazol-5-yl)pyrimidin-2-amine

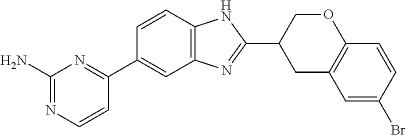

Procedures in Scheme 6 were utilized to synthesize this compound. ¹H-NMR (MeOD-d₄, 400 MHz) δ 8.57 (1H, m), 8.30 (2H, m), 7.79 (1H, d, J=8.5 Hz), 7.58 (1H, d, J=6.7 Hz), 7.36 (1H, d, J=2.3 Hz), 7.25 (1H, dd, J=2.4 Hz, 8.7 Hz), 6.79 (1H, d, J=8.7 Hz), 4.61 (1H, dd, J=2.6 Hz, 11.1 Hz), 4.47 (1H, dd, J=1.7 Hz, 11.1 Hz), 3.84 (1H, m), 3.37 (2H, m). LC/MS: C₂₀H₁₆BrN₅O (M+1) 422. Single peak at both 215 nm and 254 nm in analytical HPLC traces.

### Example 300

### 4-(2-(6-vinylchroman-3-yl)-1H-benzo[d]imidazol-5-yl)pyrimidin-2-amine

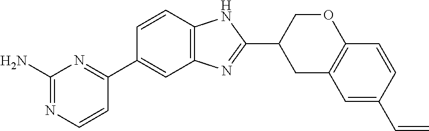

This compound was synthesized via a Suzuki coupling. The corresponding arylbromide (1.0 equiv) (Example 299) was combined with vinyl boronic acid trimer (3.0 equiv), Na₂CO₃ (3.0 equiv) and PdCl₂(PPh₃)₂ (0.10 equiv) in aqueous dioxane (4:1 dioxane:water). The solution was then sparged with argon for 10 min. The solution was then heated at 120° C. in a microwave reactor for 30 minutes, after which time the solution was poured into brine and thrice extracted with THF. The combined organic portions were dried over MgSO₄, concentrated and purified via preparatory HPLC to give the vinylchroman product. LC/MS: C₂₀H₁₉N₅O (M+1) 370. Single peak at both 215 nm and 254 nm in analytical HPLC traces.

### Example 301

### 4-(2-(6-(3-(dimethylamino)prop-1-ynyl)chroman-3-yl)-1H-benzo[d]imidazol-5-yl)pyrimidin-2-amine

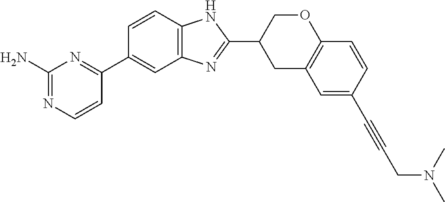

This compound was synthesized via a Sonogashira coupling. The corresponding arylbromide (1.0 equiv) (Example 299) was combined with the N,N-dimethylaminopropyne (3.0 equiv), Et₃N (5.0 equiv), CuI (0.20 equiv) and PdCl₂(PPh₃)₂ (0.20 equiv) in anhydrous dioxane. The solution was then sparged with argon for 10 min. The solution was then heated at 100° C. in a microwave reactor for 90 minutes, after which time the solution was concentrated and purified via preparatory HPLC to give the alkylnylchroman product. LC/MS: C₂₅H₂₄N₆O (M+1) 425. Single peak at both 215 nm and 254 nm in analytical HPLC traces.

### Example 302

### 4-(2-(6-(1-(isobutylamino)ethyl)chroman-3-yl)-1H-benzo[d]imidazol-5-yl)pyrimidin-2-amine

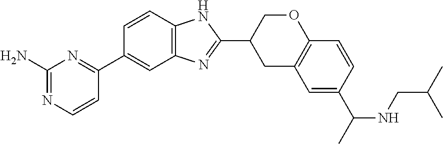

Procedures in Scheme 6 were utilized to synthesize this compound. ¹H-NMR (MeOD-d₄, 400 MHz) δ 8.68 (1H, s), 8.40 (1H, d, J=8.4 Hz), 8.35 (1H, m), 7.90 (1H, d, J=8.8 Hz), 7.63 (1H, m), 7.34 (1H, s), 7.28 (1H, m), 6.98 (1H, d, J=8.4 Hz), 4.67 (1H, m), 4.59 (1H, m), 4.31 (1H, q, J=6.6 Hz), 4.03 (1H, m), 3.50 (2H, m), 2.77 (1H, m), 2.56 (1H, m), 1.94 (1H, m), 1.66 (3H, d, J=6.5 Hz), 0.97 (6H, m). LC/MS: C₂₆H₃₀N₆O (M+1) 443. Single peak at both 215 nm and 254 nm in analytical HPLC traces.

### Example 303

### 4-(2-(6-(1-(cyclopropylamino)ethyl)chroman-3-yl)-1H-benzo[d]imidazol-5-yl)pyrimidin-2-amine

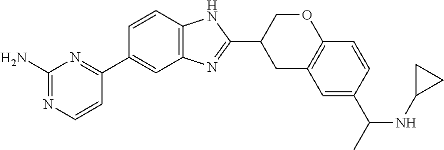

Procedures in Scheme 6 were utilized to synthesize this compound. ¹H-NMR (MeOD-d₄, 400 MHz) δ 8.63 (1H, s), 8.33 (2H, m), 7.83 (1H, d, J=8.7 Hz), 7.61 (1H, d, J=6.7 Hz), 7.33 (1H, m), 7.28 (1H, dd, J=2.3 Hz, 8.5 Hz), 6.97 (1H, d, J=8.5 Hz), 4.66 (1H, dd, J=3.0 Hz, 11.1 Hz), 4.54 (1H, dd, J=7.8 Hz, 11.1 Hz), 4.43 (1H, q, J=6.9 Hz), 3.93 (1H, m), 3.46 (2H, m), 2.56 (1H, m), 1.68 (3H, d, J=6.9 Hz), 0.80 (4H, m). LC/MS: C₂₅H₂₆N₆O (M+1) 427. Single peak at both 215 nm and 254 nm in analytical HPLC traces.

### Example 304

### 4-(2-(6-(1-(2-(thiophen-2-yl)ethylamino)ethyl)chroman-3-yl)-1H-benzo[d]imidazol-5-yl)pyrimidin-2-amine

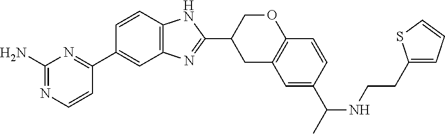

Procedures in Scheme 6 were utilized to synthesize this compound. LC/MS: C₂₈H₂₈N₆OS (M+1) 497. Single peak at both 215 nm and 254 nm in analytical HPLC traces.

### Example 305

### 4-(2-(6-(1-(2-methoxyethylamino)ethyl)chroman-3-yl)-1H-benzo[d]imidazol-5-yl)pyrimidin-2-amine

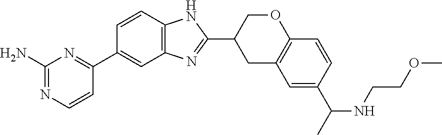

Procedures in Scheme 6 were utilized to synthesize this compound. ¹H-NMR (MeOD-d₄, 400 MHz) δ 8.65 (1H, s), 8.34 (2H, m), 7.86 (1H, d, J=8.7 Hz), 7.61 (1H, d, J=6.7 Hz), 7.32 (1H, m), 7.27 (1H, m), 6.97 (1H, d, J=8.5 Hz), 4.66 (1H, dd, J=2.9 Hz, 11.1 Hz), 4.55 (1H, ddd, J=1.3 Hz, 7.6 Hz, 11.1 Hz), 4.34 (1H, q, J=6.8 Hz), 3.96 (1H, m), 3.56 (2H, m), 3.46 (2H, m), 3.36 (3H, s), 3.09 (1H, ddd, J=3.5 Hz, 6.5 Hz, 13.2 Hz), 2.95 (1H, m), 1.66 (3H, d, J=6.8 Hz). LC/MS: C₂₅H₂₈N₆O₂ (M+1) 445. Single peak at both 215 nm and 254 nm in analytical HPLC traces.

### Example 306

### 2-(3-(5-(2-aminopyrimidin-4-yl)-1H-benzo[d]imidazol-2-yl)chroman-6-yl)-N-cyclopropylacetamide

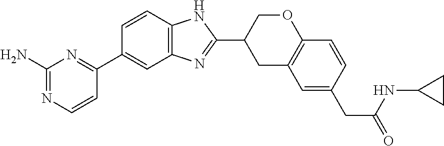

Procedures in Scheme 6 were utilized to synthesize this compound. ¹H-NMR (MeOD-d₄, 400 MHz) δ 8.56 (1H, d, J=1.1 Hz), 8.33 (2H, m), 7.83 (1H, d, J=8.6 Hz), 7.58 (1H, d, J=6.6 Hz), 7.11 (1H, s), 7.03 (1H, d, J=8.3 Hz), 6.80 (1H, d, J=8.4 Hz), 4.59 (1H, dd, J=2.4 Hz, 11.0 Hz), 4.52 (1H, dd, J=6.8 Hz, 11.2 Hz), 3.91 (1H, m), 3.42 (4H, m), 2.65 (1H, m), 0.71 (2H, m), 0.47 (2H, m). LC/MS: C₂₅H₂₄N₆O₂ (M+1) 441. Single peak at both 215 nm and 254 nm in analytical HPLC traces.

### Example 307

### 2-(3-(5-(2-aminopyrimidin-4-yl)-1H-benzo[d]imidazol-2-yl)chroman-6-yl)-N-(2-methoxyethyl)acetamide

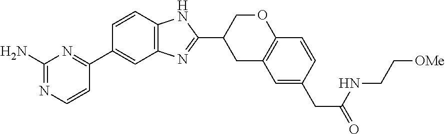

Procedures in Scheme 6 were utilized to synthesize this compound. ¹H-NMR (MeOD-d₄, 400 MHz) δ 8.59 (1H, d, J=1.1 Hz), 8.33 (2H, m), 7.83 (1H, d, J=8.7 Hz), 7.58 (1H, d, J=6.6 Hz), 7.12 (1H, s), 7.05 (1H, dd, J=2.0 Hz, 8.4 Hz), 6.80 (1H, d, J=8.4 Hz), 4.59 (1H, dd, J=2.5 Hz, 11.0 Hz), 4.52 (1H, dd, J=6.9 Hz, 11.2 Hz), 3.91 (1H, m), 3.44 (5H, m), 3.36 (3H, m), 2.81 (3H, s). LC/MS: C₂₅H₂₆N₆O₃ (M+1) 459. Single peak at both 215 nm and 254 nm in analytical HPLC traces.

### Example 308

### 2-(3-(5-(2-aminopyrimidin-4-yl)-1H-benzo[d]imidazol-2-yl)chroman-6-yl)-N-isobutylacetamide

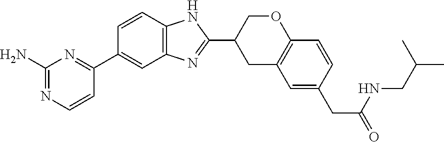

Procedures in Scheme 6 were utilized to synthesize this compound. ¹H-NMR (MeOD-d₄, 400 MHz) δ 8.60 (1H, d, J=1.3 Hz), 8.34 (2H, m), 7.84 (1H, d, J=8.7 Hz), 7.59 (1H, d, J=6.7 Hz), 7.13 (1H, s), 7.06 (1H, dd, J=2.0 Hz, 8.4 Hz), 6.81 (1H, d, J=8.4 Hz), 4.60 (1H, dd, J=2.8 Hz, 11.1 Hz), 4.53 (1H, dd, J=6.8 Hz, 11.1 Hz), 3.94 (1H, m), 3.44 (4H, m), 2.99 (2H, d, J=6.9 Hz), 1.75 (1H, m), 0.87 (6H, d, J=6.7 Hz). LC/MS: C₂₆H₂₈N₆O₂ (M+1) 457. Single peak at both 215 nm and 254 nm in analytical HPLC traces.

### Example 309

### 2-(3-(5-(2-aminopyrimidin-4-yl)-1H-benzo[d]imidazol-2-yl)chroman-6-yl)-N-(pyridin-3-ylmethyl)acetamide

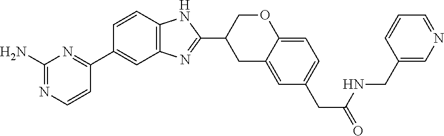

Procedures in Scheme 6 were utilized to synthesize this compound. ¹H-NMR (MeOD-d₄, 400 MHz) δ 8.69 (2H, m), 8.57 (1H, s), 8.33 (3H, m), 7.92 (1H, dd, J=5.9 Hz, 8.0 Hz), 7.80 (1H, d, J=8.7 Hz), 7.57 (1H, d, J=6.6 Hz), 7.13 (1H, s), 7.07 (1H, d, J=8.2 Hz), 6.82 (1H, d, J=8.4 Hz), 4.60 (1H, dd, J=2.9 Hz, 11.1 Hz), 4.53 (2H, s), 4.48 (1H, dd, J=7.7 Hz, 11.0 Hz), 3.86 (1H, m), 3.52 (2H, s), 3.38 (2H, m). LC/MS: C₂₈H₂₅N₇O₂ (M+1) 492. Single peak at both 215 nm and 254 nm in analytical HPLC traces.

### Example 310

### 4 2-(3-(5-(2-aminopyrimidin-4-yl)-1H-benzo[d]imidazol-2-yl)chroman-6-yl)-N-(3-(dimethylamino)propyl)acetamide

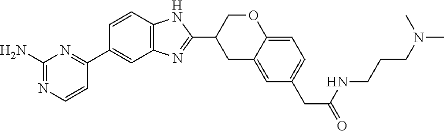

Procedures in Scheme 6 were utilized to synthesize this compound. LC/MS: C₂₇H₃₁N₇O₂ (M+1) 486. Single peak at both 215 nm and 254 nm in analytical HPLC traces.

### Example 311

### 4-(2-(m-tolyloxymethyl)-1H-benzo[d]imidazol-5-yl)pyrimidin-2-amine

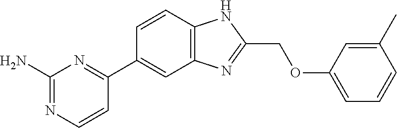

Procedures in Scheme 10 were utilized to synthesize this compound. ¹H-NMR (MeOD-d₄, 400 MHz) δ 8.61 (1H, s), 8.31 (2H, m), 7.82 (1H, d, J=8.7 Hz), 7.59 (1H, d, J=6.7 Hz), 7.21 (1H, m), 6.95 (1H, s), 6.88 (1H, m), 5.48 (2H, s), 2.34 (3H, s). LC/MS: C₁₉H₁₇N₅O (M+1) 332. Single peak at both 215 nm and 254 nm in analytical HPLC traces.

### Example 312

### 2-(4-(2-(5-(2-aminopyrimidin-4-yl)-1H-benzo[d]imidazol-2-yl)ethoxy)phenyl)-N-cyclopropylacetamide

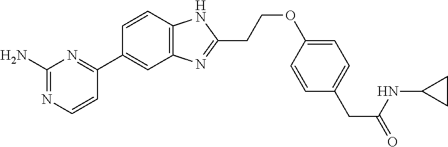

Procedures in Scheme 10 were utilized to synthesize this compound. LC/MS: C₂₄H₂₄N₆O₂ (M+1) 429. Single peak at both 215 nm and 254 nm in analytical HPLC traces.

### Example 313

### 2-(4-(2-(5-(2-aminopyrimidin-4-yl)-1H-benzo[d]imidazol-2-yl)ethoxy)phenyl)-N-(2-methoxyethyl)acetamide

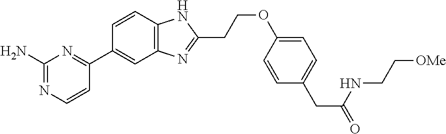

Procedures in Scheme 10 were utilized to synthesize this compound. ¹H-NMR (MeOD-d₄, 400 MHz) δ 8.60 (1H, s), 8.36 (2H, m), 7.87 (1H, d, J=8.7 Hz), 7.55 (1H, d, J=6.4 Hz), 7.20 (2H, d, J=8.5 Hz), 6.91 (2H, d, J=8.6 Hz), 4.51 (2H, t, J=5.9 Hz), 3.64 (2H, t, J=5.9 Hz), 3.42 (4H, m), 3.33 (2H, m), 2.81 (2H, s). LC/MS: C₂₄H₂₆N₆O₃ (M+1) 447. Single peak at both 215 nm and 254 nm in analytical HPLC traces.

### Example 314

### 2-(4-(2-(5-(2-aminopyrimidin-4-yl)-1H-benzo[d]imidazol-2-yl)ethoxy)phenyl)-N-(2-(pyridin-3-yl)ethyl)acetamide

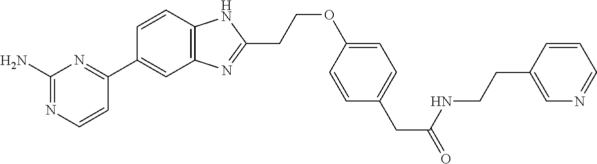

Procedures in Scheme 10 were utilized to synthesize this compound. ¹H-NMR (MeOD-d₄, 400 MHz) δ 8.65 (3H, m), 8.34 (3H, m), 7.85 (2H, m), 7.56 (1H, d, J=6.5 Hz), 7.11 (2H, d, J=8.4 Hz), 6.90 (2H, d, J=8.3 Hz), 4.52 (2H, t, J=5.9 Hz), 3.66 (2H, t, J=5.9 Hz), 3.51 (2H, t, J=6.6 Hz), 3.34 (2H, s), 2.99 (2H, t, J=6.7 Hz). LC/MS: C₂₈H₂₇N₇O₂ (M+1) 494. Single peak at both 215 nm and 254 nm in analytical HPLC traces.

### Example 315

### 2-(4-(2-(5-(2-aminopyrimidin-4-yl)-1H-benzo[d]imidazol-2-yl)ethoxy)phenyl)-N-(2-(dimethylamino)ethyl)acetamide

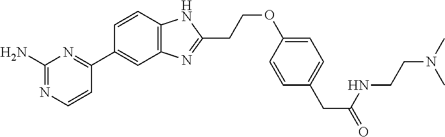

Procedures in Scheme 10 were utilized to synthesize this compound. LC/MS: C₂₅H₂₉N₇O₂ (M+1) 460. Single peak at both 215 nm and 254 nm in analytical HPLC traces.

### Example 316

### 4-(2-((3-methoxyphenoxy)methyl)-1H-benzo[d]imidazol-5-yl)pyrimidin-2-amine

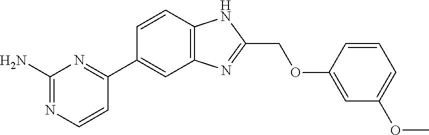

Procedures in Scheme 10 were utilized to synthesize this compound. ¹H-NMR (MeOD-d₄, 400 MHz) δ 8.61 (1H, s), 8.30 (2H, m), 7.81 (1H, d, J=8.7 Hz), 7.60 (1H, d, J=6.7 Hz), 7.23 (1H, m), 6.68 (2H, m), 6.62 (1H, m), 5.48 (2H, s), 3.79 (3H, s). LC/MS: C₁₉H₁₇N₅O₂ (M+1) 348. Single peak at both 215 nm and 254 nm in analytical HPLC traces.

### Example 317

### 4-(2-((2-methoxyphenoxy)methyl)-1H-benzo[d]imidazol-5-yl)pyrimidin-2-amine

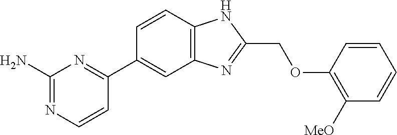

Procedures in Scheme 10 were utilized to synthesize this compound. LC/MS: C₁₉H₁₇N₅O₂ (M+1) 348. Single peak at both 215 nm and 254 nm in analytical HPLC traces.

### Example 318

### 4-(2-(o-tolyloxymethyl)-1H-benzo[d]imidazol-5-yl)pyrimidin-2-amine

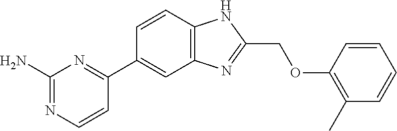

Procedures in Scheme 10 were utilized to synthesize this compound. LC/MS: C₁₉H₁₇N₅O (M+1) 332. Single peak at both 215 nm and 254 nm in analytical HPLC traces.

### Example 319

### 4-(2-((3-methoxyphenylamino)methyl)-1H-benzo[d]imidazol-5-yl)pyrimidin-2-amine

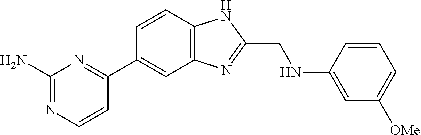

Procedures in Scheme 10 were utilized to synthesize this compound. ¹H-NMR (MeOD-d₄, 400 MHz) δ 8.59 (1H, s), 8.36 (2H, m), 7.85 (1H, d, J=8.4 Hz), 7.55 (1H, d, J=6.4 Hz), 7.06 (1H, m), 6.27 (3H, m), 4.88 (2H, s), 3.72 (3H, s). LC/MS: C₁₉H₁₈N₆O (M+1) 347. Single peak at both 215 nm and 254 nm in analytical HPLC traces.

### Example 320

### 2-(((5-(2-aminopyrimidin-4-yl)-1H-benzo[d]imidazol-2-yl)methyl)(3-methoxyphenyl)amino)acetic acid

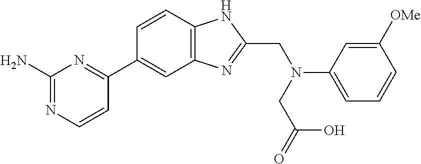

Procedures in Scheme 9 were utilized to synthesize this compound. LC/MS: C₂₁H₂₀N₆O₃ (M+1) 405. Single peak at both 215 nm and 254 nm in analytical HPLC traces.

### Example 321

### 2-(((5-(2-aminopyrimidin-4-yl)-1H-benzo[d]imidazol-2-yl)methyl)(3-methoxyphenyl)amino)acetamide

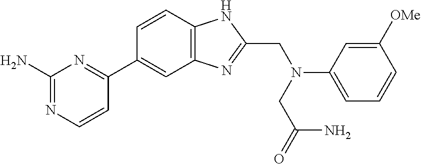

Procedures in Scheme 9 were utilized to synthesize this compound. LC/MS: C₂₁H₂₁N₇O₂ (M+1) 404. Single peak at both 215 nm and 254 nm in analytical HPLC traces.

### Example 322

### 2-(((5-(2-aminopyrimidin-4-yl)-1H-benzo[d]imidazol-2-yl)methyl)(3-methoxyphenyl)amino)-N,N-dimethylacetamide

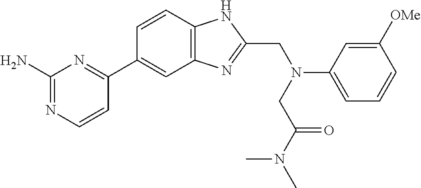

Procedures in Scheme 9 were utilized to synthesize this compound. ¹H-NMR (MeOD-d₄, 400 MHz) δ 8.60 (1H, s), 8.31 (2H, m), 7.85 (1H, d, J=8.7 Hz), 7.54 (1H, d, J=6.5 Hz), 7.08 (1H, t, J=8.3 Hz), 6.35 (1H, dd, J=1.7 Hz, 8.2 Hz), 6.19 (1H, dd, J=2.4 Hz, 8.3 Hz), 6.09 (1H, s), 5.17 (2H, s), 4.65 (2H, s), 3.67 (3H, s), 3.23 (3H, s), 3.12 (3H, s). LC/MS: C₂₃H₂₅N₇O₂ (M+1) 432. Single peak at both 215 nm and 254 nm in analytical HPLC traces.

### Example 323

### 2-(((5-(2-aminopyrimidin-4-yl)-1H-benzo[d]imidazol-2-yl)methyl)(3-methoxyphenyl)amino)-1-morpholinoethanone

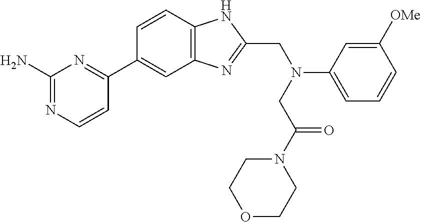

Procedures in Scheme 9 were utilized to synthesize this compound. LC/MS: C₂₅H₂₇N₇O₃ (M+1) 474. Single peak at both 215 nm and 254 nm in analytical HPLC traces.

### Example 324

### 2-(((5-(2-aminopyrimidin-4-yl)-1H-benzo[d]imidazol-2-yl)methyl)(3-methoxyphenyl)amino)-1-morpholinoethanone

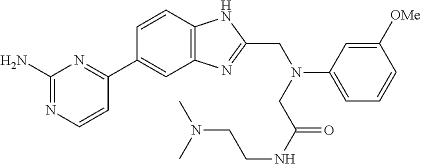

Procedures in Scheme 9 were utilized to synthesize this compound. ¹H-NMR (MeOD-d₄, 400 MHz) δ 8.54 (1H, s), 8.28 (2H, m), 7.77 (1H, d, J=8.6 Hz), 7.54 (1H, d, J=6.5 Hz), 7.10 (1H, t, J=8.3 Hz), 6.38 (1H, dd, J=1.9 Hz, 8.2 Hz), 6.26 (1H, dd, J=2.5 Hz, 8.4 Hz), 6.18 (1H, s), 5.11 (2H, s), 4.39 (2H, s), 3.69 (5H, m), 3.35 (2H, t, J=6.0 Hz), 2.96 (6H, s). LC/MS: C₂₅H₃₀N₈O₂ (M+1) 475. Single peak at both 215 nm and 254 nm in analytical HPLC traces.

### Example 325

### 2-(((5-(2-aminopyrimidin-4-yl)-1H-benzo[d]imidazol-2-yl)methyl)(3-methoxyphenyl)amino)-N-cyclopropylacetamide

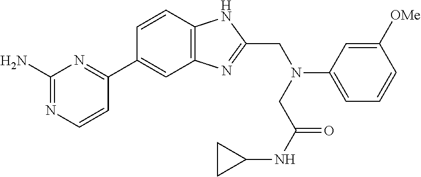

Procedures in Scheme 9 were utilized to synthesize this compound. LC/MS: C₂₄H₂₅N₇O₂ (M+1) 444. Single peak at both 215 nm and 254 nm in analytical HPLC traces.

### Example 326

### 2-(((5-(2-aminopyrimidin-4-yl)-1H-benzo[d]imidazol-2-yl)methyl)(3-methoxyphenyl)amino)-N-(2-(dimethylamino)ethyl)-N-methylacetamide

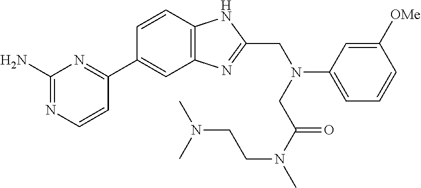

Procedures in Scheme 9 were utilized to synthesize this compound. ¹H-NMR (MeOD-d₄, 400 MHz) δ 8.58 (1H, s), 8.31 (2H, m), 7.82 (1H, d, J=8.7 Hz), 7.55 (1H, d, J=6.5 Hz), 7.10 (1H, t, J=8.3 Hz), 6.37 (1H, dd, J=2.0 Hz, 8.2 Hz), 6.27 (1H, dd, J=2.4 Hz, 8.3 Hz), 6.16 (1H, m), 5.14 (2H, s), 4.64 (2H, s), 3.90 (2H, t, J=6.1 Hz), 3.68 (3H, s), 3.42 (2H, t, J=6.1 Hz), 3.23 (3H, s), 2.98 (6H, s). LC/MS: C₂₆H₃₂N₈O₂ (M+1) 489. Single peak at both 215 nm and 254 nm in analytical HPLC traces.

### Example 327

### 2-(((5-(2-aminopyrimidin-4-yl)-1H-benzo[d]imidazol-2-yl)methyl)(3-methoxyphenyl)amino)-N-(3-(dimethylamino)propyl)acetamide

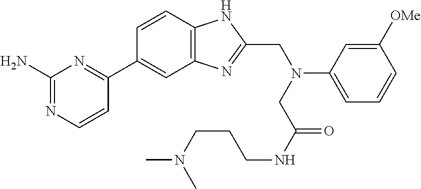

Procedures in Scheme 9 were utilized to synthesize this compound. LC/MS: C₂₆H₃₂N₈O₂ (M+1) 489. Single peak at both 215 nm and 254 nm in analytical HPLC traces.

### Example 328

### 2-(2-(6-methoxychroman-3-yl)-5-(pyridin-4-yl)-1H-benzo[d]imidazol-7-yloxy)-N,N-dimethylethanamine

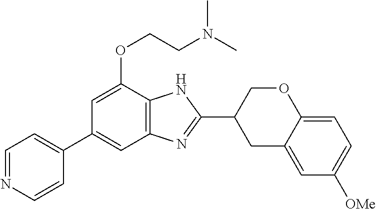

Procedures in Scheme 11 were utilized to synthesize this compound. ¹H-NMR (MeOD-d₄, 400 MHz) δ 8.83 (2H, d, J=5.9 Hz), 8.40 (2H, d, J=5.9 Hz), 7.84 (1H, s), 7.40 (1H, s), 6.75 (3H, m), 4.68 (2H, t, J=5.0 Hz), 4.54 (1H, m), 4.35 (1H, m), 3.74 (6H, m), 3.35 (2H, m), 3.06 (6H, s). LC/MS: C₂₆H₂₈N₄O₃ (M+1) 445. Single peak at both 215 nm and 254 nm in analytical HPLC traces.

### Example 329

### 2-(2-(6-methoxychroman-3-yl)-5-(1H-pyrazol-4-yl)-1H-benzo[d]imidazol-7-yloxy)-N,N-dimethylethanamine

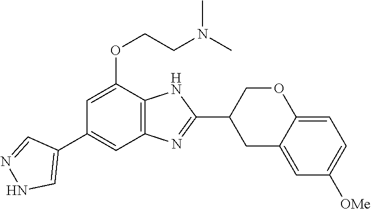

Procedures in Scheme 11 were utilized to synthesize this compound. ¹H-NMR (MeOD-d₄, 400 MHz) δ 8.05 (2H, s), 7.44 (1H, s), 7.22 (1H, s), 6.76 (3H, m), 4.64 (2H, t, J=4.9 Hz), 4.52 (1H, dd, J=2.8 Hz, 10.8 Hz), 4.39 (1H, dd, J=8.2 Hz, 10.7 Hz), 3.82 (1H, m), 3.74 (3H, s), 3.72 (2H, t, J=5.0 Hz), 3.35 (2H, m), 3.05 (6H, s). LC/MS: C₂₄H₂₇N₅O₃ (M+1) 434. Single peak at both 215 nm and 254 nm in analytical HPLC traces.

### Example 330

### 2-(2-(6-methoxychroman-3-yl)-5-(3-methyl-1H-pyrazol-4-yl)-1H-benzo[d]imidazol-7-yloxy)-N,N-dimethylethanamine

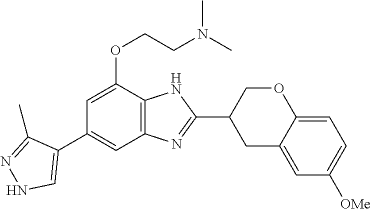

Procedures in Scheme 11 were utilized to synthesize this compound. LC/MS: C₂₅H₂₉N₅O₃ (M+1) 448. Single peak at both 215 nm and 254 nm in analytical HPLC traces.

### Example 331

### 3-(2-(6-methoxychroman-3-yl)-5-(pyridin-4-yl)-1H-benzo[d]imidazol-7-yloxy)propan-1-ol

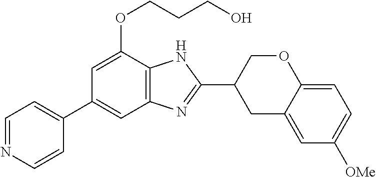

Procedures in Scheme 11 were utilized to synthesize this compound. ¹H-NMR (MeOD-d₄, 400 MHz) δ 8.83 (2H, d, J=6.6 Hz), 8.35 (2H, d, J=6.7 Hz), 7.82 (1H, s), 7.48 (1H, s), 6.76 (3H, m), 4.51 (3H, m), 4.41 (1H, dd, J=8.2 Hz, 10.2 Hz), 3.85 (3H, m), 3.74 (3H, s), 3.36 (2H, m), 2.17 (2H, m, J=6.1 Hz). LC/MS: C₂₅H₂₅N₃O₄ (M+1) 432. Single peak at both 215 nm and 254 nm in analytical HPLC traces.

### Example 332

### 3-(2-(6-methoxychroman-3-yl)-5-(1H-pyrazol-4-yl)-1H-benzo[d]imidazol-7-yloxy)propan-1-ol

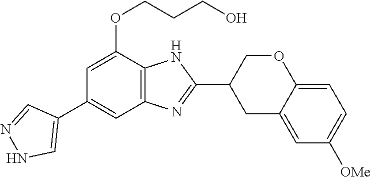

Procedures in Scheme 11 were utilized to synthesize this compound. —NMR (MeOD-d₄, 400 MHz) δ 8.08 (2H, s), 7.44 (1H, s), 7.34 (1H, s), 6.78 (3H, m), 4.49 (4H, m), 3.93 (1H, m), 3.84 (2H, t, J=6.1 Hz), 3.74 (3H, s), 3.40 (2H, m), 2.15 (2H, m). LC/MS: C₂₃H₂₄N₄O₄ (M+1) 421. Single peak at both 215 nm and 254 nm in analytical HPLC traces.

### Example 333

### 3-(2-(6-methoxychroman-3-yl)-5-(3-methyl-1H-pyrazol-4-yl)-1H-benzo[d]imidazol-7-yloxy)propan-1-ol

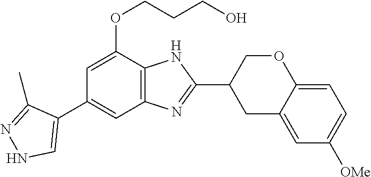

Procedures in Scheme 11 were utilized to synthesize this compound. LC/MS: C₂₄H₂₆N₄O₄ (M+1) 435. Single peak at both 215 nm and 254 nm in analytical HPLC traces.

### Example 334

### 3-(5-(2-aminopyrimidin-4-yl)-2-(6-methoxychroman-3-yl)-1H-benzo[d]imidazol-7-yloxy)propan-1-ol

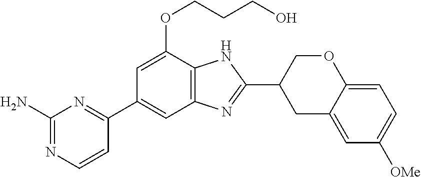

Procedures in Scheme 11 were utilized to synthesize this compound. LC/MS: C₂₄H₂₅N₅O₄ (M+1) 448. Single peak at both 215 nm and 254 nm in analytical HPLC traces.

Examples 335 to 354 are listed below:

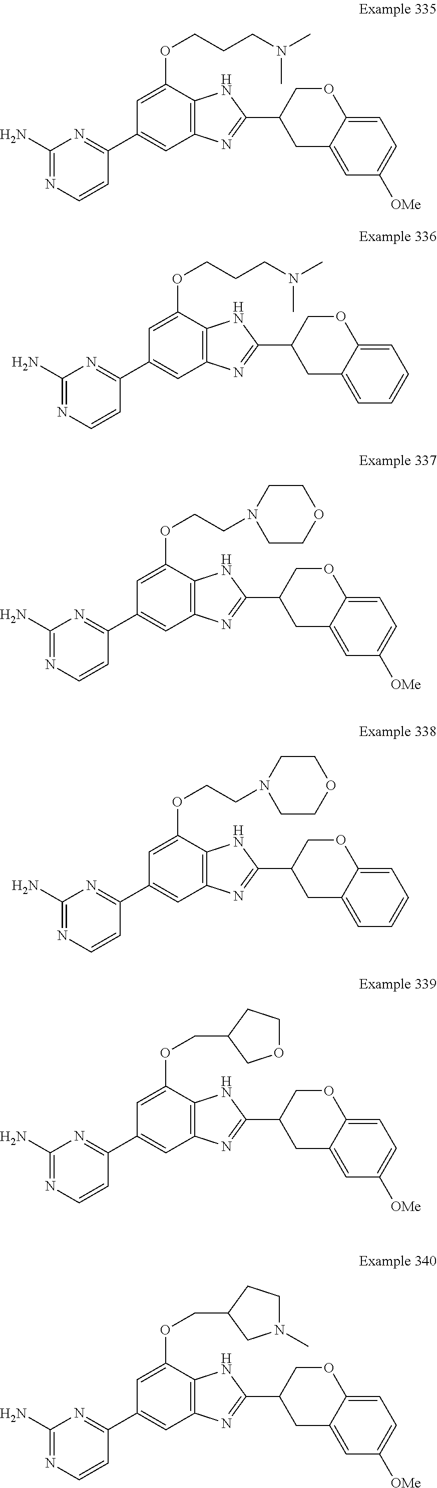

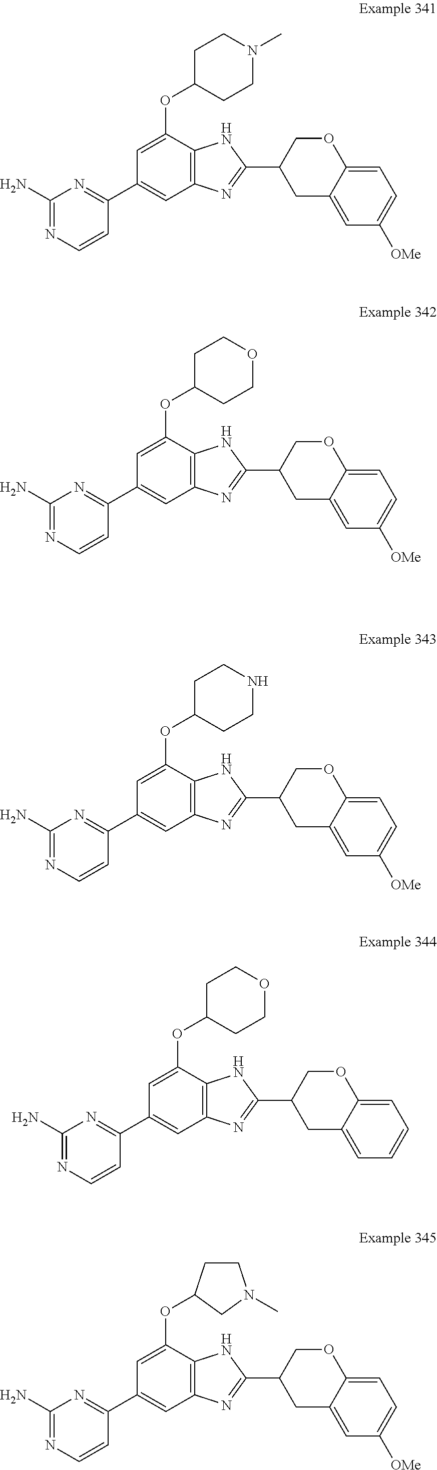

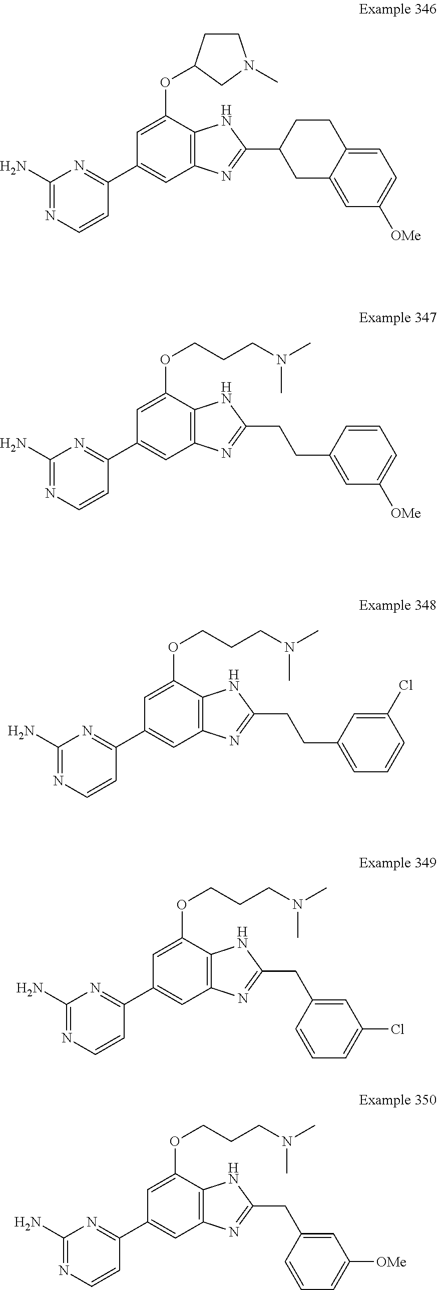

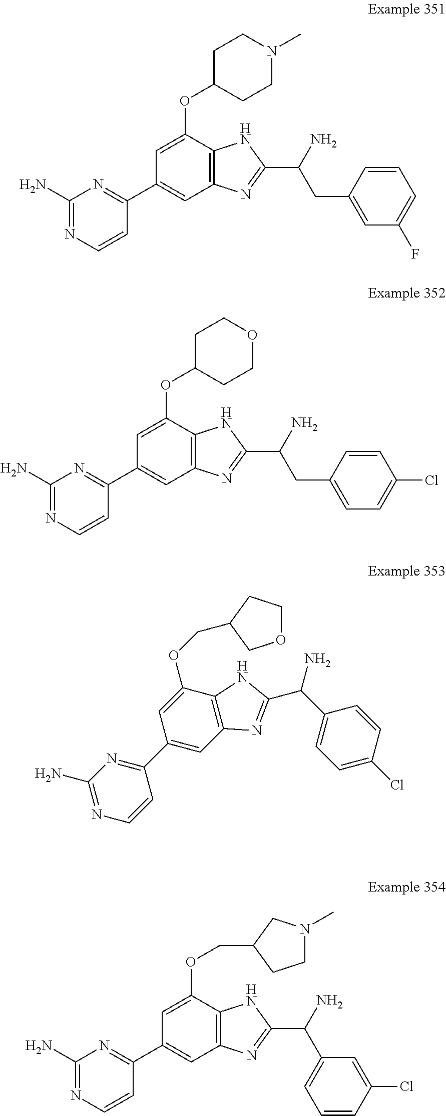

### Example 355

### (±) 2-((3R,4S)-1-benzyl-4-(4-methoxyphenyl)pyrrolidin-3-yl)-5-(1H-pyrazol-4-yl)-1H-benzo[d]imidazole

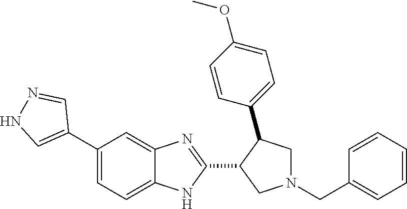

Procedures in Scheme 10 were utilized to synthesize this compound. LC/MS: C₂₈H₂₈N₅O (M+1) 450. Single peak at both 215 nm and 254 nm in analytical HPLC traces.

### Example 356

### (±) 2-((3R,4S)-4-(4-methoxyphenyl)pyrrolidin-3-yl)-5-(1H-pyrazol-4-yl)-1H-benzo[d]imidazole

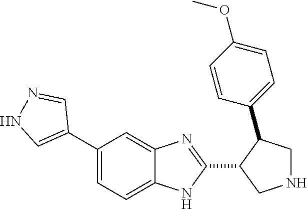

A solution of Example 355, Pd(OH)₂ (cat), and AcOH in MeOH was allowed to react at room temperature for 2 days. Analysis using LC-MS showed that a 1.5:1 mixture of products Example 356 and Example 357 was obtained. The reaction mixture was then concentrated in vacuo and the residue was separated by preparative HPLC to give pure Example 356 and Example 357. Example 356: LC/MS: C₂₁H₂₂N₅O₁ (M+1) 360. Single peak at both 215 nm and 254 nm in analytical HPLC traces.

### Example 357

### (±) 2-(3R,4S)-4-(4-methoxyphenyl)-1-methylpyrrolidin-3-yl)-5-(1H-pyrazol-4-yl)-1H-benzo[d]imidazole

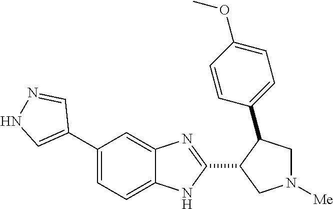

Procedures see Example 356. LC/MS: C₂₂H₂₄N₅O(M+1) 374. Single peak at both 215 nm and 254 nm in analytical HPLC traces.

### Example 358

### (±) 2-((3R,4S)-4-(4-methoxyphenyl)pyrrolidin-3-yl)-5-(1H-pyrazol-4-yl)-1H-benzo[d]imidazole

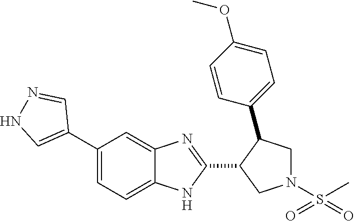

This compound was prepared by reacting Example 356 and methane sulfonyl chloride in DCM, and then subjected to preparative reverse-phase HPLC. LC/MS: C₂₂H₂₄N₅O₃S (M+1) 438. Single peak at both 215 nm and 254 nm in analytical HPLC traces.

### Example 359

### (±)2-((3R,4S)-1-benzyl-4-(4-methoxyphenyl)pyrrolidin-3-yl)-5-(3-methyl-1H-pyrazol-4-yl)-1H-benzo[d]imidazole

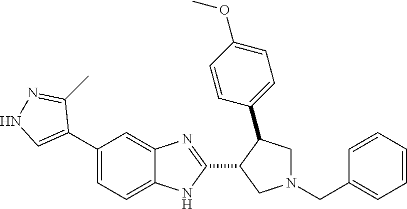

Procedures in Scheme 10 were utilized to synthesize this compound. LC/MS: C₂₉H₃₀N₅O (M+1) 464. Single peak at both 215 nm and 254 nm in analytical HPLC traces. ¹H-HMR (DMSO-d₆, 400 MHz) δ 8.40 (s, 1H), 8.29 (d, J=1.6 Hz, 1H), 8.20 (s, 1H), 8.09 (dd, J=1.6, 8.4 Hz, 1H), 7.72 (d, J=8.8 Hz, 1H), 7.30 (q, J=7.2 Hz, 1H), 7.15-7.03 (m, 3H), 6.99 (d, J=7.6 Hz, 1H), 4.57 (s, 1H), 3.84 (t, J=7.6 Hz, 2H), 3.54 (t, J=6.0 Hz, 2H), 3.41 (s, 3H), 3.31 (ddd, J=6.8, 13.6, 35.2 Hz), 3.11 (ddd, J=5.9, 14.2, 24.5 Hz, 1H), 1.91 (ddd, J=0.7, 6.0, 12.8 Hz, 2H);

Enzymatic Rho kinase (ROCK I and ROCK II) Assays.

The assay is based on ability of Rhok2 to phosphorylate a specific peptide sequence derived from its substrate—ribosomal protein S6 (amino acid residues 229-239, (LCB-AKRRRLSSLRA-NH₂)). Rhok2 uses ATP as a donor of phosphate for the phosphorylation of the substrate, which leads to the depletion of ATP in the reaction mix. An assay kit (“Kinase-Glo”, Promega) was used to quantify enzyme activity. Using this kit, residual amounts of ATP are measured by a secondary enzymatic reaction, through which luciferase utilizes the remaining ATP to produce luminescence. Luminescent signal is directly proportional to ATP concentration and inversely proportional to Rhok2 activity.

This dose response assay was conducted in 1536 well plate format. Each concentration was tested nominally in triplicate. Protocol Summary: 1.25 microliters of solution containing 20 micromolar ATP and 20 micromolar S6 peptide (substrate) in assay buffer (50 millimolar HEPES pH 7.3, 10 millimolar MgCl₂, 0.1% BSA, 2 millimolar DTT) were dispensed in 1536 microtiter plate. 15 nanoliters of test compound or positive and negative control (2.12 millimolar Y-27632 and DMSO, respectively) were then added to the appropriate wells. Each compound dilution was assayed in triplicate, for a nominal total of 30 data points per dose response curve. The enzymatic reaction was initiated by dispensing 1.25 microliters of 8 nanomolar Rhok2 solution in assay buffer (50 millimolar HEPES pH 7.3, 10 millimolar MgCl₂, 0.1% BSA, 2 millimolar DTT). After 2 hours of incubation at 25 degrees Celsius, 2.5 microliters of Kinase Glo reagent (Promega Corporation, Madison, Wis.) was added to each well. Plates were incubated for 10 minutes and luminescence was read on Perkin-Elmer Viewlux for 60 seconds. Each compound was tested in triplicate. The percent inhibition for each well has been calculated as follows: % inhibition=(test compound−median_negative_control)/(median_positive_control−median_negative_control)*100 where the positive control is Y-27632 (13 micromolar) and negative control is DMSO only. The IC₅₀s of all examples were in the range of 0.1 nM-20 μM. For example, the IC₅₀ of Example 169 is 6 nM.

Myosin Light Chain Double Phosphorylation Assays (ppMLC, cell assay).

Serum starved smooth muscle cells were incubated with compound for 1 h before induction of myosin light chain phosphorylation by LPA for 30 min. Cells were washed and fixed before staining for phosphorylated myosin light chain and DNA. Phosphorylation status was quantitated with the LI-COR Odyssey Imager. The IC₅₀s of all examples were in the range of 1 nM-20 μM. For example, the IC₅₀ of Example 169 is 6 nM.

### Neurite Length Assay (N2a, Cell Assay).

N2a cells are maintained in DMEM/FBS at 37 C and 5% CO₂. For the experiment the cells were plated on a poly-D-lysine coated 96-well tissue culture plate. After attachment, cell differentiation was induced for 2 days by addition of 10 uM retinoic acid. Cells were treated for 1 h with a dilution of compounds in 0.3% DMSO final concentration before neurite retraction was induced by 5 uM LPA. Cells were stained for tubulin and nuclei and images were acquired on a INCell 1000 workstation. Images were analyzed using the developer toolbox and neurite length was quantitated. The IC₅₀s of all examples selected for testing were in the range of 1 nM-1 μM. For example, the IC₅₀ of Example 169 is 4 nM.

All references cited herein are incorporated by reference. The present invention may be embodied in other specific forms without departing from the spirit or essential attributes thereof and, accordingly, reference should be made to the appended claims, rather than to the foregoing specification, as indicating the scope of the invention.

1. A compound of formula IA or IB: 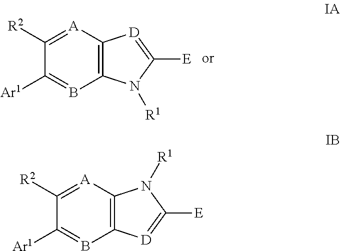
wherein:
A is CR² or N;
B is CR² or N;
D is CR² or N;
Ar¹ is an optionally substituted 5- or 6-membered monocyclic or 8-, 9-, or 10-membered fused bicyclic heterocycle, the ring atoms of which are carbon atoms and one, two, three, or four nitrogen atoms, wherein the optional substitutents are independently at each occurrence selected from the group consisting of (C₁-C₆)alkyl, (C₂-C₆)alkenyl; (C₂-C₆)alkynyl; halogen; —C≡N; —NO₂; —C(═O)R³; —C(═O)OR³; —C(═O)NR³₂; —OR³; —OC(═O)(C₁-C₆)alkyl; —NR³₂, —NR³C(═O)R³; and (C₁-C₃)perfluoroalkyl;
E is selected from the group consisting of, wherein a wavy line signifies a point of attachment, 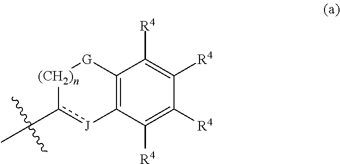
wherein
n is 0 to 2;
G is CH₂, O, S, NR⁵, or CHNHR⁵;
J is CH, CH₂, O, S, NR⁵, CNHR⁵, or CHNHR⁵; and
a dashed line indicates a double bond is present or absent, provided that when J is O, S, NR⁵, or CHNR⁵, the double bond is absent, and when J is CH or CNHR⁵, the double bond is present; 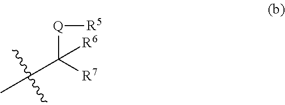
wherein Q is NH or O; 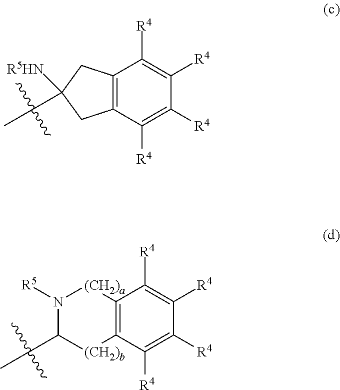
wherein
a is 2 and b is 0; or
a is 1 and b is 1; 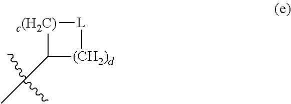
wherein
L is NR⁵, or CHNHR⁵;
c is 0, 1, or 2;
d is 1, 2, 3, 4, or 5;
provided that the sum of c and d is 3, 4, or 5; 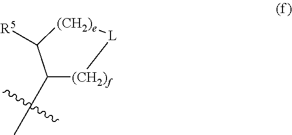
wherein
L is NR⁵, or CHNHR⁵;
e is 0 or 1;
f is 1 or 2;
provided that the sum of e and f is 2 or 3; and 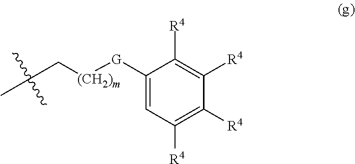
wherein
m is 0 to 2;
G is CH₂, O, S, NR⁵, or CHNHR⁵;
R¹ is hydrogen, (C₁-C₆)alkyl, (C₂-C₆)alkenyl, cycloalkyl, (C₁-C₆)alkylene-cycloalkyl, Ar², —(C₁-C₆)alkylene-Ar², —(C₁-C₆)alkylene-NR³₂, —(C₁-C₆)alkylene-OR³, heterocyclyl, or (C₁-C₆)alkylene-heterocyclyl;
Ar² is unsubstituted aryl, unsubstituted heteroaryl, aryl substituted with one or more substituents selected from R^{a}, or heteroaryl substituted with one or more substituents selected from R^{a};
R^{a} is (C₁-C₆)alkyl, (C₂-C₆)alkenyl; (C₂-C₆)alkynyl; halogen; —C≡N; —NO₂; —C(═O)R³; —C(═O)OR³; —C(═O)NR³₂; —C(═NR³)NR³₂; —OR³; —OC(═O)(C₁-C₆)alkyl; —OC(═O)O(C₁-C₆)alkyl; —OC(═O)NR³₂; —NR³₂; —NR³C(═O)R³; —NR³C(═O)O(C₁-C₆)alkyl; —NR³C(═O)NR³₂; —NR³SO₂R³; —SR³; —S(O)R³; —SO₂R³; —OSO₂(C₁-C₆)alkyl; —SO₂NR³₂; phenyl; pyridyl; 1H-pyrazolyl; 3,5-dimethyl-1H-pyrazolyl; or (C₁-C₃)perfluoroalkyl;
each R² is independently hydrogen, (C₁-C₆)alkyl, (C₂-C₆)alkenyl; (C₂-C₆)alkynyl; halogen; —C≡N; —NO₂; —C(═O)R³; —C(═O)OR³; —C(═O)NR³₂; —C(═NR³)NR³₂; —OR³; —OC(═O)(C₁-C₆)alkyl; —OC(═O)O(C₁-C₆)alkyl; —OC(═O)NR³₂; —NR³₂; —NR³C(═O)R³; —NR³C(═O)O(C₁-C₆)alkyl; —NR³C(═O)NR³₂; —NR³SO₂R³; —SR³; —S(O)R³; —SO₂R³; —OSO₂(C₁-C₆)alkyl; —SO₂NR³₂; or (C₁-C₃)perfluoroalkyl;
each R³ is independently hydrogen, (C₁-C₆)alkyl, OR³, (C₁-C₆)alkylene-OR³, N(R³)₂, (C₁-C₆)alkylene-N(R³)₂, (C₁-C₆)alkylene-C(═O)OR³, (C₁-C₆)alkylene-C(═O)N(R³)₂, (C₃-C₇)cycloalkyl, (C₁-C₆)alkylene-(C₃-C₇)cycloalkyl, (C₃-C₇)-heterocyclyl, (C₁-C₆)alkylene-(C₃-C₇)-heterocyclyl, aryl, (C₁-C₆)alkylene-aryl, heteroaryl, or (C₁-C₆)alkylene-heteroaryl, wherein any alkyl, cycloalkyl, heterocyclyl, aryl, or heteroaryl is substituted with 0-3 J; wherein two R³ groups connected a nitrogen atom in an —NR³₂ moiety may in combination be —(CH₂)ₑ— or —(CH₂)_{f}M(CH₂)₂—; wherein e is 4, 5, or 6; each f is 2 or 3; and M is O, S, NH, N(C₁-C₆)alkyl or NC(═O)(C₁-C₆)alkyl;
each R⁴ is independently selected from the group consisting of hydrogen, (C₁-C₆)alkyl, hydroxy(C₁-C₆)alkyl, (C₂-C₆)alkenyl; (C₂-C₆)alkynyl; halogen; —C≡N; —NO₂; —C(═O)R³; —C(═O)OR³; (C₁-C₆)alkylene-C(═O)OR³; —C(═O)NR³₂; (C₁-C₆)alkylene-C(═O)NR³₂; —C(═NR³)NR³₂; —OR³; (C₁-C₆)alkylene-OR³; —OC(═O)(C₁-C₆)alkyl; —OC(═O)O(C₁-C₆)alkyl; —OC(═O)NR³₂; —NR³₂; —NR³C(═O)R³; —NR³C(═O)O(C₁-C₆)alkyl; —NR³C(═O)NR³₂; —NR³(C₁-C₆)alkylene-NR³₂; —NR³(C₁-C₆)alkylene-OR³; —NR³(C₁-C₆)alkylene-Ar²; —NR³SO₂R³; —SR³; —S(O)R³; —SO₂R³; —OSO₂(C₁-C₆)alkyl; —SO₂NR³₂; (C₁-C₃)perfluoroalkyl; —O(C₁-C₃)perfluoroalkyl; pyrazolyl; triazolyl; and tetrazolyl; or two R⁴ groups taken together form a fused cycloalkyl, heterocyclyl, aryl or heteroaryl ring;
R⁵ is hydrogen, (C₁-C₆)alkyl, (C₁-C₆)alkenyl, C(═O)(C₁-C₆)alkyl, C(═O)O(C₁-C₆)alkyl, Ar², —(C₁-C₆)alkylene-Ar², —(C₁-C₆)C(═O)OR³, or —(C₁-C₆)C(═O)N(R³)₂;
R⁶ is Ar² or —(C₁-C₆)alkylene-Ar²;
R⁷ is hydrogen or (C₁-C₆)alkyl;
or any tautomer, salt, stereoisomer, hydrate, solvent, or prodrug thereof.

2. The compound of claim 1 wherein A and B are both CR² and D is N.

3. The compound of claim 1 wherein one of A and B is N, one of A and B is CR², and D is N.

4. The compound of claim 1 wherein A is N.

5. The compound of claim 1 wherein B is N.

6. The compound of claim 1 wherein Ar¹ is an optionally substituted heterocycle selected from the group consisting of optionally substituted pyridyl, pyrimidinyl, 1H-pyrazolyl, 1H-pyrrolo[2,3-b]pyridinyl, 7H-pyrrolo[2,3-d]pyrimidinyl, 1H-pyrazolo[3,4-b]pyridinyl and 1H-pyrazolo[3,4-d]pyrimidinyl.

7. The compound of claim 1 wherein Ar¹ is an optionally substituted heterocycle selected from the group consisting of optionally substituted 4-pyridyl, pyrimidin-4-yl, 1H-pyrazol-4-yl, 1H-pyrrolo[2,3-b]pyridin-4-yl, 7H-pyrrolo[2,3-d]pyrimidin-4-yl, 1H-pyrazolo[3,4-b]pyridin-4-yl and 1H-pyrazolo[3,4-d]pyrimidin-4-yl.

8. The compound of claim 6 wherein Ar¹ is substituted with (C₁-C₆)alkyl, (C₂-C₆)alkenyl; (C₂-C₆)alkynyl; halogen; —C≡N; —NO₂; —C(═O)R³; —C(═O)OR³; —C(═O)NR³₂; —OR³; —OC(═O)(C₁-C₆)alkyl; —NR³₂, —NR³C(═O)R³; or (C₁-C₃)perfluoro alkyl.

9. The compound of claim 1 wherein R¹ is hydrogen or (C₁-C₆)alkyl.

10. The compound of claim 1 wherein each R² is hydrogen.

11. The compound of claim 1 wherein E is:

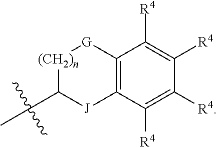

12. The compound of claim 11 wherein G is O.

13. The compound of claim 11 wherein J is CH₂, O, or CHNHR⁵ and the double bond is absent.

14. The compound of claim 11 wherein J is O and the double bond is absent.

15. The compound of claim 14 wherein G is CH₂, O, or CHNHR⁵.

16. The compound of claim 1 wherein R⁵ is hydrogen.

17. The compound of claim 11 wherein J is CH or CNHR⁵ and the double bond is present.

18. The compound of claim 17 wherein G is O or CH₂.

19. The compound of claim 11 wherein G is NR⁵.

20. The compound of claim 19 wherein R⁵ is hydrogen, (C₁-C₆)alkyl, (C₁-C₆)alkenyl, or —(C₁-C₆)alkylene-Ar².

21. The compound of claim 1 wherein E is:

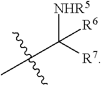

22. The compound of claim 21 wherein R⁷ is hydrogen.

23. The compound of claim 22 wherein Ar² is unsubstituted or substituted phenyl.

24. The compound of claim 21 wherein R⁶ is CH₂Ar².

25. The compound of claim 24 wherein Ar² is unsubstituted or substituted phenyl.

26. The compound of claim 1 wherein E is:

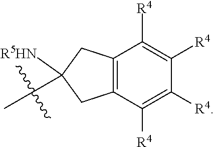

27. The compound of claim 26 wherein each R⁴ is hydrogen.

28. The compound of claim 1 wherein E is:

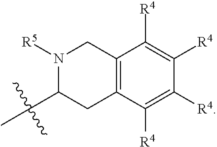

29. The compound of claim 28 wherein each R⁴ is independently hydrogen, (C₁-C₆)alkyl, hydroxy(C₁-C₆)alkyl, (C₂-C₆)alkenyl; (C₂-C₆)alkynyl; halogen; —C≡N; —NO₂; —C(═O)R³; —C(═O)OR³; (C₁-C₆)alkylene-C(═O)OR³; —C(═O)NR³₂; (C₁-C₆)alkylene-C(═O)NR³₂; —C(═NR³)NR³₂; —OR³; (C₁-C₆)alkylene-OR³; —OC(═O)(C₁-C₆)alkyl; —OC(═O)O(C₁-C₆)alkyl; —OC(═O)NR³₂; —NR³₂; —NR³C(═O)R³; —NR³C(═O)O(C₁-C₆)alkyl; —NR³C(═O)NR³₂; —NR³(C₁-C₆)alkylene-NR³₂; —NR³(C₁-C₆)alkylene-OR³; —NR³(C₁-C₆)alkylene-Ar²; —NR³SO₂R³; —SR³; —S(O)R³; —SO₂R³; —OSO₂(C₁-C₆)alkyl; —SO₂NR³₂; (C₁-C₃)perfluoroalkyl; or —O(C₁-C₃)perfluoroalkyl.

30. The compound of claim 1 wherein E is:

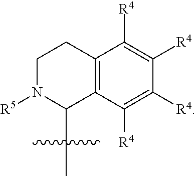

31. The compound of claim 30 wherein each R⁴ is independently hydrogen, (C₁-C₆)alkyl, hydroxy(C₁-C₆)alkyl, (C₂-C₆)alkenyl; (C₂-C₆)alkynyl; halogen; —C≡N; —NO₂; —C(═O)R³; —C(═O)OR³; (C₁-C₆)alkylene-C(═O)OR³; —C(═O)NR³₂; (C₁-C₆)alkylene-C(═O)NR³₂; —C(═NR³)NR³₂; —OR³; (C₁-C₆)alkylene-OR³; —OC(═O)(C₁-C₆)alkyl; —OC(═O)O(C₁-C₆)alkyl; —OC(═O)NR³₂; —NR³₂; —NR³C(═O)R³; —NR³C(═O)O(C₁-C₆)alkyl; —NR³C(═O)NR³₂; —NR³(C₁-C₆)alkylene-NR³₂; —NR³(C₁-C₆)alkylene-OR³; —NR³(C₁-C₆)alkylene-Ar²; —NR³SO₂R³; —SR³; —S(O)R³; —SO₂R³; —OSO₂(C₁-C₆)alkyl; —SO₂NR³₂; (C₁-C₃)perfluoroalkyl; or —O(C₁-C₃)perfluoroalkyl.

32. The compound of claim 1 wherein E is: 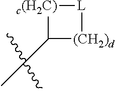
wherein
L is NR⁵, or CHNHR⁵;
c is 0, 1, or 2;
d is 1, 2, 3, 4, or 5;
provided that the sum of c and d is 3, 4, or 5.

33. The compound of claim 32 wherein L is selected from the group consisting of NR⁵ and CHNHR⁵, and wherein R⁵ is hydrogen.

34. The compound of claim 1 wherein E is: 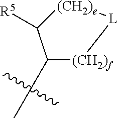
wherein
L is NR⁵, or CHNHR⁵;
e is 0 or 1;
f is 1 or 2;
provided that the sum of e and f is 2 or 3.

35. The compound of claim 1 wherein E is: 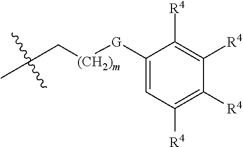
wherein
m is 0 to 2; and
G is CH₂, O, S, NR⁵, or CHNHR⁵.

36. The compound of claim 1, which has the formula I-1, or a salt thereof: 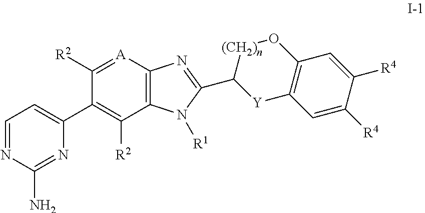
wherein:
A is selected from the group consisting of CR² and N;
Y is CH₂, O, S, or NR⁵; and
n is 0 to 2.

37. A compound according to claim 1, which has the formula I-2, or a salt thereof: 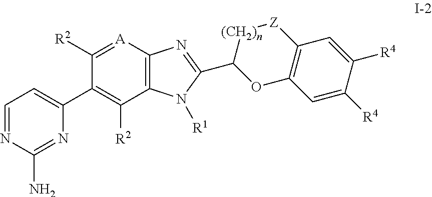
wherein:
A is selected from the group consisting of CR² and N;
Z is CH₂, O, S, or NR⁵; and
n is 0 to 2.

38. The compound of claim 1 wherein the compound is any of the following: 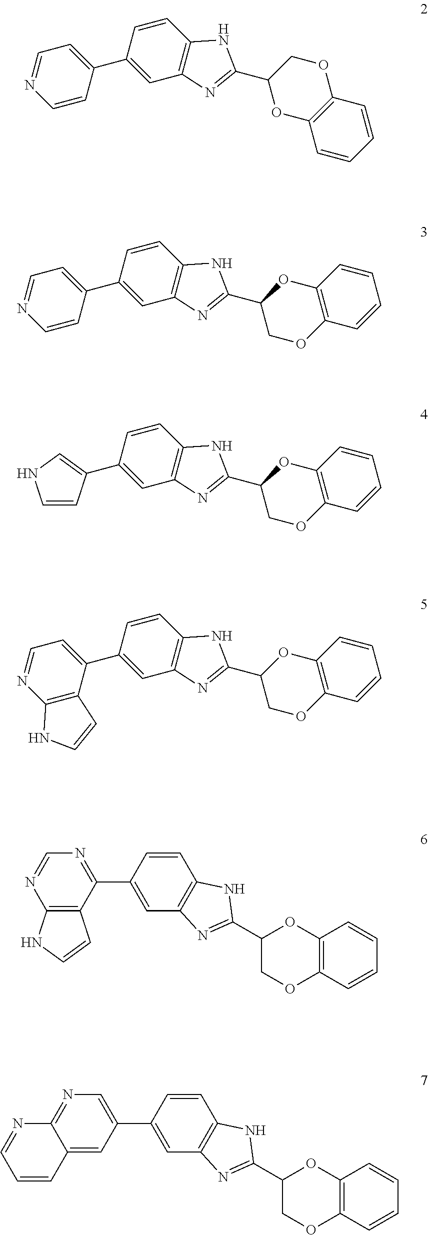 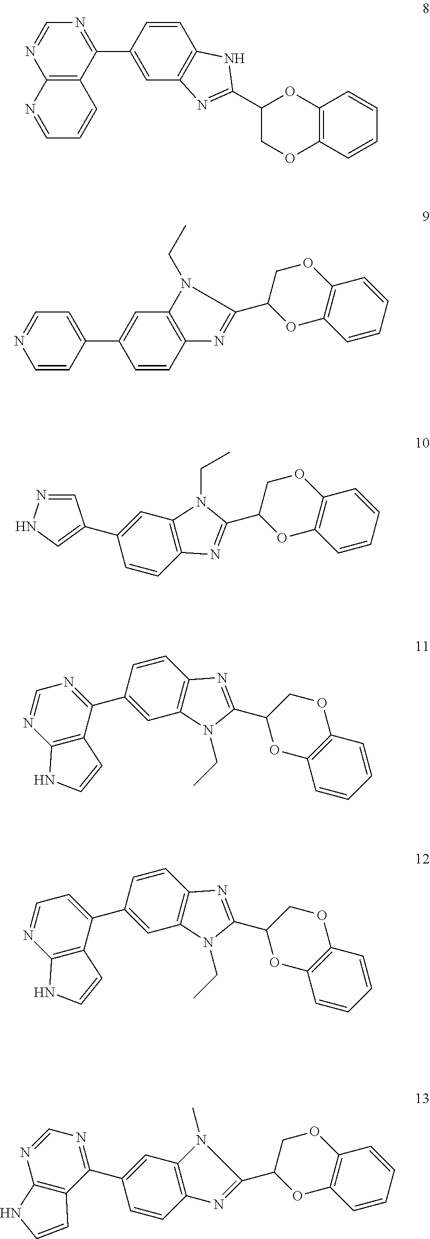 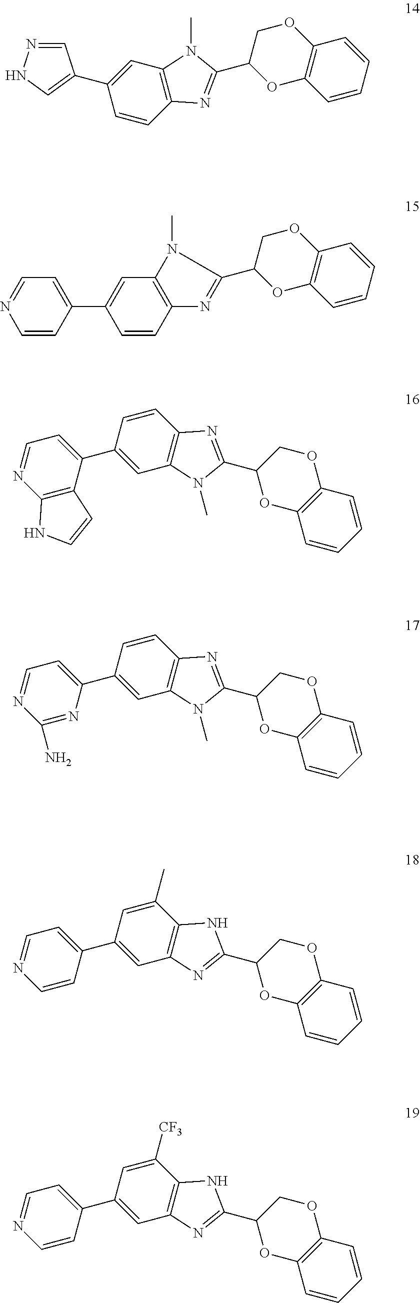 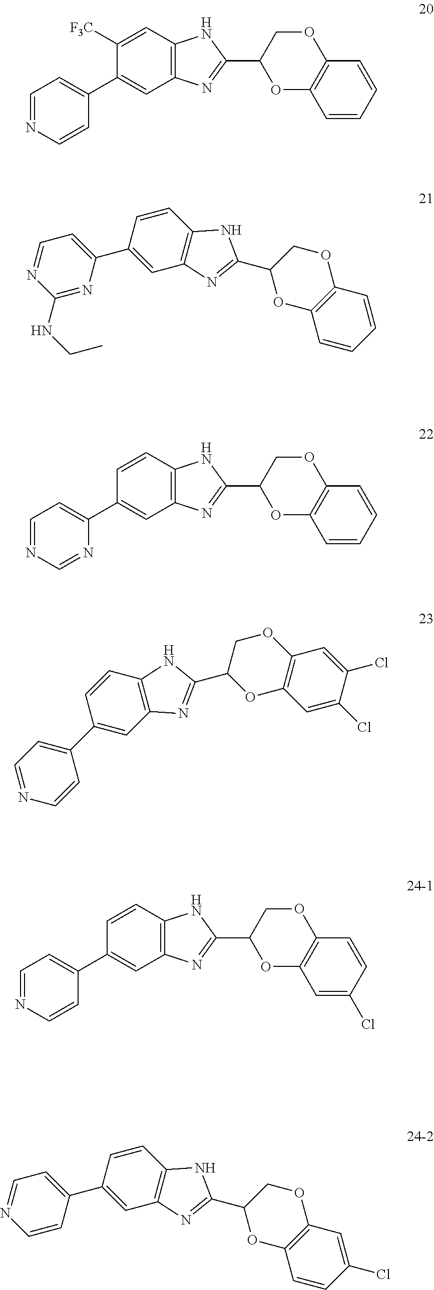 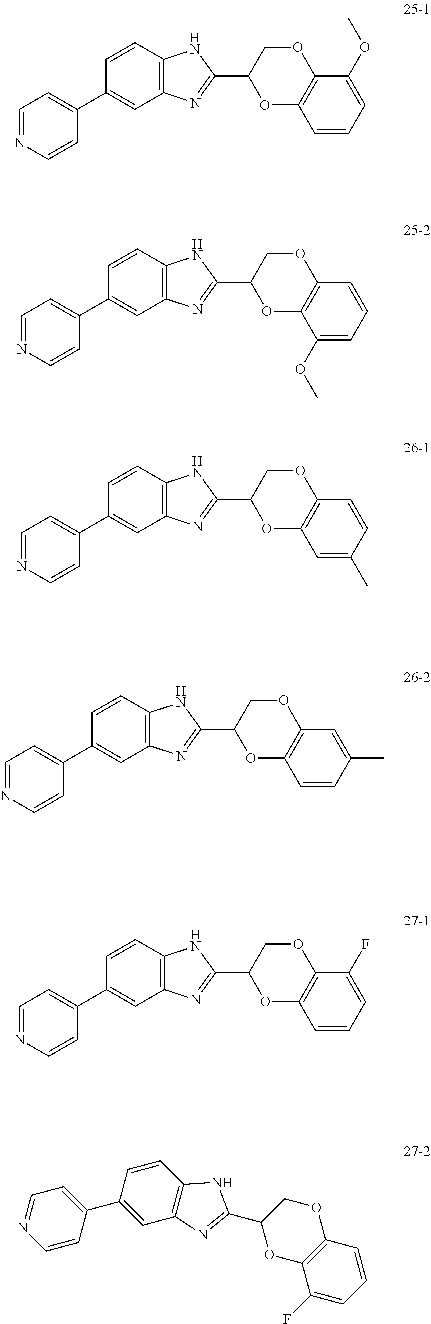 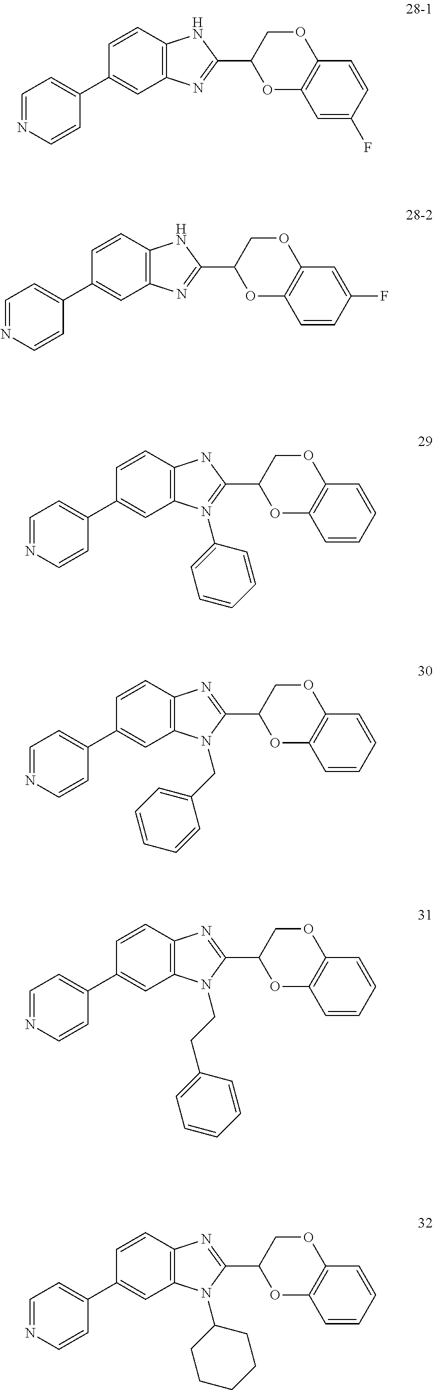 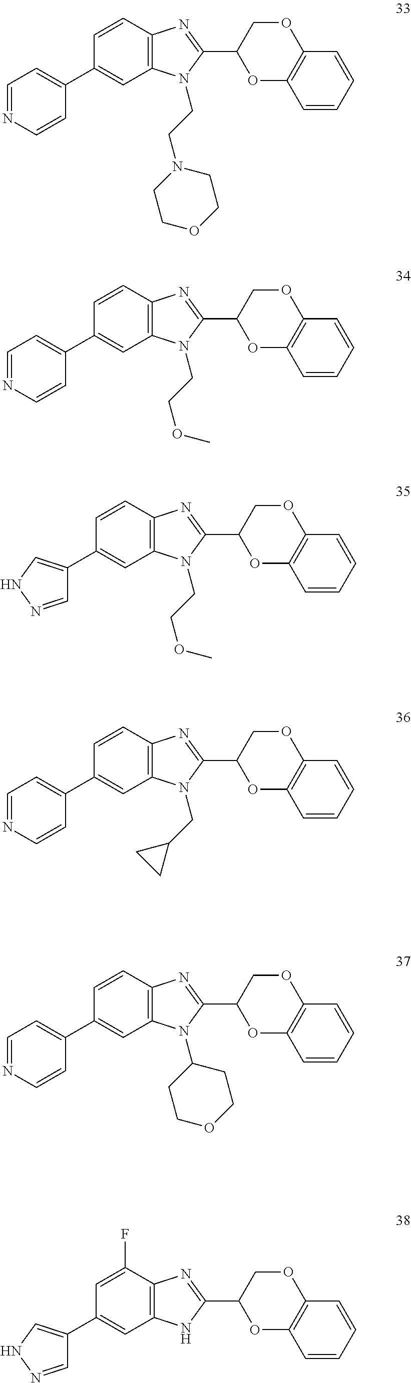 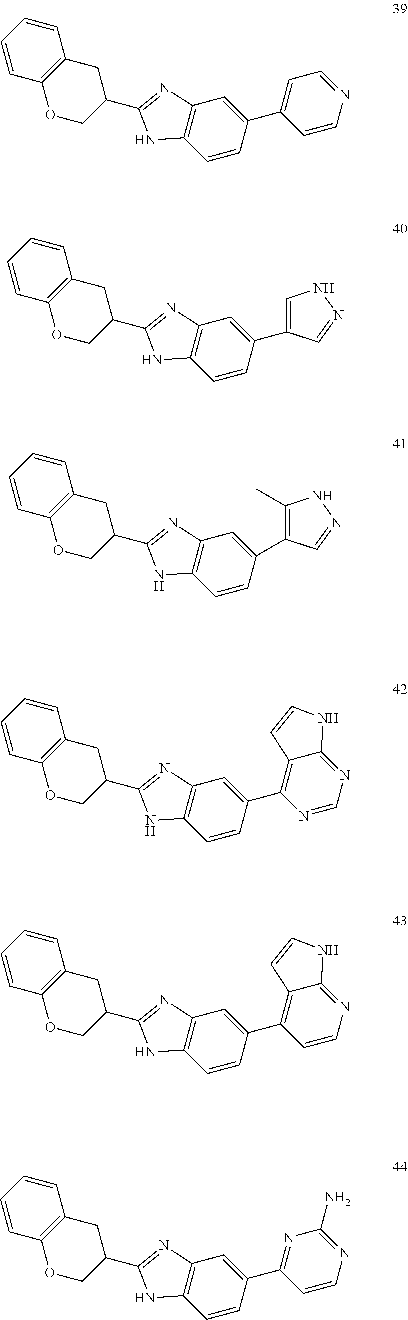 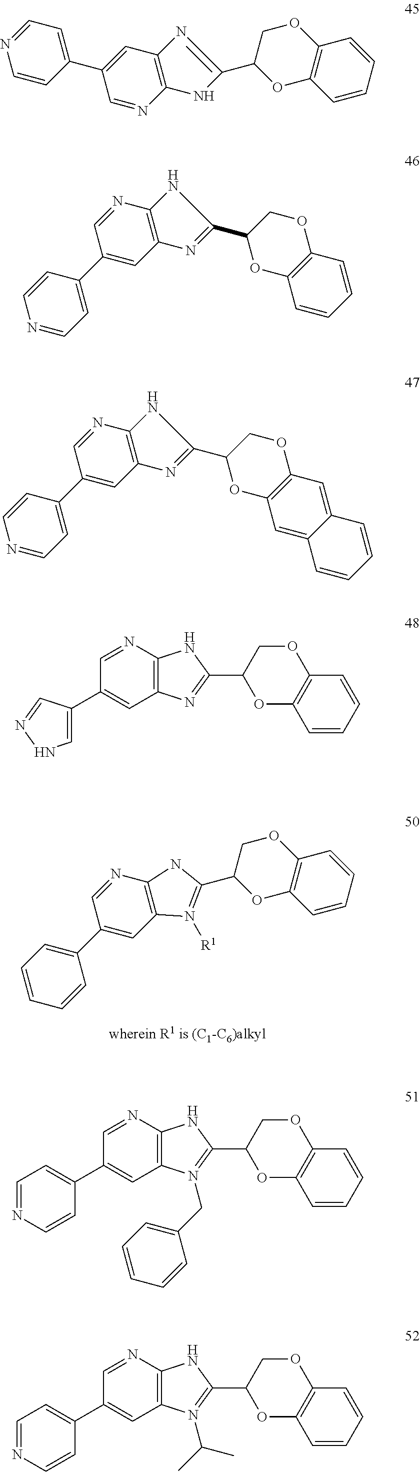 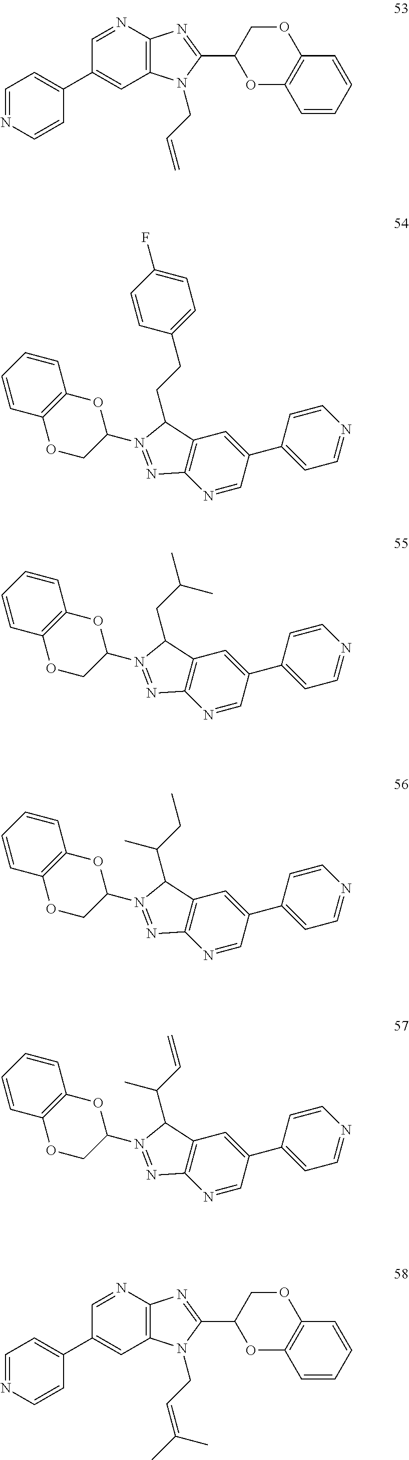 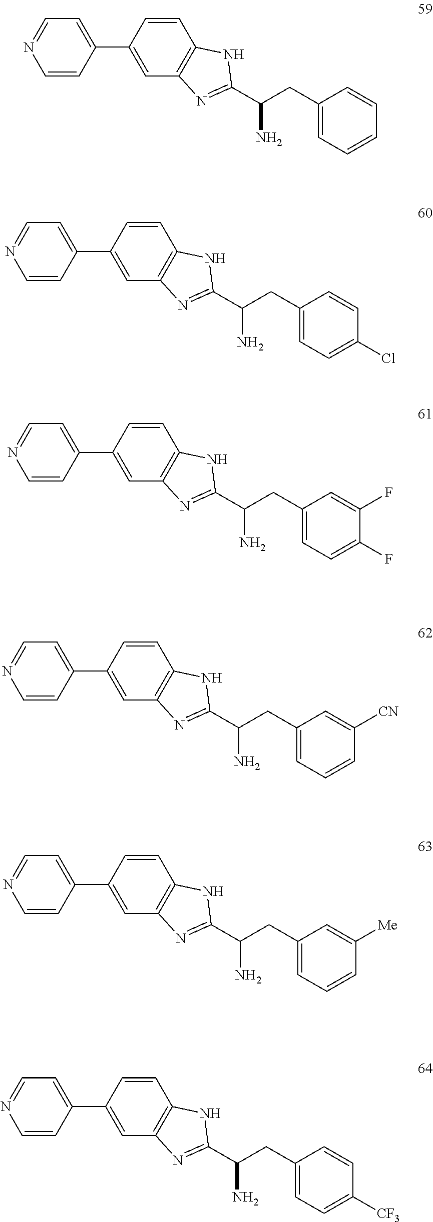 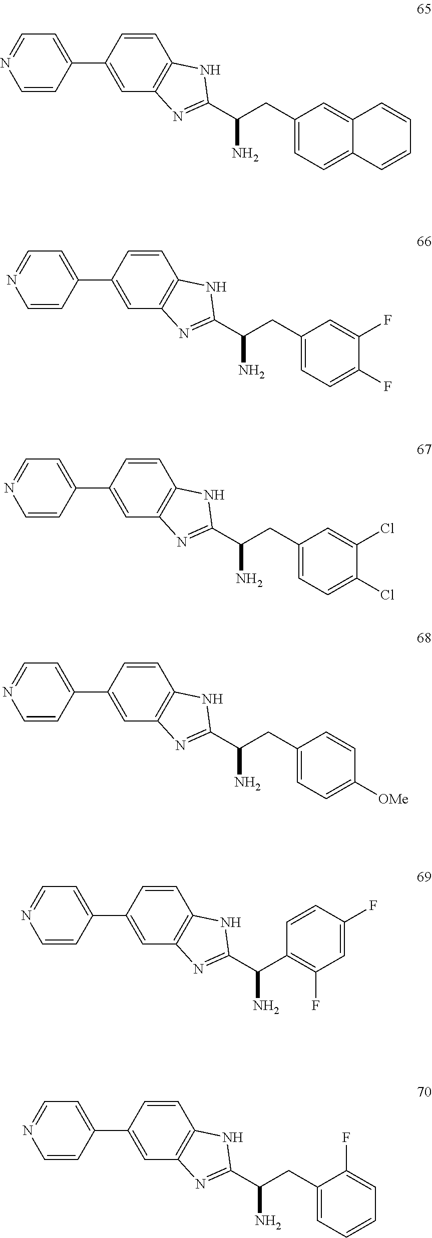 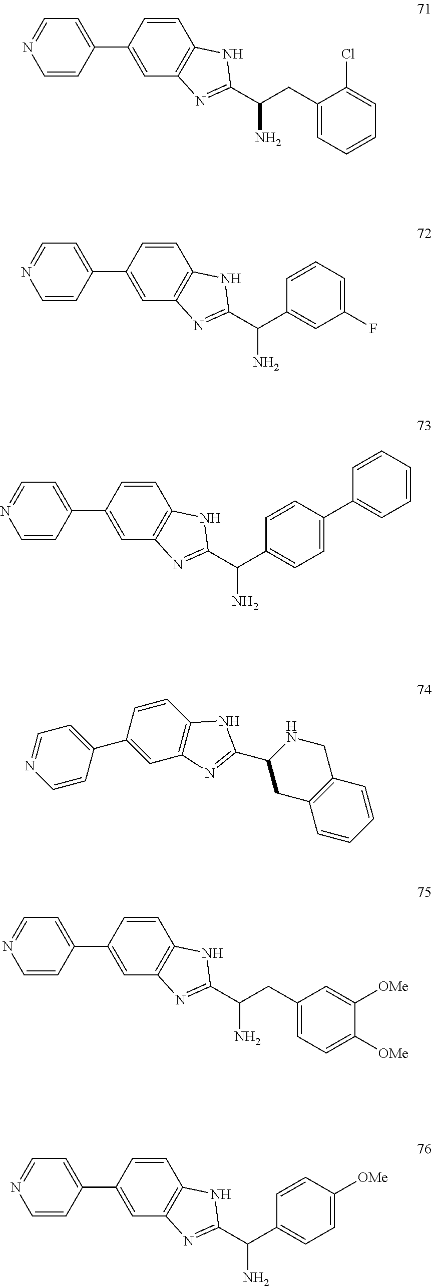 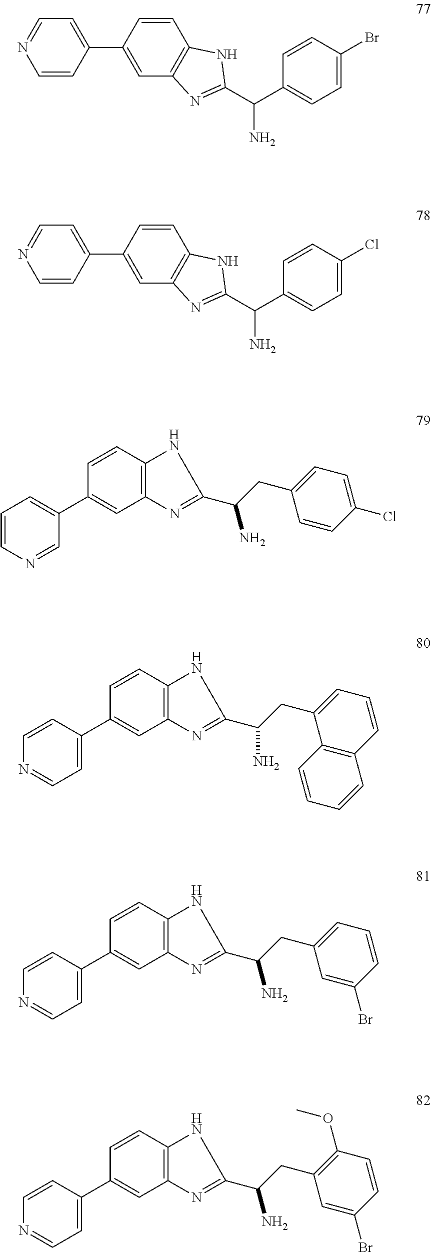 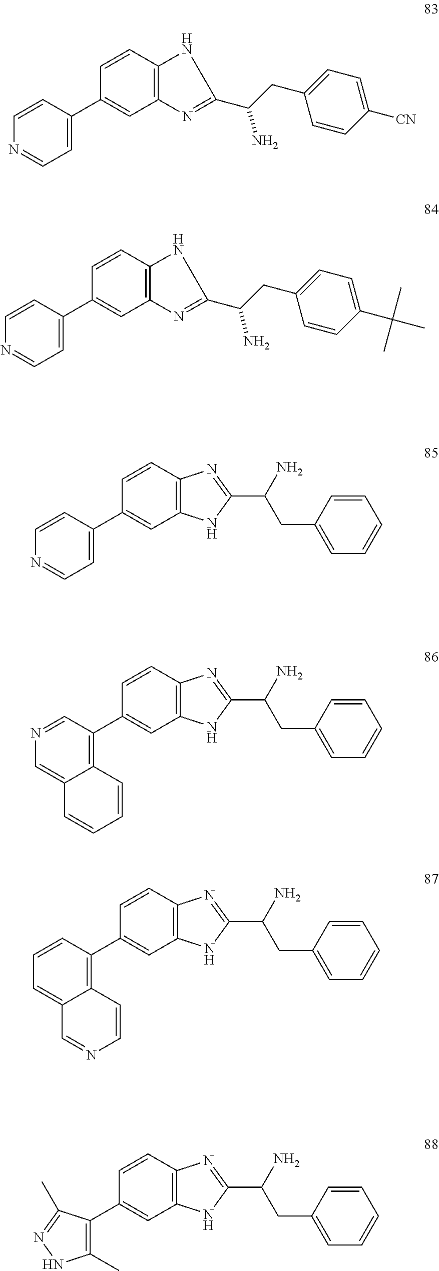 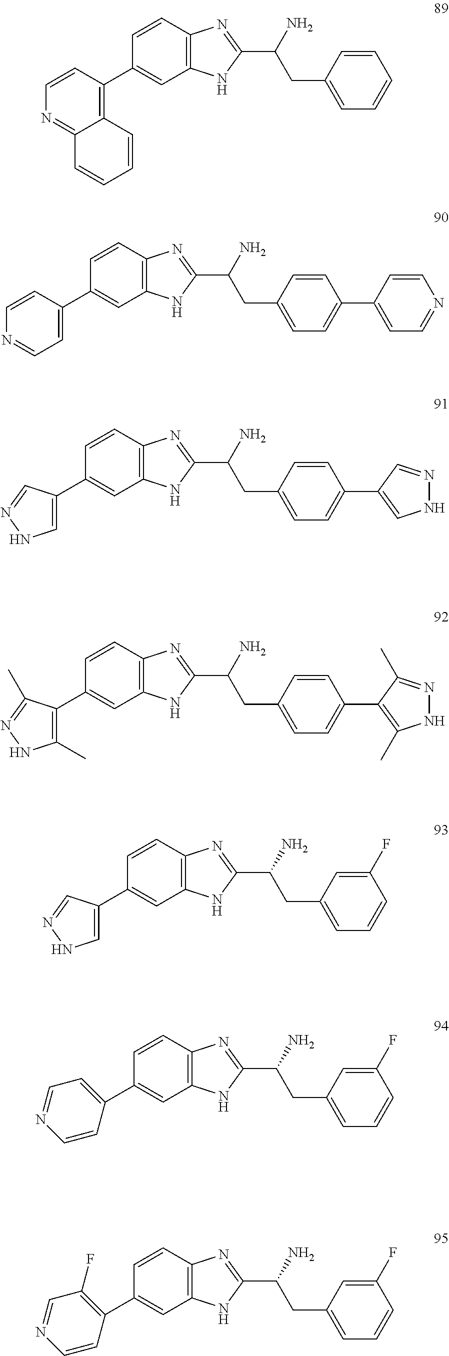 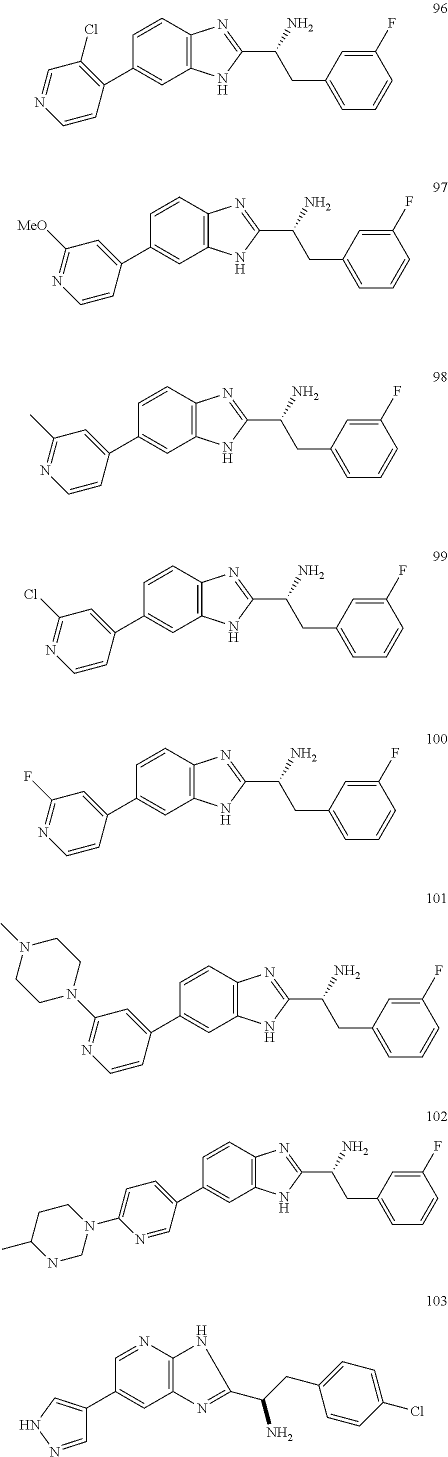 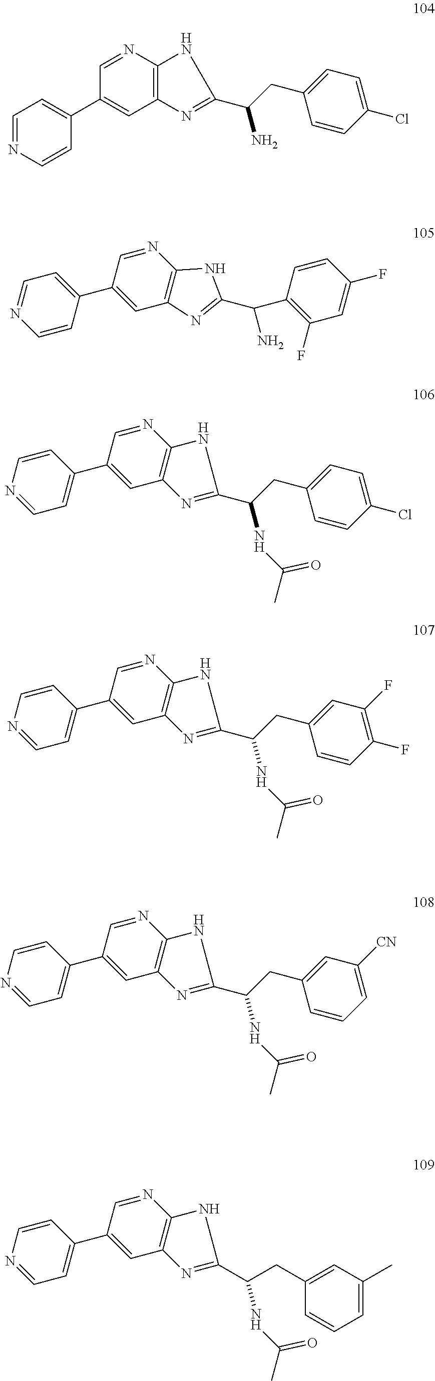 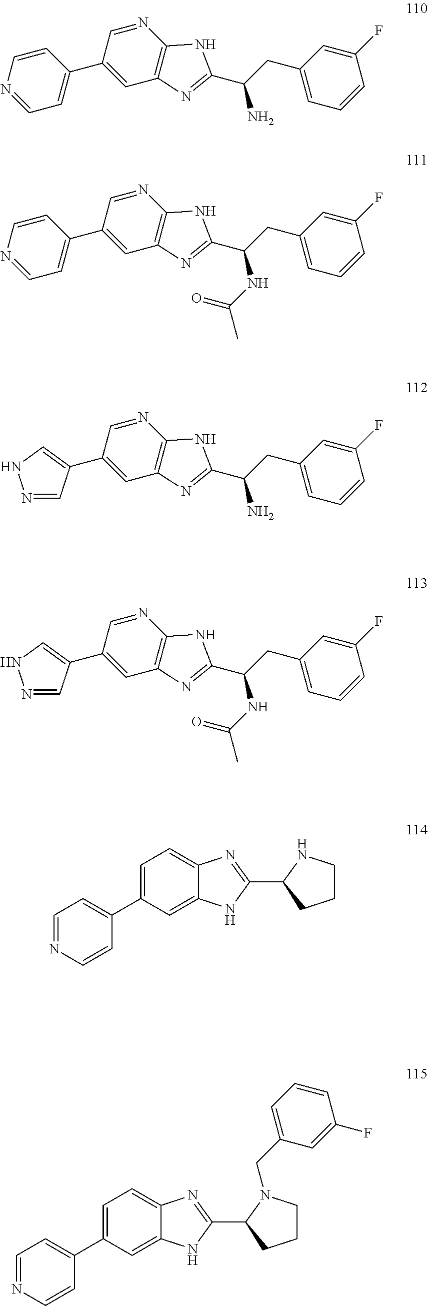 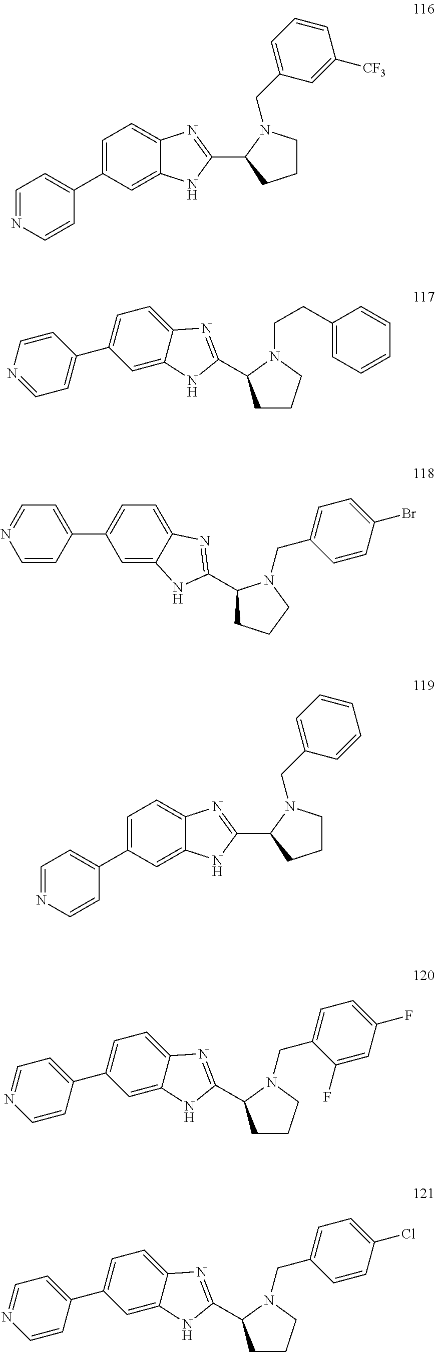 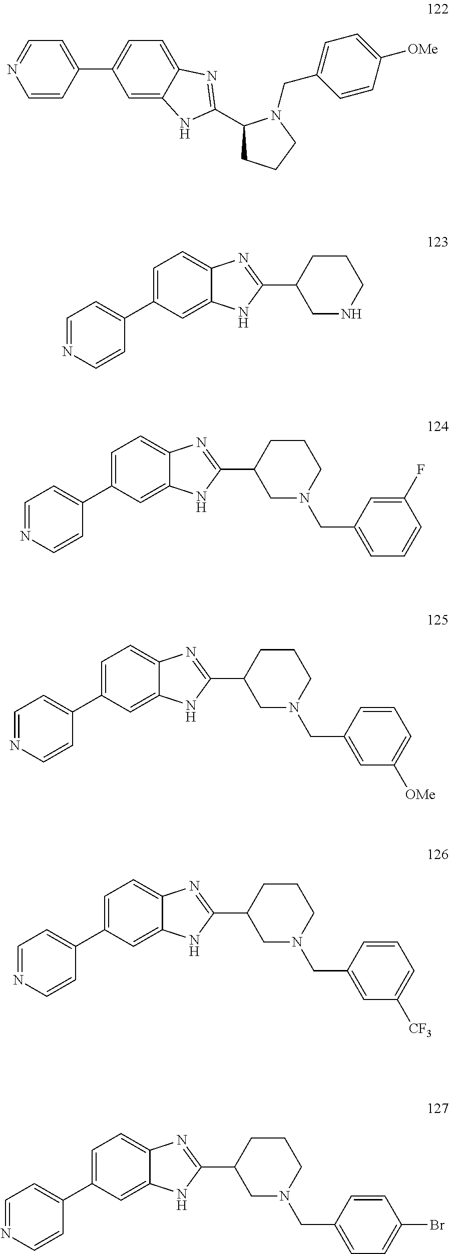 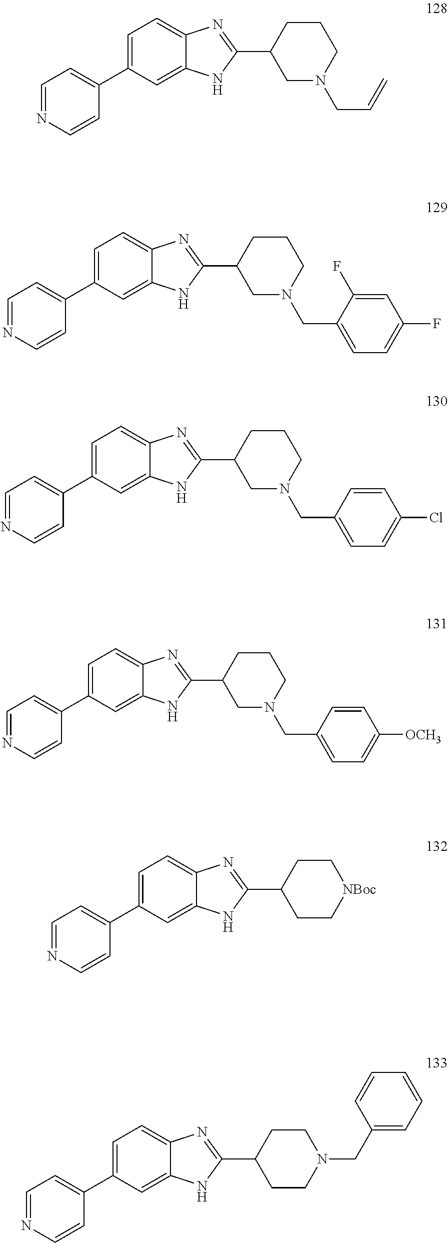 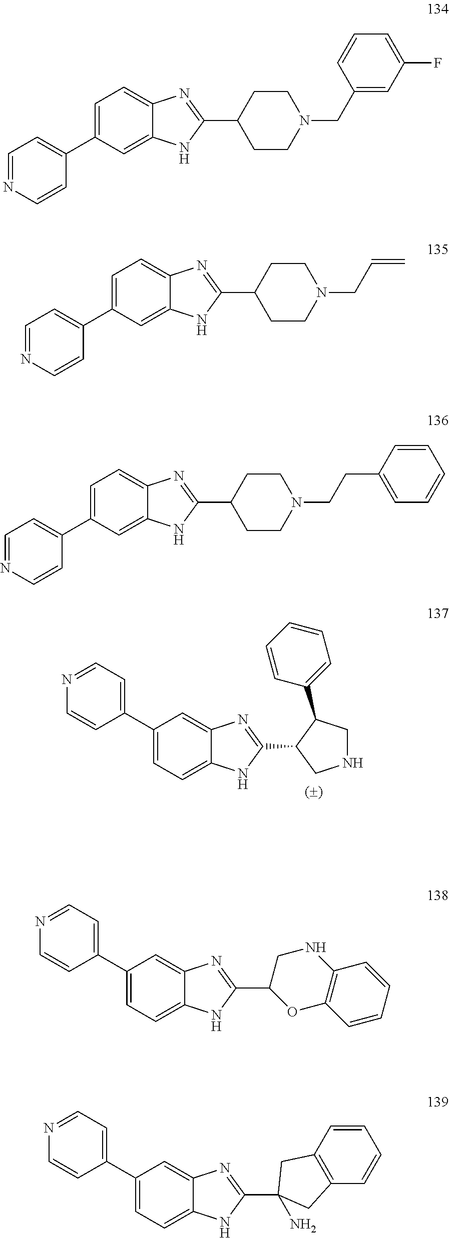 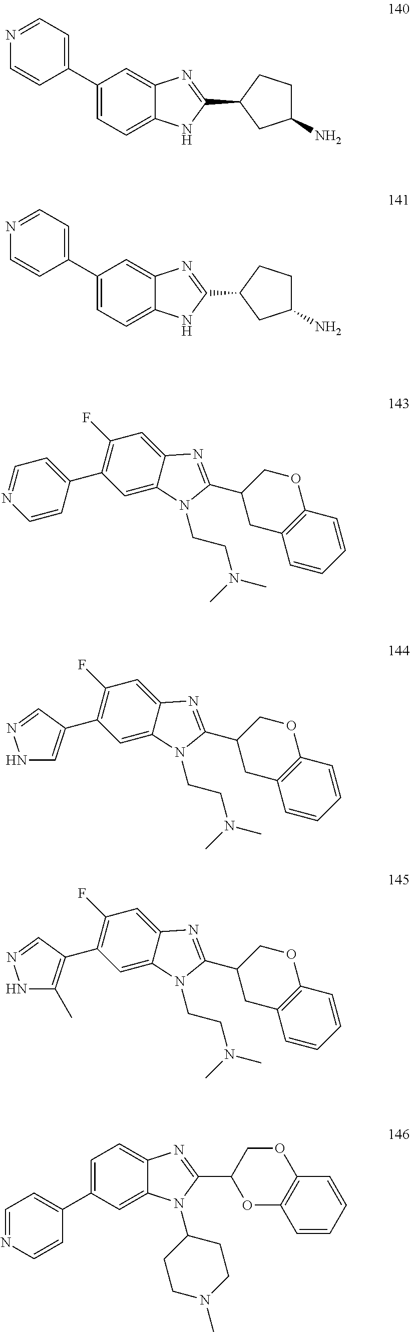 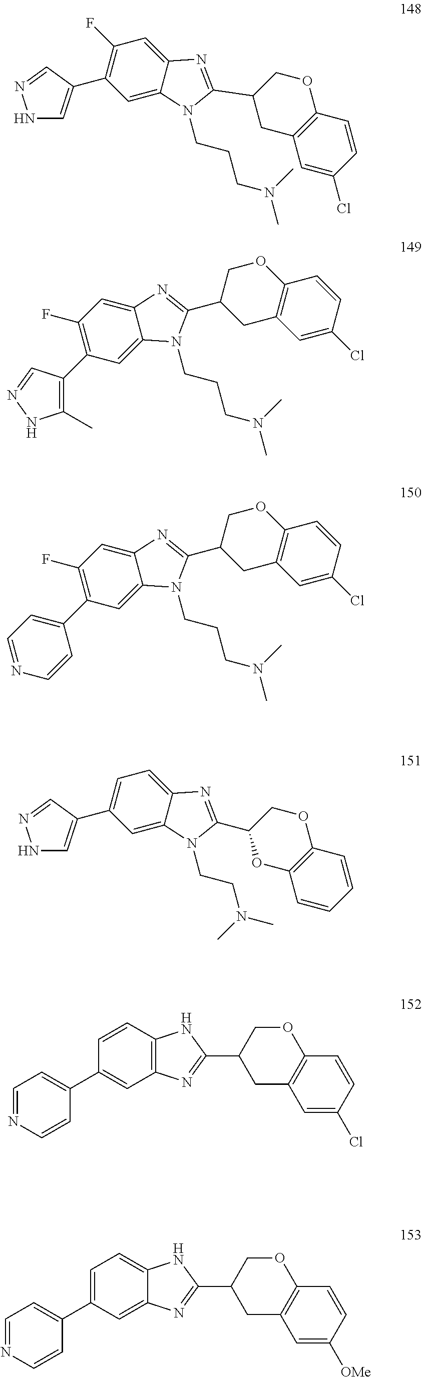 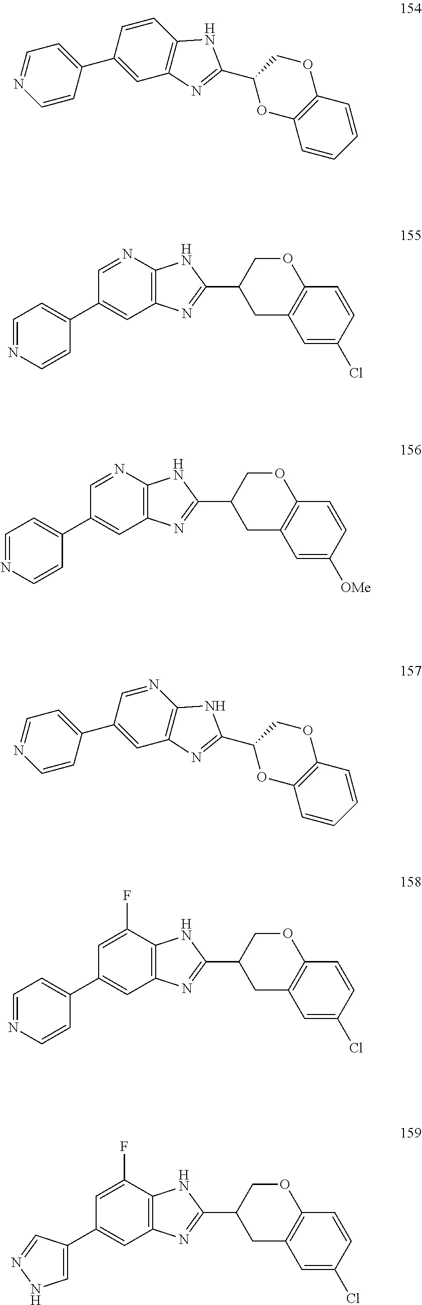 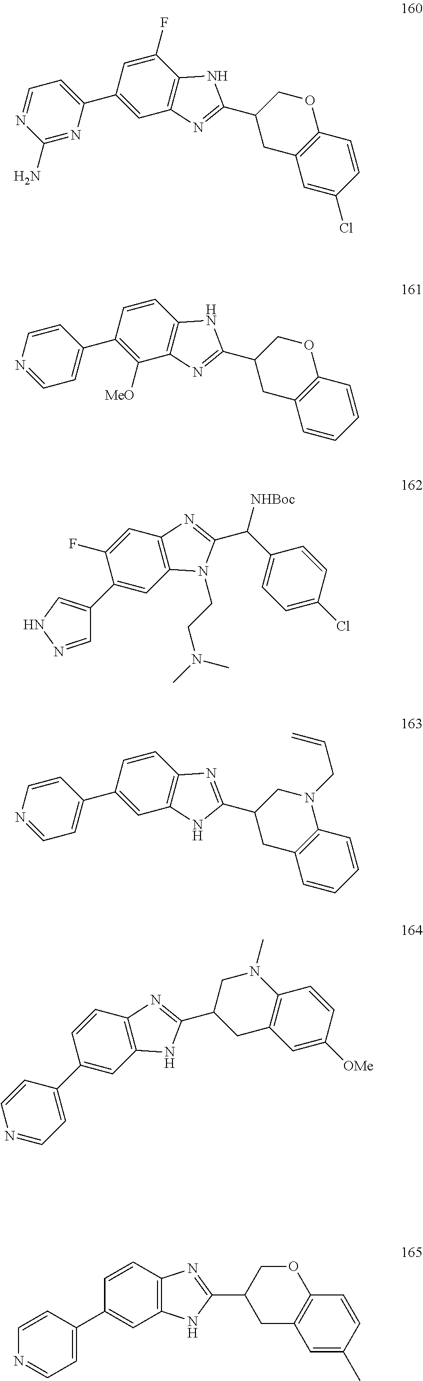 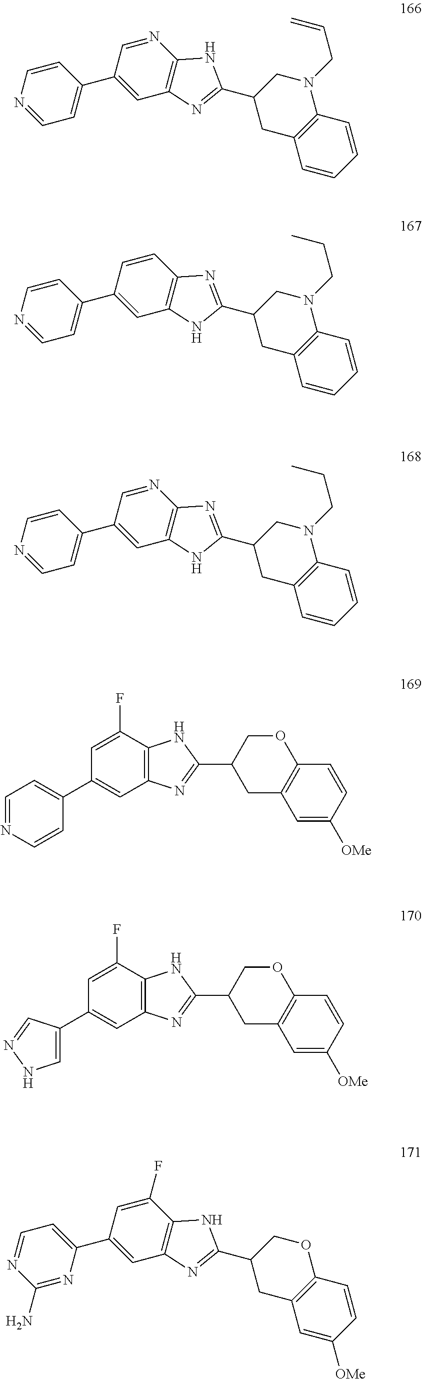 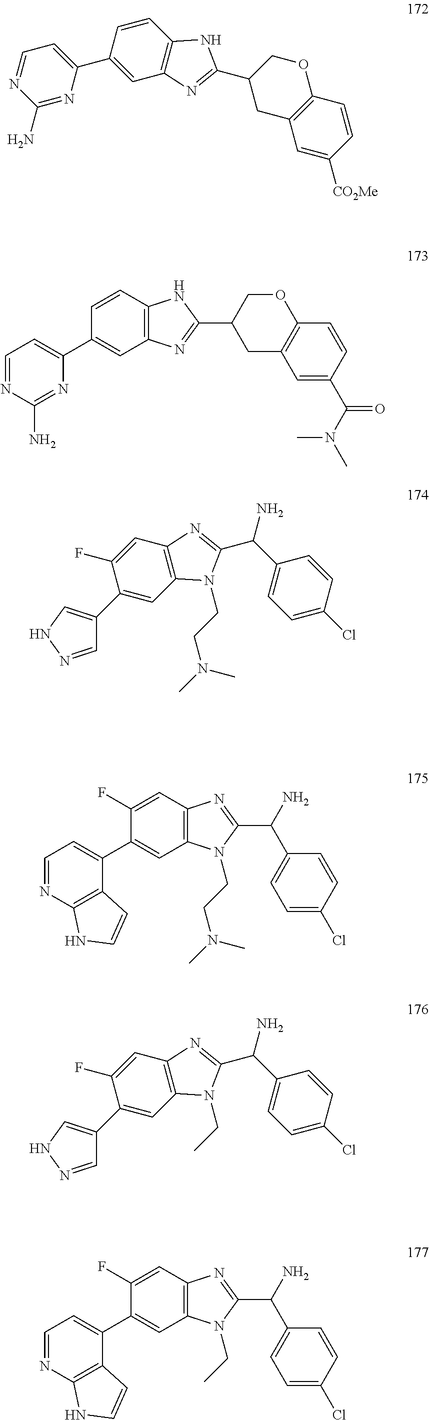 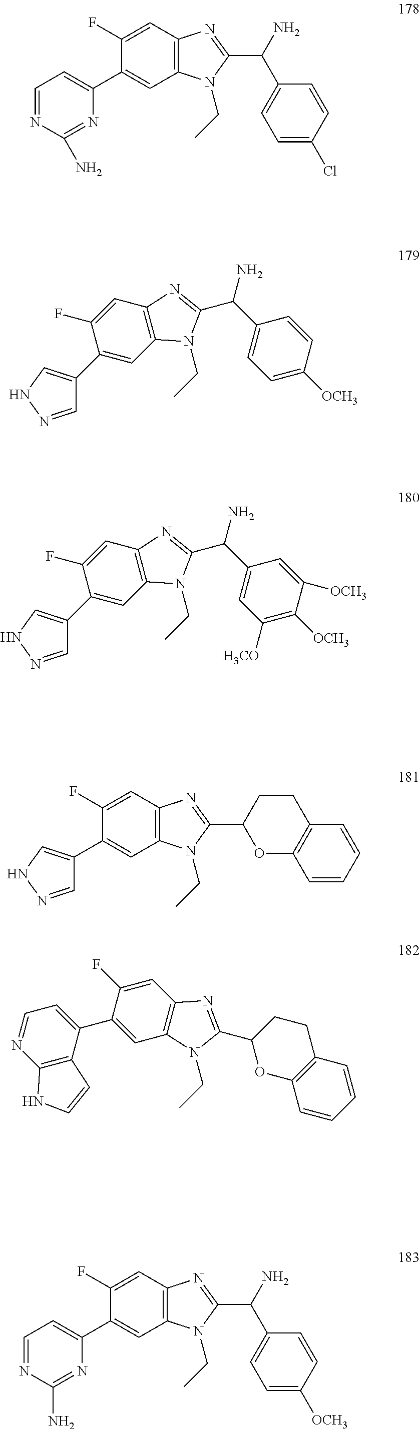 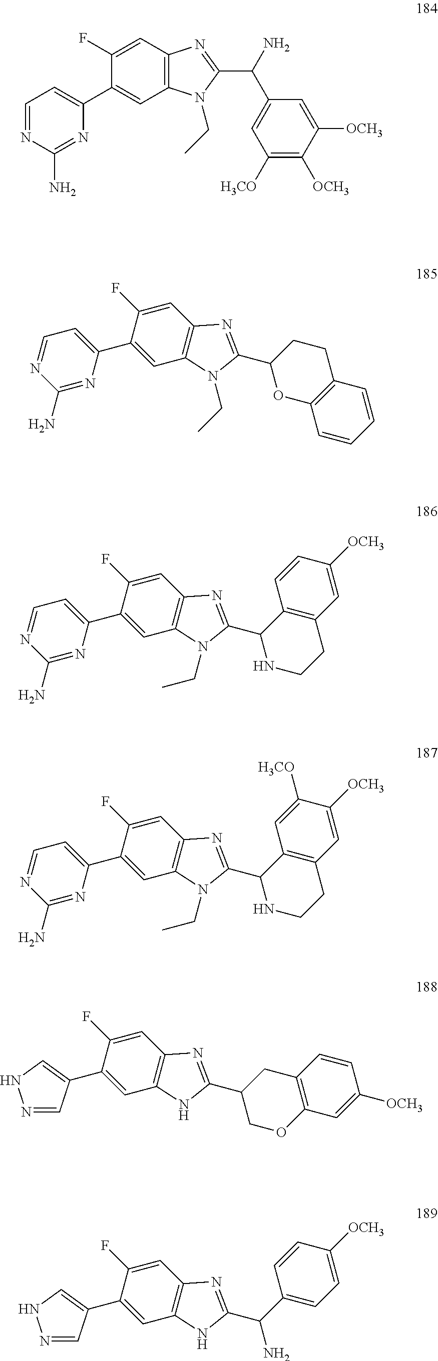 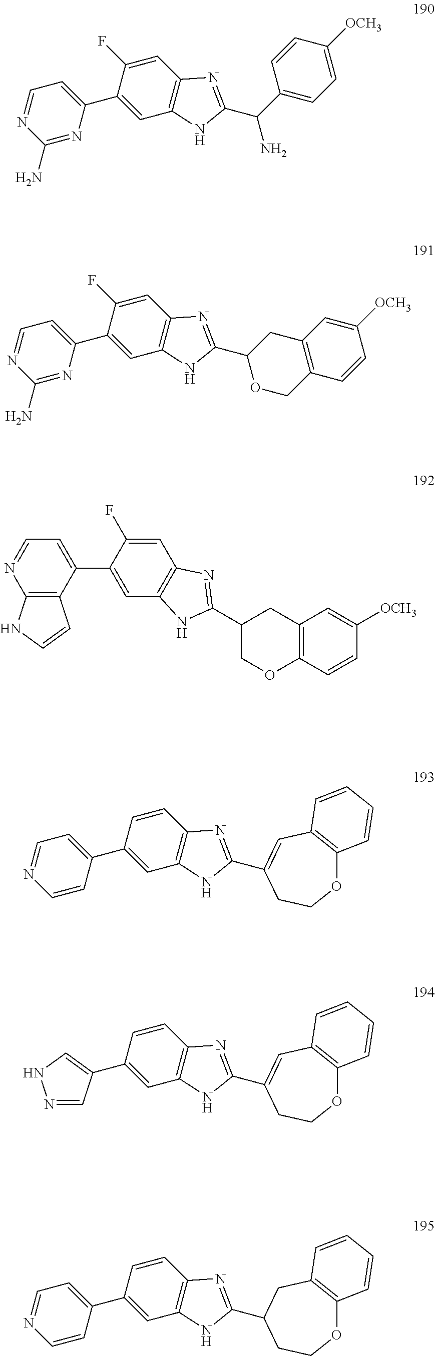 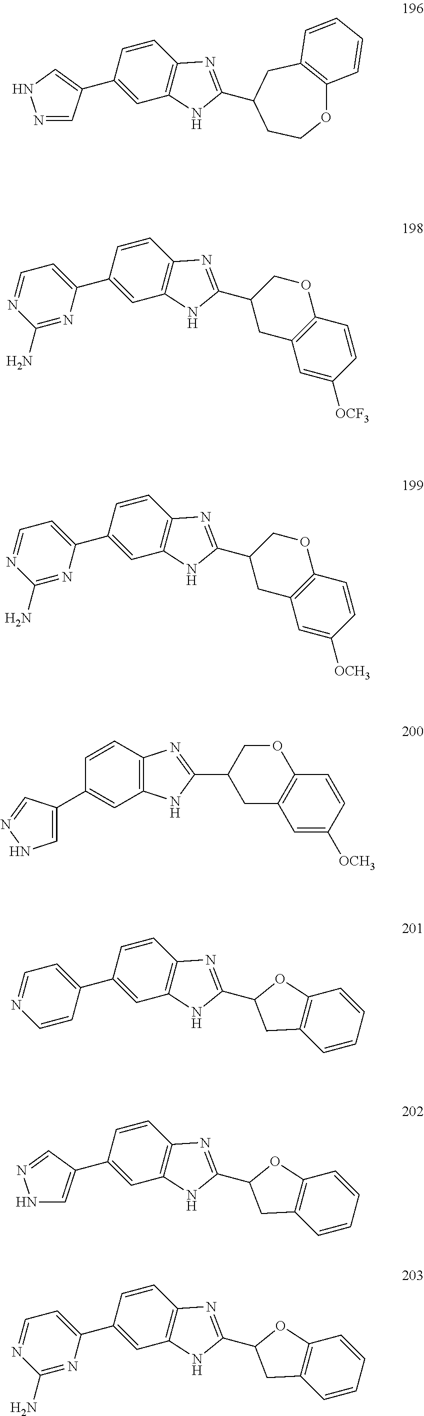 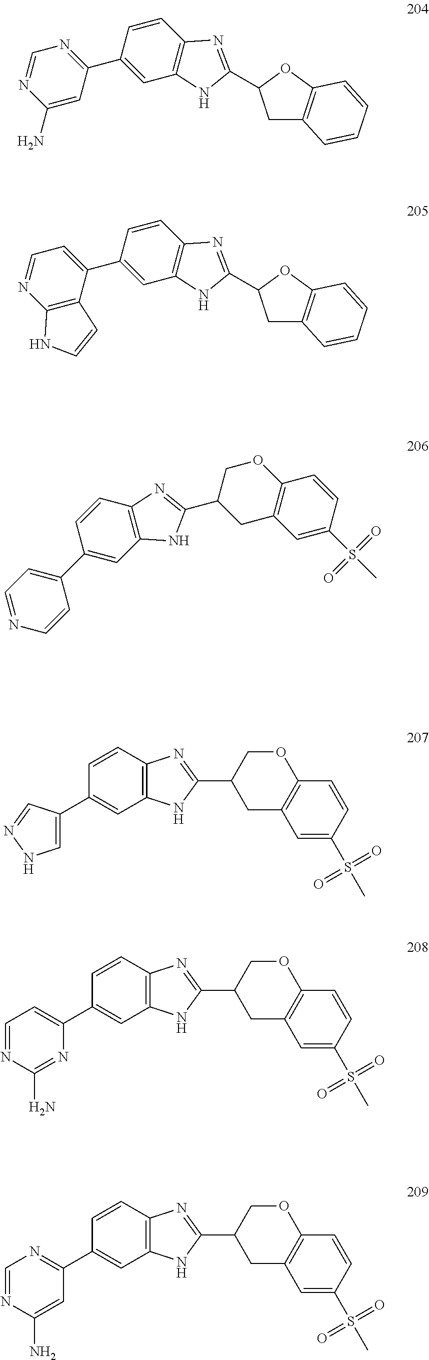 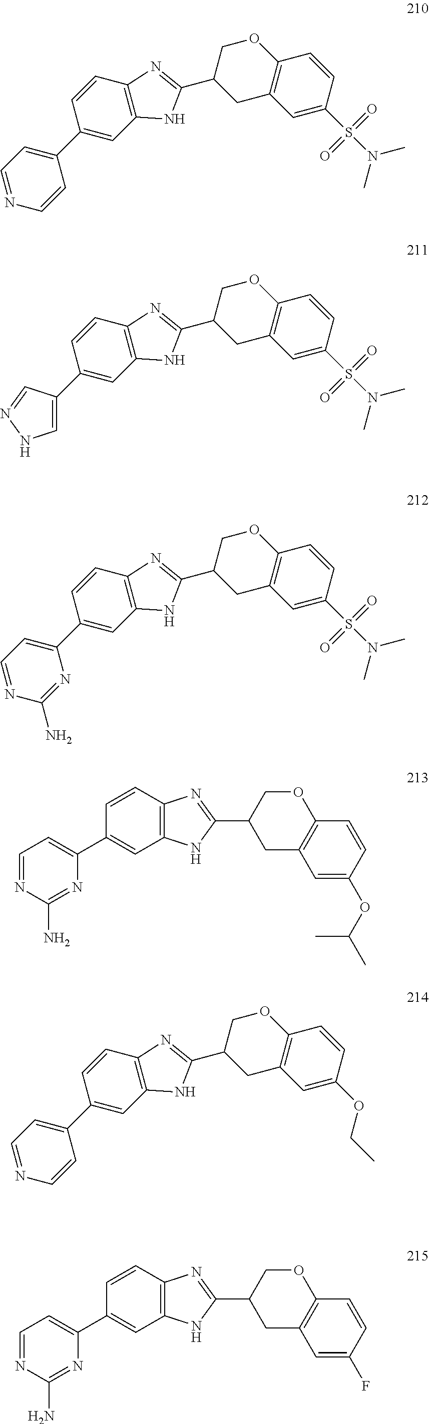 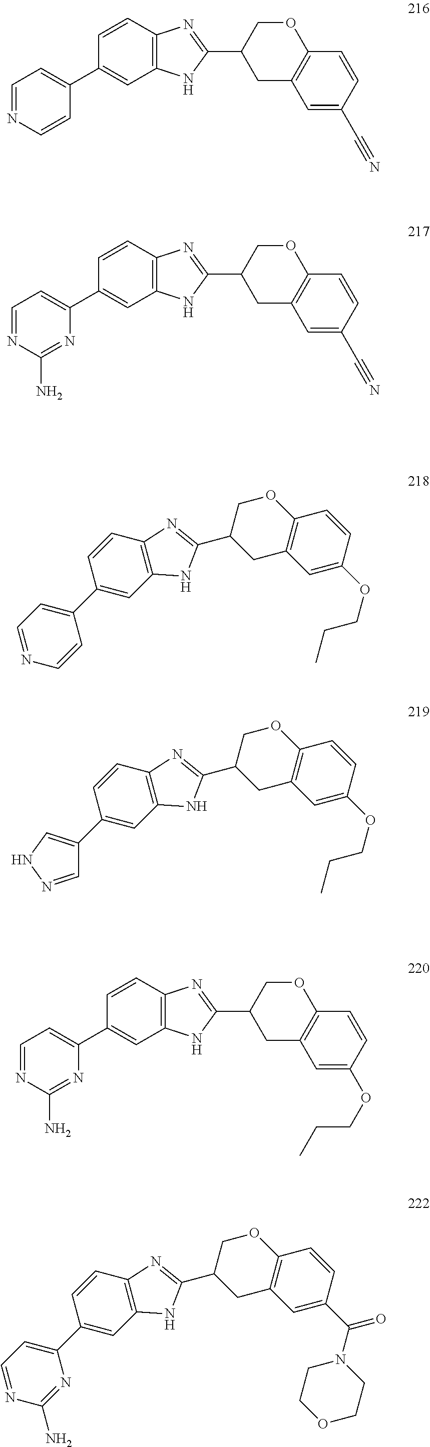 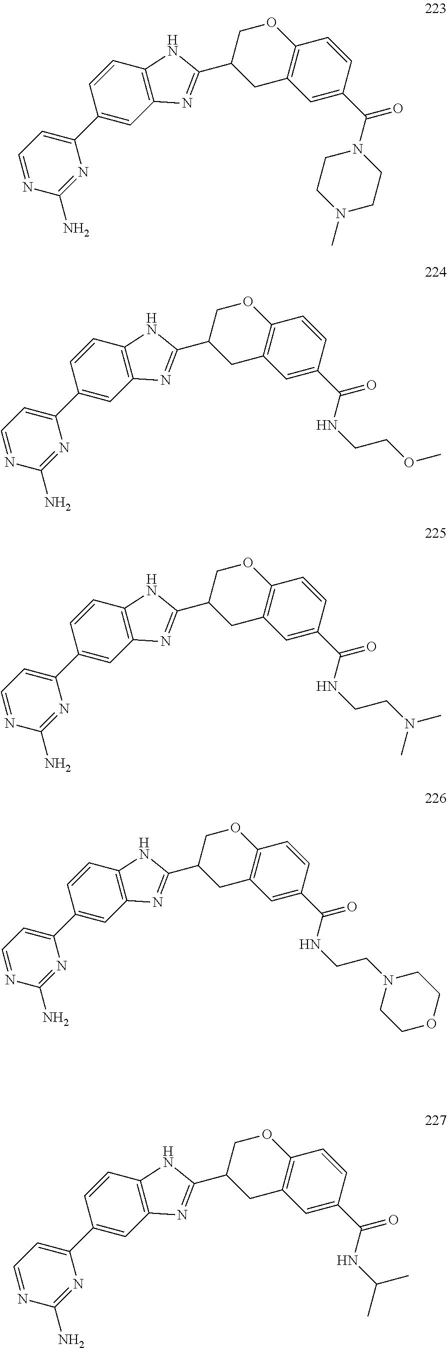 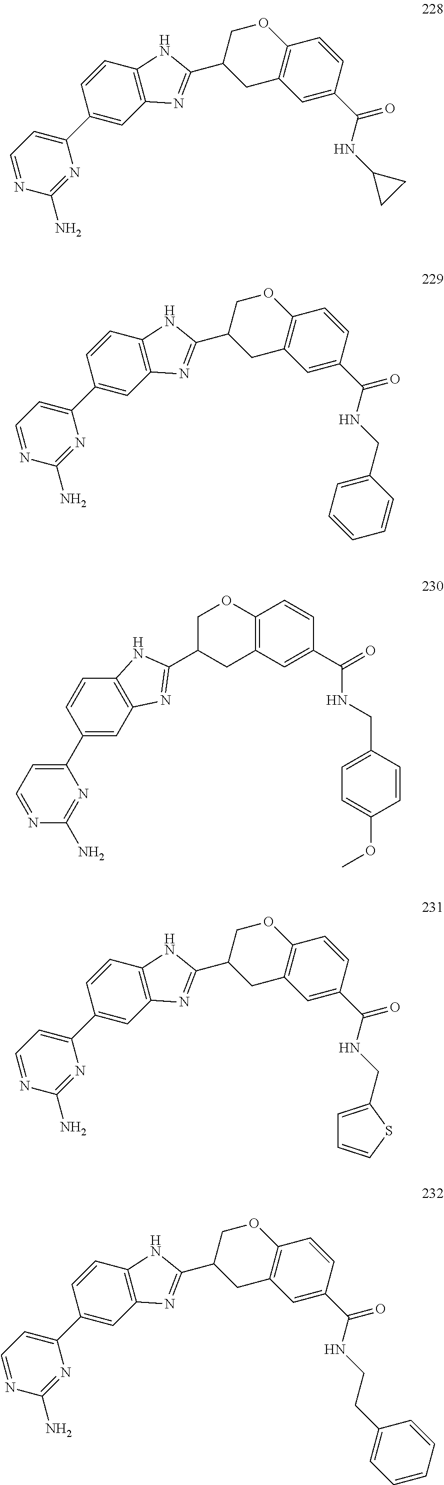 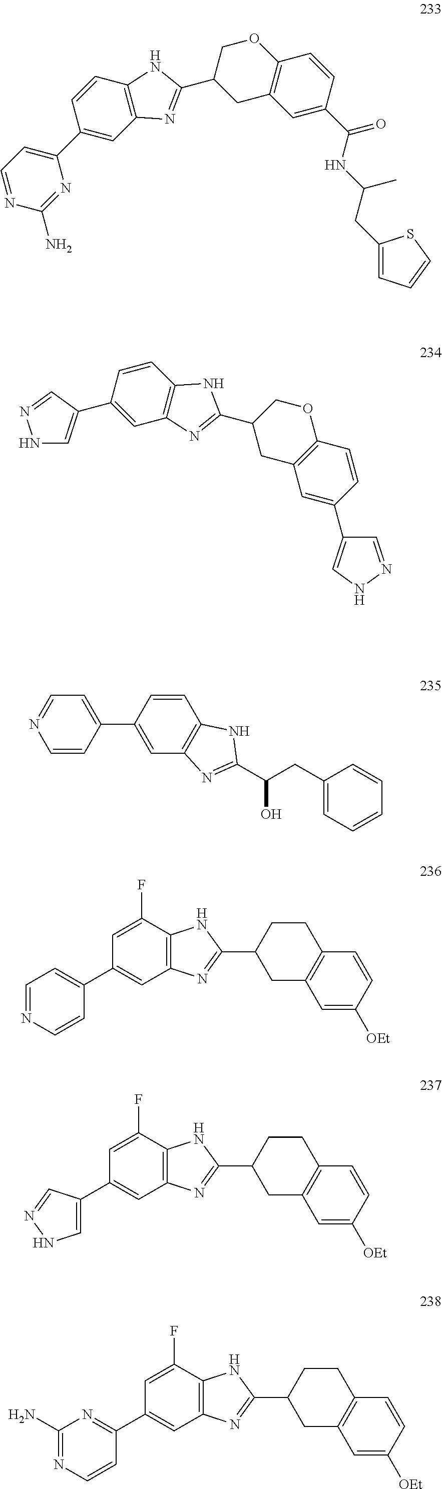 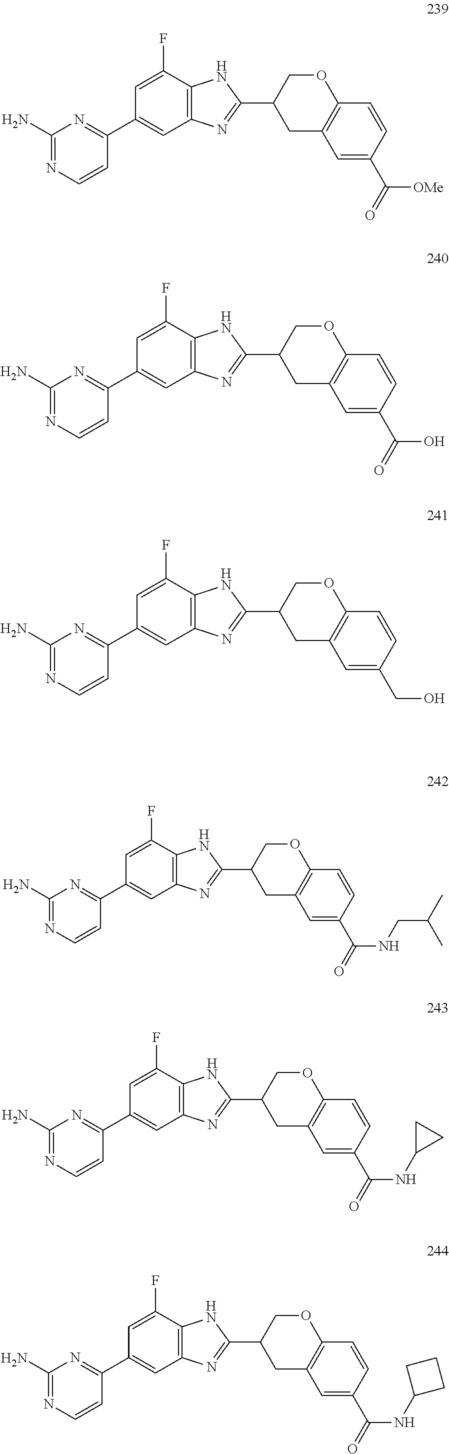 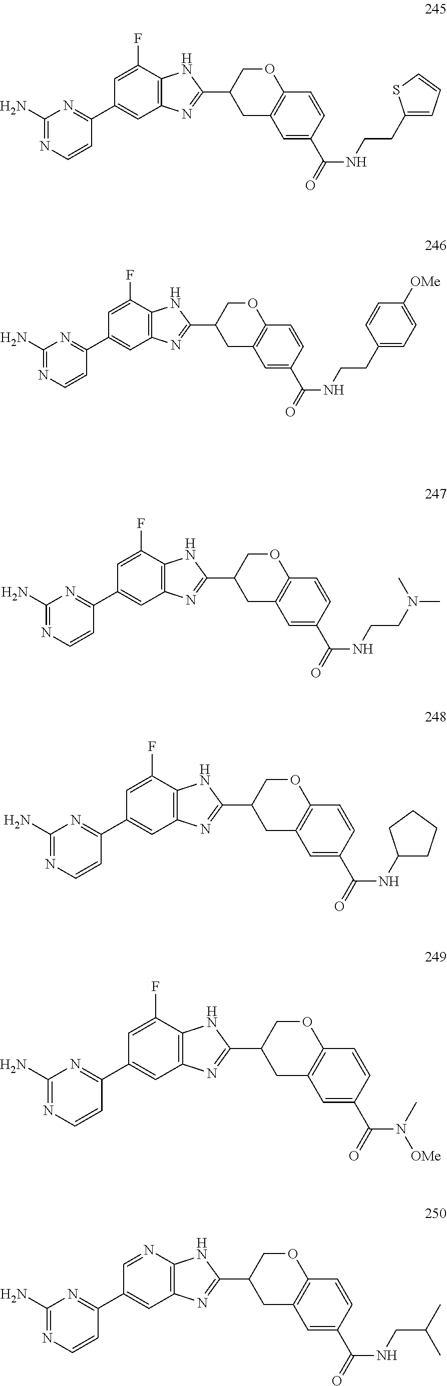 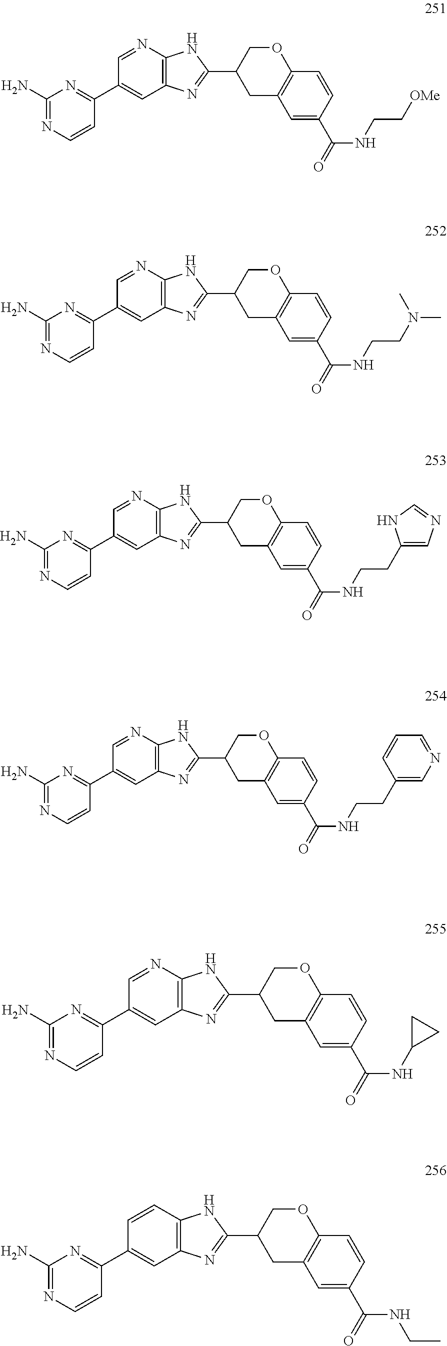 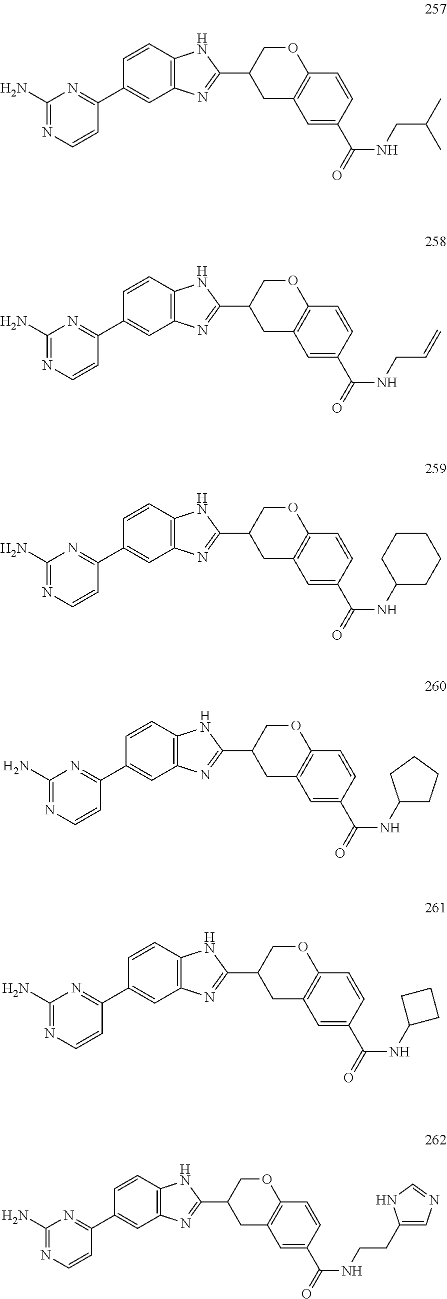 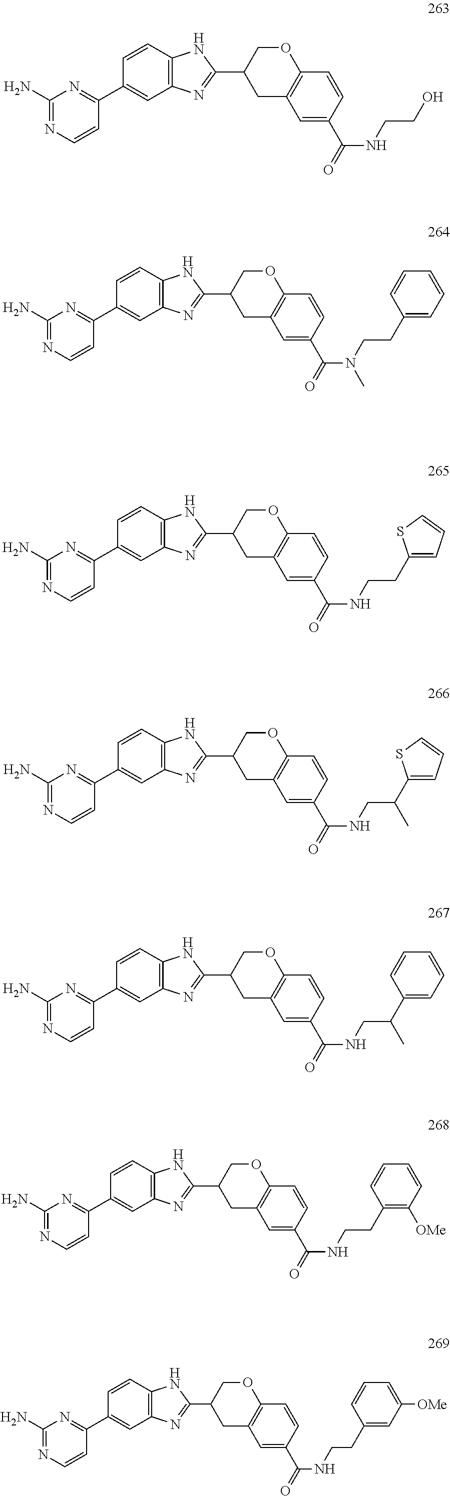 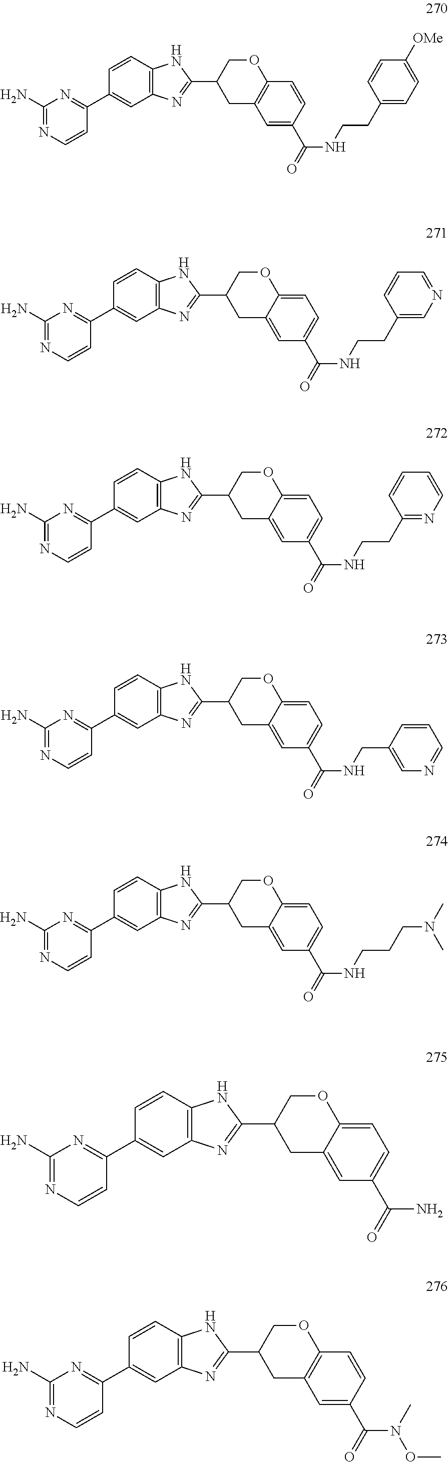 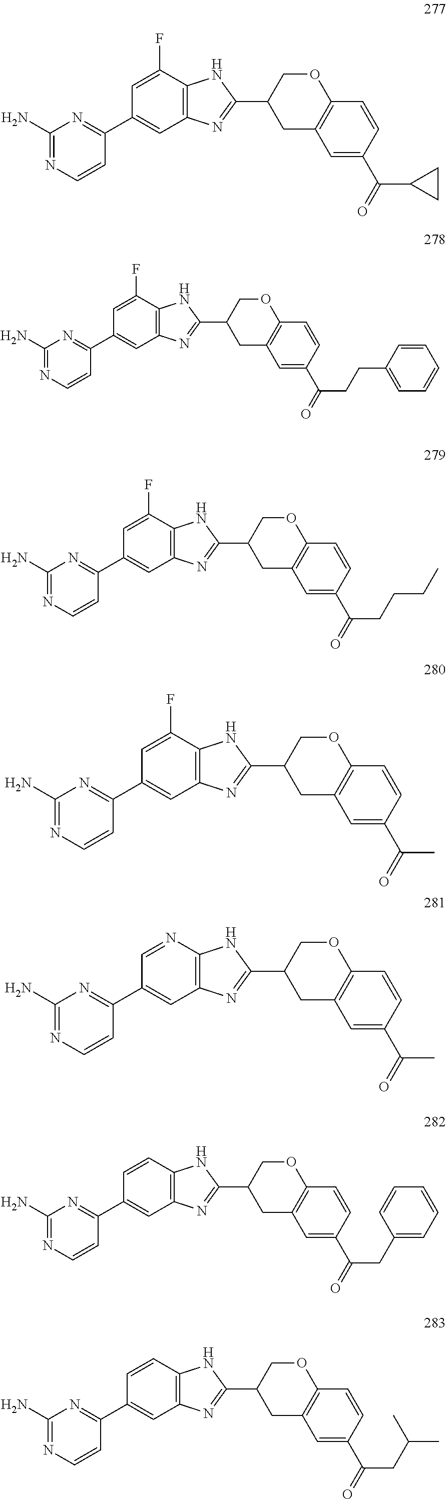 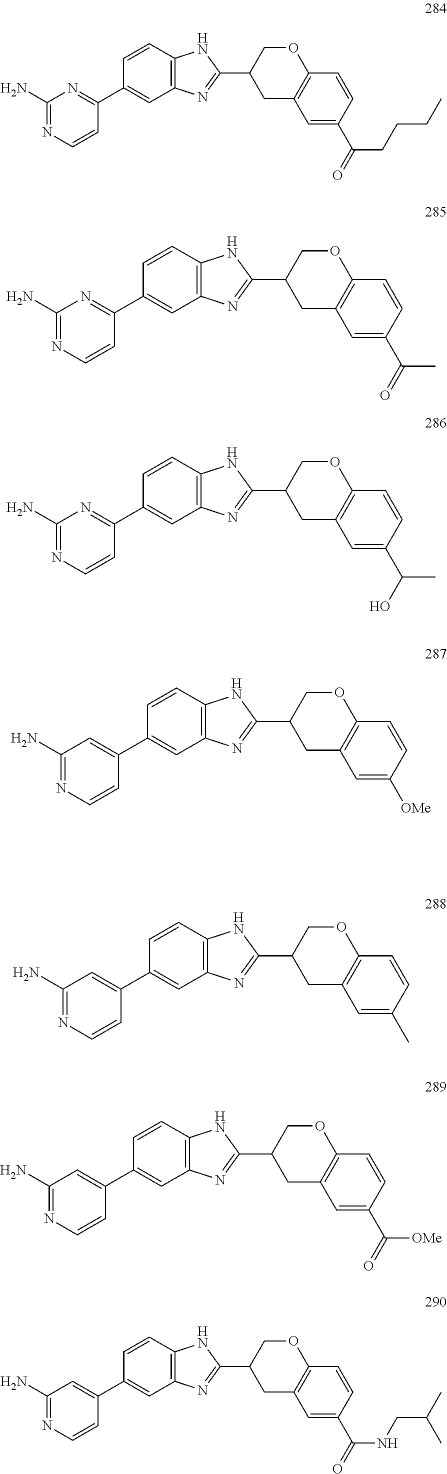 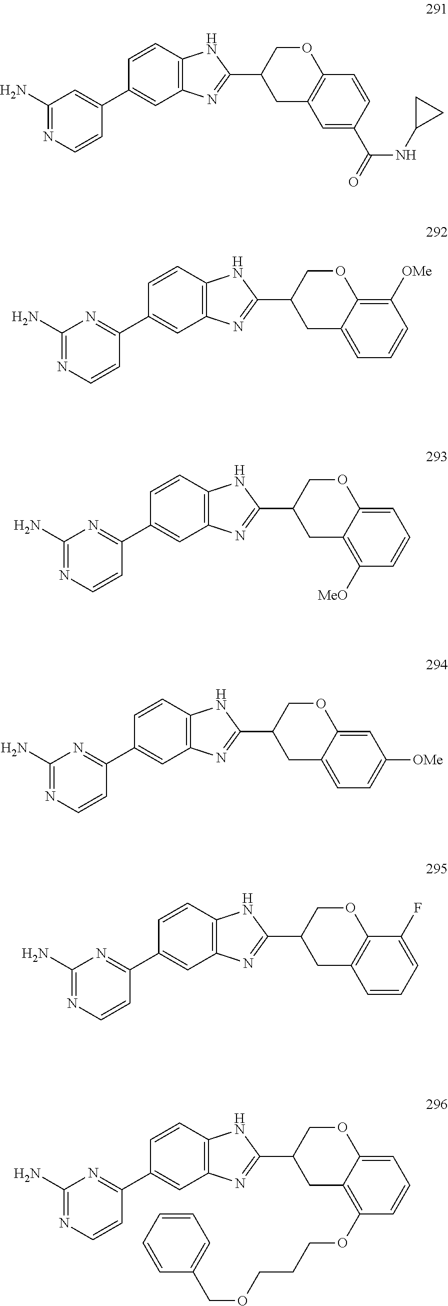 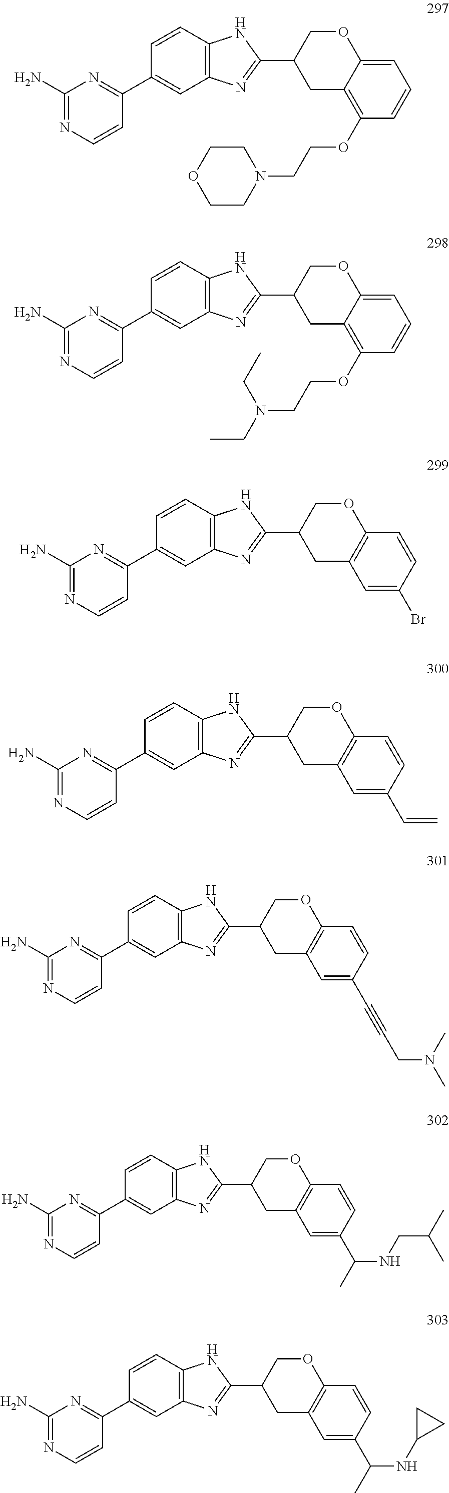 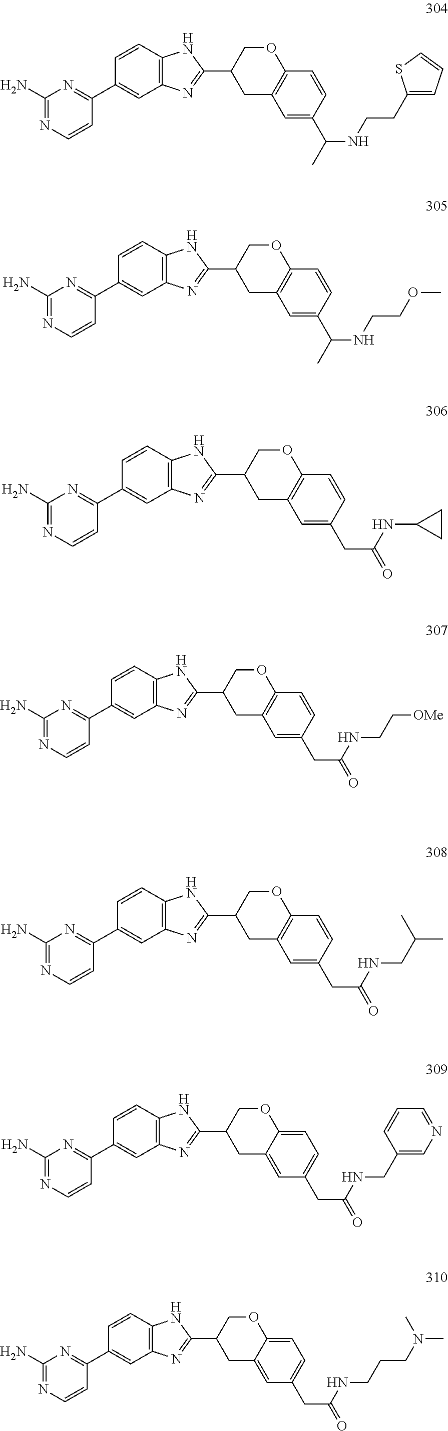 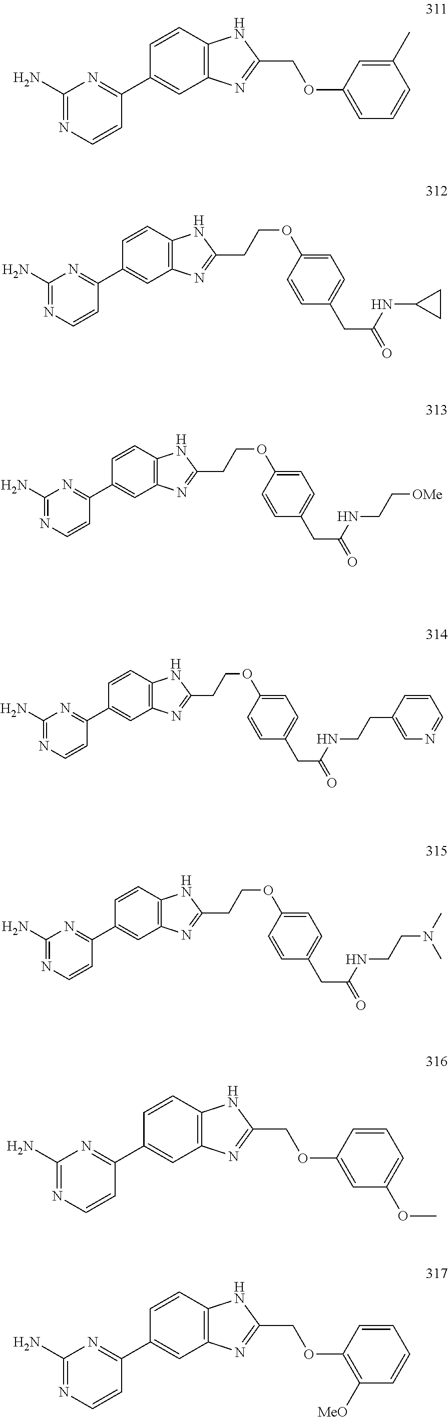 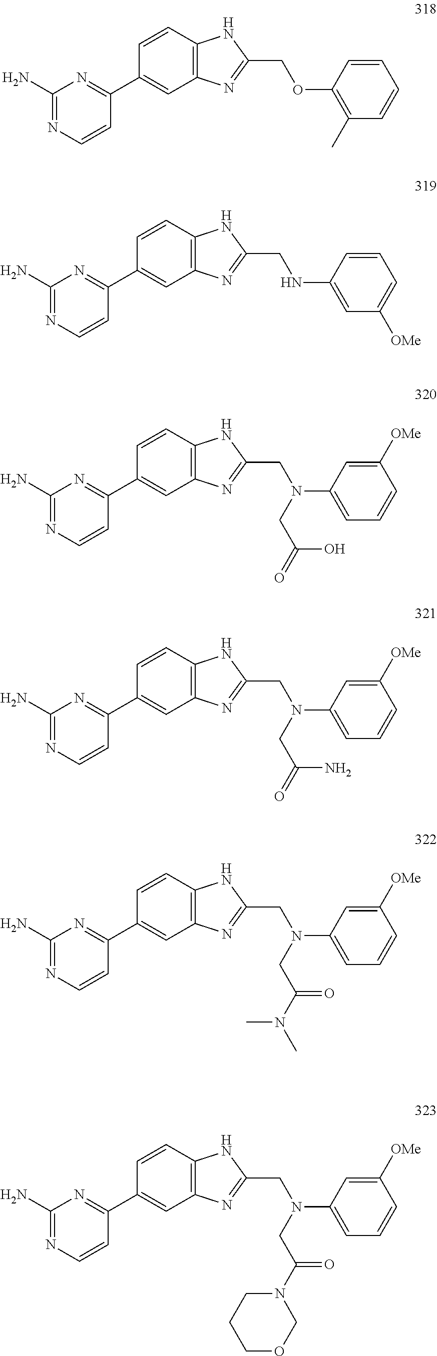 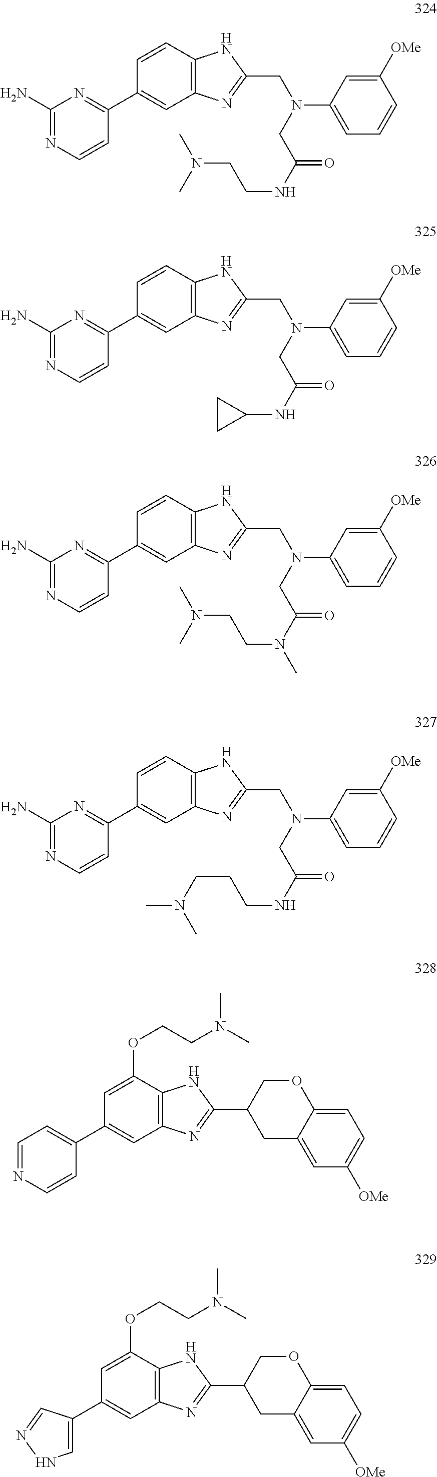 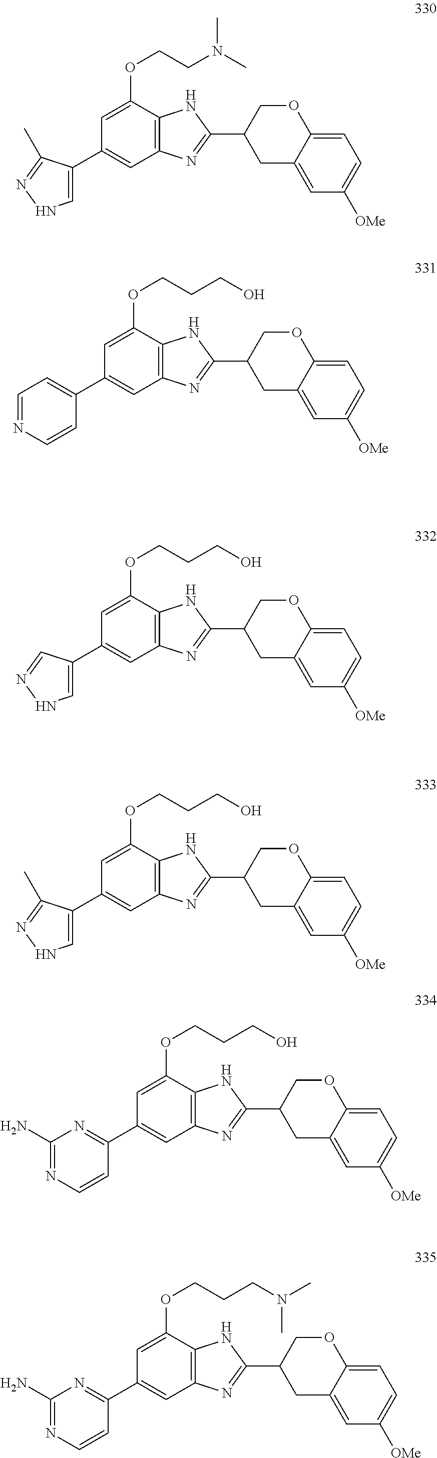 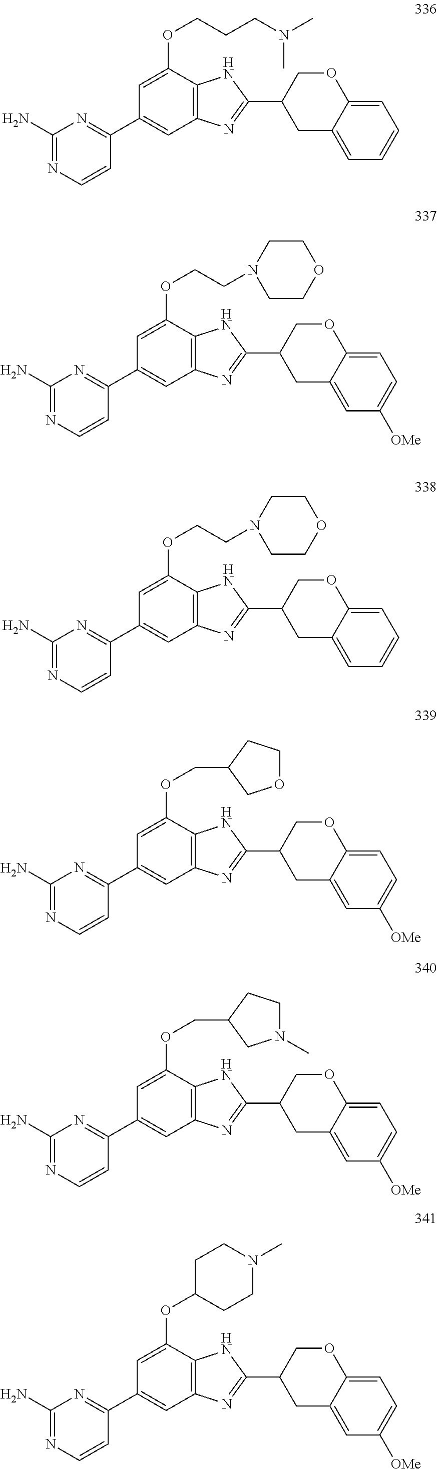 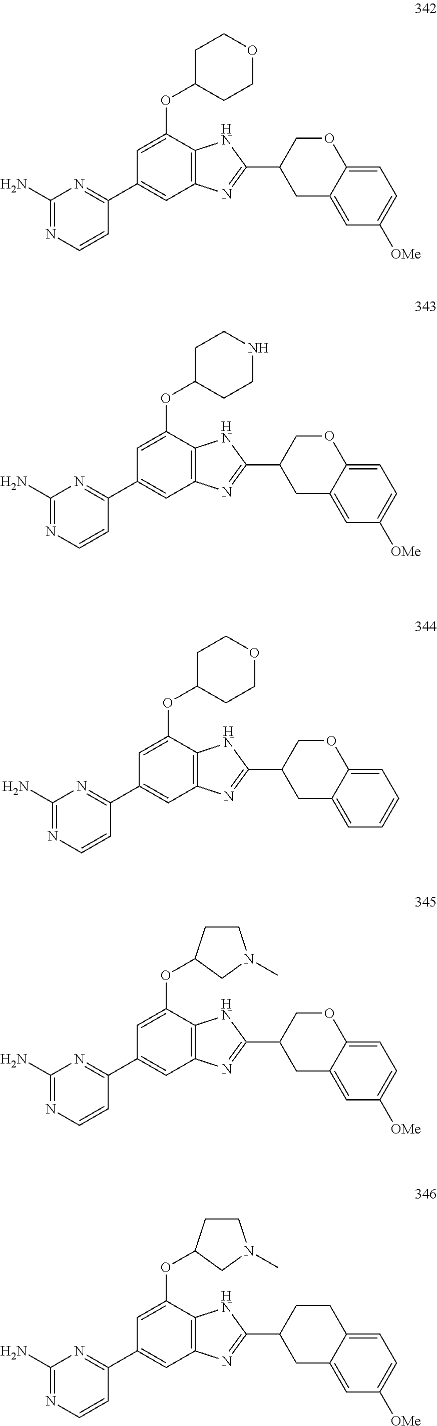 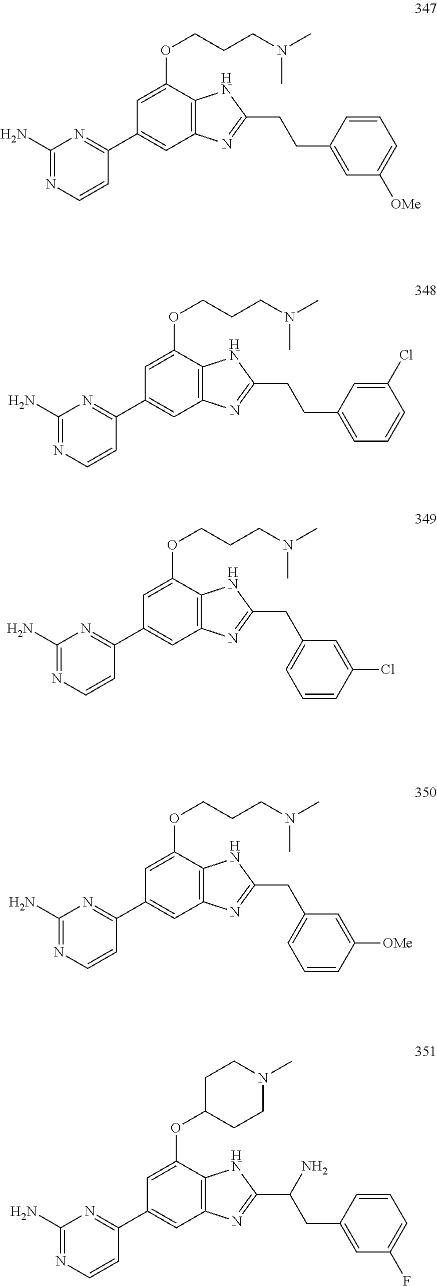 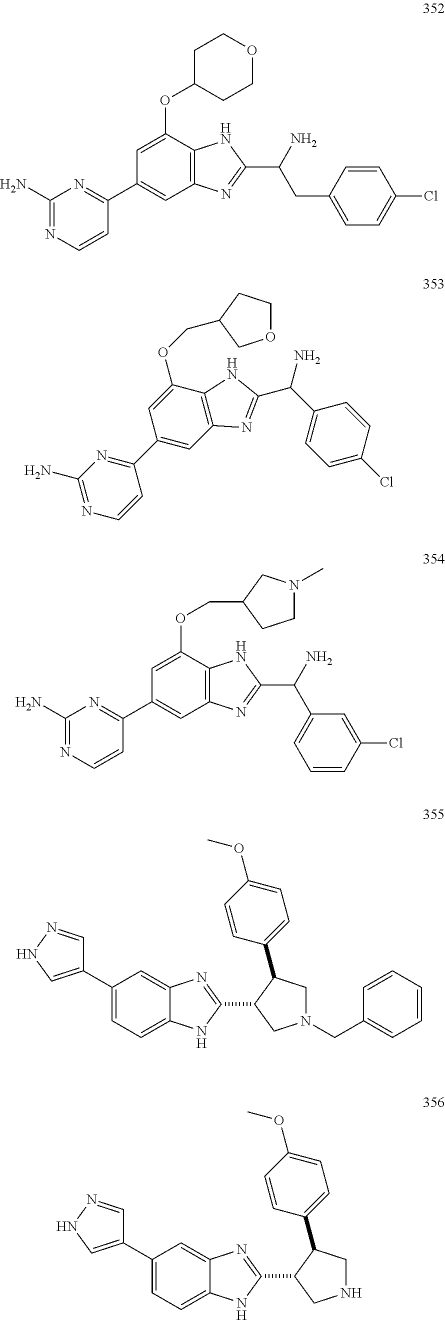 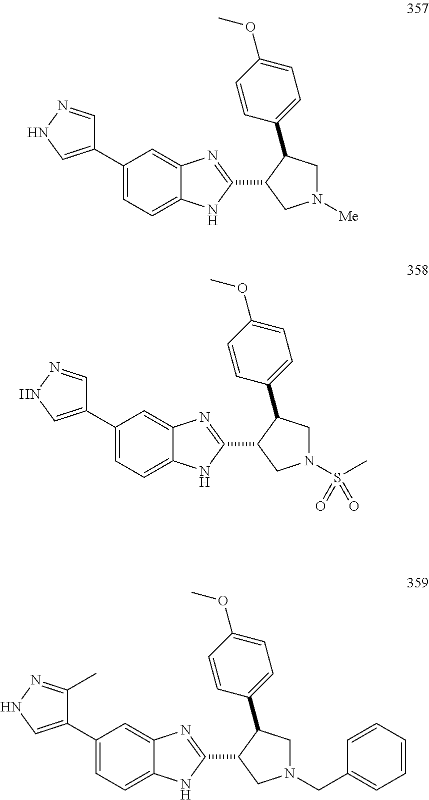
or any tautomer, salt, stereoisomer, hydrate, solvent, or prodrug thereof.

39. A pharmaceutical composition comprising a compound of claim 1 and a pharmaceutically acceptable excipient.

40. A pharmaceutical combination comprising a compound of claim 1 and an effective amount of a second medicament.

41.-50. (canceled)

51. A pharmaceutical composition comprising the combination of claim 40 and a suitable excipient.

52. A method of treatment of a malcondition in a patient in need thereof, comprising administering a therapeutically effective amount of the compound of claim 1 to the patient at a frequency of administration and for a duration of time sufficient to provide a beneficial effect to the patient.

53. The method of claim 52 wherein the malcondition comprises cardiovascular disease, neurogenic pain, hypertension, atherosclerosis, angina, stroke, arterial obstruction, peripheral arterial disease, peripheral circulation disorder, erectile dysfunction, acute or chronic pain, dementia, Alzheimer's disease, Parkinson's disease, neuronal degeneration, asthma, amyotrophic lateral sclerosis, spinal cord injury, rheumatoid arthritis, osteoarthritis, osteoporosis, psoriasis, cerebral vasospasm, glaucoma, multiple sclerosis, pulmonary hypertension, acute respiratory distress syndrome, inflammation, diabetes, urinary organ diseases such as overactive bladder (OAB) and benign prostatic hypertrophy (BPH), metastasis, cancer, glaucoma, ocular hypertension, retinopathy, autoimmune disease and viral infection, or myocardial pathology, or any combination thereof.

54. The method of claim 52 for which binding of a ligand to a Rho kinase or inhibition of a bioactivity of a Rho kinase, or both, is medically indicated.

55.-57. (canceled)

58. The method of claim 52 further comprising administration of an effective amount of an additional medicament.

59. The method of claim 58 wherein the additional medicament comprises an anti-proliferative agent, an anti-glaucoma agent, an anti-hypertensive agent, an anti-atherosclerotic agent, an anti-multiple sclerosis agent, an anti-angina agent, an anti-erectile dysfunction agent, an anti-stroke agent, or an anti-asthma agent.

60. The method of claim 59 wherein the anti-proliferative agent comprises an alkylating agent, an anti-metabolite, a vinca alkaloid, a terpenoid, a topoisomerase inhibitor, a monoclonal antibody, a kinase inhibitor, carboplatin, cisplatin, taxol, leucovorin, 5-fluorouracil, eloxatin, cyclophosphamide, chlorambucil, avastin, or imatinib mesylate.

61. The method of claim 59 wherein the anti-glaucoma agent comprises a beta receptor-blocker, a prostaglandin, an alpha-adrenergic agonist, a parasympathomimetic (cholinergic agonist), or a carbonic anhydrase inhibitor.

62. The method of claim 59 wherein the anti-hypertensive agent comprises a beta receptor-blocker, a calcium channel blocker, a diueretic, an angiotensin converting enzyme (ACE) inhibitor, a renin inhibitor, or an angiotensin receptor antagonist.

63. The method of claim 59 wherein the anti-atherosclerotic agent comprises a 3-HMG-coA-reductase inhibitor, a statin, atorvastatin, simvastatin, niacin, or a combination drug such as vytorin.

64. The method of claim 59 wherein the anti-multiple sclerosis agent comprises beta-inteferon, tysabri, or glatirimar acetate.

65. The method of claim 59 wherein the anti-angina agent comprises a beta receptor-blocker, a calcium channel blocker, nitroglycerin, isosoribide mononitrate, nicorandil, or ranolanzine.

66. The method of claim 59 wherein the anti-erectile dysfunction agent comprises a phosphodiesterase-5 inhibitor.

67. The method of claim 59 wherein the anti-stroke agent comprises tissue plasminogen activator.

68. The method of claim 59 wherein the anti-asthma agent comprises a bronchodilator, an inhaled corticosteroid, a leukotrine blockers, cromolyn, nedocromil, or theophylline.

69.-74. (canceled)
